SEQUENCE LISTING (910 sequences)

SEQ ID NO: 1 - DNA, 64 bp, Artificial Sequence
  Description of Artificial Sequence Synthetic oligonucleotide
gttggaggctcatcgttcctatattccacaccacttattattacagatgttatgctcgcaggtc

SEQ ID NO: 2 - DNA, 68 bp, Artificial Sequence
  Description of Artificial Sequence Synthetic oligonucleotide
gttggaggctcatcgttcctatattcctgactcctcattgatgattacagatgttatgctcgcaggtc

SEQ ID NO: 3 - DNA, 120 bp, Artificial Sequence
  Description of Artificial Sequence Synthetic probe
gtggtatggctgattatgatctagagtgttggaggctcatcgttcctatattcctgactcctcattgatgattacagatgttatgctcgcaggtcgagtttggacaaaccacaactagaa

SEQ ID NO: 4 - DNA, 108 bp, Artificial Sequence
  Description of Artificial Sequence Synthetic probe
gctgtcaccgtccagacgctgttggcgttggaggctcatcgttcctatattccacaccacttattattacagatgttatgctcgcaggtctccgtgccttcaagcgcg

SEQ ID NO: 5 - DNA, 110 bp, Artificial Sequence
  Description of Artificial Sequence Synthetic probe
gactccgcagaatacggcaccgtgcgcagttggaggctcatcgttcctatattccacaccacttattattacagatgttatgctcgcaggtcgcgtacaggccagtcagc

SEQ ID NO: 6 - DNA, 105 bp, Artificial Sequence
  Description of Artificial Sequence Synthetic probe
gcagtcggtaacctcgcgcgttggaggctcatcgttcctatattccacaccacttattattacagatgttatgctcgcaggtcgcgctatctctgctctcactgc

SEQ ID NO: 7 - DNA, 105 bp, Artificial Sequence
  Description of Artificial Sequence Synthetic probe
gctgtcctggctgcaagcctgggttggaggctcatcgttcctatattccacaccacttattattacagatgttatgctcgcaggtcccgaactgctgatggacgt

SEQ ID NO: 8 - DNA, 115 bp, Artificial Sequence
  Description of Artificial Sequence Synthetic probe
gacagcagactcaccggctggttccgctgttggaggctcatcgttcctatattccacaccacttattattacagatgttatgctcgcaggtcgcaagatgctgctggccacactg

SEQ ID NO: 9 - DNA, 104 bp, Artificial Sequence
  Description of Artificial Sequence Synthetic probe
gacagaacaagttccgctccgggttggaggctcatcgttcctatattccacaccacttattattacagatgttatgctcgcaggtccacggatacgccgcgcat

SEQ ID NO: 10 - DNA, 104 bp, Artificial Sequence
  Description of Artificial Sequence Synthetic probe
gaggaccgaaggagctaaccggttggaggctcatcgttcctatattccacaccacttattattacagatgttatgctcgcaggtccgccgcatacactattctc

SEQ ID NO: 11 - DNA, 118 bp, Artificial Sequence
  Description of Artificial Sequence Synthetic probe
gctgtaatgcaagtagcgtatgcgctcgttggaggctcatcgttcctatattccacaccacttattattacagatgttatgctcgcaggtcgaacagcaaggccgccaatgcctgacg

SEQ ID NO: 12 - DNA, 108 bp, Artificial Sequence
  Description of Artificial Sequence Synthetic probe
gaacgtctggcgctggtcgcctgccgttggaggctcatcgttcctatattccacaccacttattattacagatgttatgctcgcaggtcgcacaggtgctgacgtggt

SEQ ID NO: 13 - DNA, 115 bp, Artificial Sequence
  Description of Artificial Sequence Synthetic probe
cgcatatgctgaatgattatctcgttgcgttggaggctcatcgttcctatattccacaccacttattattacagatgttatgctcgcaggtcatcttgctcaatgaggttattca

SEQ ID NO: 14 - DNA, 102 bp, Artificial Sequence
  Description of Artificial Sequence Synthetic probe
gacgacagatgcaggttgagttggaggctcatcgttcctatattccacaccacttattattacagatgttatgctcgcaggtccgcatcgccgatgctcatc

SEQ ID NO: 15 - DNA, 116 bp, Artificial Sequence
  Description of Artificial Sequence Synthetic probe
cgcctgctccagtgcatccagcacgaatgttggaggctcatcgttcctatattccacaccacttattattacagatgttatgctcgcaggtcatgctctccgccatcgcgttgtca

SEQ ID NO: 16 - DNA, 109 bp, Artificial Sequence
  Description of Artificial Sequence Synthetic probe
agtgcgttcaccgaatacgtgcgcagttggaggctcatcgttcctatattccacaccacttattattacagatgttatgctcgcaggtccaggttatgccgctcaattc

SEQ ID NO: 17 - DNA, 111 bp, Artificial Sequence
  Description of Artificial Sequence Synthetic probe
aatccaggtcctgaccgttctgtccgtgttggaggctcatcgttcctatattccacaccacttattattacagatgttatgctcgcaggtcacctccgttgagctgatgga

SEQ ID NO: 18 - DNA, 117 bp, Artificial Sequence
  Description of Artificial Sequence Synthetic probe
gaggtggccaacaccatgtgtgaccgttggaggctcatcgttcctatattccacaccacttattattacagatgttatgctcgcaggtcgacgccggtatatcggtatcgagctgct

SEQ ID NO: 19 - DNA, 119 bp, Artificial Sequence
  Description of Artificial Sequence Synthetic probe
cgcatatgctgaatgattatctcgttggttggaggctcatcgttcctatattccacaccacttattattacagatgttatgctcgcaggtcacggtgatcttgctcaatgaggttattc

SEQ ID NO: 20 - DNA, 106 bp, Artificial Sequence
  Description of Artificial Sequence Synthetic probe
gaagtgccggacttctgcagagttggaggctcatcgttcctatattccacaccacttattattacagatgttatgctcgcaggtcgcacggcctgatggaggccgc

SEQ ID NO: 21 - DNA, 109 bp, Artificial Sequence
  Description of Artificial Sequence Synthetic probe
gctaatcgcataacagctacgttggaggctcatcgttcctatattccacaccacttattattacagatgttatgctcgcaggtccatcacgtaacttattgatgatatt

SEQ ID NO: 22 - DNA, 108 bp, Artificial Sequence
  Description of Artificial Sequence Synthetic probe
gctgcggtattccacggtcggccgttggaggctcatcgttcctatattccacaccacttattattacagatgttatgctcgcaggtcgcaggaacgctgcctgtggtc

SEQ ID NO: 23 - DNA, 110 bp, Artificial Sequence
  Description of Artificial Sequence Synthetic probe
gaatcaattatcttcttcattattgatgttggaggctcatcgttcctatattccacaccacttattattacagatgttatgctcgcaggtcctgcggctcaactcaagca

SEQ ID NO: 24 - DNA, 103 bp, Artificial Sequence
  Description of Artificial Sequence Synthetic probe
gtcacacgtcacgcagtccgttggaggctcatcgttcctatattccacaccacttattattacagatgttatgctcgcaggtcgcattcatggcgctgatggc

SEQ ID NO: 25 - DNA, 115 bp, Artificial Sequence
  Description of Artificial Sequence Synthetic probe
gtgttactcggtagaatgctcgcaagggttggaggctcatcgttcctatattccacaccacttattattacagatgttatgctcgcaggtcactagatgacatatcatgtaagtt

SEQ ID NO: 26 - DNA, 101 bp, Artificial Sequence
  Description of Artificial Sequence Synthetic probe
cggaactgcctgctcgtatgttggaggctcatcgttcctatattccacaccacttattattacagatgttatgctcgcaggtcaacgatatagtccgttat

SEQ ID NO: 27 - DNA, 107 bp, Artificial Sequence
  Description of Artificial Sequence Synthetic probe
gctctccgactcctggtacgtcaggttggaggctcatcgttcctatattccacaccacttattattacagatgttatgctcgcaggtcgcgcgcattaatgaagcac

SEQ ID NO: 28 - DNA, 113 bp, Artificial Sequence
  Description of Artificial Sequence Synthetic probe
gatgttgcgattacttcgccaactattggttggaggctcatcgttcctatattccacaccacttattattacagatgttatgctcgcaggtcgctgtaattatgacgacgccg

SEQ ID NO: 29 - DNA, 112 bp, Artificial Sequence
  Description of Artificial Sequence Synthetic probe
ctcattccagaagcaacttcttcttgttggaggctcatcgttcctatattccacaccacttattattacagatgttatgctcgcaggtcggatagccatggctacaagaata

SEQ ID NO: 30 - DNA, 116 bp, Artificial Sequence
  Description of Artificial Sequence Synthetic probe
gcaataccaggaaggaagtcttactggttggaggctcatcgttcctatattccacaccacttattattacagatgttatgctcgcaggtcgtcattggagaacagatgattgatgt

SEQ ID NO: 31 - DNA, 107 bp, Artificial Sequence
  Description of Artificial Sequence Synthetic probe
gtatcgccacaataactgccggaagttggaggctcatcgttcctatattccacaccacttattattacagatgttatgctcgcaggtcaacgatatagtccgttatg

SEQ ID NO: 32 - DNA, 109 bp, Artificial Sequence
  Description of Artificial Sequence Synthetic probe
gctgtggcacaggctgaacgccggttggaggctcatcgttcctatattccacaccacttattattacagatgttatgctcgcaggtcggtgatgtcattctggttaaga

SEQ ID NO: 33 - DNA, 110 bp, Artificial Sequence
  Description of Artificial Sequence Synthetic probe
acataatctgaatctgagacaacatcgttggaggctcatcgttcctatattccacaccacttattattacagatgttatgctcgcaggtcacgcactctggccacactgg

SEQ ID NO: 34 - DNA, 106 bp, Artificial Sequence
  Description of Artificial Sequence Synthetic probe
gtgaagcgcatccggtcaccgttggaggctcatcgttcctatattccacaccacttattattacagatgttatgctcgcaggtcatggcataggccaggtcaatat

SEQ ID NO: 35 - DNA, 111 bp, Artificial Sequence
  Description of Artificial Sequence Synthetic probe
ggttctggaccagttgcgtgagcgcgttggaggctcatcgttcctatattccacaccacttattattacagatgttatgctcgcaggtccgtaacatcgttgctgctccat

SEQ ID NO: 36 - DNA, 106 bp, Artificial Sequence
  Description of Artificial Sequence Synthetic probe
cgctggatttcacgccataggcgttggaggctcatcgttcctatattccacaccacttattattacagatgttatgctcgcaggtctgtcgctaccgttgatgatt

SEQ ID NO: 37 - DNA, 108 bp, Artificial Sequence
  Description of Artificial Sequence Synthetic probe
cgtataggtggctaagtgcagcgttggaggctcatcgttcctatattccacaccacttattattacagatgttatgctcgcaggtcgtaactcattcctgagggtttc

SEQ ID NO: 38 - DNA, 107 bp, Artificial Sequence
  Description of Artificial Sequence Synthetic probe
gtacatactcgatcgaagcacgagttggaggctcatcgttcctatattccacaccacttattattacagatgttatgctcgcaggtcccggaatagcggaagctttc

SEQ ID NO: 39 - DNA, 109 bp, Artificial Sequence
  Description of Artificial Sequence Synthetic probe
aaggtcgaagcaggtacatactcggttggaggctcatcgttcctatattccacaccacttattattacagatgttatgctcgcaggtcagacatgagctcaagtccaat

SEQ ID NO: 40 - DNA, 110 bp, Artificial Sequence
  Description of Artificial Sequence Synthetic probe
gaagctttcatagcgtcgcctaggttggaggctcatcgttcctatattccacaccacttattattacagatgttatgctcgcaggtcttagctagcttgtaagcaaattg

SEQ ID NO: 41 - DNA, 108 bp, Artificial Sequence
  Description of Artificial Sequence Synthetic probe
gaagctttcatggcatcgcctaggttggaggctcatcgttcctatattccacaccacttattattacagatgttatgctcgcaggtcagctagcttgtaagcaaactg

SEQ ID NO: 42 - DNA, 106 bp, Artificial Sequence
  Description of Artificial Sequence Synthetic probe
cgctaccggtagtattgcccttgttggaggctcatcgttcctatattccacaccacttattattacagatgttatgctcgcaggtcagaatatcccgacggctttc

SEQ ID NO: 43 - DNA, 106 bp, Artificial Sequence
  Description of Artificial Sequence Synthetic probe
atcgccacgttatcgctgtactgttggaggctcatcgttcctatattccacaccacttattattacagatgttatgctcgcaggtctttacccagcgtcagattcc

SEQ ID NO: 44 - DNA, 109 bp, Artificial Sequence
  Description of Artificial Sequence Synthetic probe
caagtactgttcctgtacgtcagcgttggaggctcatcgttcctatattccacaccacttattattacagatgttatgctcgcaggtctcgccagtaactggtctattc

SEQ ID NO: 45 - DNA, 103 bp, Artificial Sequence
  Description of Artificial Sequence Synthetic probe
caacgtctgcgccatcgccgttggaggctcatcgttcctatattccacaccacttattattacagatgttatgctcgcaggtccgcaatatcattggtggtgc

SEQ ID NO: 46 - DNA, 103 bp, Artificial Sequence
  Description of Artificial Sequence Synthetic probe
gccgcccgaaggacatcaacgttggaggctcatcgttcctatattccacaccacttattattacagatgttatgctcgcaggtccagacgggacgtacacaac

SEQ ID NO: 47 - DNA, 107 bp, Artificial Sequence
  Description of Artificial Sequence Synthetic probe
cgtgctggctattgccttagggttggaggctcatcgttcctatattccacaccacttattattacagatgttatgctcgcaggtcgtaatactcctagcaccaaatc

SEQ ID NO: 48 - DNA, 108 bp, Artificial Sequence
  Description of Artificial Sequence Synthetic probe
cattaggagttgtcgtatccctcagttggaggctcatcgttcctatattccacaccacttattattacagatgttatgctcgcaggtcaatactccgagcaccaaatc

SEQ ID NO: 49 - DNA, 106 bp, Artificial Sequence
  Description of Artificial Sequence Synthetic probe
aaattgcagttcgcgcttagcgttggaggctcatcgttcctatattccacaccacttattattacagatgttatgctcgcaggtcgttccatagcgttaaggtttc

SEQ ID NO: 50 - DNA, 103 bp, Artificial Sequence
  Description of Artificial Sequence Synthetic probe
gcgccaaacagaccaatgctgttggaggctcatcgttcctatattccacaccacttattattacagatgttatgctcgcaggtcgatttcacgccataggctc

SEQ ID NO: 51 - DNA, 106 bp, Artificial Sequence
  Description of Artificial Sequence Synthetic probe
gtataggtggctaagtgcagcagttggaggctcatcgttcctatattccacaccacttattattacagatgttatgctcgcaggtctcgtaactcattcctgaggg

SEQ ID NO: 52 - DNA, 104 bp, Artificial Sequence
  Description of Artificial Sequence Synthetic probe
gtcatcgcctcttcgtagctcgttggaggctcatcgttcctatattccacaccacttattattacagatgttatgctcgcaggtcgccatatcgataacgctgg

SEQ ID NO: 53 - DNA, 110 bp, Artificial Sequence
  Description of Artificial Sequence Synthetic probe
agtatcttacctgaaattccctcacgttggaggctcatcgttcctatattccacaccacttattattacagatgttatgctcgcaggtccctctcgtcataagtcgaatg

SEQ ID NO: 54 - DNA, 102 bp, Artificial Sequence
  Description of Artificial Sequence Synthetic probe
catcacgaagcccgccacagttggaggctcatcgttcctatattccacaccacttattattacagatgttatgctcgcaggtcgcccttgagcggaagtatc

SEQ ID NO: 55 - DNA, 104 bp, Artificial Sequence
  Description of Artificial Sequence Synthetic probe
accaatacgccagtagcgagagttggaggctcatcgttcctatattccacaccacttattattacagatgttatgctcgcaggtcgcaacgtagctgccaaatc

SEQ ID NO: 56 - DNA, 107 bp, Artificial Sequence
  Description of Artificial Sequence Synthetic probe
caatcagtgtgtttgatttgcaccgttggaggctcatcgttcctatattccacaccacttattattacagatgttatgctcgcaggtctacccggaatagcctgctc

SEQ ID NO: 57 - DNA, 104 bp, Artificial Sequence
  Description of Artificial Sequence Synthetic probe
cggataacgccacgggatgagttggaggctcatcgttcctatattccacaccacttattattacagatgttatgctcgcaggtcaccgggtcaaagaattcctc

SEQ ID NO: 58 - DNA, 100 bp, Artificial Sequence
  Description of Artificial Sequence Synthetic probe
gcggcgtggtggtgtctcgttggaggctcatcgttcctatattccacaccacttattattacagatgttatgctcgcaggtccgctgccggtcttatcac

SEQ ID NO: 59 - DNA, 101 bp, Artificial Sequence
  Description of Artificial Sequence Synthetic probe
gccacgtcaccagctgcggttggaggctcatcgttcctatattccacaccacttattattacagatgttatgctcgcaggtccggctgggtgaagtaagtc

SEQ ID NO: 60 - DNA, 101 bp, Artificial Sequence
  Description of Artificial Sequence Synthetic probe
gctcgtagcgtcgcgtctcgttggaggctcatcgttcctatattccacaccacttattattacagatgttatgctcgcaggtcttgaccgacagaggcaac

SEQ ID NO: 61 - DNA, 102 bp, Artificial Sequence
  Description of Artificial Sequence Synthetic probe
cagcaggtccgccaatttctcgttggaggctcatcgttcctatattccacaccacttattattacagatgttatgctcgcaggtcagtggacgtcagtgcgc

SEQ ID NO: 62 - DNA, 106 bp, Artificial Sequence
  Description of Artificial Sequence Synthetic probe
ccatagaggactttagccacagtgttggaggctcatcgttcctatattccacaccacttattattacagatgttatgctcgcaggtctacaccgctacagcgtaat

SEQ ID NO: 63 - DNA, 106 bp, Artificial Sequence
  Description of Artificial Sequence Synthetic probe
catatgcagagtgagcggtccgttggaggctcatcgttcctatattccacaccacttattattacagatgttatgctcgcaggtctcaattctttcaaagaccagc

SEQ ID NO: 64 - DNA, 107 bp, Artificial Sequence
  Description of Artificial Sequence Synthetic probe
ccattaacttcttcaaacgatgtatggttggaggctcatcgttcctatattccacaccacttattattacagatgttatgctcgcaggtcacccgtgctgtcgctat

SEQ ID NO: 65 - DNA, 108 bp, Artificial Sequence
  Description of Artificial Sequence Synthetic probe
gtgctgtcgctatggaaatgtggttggaggctcatcgttcctatattccacaccacttattattacagatgttatgctcgcaggtcaaccaaaccactaggttatctt

SEQ ID NO: 66 - DNA, 108 bp, Artificial Sequence
  Description of Artificial Sequence Synthetic probe
gtcagtgtttacaagaaccaccagttggaggctcatcgttcctatattccacaccacttattattacagatgttatgctcgcaggtcatgcatacgtgggaatagatt

SEQ ID NO: 67 - DNA, 100 bp, Artificial Sequence
  Description of Artificial Sequence Synthetic probe
cggaagtatccgcgcgccgttggaggctcatcgttcctatattccacaccacttattattacagatgttatgctcgcaggtcttcgatcacggcacgatc

SEQ ID NO: 68 - DNA, 102 bp, Artificial Sequence
  Description of Artificial Sequence Synthetic probe
cgaaccagcttggttcccaaggttggaggctcatcgttcctatattccacaccacttattattacagatgttatgctcgcaggtctcactgcgtgttcgctc

SEQ ID NO: 69 - DNA, 106 bp, Artificial Sequence
  Description of Artificial Sequence Synthetic probe
gatgctgtactttgtgatgcctagttggaggctcatcgttcctatattccacaccacttattattacagatgttatgctcgcaggtccgcttggcaagtactgttc

SEQ ID NO: 70 - DNA, 106 bp, Artificial Sequence
  Description of Artificial Sequence Synthetic probe
gcaagaaagcccttgaatgagcgttggaggctcatcgttcctatattccacaccacttattattacagatgttatgctcgcaggtcgcgttatcactgtattgcac

SEQ ID NO: 71 - DNA, 105 bp, Artificial Sequence
  Description of Artificial Sequence Synthetic probe
aatcaacaaactgctgccgctgttggaggctcatcgttcctatattccacaccacttattattacagatgttatgctcgcaggtcgctgtacttgtcatccttgt

SEQ ID NO: 72 - DNA, 99 bp, Artificial Sequence
  Description of Artificial Sequence Synthetic probe
ccagtctgccggcaccgcgttggaggctcatcgttcctatattccacaccacttattattacagatgttatgctcgcaggtctcgagcgcgagtctagc

SEQ ID NO: 73 - DNA, 100 bp, Artificial Sequence
  Description of Artificial Sequence Synthetic probe
ccgactgcccagtctgccggttggaggctcatcgttcctatattccacaccacttattattacagatgttatgctcgcaggtccgagcgcgagtctagcc

SEQ ID NO: 74 - DNA, 109 bp, Artificial Sequence
  Description of Artificial Sequence Synthetic probe
gtaaatagatgatcttaatttggttcacgttggaggctcatcgttcctatattccacaccacttattattacagatgttatgctcgcaggtcttgctggccaatcgtcg

SEQ ID NO: 75 - DNA, 104 bp, Artificial Sequence
  Description of Artificial Sequence Synthetic probe
cacagcctgactttcgccgcgttggaggctcatcgttcctatattccacaccacttattattacagatgttatgctcgcaggtccaagcaggagatcaacctgc

SEQ ID NO: 76 - DNA, 100 bp, Artificial Sequence
  Description of Artificial Sequence Synthetic probe
ggtggtcgataccgcctgggttggaggctcatcgttcctatattccacaccacttattattacagatgttatgctcgcaggtcgtgaaatccgcccgacg

SEQ ID NO: 77 - DNA, 103 bp, Artificial Sequence
  Description of Artificial Sequence Synthetic probe
catgtcgagataggaagtgtgcgttggaggctcatcgttcctatattccacaccacttattattacagatgttatgctcgcaggtctgatgcgcgtgagtcac

SEQ ID NO: 78 - DNA, 102 bp, Artificial Sequence
  Description of Artificial Sequence Synthetic probe
caatctgccatcgcgcgattgttggaggctcatcgttcctatattccacaccacttattattacagatgttatgctcgcaggtccggcaatctcggtgatgc

SEQ ID NO: 79 - DNA, 109 bp, Artificial Sequence
  Description of Artificial Sequence Synthetic probe
cgaagcaggtacatactcggtcgttggaggctcatcgttcctatattccacaccacttattattacagatgttatgctcgcaggtcacgagctaaatcttgataaactt

SEQ ID NO: 80 - DNA, 108 bp, Artificial Sequence
  Description of Artificial Sequence Synthetic probe
tagaatagcggaagctttcatgggttggaggctcatcgttcctatattccacaccacttattattacagatgttatgctcgcaggtcagctagcttgtaagcaaactg

SEQ ID NO: 81 - DNA, 108 bp, Artificial Sequence
  Description of Artificial Sequence Synthetic probe
caagtccaatacgacgagctaaagttggaggctcatcgttcctatattccacaccacttattattacagatgttatgctcgcaggtcgaatagcatggattgcacttc

SEQ ID NO: 82 - DNA, 109 bp, Artificial Sequence
  Description of Artificial Sequence Synthetic probe
ggtacatactcggtcgaagcacgttggaggctcatcgttcctatattccacaccacttattattacagatgttatgctcgcaggtcaatcttgataaactgaaatagcg

SEQ ID NO: 83 - DNA, 107 bp, Artificial Sequence
  Description of Artificial Sequence Synthetic probe
ggtacatactcggtcgatgcacgttggaggctcatcgttcctatattccacaccacttattattacagatgttatgctcgcaggtctcttgataaaccggaatagcg

SEQ ID NO: 84 - DNA, 110 bp, Artificial Sequence
  Description of Artificial Sequence Synthetic probe
gtaattgaactagctaatgccgtacgttggaggctcatcgttcctatattccacaccacttattattacagatgttatgctcgcaggtcttatgacaccagtttctaggc

SEQ ID NO: 85 - DNA, 106 bp, Artificial Sequence
  Description of Artificial Sequence Synthetic probe
caagtactgttcctgtacgtcaggttggaggctcatcgttcctatattccacaccacttattattacagatgttatgctcgcaggtcgcccagttgtgatgcattc

SEQ ID NO: 86 - DNA, 107 bp, Artificial Sequence
  Description of Artificial Sequence Synthetic probe
tctctttcccattgtttcatggcgttggaggctcatcgttcctatattccacaccacttattattacagatgttatgctcgcaggtctgcggaaattctaagctgac

SEQ ID NO: 87 - DNA, 108 bp, Artificial Sequence
  Description of Artificial Sequence Synthetic probe
gtaggttatgcagttattaggttcaggttggaggctcatcgttcctatattccacaccacttattattacagatgttatgctcgcaggtcgactcagccgagtcaagc

SEQ ID NO: 88 - DNA, 108 bp, Artificial Sequence
  Description of Artificial Sequence Synthetic probe
gcagtaccaacatagctaaatgcgttggaggctcatcgttcctatattccacaccacttattattacagatgttatgctcgcaggtcaaataacaaatcacaggccac

SEQ ID NO: 89 - DNA, 105 bp, Artificial Sequence
  Description of Artificial Sequence Synthetic probe
ggtcctgtggtggtttccaccgttggaggctcatcgttcctatattccacaccacttattattacagatgttatgctcgcaggtccgcgataatggcttcattgg

SEQ ID NO: 90 - DNA, 105 bp, Artificial Sequence
  Description of Artificial Sequence Synthetic probe
taaccgctgtggtcctgtgggttggaggctcatcgttcctatattccacaccacttattattacagatgttatgctcgcaggtctgcgcaataatagcttcattg

SEQ ID NO: 91 - DNA, 105 bp, Artificial Sequence
  Description of Artificial Sequence Synthetic probe
ggaagcgttgcttgccatagtgttggaggctcatcgttcctatattccacaccacttattattacagatgttatgctcgcaggtcaaccgaagcaccatgtaatt

SEQ ID NO: 92 - DNA, 106 bp, Artificial Sequence
  Description of Artificial Sequence Synthetic probe
gttcggtgcaaagacgccggttggaggctcatcgttcctatattccacaccacttattattacagatgttatgctcgcaggtctcgcagacttcaatatcaatatt

SEQ ID NO: 93 - DNA, 104 bp, Artificial Sequence
  Description of Artificial Sequence Synthetic probe
cacctgatgcagaaccagcatgttggaggctcatcgttcctatattccacaccacttattattacagatgttatgctcgcaggtcaggccacgttatcactgtg

SEQ ID NO: 94 - DNA, 103 bp, Artificial Sequence
  Description of Artificial Sequence Synthetic probe
cagctgccgttgcgaacggttggaggctcatcgttcctatattccacaccacttattattacagatgttatgctcgcaggtccgcagataaatcaccacaatc

SEQ ID NO: 95 - DNA, 102 bp, Artificial Sequence
  Description of Artificial Sequence Synthetic probe
gctcagacgctggctggtcgttggaggctcatcgttcctatattccacaccacttattattacagatgttatgctcgcaggtcccgcagataaatcaccacg

SEQ ID NO: 96 - DNA, 101 bp, Artificial Sequence
  Description of Artificial Sequence Synthetic probe
gccagtagcagattggcggcgttggaggctcatcgttcctatattccacaccacttattattacagatgttatgctcgcaggtcgaacgggcgctcagacg

SEQ ID NO: 97 - DNA, 104 bp, Artificial Sequence
  Description of Artificial Sequence Synthetic probe
ccactgcagcagatgccgtgttggaggctcatcgttcctatattccacaccacttattattacagatgttatgctcgcaggtcgtatcccgcagataaatcacc

SEQ ID NO: 98 - DNA, 103 bp, Artificial Sequence
  Description of Artificial Sequence Synthetic probe
ttaatttgcttaagcggctgcggttggaggctcatcgttcctatattccacaccacttattattacagatgttatgctcgcaggtcccagctgttcgtcaccg

SEQ ID NO: 99 - DNA, 101 bp, Artificial Sequence
  Description of Artificial Sequence Synthetic probe
gggaaagcgttcatcggcggttggaggctcatcgttcctatattccacaccacttattattacagatgttatgctcgcaggtctcgctcatggtaatggcg

SEQ ID NO: 100 - DNA, 103 bp, Artificial Sequence
  Description of Artificial Sequence Synthetic probe
tcttatcggcgataaaccagccgttggaggctcatcgttcctatattccacaccacttattattacagatgttatgctcgcaggtccgttgccagtgctcgat

SEQ ID NO: 101 - DNA, 108 bp, Artificial Sequence
  Description of Artificial Sequence Synthetic probe
cagtccctcgatattcagatcagagttggaggctcatcgttcctatattccacaccacttattattacagatgttatgctcgcaggtcttaacaatttcgcaaccgtc

SEQ ID NO: 102 - DNA, 108 bp, Artificial Sequence
  Description of Artificial Sequence Synthetic probe
cagctgcggtaaagctcatcagttggaggctcatcgttcctatattccacaccacttattattacagatgttatgctcgcaggtccatagttaagccagtatacactc

SEQ ID NO: 103 - DNA, 112 bp, Artificial Sequence
  Description of Artificial Sequence Synthetic probe
gtcggaaagttgaccagacattagttggaggctcatcgttcctatattccacaccacttattattacagatgttatgctcgcaggtcatactaggagaagttaataaatacg

SEQ ID NO: 104 - DNA, 112 bp, Artificial Sequence
  Description of Artificial Sequence Synthetic probe
cattctctcgctttaatttattaacctgttggaggctcatcgttcctatattccacaccacttattattacagatgttatgctcgcaggtcatcgaccttctggacattatc

SEQ ID NO: 105 - DNA, 109 bp, Artificial Sequence
  Description of Artificial Sequence Synthetic probe
gtaacaactttcatgctctcctaaagttggaggctcatcgttcctatattccacaccacttattattacagatgttatgctcgcaggtccggtaactgatgccgtattt

SEQ ID NO: 106 - DNA, 106 bp, Artificial Sequence
  Description of Artificial Sequence Synthetic probe
gtgaagtgaatggtcagtatgttggttggaggctcatcgttcctatattccacaccacttattattacagatgttatgctcgcaggtcagtgcgcaggagattagc

SEQ ID NO: 107 - DNA, 105 bp, Artificial Sequence
  Description of Artificial Sequence Synthetic probe
cctgtcctacgagttgcatgatgttggaggctcatcgttcctatattccacaccacttattattacagatgttatgctcgcaggtcataatggcctgcttctcgc

SEQ ID NO: 108 - DNA, 107 bp, Artificial Sequence
  Description of Artificial Sequence Synthetic probe
cgtttccagactttacgaaacacgttggaggctcatcgttcctatattccacaccacttattattacagatgttatgctcgcaggtcacgttgtgagggtaaacaac

SEQ ID NO: 109 - DNA, 108 bp, Artificial Sequence
  Description of Artificial Sequence Synthetic probe
cgttgcttacgcaaccaaatatcgttggaggctcatcgttcctatattccacaccacttattattacagatgttatgctcgcaggtctgatcttgctcaatgaggtta

SEQ ID NO: 110 - DNA, 114 bp, Artificial Sequence
  Description of Artificial Sequence Synthetic probe
catcatgttcatatttatcagagctcgttggaggctcatcgttcctatattccacaccacttattattacagatgttatgctcgcaggtctagatttcataaagtctaacacac

SEQ ID NO: 111 - DNA, 106 bp, Artificial Sequence
  Description of Artificial Sequence Synthetic probe
gtttccacatggtgaacggtggttggaggctcatcgttcctatattccacaccacttattattacagatgttatgctcgcaggtcaaacctgtcactctgaatgtt

SEQ ID NO: 112 - DNA, 115 bp, Artificial Sequence
  Description of Artificial Sequence Synthetic probe
caaatactaaattatacagtatcagagaggttggaggctcatcgttcctatattccacaccacttattattacagatgttatgctcgcaggtcatgcaaagcgttatgaaatttc

SEQ ID NO: 113 - DNA, 115 bp, Artificial Sequence
  Description of Artificial Sequence Synthetic probe
gttcttattattataagtatctattaacagttgttggaggctcatcgttcctatattccacaccacttattattacagatgttatgctcgcaggtccattagtggctgctgcaat

SEQ ID NO: 114 - DNA, 108 bp, Artificial Sequence
  Description of Artificial Sequence Synthetic probe
catcgggaaatggaagtcgttatgttggaggctcatcgttcctatattccacaccacttattattacagatgttatgctcgcaggtcgttcaatcgtcaaagttgttc

SEQ ID NO: 115 - DNA, 108 bp, Artificial Sequence
  Description of Artificial Sequence Synthetic probe
cgtggtttgtgctgagcaaaggttggaggctcatcgttcctatattccacaccacttattattacagatgttatgctcgcaggtccaaagttaagttgtcagtttgag

SEQ ID NO: 116 - DNA, 101 bp, Artificial Sequence
  Description of Artificial Sequence Synthetic probe
gccgcccgaaggacatcaagttggaggctcatcgttcctatattccacaccacttattattacagatgttatgctcgcaggtcagacgggacgtacacaac

SEQ ID NO: 117 - DNA, 103 bp, Artificial Sequence
  Description of Artificial Sequence Synthetic probe
gcaactcatcaccatcacggagttggaggctcatcgttcctatattccacaccacttattattacagatgttatgctcgcaggtctgatgcgtacgttgccac

SEQ ID NO: 118 - DNA, 104 bp, Artificial Sequence
  Description of Artificial Sequence Synthetic probe
gcgacagccatgacagacgcgttggaggctcatcgttcctatattccacaccacttattattacagatgttatgctcgcaggtcggacaatgagaccattggac

SEQ ID NO: 119 - DNA, 104 bp, Artificial Sequence
  Description of Artificial Sequence Synthetic probe
aaacgactgcgttgcgatatggttggaggctcatcgttcctatattccacaccacttattattacagatgttatgctcgcaggtcttccgaaggacatcaacgc

SEQ ID NO: 120 - DNA, 103 bp, Artificial Sequence
  Description of Artificial Sequence Synthetic probe
atgcgaccaaacgccatcgcgttggaggctcatcgttcctatattccacaccacttattattacagatgttatgctcgcaggtcatcgtcatggaagtgcgta

SEQ ID NO: 121 - DNA, 107 bp, Artificial Sequence
  Description of Artificial Sequence Synthetic probe
gaacaggcttatgtcaactggggttggaggctcatcgttcctatattccacaccacttattattacagatgttatgctcgcaggtccataacatcaaacatcgaccc

SEQ ID NO: 122 - DNA, 103 bp, Artificial Sequence
  Description of Artificial Sequence Synthetic probe
acgaaccgaacaggcttatgtcgttggaggctcatcgttcctatattccacaccacttattattacagatgttatgctcgcaggtctaacgcgcttgctgctt

SEQ ID NO: 123 - DNA, 105 bp, Artificial Sequence
  Description of Artificial Sequence Synthetic probe
gctgtaattatgacgacgccggttggaggctcatcgttcctatattccacaccacttattattacagatgttatgctcgcaggtcctcggtgagattcagaatgc

SEQ ID NO: 124 - DNA, 106 bp, Artificial Sequence
  Description of Artificial Sequence Synthetic probe
catcatagacgcggtcaaatagagttggaggctcatcgttcctatattccacaccacttattattacagatgttatgctcgcaggtcactcatcaccatcacggac

SEQ ID NO: 125 - DNA, 108 bp, Artificial Sequence
  Description of Artificial Sequence Synthetic probe
gtgtatgtcagcgatttgtccatgttggaggctcatcgttcctatattccacaccacttattattacagatgttatgctcgcaggtctgtcatattgtcttgccgatt

SEQ ID NO: 126 - DNA, 106 bp, Artificial Sequence
  Description of Artificial Sequence Synthetic probe
gtccacctcgccaacaatcaagttggaggctcatcgttcctatattccacaccacttattattacagatgttatgctcgcaggtcatatcaacacgggaaagacct

SEQ ID NO: 127 - DNA, 105 bp, Artificial Sequence
  Description of Artificial Sequence Synthetic probe
gcgtgattatcacgttcggcagttggaggctcatcgttcctatattccacaccacttattattacagatgttatgctcgcaggtccttgcagatttaaccgacac

SEQ ID NO: 128 - DNA, 106 bp, Artificial Sequence
  Description of Artificial Sequence Synthetic probe
ggctcgacttcctgatgaatacggttggaggctcatcgttcctatattccacaccacttattattacagatgttatgctcgcaggtctgaaaccgggcagagtatt

SEQ ID NO: 129 - DNA, 108 bp, Artificial Sequence
  Description of Artificial Sequence Synthetic probe
caacgatgtatgtcaacgatttgtgttggaggctcatcgttcctatattccacaccacttattattacagatgttatgctcgcaggtcattgcgtagtccaattcgtc

SEQ ID NO: 130 - DNA, 108 bp, Artificial Sequence
  Description of Artificial Sequence Synthetic probe
caggctgtttcgggctgtgagttggaggctcatcgttcctatattccacaccacttattattacagatgttatgctcgcaggtcgggttattaataaagatgataggc

SEQ ID NO: 131 - DNA, 107 bp, Artificial Sequence
  Description of Artificial Sequence Synthetic probe
ggctcggcttcctgatgaatacgttggaggctcatcgttcctatattccacaccacttattattacagatgttatgctcgcaggtcaggcatggtattgacttcatt

SEQ ID NO: 132 - DNA, 112 bp, Artificial Sequence
  Description of Artificial Sequence Synthetic probe
taattcaagtgcaactctcgcaagttggaggctcatcgttcctatattccacaccacttattattacagatgttatgctcgcaggtctttattctctaatgcgctatatatt

SEQ ID NO: 133 - DNA, 110 bp, Artificial Sequence
  Description of Artificial Sequence Synthetic probe
ggatagttacgactttctgcttcagttggaggctcatcgttcctatattccacaccacttattattacagatgttatgctcgcaggtctgtattgctattatcgtcaacg

SEQ ID NO: 134 - DNA, 108 bp, Artificial Sequence
  Description of Artificial Sequence Synthetic probe
cagtatttcaccttgtccgtaaccgttggaggctcatcgttcctatattccacaccacttattattacagatgttatgctcgcaggtcgtttacgacttgttgcatgc

SEQ ID NO: 135 - DNA, 111 bp, Artificial Sequence
  Description of Artificial Sequence Synthetic probe
aatgtttatatctttaacgcctaaactgttggaggctcatcgttcctatattccacaccacttattattacagatgttatgctcgcaggtcatgctttggtctttctgcat

SEQ ID NO: 136 - DNA, 101 bp, Artificial Sequence
  Description of Artificial Sequence Synthetic probe
ctggcccttgaggtcgcgggttggaggctcatcgttcctatattccacaccacttattattacagatgttatgctcgcaggtccggtcttcacctcgacac

SEQ ID NO: 137 - DNA, 103 bp, Artificial Sequence
  Description of Artificial Sequence Synthetic probe
gacgtagatcgggtcgagctgttggaggctcatcgttcctatattccacaccacttattattacagatgttatgctcgcaggtcacggaaacctcggagaatt

SEQ ID NO: 138 - DNA, 102 bp, Artificial Sequence
  Description of Artificial Sequence Synthetic probe
ggcgtactgctgcttgctcagttggaggctcatcgttcctatattccacaccacttattattacagatgttatgctcgcaggtctgacgtcgacgtagatcg

SEQ ID NO: 139 - DNA, 100 bp, Artificial Sequence
  Description of Artificial Sequence Synthetic probe
cctgttcctgggtcgaagccgttggaggctcatcgttcctatattccacaccacttattattacagatgttatgctcgcaggtccttcggtcaccgcgga

SEQ ID NO: 140 - DNA, 115 bp, Artificial Sequence
  Description of Artificial Sequence Synthetic probe
gtcaggctaaatatagctatcttatcggttggaggctcatcgttcctatattccacaccacttattattacagatgttatgctcgcaggtctcagttactgctatagaaattgat

SEQ ID NO: 141 - DNA, 114 bp, Artificial Sequence
  Description of Artificial Sequence Synthetic probe
catcctaagccaagtgtagactcgttggaggctcatcgttcctatattccacaccacttattattacagatgttatgctcgcaggtcaagatatatggtaatattccttataac

SEQ ID NO: 142 - DNA, 109 bp, Artificial Sequence
  Description of Artificial Sequence Synthetic probe
gtttataagtgggtaaaccgtgaatgttggaggctcatcgttcctatattccacaccacttattattacagatgttatgctcgcaggtcgaaacgagctttaggtttgc

SEQ ID NO: 143 - DNA, 114 bp, Artificial Sequence
  Description of Artificial Sequence Synthetic probe
gcagcacttgaccgccatgagtgaccagttggaggctcatcgttcctatattccacaccacttattattacagatgttatgctcgcaggtccatcgcaccaacaacaataatcg

SEQ ID NO: 144 - DNA, 114 bp, Artificial Sequence
  Description of Artificial Sequence Synthetic probe
gtgatcactgatgcaccagatgaagtgttggaggctcatcgttcctatattccacaccacttattattacagatgttatgctcgcaggtcatcttgatattcaagtctatgacg

SEQ ID NO: 145 - DNA, 113 bp, Artificial Sequence
  Description of Artificial Sequence Synthetic probe
gatattattgatcatggtgccaagccaagttggaggctcatcgttcctatattccacaccacttattattacagatgttatgctcgcaggtccaatatgaagctgacgacgcg

SEQ ID NO: 146 - DNA, 112 bp, Artificial Sequence
  Description of Artificial Sequence Synthetic probe
gctgagcgtgaaggttcatggattattagttggaggctcatcgttcctatattccacaccacttattattacagatgttatgctcgcaggtcggtaaggcttacggtctcat

SEQ ID NO: 147 - DNA, 118 bp, Artificial Sequence
  Description of Artificial Sequence Synthetic probe
gcatcttgtgcagcctgaatagcagcgtgttggaggctcatcgttcctatattccacaccacttattattacagatgttatgctcgcaggtcaccacgttgaatatcaccttcggcat

SEQ ID NO: 148 - DNA, 118 bp, Artificial Sequence
  Description of Artificial Sequence Synthetic probe
aagtccataattgcttgagtgtagtcatgttggaggctcatcgttcctatattccacaccacttattattacagatgttatgctcgcaggtcatcttcgcactgaataataagaacat

SEQ ID NO: 149 - DNA, 113 bp, Artificial Sequence
  Description of Artificial Sequence Synthetic probe
gcttgctggttctgcacgtagcttactggttggaggctcatcgttcctatattccacaccacttattattacagatgttatgctcgcaggtcaagatgaacaggctactgcaa

SEQ ID NO: 150 - DNA, 116 bp, Artificial Sequence
  Description of Artificial Sequence Synthetic probe
gcagcgctgtgcaagttcaatgtattctgttggaggctcatcgttcctatattccacaccacttattattacagatgttatgctcgcaggtcctcgtgcgagtattccttaagtgt

SEQ ID NO: 151 - DNA, 116 bp, Artificial Sequence
  Description of Artificial Sequence Synthetic probe
gtataacactcggccagcgccaaggttcgttggaggctcatcgttcctatattccacaccacttattattacagatgttatgctcgcaggtcgttcacacatcgccacaatatgat

SEQ ID NO: 152 - DNA, 109 bp, Artificial Sequence
  Description of Artificial Sequence Synthetic probe
accatgcagatacaatgaaccagttggaggctcatcgttcctatattccacaccacttattattacagatgttatgctcgcaggtcggatgataagacacatccaattc

SEQ ID NO: 153 - DNA, 112 bp, Artificial Sequence
  Description of Artificial Sequence Synthetic probe
catcaacagcttcttgaagcattcgttggaggctcatcgttcctatattccacaccacttattattacagatgttatgctcgcaggtcgtccaacaactataacagaacgtc

SEQ ID NO: 154 - DNA, 117 bp, Artificial Sequence
  Description of Artificial Sequence Synthetic probe
aacatatcacctgatattctagtatcgttggaggctcatcgttcctatattccacaccacttattattacagatgttatgctcgcaggtcattccattatattcaacaggattgtga

SEQ ID NO: 155 - DNA, 105 bp, Artificial Sequence
  Description of Artificial Sequence Synthetic probe
gctgttgcttgcggatactggttggaggctcatcgttcctatattccacaccacttattattacagatgttatgctcgcaggtccgtatatgtagctcaagttgc

SEQ ID NO: 156 - DNA, 115 bp, Artificial Sequence
  Description of Artificial Sequence Synthetic probe
aagagctaatgcagctattgcacttatgttggaggctcatcgttcctatattccacaccacttattattacagatgttatgctcgcaggtccatacacttcagctataagaccat

SEQ ID NO: 157 - DNA, 110 bp, Artificial Sequence
  Description of Artificial Sequence Synthetic probe
aacaagagcagaagttacagacgtgttggaggctcatcgttcctatattccacaccacttattattacagatgttatgctcgcaggtcgtataatggtggctagaggtga

SEQ ID NO: 158 - DNA, 110 bp, Artificial Sequence
  Description of Artificial Sequence Synthetic probe
actcgtgaagaccatgcagatacaagttggaggctcatcgttcctatattccacaccacttattattacagatgttatgctcgcaggtcaatacttacaatgcctgagga

SEQ ID NO: 159 - DNA, 107 bp, Artificial Sequence
  Description of Artificial Sequence Synthetic probe
accatgcagatacaatgaaccgttggaggctcatcgttcctatattccacaccacttattattacagatgttatgctcgcaggtccctgaggatgataagacacatc

SEQ ID NO: 160 - DNA, 116 bp, Artificial Sequence
  Description of Artificial Sequence Synthetic probe
gcatctgctgcttctattgctcctactgttggaggctcatcgttcctatattccacaccacttattattacagatgttatgctcgcaggtcacatgaactgatattagttctccaa

SEQ ID NO: 161 - DNA, 111 bp, Artificial Sequence
  Description of Artificial Sequence Synthetic probe
gcacaagctggagataacatcgggttggaggctcatcgttcctatattccacaccacttattattacagatgttatgctcgcaggtcgtagaggacgtattcacaatcact

SEQ ID NO: 162 - DNA, 114 bp, Artificial Sequence
  Description of Artificial Sequence Synthetic probe
ctctatcagcttctactgcttcttcgttggaggctcatcgttcctatattccacaccacttattattacagatgttatgctcgcaggtcccatctcatccacagttaatatatc

SEQ ID NO: 163 - DNA, 116 bp, Artificial Sequence
  Description of Artificial Sequence Synthetic probe
agatgagattcatactatcgttggagctgttggaggctcatcgttcctatattccacaccacttattattacagatgttatgctcgcaggtcagcagagagaatagtaagaggaga

SEQ ID NO: 164 - DNA, 109 bp, Artificial Sequence
  Description of Artificial Sequence Synthetic probe
catcaacagcttcttgaagcattgttggaggctcatcgttcctatattccacaccacttattattacagatgttatgctcgcaggtcgtccaacaactataacagaacg

SEQ ID NO: 165 - DNA, 111 bp, Artificial Sequence
  Description of Artificial Sequence Synthetic probe
gtcagcaatacgccaccaagctcctatgttggaggctcatcgttcctatattccacaccacttattattacagatgttatgctcgcaggtcgtggtggatatcctgttacc

SEQ ID NO: 166 - DNA, 119 bp, Artificial Sequence
  Description of Artificial Sequence Synthetic probe
gcgcaatagagttgtataagagtgctggttggaggctcatcgttcctatattccacaccacttattattacagatgttatgctcgcaggtcagcattaattatagattataatgtataa

SEQ ID NO: 167 - DNA, 110 bp, Artificial Sequence
  Description of Artificial Sequence Synthetic probe
ggcataataggatggatagatgagttggaggctcatcgttcctatattccacaccacttattattacagatgttatgctcgcaggtcactaatccaacttctactgctat

SEQ ID NO: 168 - DNA, 115 bp, Artificial Sequence
  Description of Artificial Sequence Synthetic probe
gtacattcacatatagaccatcttaagttggaggctcatcgttcctatattccacaccacttattattacagatgttatgctcgcaggtcacataggtgcaggtagaatagtata

SEQ ID NO: 169 - DNA, 113 bp, Artificial Sequence
  Description of Artificial Sequence Synthetic probe
ccataccagtatcttggcatattggttggaggctcatcgttcctatattccacaccacttattattacagatgttatgctcgcaggtcataatgaataacagcaggtgtatta

SEQ ID NO: 170 - DNA, 109 bp, Artificial Sequence
  Description of Artificial Sequence Synthetic probe
agatgaagcacaagctggagataagttggaggctcatcgttcctatattccacaccacttattattacagatgttatgctcgcaggtcaggacgtattcacaatcactg

SEQ ID NO: 171 - DNA, 111 bp, Artificial Sequence
  Description of Artificial Sequence Synthetic probe
ataatcattcacctccatcattcataagttggaggctcatcgttcctatattccacaccacttattattacagatgttatgctcgcaggtcactgaatatggttcgtctca

SEQ ID NO: 172 - DNA, 111 bp, Artificial Sequence
  Description of Artificial Sequence Synthetic probe
gtacattcacatatagaccatcttagttggaggctcatcgttcctatattccacaccacttattattacagatgttatgctcgcaggtcacataggtgcaggtagaatagt

SEQ ID NO: 173 - DNA, 104 bp, Artificial Sequence
  Description of Artificial Sequence Synthetic probe
actccaccaggatgttgtccgttggaggctcatcgttcctatattccacaccacttattattacagatgttatgctcgcaggtcgtaggaccgtcgtgtccaag

SEQ ID NO: 174 - DNA, 119 bp, Artificial Sequence
  Description of Artificial Sequence Synthetic probe
gcaatatcaatggtatcgaaggcactatgttggaggctcatcgttcctatattccacaccacttattattacagatgttatgctcgcaggtcgtattgaaggtactattagcgatatgc

SEQ ID NO: 175 - DNA, 111 bp, Artificial Sequence
  Description of Artificial Sequence Synthetic probe
gtgccggtctcggttactcaatggttggaggctcatcgttcctatattccacaccacttattattacagatgttatgctcgcaggtcggattattataatgcagctagaag

SEQ ID NO: 176 - DNA, 111 bp, Artificial Sequence
  Description of Artificial Sequence Synthetic probe
gtacattcacatatagaccatcttgttggaggctcatcgttcctatattccacaccacttattattacagatgttatgctcgcaggtcacataggtgcaggtagaatagta

SEQ ID NO: 177 - DNA, 119 bp, Artificial Sequence
  Description of Artificial Sequence Synthetic probe
agttccttcatatgactcagttgattgagttggaggctcatcgttcctatattccacaccacttattattacagatgttatgctcgcaggtcgttatatcttcaattatacattcctgc

SEQ ID NO: 178 - DNA, 108 bp, Artificial Sequence
  Description of Artificial Sequence Synthetic probe
cagcagttgttgctagaggtatggttggaggctcatcgttcctatattccacaccacttattattacagatgttatgctcgcaggtcgcatcaccaggtgcagcaagt

SEQ ID NO: 179 - DNA, 117 bp, Artificial Sequence
  Description of Artificial Sequence Synthetic probe
gcaattctctgttgttgtcctccactcagttggaggctcatcgttcctatattccacaccacttattattacagatgttatgctcgcaggtcagtaagagcctcttcttggtcatga

SEQ ID NO: 180 - DNA, 115 bp, Artificial Sequence
  Description of Artificial Sequence Synthetic probe
ctattcctgataataagtgtgtcctcatgttggaggctcatcgttcctatattccacaccacttattattacagatgttatgctcgcaggtccggcatcatctaacaattcttct

SEQ ID NO: 181 - DNA, 113 bp, Artificial Sequence
  Description of Artificial Sequence Synthetic probe
gtaattccaattacttctagctctggtggttggaggctcatcgttcctatattccacaccacttattattacagatgttatgctcgcaggtctaccatcttctccatgtgtat

SEQ ID NO: 182 - DNA, 109 bp, Artificial Sequence
  Description of Artificial Sequence Synthetic probe
ccatgcagatacaatgaaccaggttggaggctcatcgttcctatattccacaccacttattattacagatgttatgctcgcaggtcgatgataagacacatccaattcc

SEQ ID NO: 183 - DNA, 112 bp, Artificial Sequence
  Description of Artificial Sequence Synthetic probe
ccttctgccattgtagaacaagctccatgttggaggctcatcgttcctatattccacaccacttattattacagatgttatgctcgcaggtccctgtaactgtccactgagc

SEQ ID NO: 184 - DNA, 113 bp, Artificial Sequence
  Description of Artificial Sequence Synthetic probe
caatcatgatagaattagatggaacgttggaggctcatcgttcctatattccacaccacttattattacagatgttatgctcgcaggtcagcaatagttccatcaggagcatc

SEQ ID NO: 185 - DNA, 108 bp, Artificial Sequence
  Description of Artificial Sequence Synthetic probe
agtggtgaaggtgttcaacaaggttggaggctcatcgttcctatattccacaccacttattattacagatgttatgctcgcaggtcactgaagctggatatgttggag

SEQ ID NO: 186 - DNA, 108 bp, Artificial Sequence
  Description of Artificial Sequence Synthetic probe
cgcctcttcagaagcggatatcagttggaggctcatcgttcctatattccacaccacttattattacagatgttatgctcgcaggtcgccagacttccgccacaacct

SEQ ID NO: 187 - DNA, 112 bp, Artificial Sequence
  Description of Artificial Sequence Synthetic probe
ggcataataggatggatagatgagcgttggaggctcatcgttcctatattccacaccacttattattacagatgttatgctcgcaggtcgcagcagttgtacctacaactaa

SEQ ID NO: 188 - DNA, 119 bp, Artificial Sequence
  Description of Artificial Sequence Synthetic probe
agttccttcatatgactcagttgattggttggaggctcatcgttcctatattccacaccacttattattacagatgttatgctcgcaggtcgttatatcttcaattatacattcctgcg

SEQ ID NO: 189 - DNA, 115 bp, Artificial Sequence
  Description of Artificial Sequence Synthetic probe
gcatggtagttcgccagccgctggaacgttggaggctcatcgttcctatattccacaccacttattattacagatgttatgctcgcaggtcacagcaaccgcaagttcttgacat

SEQ ID NO: 190 - DNA, 116 bp, Artificial Sequence
  Description of Artificial Sequence Synthetic probe
aatatcatggtcgtgtccaggcactggcgttggaggctcatcgttcctatattccacaccacttattattacagatgttatgctcgcaggtcgttctggtagctgcttctactgta

SEQ ID NO: 191 - DNA, 114 bp, Artificial Sequence
  Description of Artificial Sequence Synthetic probe
aacttacaactacgcgcacttgaatcggttggaggctcatcgttcctatattccacaccacttattattacagatgttatgctcgcaggtcgagtgttgtatgatagtctcggt

SEQ ID NO: 192 - DNA, 118 bp, Artificial Sequence
  Description of Artificial Sequence Synthetic probe
gcaagttgaggagatgctggcatgattcgttggaggctcatcgttcctatattccacaccacttattattacagatgttatgctcgcaggtcacatggctctggaagatgtgctgatc

SEQ ID NO: 193 - DNA, 117 bp, Artificial Sequence
  Description of Artificial Sequence Synthetic probe
gcgataattgtaatgattcgtggtgttagttggaggctcatcgttcctatattccacaccacttattattacagatgttatgctcgcaggtcccgttgtcaatccagttagtagact

SEQ ID NO: 194 - DNA, 115 bp, Artificial Sequence
  Description of Artificial Sequence Synthetic probe
actgtggcagtctatgttccaattgtagttggaggctcatcgttcctatattccacaccacttattattacagatgttatgctcgcaggtccttatcgacataatcctgataatc

SEQ ID NO: 195 - DNA, 106 bp, Artificial Sequence
  Description of Artificial Sequence Synthetic probe
gcgtcgcttcttgcgctcgccgttggaggctcatcgttcctatattccacaccacttattattacagatgttatgctcgcaggtcaatgtattcataccgtcaagt

SEQ ID NO: 196 - DNA, 112 bp, Artificial Sequence
  Description of Artificial Sequence Synthetic probe
gccttcacaactacgttggaaggtcttcgttggaggctcatcgttcctatattccacaccacttattattacagatgttatgctcgcaggtcctaacagtcctgccgactac

SEQ ID NO: 197 - DNA, 112 bp, Artificial Sequence
  Description of Artificial Sequence Synthetic probe
gccttcacaactacgttggaaggtcttgttggaggctcatcgttcctatattccacaccacttattattacagatgttatgctcgcaggtcctaacagtcctgccgactact

SEQ ID NO: 198 - DNA, 115 bp, Artificial Sequence
  Description of Artificial Sequence Synthetic probe
gccgctgagcggcggcaagccgatggcgttggaggctcatcgttcctatattccacaccacttattattacagatgttatgctcgcaggtcgaatggcaggccaagctgaaggcg

SEQ ID NO: 199 - DNA, 119 bp, Artificial Sequence
  Description of Artificial Sequence Synthetic probe
gccaagcggcattctggcgccagtggagttggaggctcatcgttcctatattccacaccacttattattacagatgttatgctcgcaggtcccagaccggagtggacaacgtcgaggcg

SEQ ID NO: 200 - DNA, 113 bp, Artificial Sequence
  Description of Artificial Sequence Synthetic probe
gccgtatatcatcggcaataaccgcacggttggaggctcatcgttcctatattccacaccacttattattacagatgttatgctcgcaggtcgcatgatggtcaacaaggtgc

SEQ ID NO: 201 - DNA, 115 bp, Artificial Sequence
  Description of Artificial Sequence Synthetic probe
acgagccgagataggtctgcagcgtacgttggaggctcatcgttcctatattccacaccacttattattacagatgttatgctcgcaggtcgtactgatattcaccatactgccg

SEQ ID NO: 202 - DNA, 112 bp, Artificial Sequence
  Description of Artificial Sequence Synthetic probe
gcaatatcttcaccggcagccaccgcggttggaggctcatcgttcctatattccacaccacttattattacagatgttatgctcgcaggtcggtatatggcacgccaatcgc

SEQ ID NO: 203 - DNA, 111 bp, Artificial Sequence
  Description of Artificial Sequence Synthetic probe
aataaccttaacgtcgccaacacggttggaggctcatcgttcctatattccacaccacttattattacagatgttatgctcgcaggtcctcggtgaacacctcctggcacg

SEQ ID NO: 204 - DNA, 113 bp, Artificial Sequence
  Description of Artificial Sequence Synthetic probe
gcggaactgcttggcgtagtaagcgttggaggctcatcgttcctatattccacaccacttattattacagatgttatgctcgcaggtccatgtagtgccgtagaccttcacca

SEQ ID NO: 205 - DNA, 119 bp, Artificial Sequence
  Description of Artificial Sequence Synthetic probe
gcgagaccggcggcaccatcgtctccaggttggaggctcatcgttcctatattccacaccacttattattacagatgttatgctcgcaggtcttctgcctgatggacgtctccggctcg

SEQ ID NO: 206 - DNA, 120 bp, Artificial Sequence
  Description of Artificial Sequence Synthetic probe
gcggttcacctgttcgccttcgaacacggttggaggctcatcgttcctatattccacaccacttattattacagatgttatgctcgcaggtcgcgcagcatctgacgcaggatggtctcg

SEQ ID NO: 207 - DNA, 119 bp, Artificial Sequence
  Description of Artificial Sequence Synthetic probe
actccatcgccatcaaggacatggccgggttggaggctcatcgttcctatattccacaccacttattattacagatgttatgctcgcaggtcatcgacgtgttccgcatcttcgacgcg

SEQ ID NO: 208 - DNA, 114 bp, Artificial Sequence
  Description of Artificial Sequence Synthetic probe
gcctgatgcactacagcgcctgggttggaggctcatcgttcctatattccacaccacttattattacagatgttatgctcgcaggtctaccacatggtcgatctcgacgactgc

SEQ ID NO: 209 - DNA, 111 bp, Artificial Sequence
  Description of Artificial Sequence Synthetic probe
gcgcatccaggacggcgagtacggttggaggctcatcgttcctatattccacaccacttattattacagatgttatgctcgcaggtccttcgagtgcctgcacgagctgaa

SEQ ID NO: 210 - DNA, 115 bp, Artificial Sequence
  Description of Artificial Sequence Synthetic probe
gctggagaacgtcaaggtggtgatcatcgttggaggctcatcgttcctatattccacaccacttattattacagatgttatgctcgcaggtcaccgataacgacgaccgcatcaa

SEQ ID NO: 211 - DNA, 109 bp, Artificial Sequence
  Description of Artificial Sequence Synthetic probe
acgattggagaaggcagtgtgattgggttggaggctcatcgttcctatattccacaccacttattattacagatgttatgctcgcaggtcggacagattacaattggcg

SEQ ID NO: 212 - DNA, 106 bp, Artificial Sequence
  Description of Artificial Sequence Synthetic probe
gccgcaataccgatattccagttggaggctcatcgttcctatattccacaccacttattattacagatgttatgctcgcaggtcccattgtccaccagctgaaccg

SEQ ID NO: 213 - DNA, 110 bp, Artificial Sequence
  Description of Artificial Sequence Synthetic probe
gtgaaggtcgtgctcctatcggtgttggaggctcatcgttcctatattccacaccacttattattacagatgttatgctcgcaggtcagatctggtgaagttcgtatgat

SEQ ID NO: 214 - DNA, 113 bp, Artificial Sequence
  Description of Artificial Sequence Synthetic probe
gctggtacttgtacttatatcgagttggaggctcatcgttcctatattccacaccacttattattacagatgttatgctcgcaggtcatcagaagatgatatcgttacgtcat

SEQ ID NO: 215 - DNA, 111 bp, Artificial Sequence
  Description of Artificial Sequence Synthetic probe
gcgcatattgcattaatggctatagatgttggaggctcatcgttcctatattccacaccacttattattacagatgttatgctcgcaggtcgccagcaggttatacactcg

SEQ ID NO: 216 - DNA, 116 bp, Artificial Sequence
  Description of Artificial Sequence Synthetic probe
gcaattcttaccacagcacgaagaacaggttggaggctcatcgttcctatattccacaccacttattattacagatgttatgctcgcaggtcatctagatgaagataatgaagtcg

SEQ ID NO: 217 - DNA, 113 bp, Artificial Sequence
  Description of Artificial Sequence Synthetic probe
gcatcttcatacaatacttctagcttacgttggaggctcatcgttcctatattccacaccacttattattacagatgttatgctcgcaggtccacaataccagttgtattacg

SEQ ID NO: 218 - DNA, 115 bp, Artificial Sequence
  Description of Artificial Sequence Synthetic probe
gcttcagcgccattaccgccaccagctgttggaggctcatcgttcctatattccacaccacttattattacagatgttatgctcgcaggtcactcttgatatattcttgtaagcg

SEQ ID NO: 219 - DNA, 118 bp, Artificial Sequence
  Description of Artificial Sequence Synthetic probe
gttcacacaacgcgccgactagaatccgttggaggctcatcgttcctatattccacaccacttattattacagatgttatgctcgcaggtccacgatatccaagataatgattggcta

SEQ ID NO: 220 - DNA, 119 bp, Artificial Sequence
  Description of Artificial Sequence Synthetic probe
gcgcacctacaatcgccattactacacgttggaggctcatcgttcctatattccacaccacttattattacagatgttatgctcgcaggtcactcattatcgactgttacatcgactga

SEQ ID NO: 221 - DNA, 115 bp, Artificial Sequence
  Description of Artificial Sequence Synthetic probe
agcgcacatgtgacagcgtgtaggttagttggaggctcatcgttcctatattccacaccacttattattacagatgttatgctcgcaggtcgtgccttagattgttcagaacaat

SEQ ID NO: 222 - DNA, 112 bp, Artificial Sequence
  Description of Artificial Sequence Synthetic probe
cgaatggatatgtaccatggtcgatatcgttggaggctcatcgttcctatattccacaccacttattattacagatgttatgctcgcaggtcctctctaatatgatgtccat

SEQ ID NO: 223 - DNA, 112 bp, Artificial Sequence
  Description of Artificial Sequence Synthetic probe
actacaacagcaaccgcattacaatggcgttggaggctcatcgttcctatattccacaccacttattattacagatgttatgctcgcaggtcggtgctaagaggtcatcgga

SEQ ID NO: 224 - DNA, 116 bp, Artificial Sequence
  Description of Artificial Sequence Synthetic probe
agcttcagataagtacctatctgagttggaggctcatcgttcctatattccacaccacttattattacagatgttatgctcgcaggtcggaagaatagttattcttgataatgtat

SEQ ID NO: 225 - DNA, 106 bp, Artificial Sequence
  Description of Artificial Sequence Synthetic probe
cgtattgctcgaatacatgatagttggaggctcatcgttcctatattccacaccacttattattacagatgttatgctcgcaggtcacaatgtatcaaggccagct

SEQ ID NO: 226 - DNA, 110 bp, Artificial Sequence
  Description of Artificial Sequence Synthetic probe
gcgaccagttgttatcgaccgtgtgttggaggctcatcgttcctatattccacaccacttattattacagatgttatgctcgcaggtccagaacgatacggtgctgtata

SEQ ID NO: 227 - DNA, 113 bp, Artificial Sequence
  Description of Artificial Sequence Synthetic probe
caattacattgtctgttgcgtagataccgttggaggctcatcgttcctatattccacaccacttattattacagatgttatgctcgcaggtcgttgtggctaatgtgccagtt

SEQ ID NO: 228 - DNA, 116 bp, Artificial Sequence
  Description of Artificial Sequence Synthetic probe
gcaccactctatagcagtagcgtattggttggaggctcatcgttcctatattccacaccacttattattacagatgttatgctcgcaggtcacagccaatgtcacctaagtcaaca

SEQ ID NO: 229 - DNA, 117 bp, Artificial Sequence
  Description of Artificial Sequence Synthetic probe
acagtccgaataagatacgactattcgagttggaggctcatcgttcctatattccacaccacttattattacagatgttatgctcgcaggtccgttgtaacgtatatgaatagttga

SEQ ID NO: 230 - DNA, 112 bp, Artificial Sequence
  Description of Artificial Sequence Synthetic probe
agatgcaataacaggtcgaatattaattgttggaggctcatcgttcctatattccacaccacttattattacagatgttatgctcgcaggtcgccatagtgagagtagtgaa

SEQ ID NO: 231 - DNA, 112 bp, Artificial Sequence
  Description of Artificial Sequence Synthetic probe
agatgcaataacaggtcgaatattaagttggaggctcatcgttcctatattccacaccacttattattacagatgttatgctcgcaggtcacacatacggccatagtgagag

SEQ ID NO: 232 - DNA, 109 bp, Artificial Sequence
  Description of Artificial Sequence Synthetic probe
gaacataacgcgacgttccagctggttggaggctcatcgttcctatattccacaccacttattattacagatgttatgctcgcaggtcgcttcagaggtgttgtagtcg

SEQ ID NO: 233 - DNA, 111 bp, Artificial Sequence
  Description of Artificial Sequence Synthetic probe
gcgctggcgcagtatcgtgaactgggttggaggctcatcgttcctatattccacaccacttattattacagatgttatgctcgcaggtcaccaacgtaatctctattaccg

SEQ ID NO: 234 - DNA, 112 bp, Artificial Sequence
  Description of Artificial Sequence Synthetic probe
gctgtaatgcaagtagcgtatgcgctcagttggaggctcatcgttcctatattccacaccacttattattacagatgttatgctcgcaggtcaaggccgccaatgcctgacg

SEQ ID NO: 235 - DNA, 112 bp, Artificial Sequence
  Description of Artificial Sequence Synthetic probe
gcctgtagcaacagtaccacgaccagtgttggaggctcatcgttcctatattccacaccacttattattacagatgttatgctcgcaggtccaccacgtaataatgcaccaa

SEQ ID NO: 236 - DNA, 112 bp, Artificial Sequence
  Description of Artificial Sequence Synthetic probe
actacgctgaagctggtgacaacattggttggaggctcatcgttcctatattccacaccacttattattacagatgttatgctcgcaggtcgttgaggacgtattctcaatc

SEQ ID NO: 237 - DNA, 105 bp, Artificial Sequence
  Description of Artificial Sequence Synthetic probe
gctggtacttacgttcagatgttggaggctcatcgttcctatattccacaccacttattattacagatgttatgctcgcaggtcacggtgaacgccgttacatcc

SEQ ID NO: 238 - DNA, 110 bp, Artificial Sequence
  Description of Artificial Sequence Synthetic probe
gcaattcttaccacagcacgaagttggaggctcatcgttcctatattccacaccacttattattacagatgttatgctcgcaggtcatctagatgaagataatgaagtcg

SEQ ID NO: 239 - DNA, 108 bp, Artificial Sequence
  Description of Artificial Sequence Synthetic probe
gcggcggcaggcggtaacgccaggttggaggctcatcgttcctatattccacaccacttattattacagatgttatgctcgcaggtcacgcggttatctaccacggcg

SEQ ID NO: 240 - DNA, 112 bp, Artificial Sequence
  Description of Artificial Sequence Synthetic probe
gcacctacttgtccagcaccagccatgttggaggctcatcgttcctatattccacaccacttattattacagatgttatgctcgcaggtcaataccaccaccaatacaagca

SEQ ID NO: 241 - DNA, 108 bp, Artificial Sequence
  Description of Artificial Sequence Synthetic probe
gcgcggtaacatgccatattctgcgttggaggctcatcgttcctatattccacaccacttattattacagatgttatgctcgcaggtccctgaatgacatcacagtcg

SEQ ID NO: 242 - DNA, 112 bp, Artificial Sequence
  Description of Artificial Sequence Synthetic probe
aatcaggtcaaggaactgcaagcgttggaggctcatcgttcctatattccacaccacttattattacagatgttatgctcgcaggtcgtctcaatcatatgcaccggaatac

SEQ ID NO: 243 - DNA, 117 bp, Artificial Sequence
  Description of Artificial Sequence Synthetic probe
gaacatatgtgtatgacgatgcgcgggttggaggctcatcgttcctatattccacaccacttattattacagatgttatgctcgcaggtcgtacatgtcgcttatctgccagaaggt

SEQ ID NO: 244 - DNA, 116 bp, Artificial Sequence
  Description of Artificial Sequence Synthetic probe
cgtgtgcgtagtgacgagttggagagttggaggctcatcgttcctatattccacaccacttattattacagatgttatgctcgcaggtcagaatacgatgatgtaaggtacaccta

SEQ ID NO: 245 - DNA, 106 bp, Artificial Sequence
  Description of Artificial Sequence Synthetic probe
caggagttacttctgttccatgttggaggctcatcgttcctatattccacaccacttattattacagatgttatgctcgcaggtcttgaacaattagatcacctcg

SEQ ID NO: 246 - DNA, 117 bp, Artificial Sequence
  Description of Artificial Sequence Synthetic probe
cgtaatctccattaccgatggtcagatcgttggaggctcatcgttcctatattccacaccacttattattacagatgttatgctcgcaggtcacgtattctacctccactctcgtct

SEQ ID NO: 247 - DNA, 114 bp, Artificial Sequence
  Description of Artificial Sequence Synthetic probe
cattcgacgttctggtattacttgttggaggctcatcgttcctatattccacaccacttattattacagatgttatgctcgcaggtccacgctccgcatcagcagcaccacgtt

SEQ ID NO: 248 - DNA, 112 bp, Artificial Sequence
  Description of Artificial Sequence Synthetic probe
ctgaaccacggattactggagtgtcgttggaggctcatcgttcctatattccacaccacttattattacagatgttatgctcgcaggtcgcctgttactactgtaccacgac

SEQ ID NO: 249 - DNA, 111 bp, Artificial Sequence
  Description of Artificial Sequence Synthetic probe
gaatcgaacggtctcattaacagatgttggaggctcatcgttcctatattccacaccacttattattacagatgttatgctcgcaggtcgctttccagggatataagacgc

SEQ ID NO: 250 - DNA, 106 bp, Artificial Sequence
  Description of Artificial Sequence Synthetic probe
cccgcagagtcacactcggagttggaggctcatcgttcctatattccacaccacttattattacagatgttatgctcgcaggtcactcttggtactactcactagc

SEQ ID NO: 251 - DNA, 115 bp, Artificial Sequence
  Description of Artificial Sequence Synthetic probe
gagtctctttcaacctggattagatatgttggaggctcatcgttcctatattccacaccacttattattacagatgttatgctcgcaggtcaagattaatagcgtactttactcc

SEQ ID NO: 252 - DNA, 114 bp, Artificial Sequence
  Description of Artificial Sequence Synthetic probe
atcccgcagatactaggttcttaatgttggaggctcatcgttcctatattccacaccacttattattacagatgttatgctcgcaggtcgaactattcatattacaccctaagg

SEQ ID NO: 253 - DNA, 111 bp, Artificial Sequence
  Description of Artificial Sequence Synthetic probe
cagtgggctatcctaagccaaaggttggaggctcatcgttcctatattccacaccacttattattacagatgttatgctcgcaggtccataagcgaactaactatcactta

SEQ ID NO: 254 - DNA, 113 bp, Artificial Sequence
  Description of Artificial Sequence Synthetic probe
acaaagcgttctaaacgattagaactgttggaggctcatcgttcctatattccacaccacttattattacagatgttatgctcgcaggtccgagaaaggaaacaggatagtac

SEQ ID NO: 255 - DNA, 114 bp, Artificial Sequence
  Description of Artificial Sequence Synthetic probe
ccaatggagaagtctaaatgtccaagttggaggctcatcgttcctatattccacaccacttattattacagatgttatgctcgcaggtcttatcagagatacatgactcttagg

SEQ ID NO: 256 - DNA, 115 bp, Artificial Sequence
  Description of Artificial Sequence Synthetic probe
cgaatcactggactacatttatatttctgttggaggctcatcgttcctatattccacaccacttattattacagatgttatgctcgcaggtcagcgaacctttatatttgaccat

SEQ ID NO: 257 - DNA, 115 bp, Artificial Sequence
  Description of Artificial Sequence Synthetic probe
ctcaagtcttgccctgatagaattatgttggaggctcatcgttcctatattccacaccacttattattacagatgttatgctcgcaggtctcacgacttatctactttagaaatc

SEQ ID NO: 258 - DNA, 111 bp, Artificial Sequence
  Description of Artificial Sequence Synthetic probe
ggtgatcgttattatgatagtacggcgttggaggctcatcgttcctatattccacaccacttattattacagatgttatgctcgcaggtcctcggttaagggaattacgac

SEQ ID NO: 259 - DNA, 113 bp, Artificial Sequence
  Description of Artificial Sequence Synthetic probe
actcggatggtaggtttattaaagcgttggaggctcatcgttcctatattccacaccacttattattacagatgttatgctcgcaggtcgtgatcgttattatgatagtacgg

SEQ ID NO: 260 - DNA, 108 bp, Artificial Sequence
  Description of Artificial Sequence Synthetic probe
acaatcgttgtcgcactgcataggttggaggctcatcgttcctatattccacaccacttattattacagatgttatgctcgcaggtcgaacttggtctaccgtaccac

SEQ ID NO: 261 - DNA, 111 bp, Artificial Sequence
  Description of Artificial Sequence Synthetic probe
ggataatacaatcctaatacgtacggagttggaggctcatcgttcctatattccacaccacttattattacagatgttatgctcgcaggtcgctgctgtaactagggtagc

SEQ ID NO: 262 - DNA, 111 bp, Artificial Sequence
  Description of Artificial Sequence Synthetic probe
ctatattcaacgggtcacgggtaggttggaggctcatcgttcctatattccacaccacttattattacagatgttatgctcgcaggtctcattgattcgatctcgtaactc

SEQ ID NO: 263 - DNA, 108 bp, Artificial Sequence
  Description of Artificial Sequence Synthetic probe
aatgttattgtggttgcgtgttcggttggaggctcatcgttcctatattccacaccacttattattacagatgttatgctcgcaggtctactttggaagtgccctgac

SEQ ID NO: 264 - DNA, 109 bp, Artificial Sequence
  Description of Artificial Sequence Synthetic probe
catgtcttctagtacaggtttgccggttggaggctcatcgttcctatattccacaccacttattattacagatgttatgctcgcaggtctgtaagaggccgctaacttc

SEQ ID NO: 265 - DNA, 107 bp, Artificial Sequence
  Description of Artificial Sequence Synthetic probe
ctctggctcgtgggctcgggttggaggctcatcgttcctatattccacaccacttattattacagatgttatgctcgcaggtcttcttgagatagtccggtataatc

SEQ ID NO: 266 - DNA, 106 bp, Artificial Sequence
  Description of Artificial Sequence Synthetic probe
attcgatcacgatgggctggggttggaggctcatcgttcctatattccacaccacttattattacagatgttatgctcgcaggtcaatttcctgtgtcatacacgc

SEQ ID NO: 267 - DNA, 109 bp, Artificial Sequence
  Description of Artificial Sequence Synthetic probe
caattgatttagccactacaccttacgttggaggctcatcgttcctatattccacaccacttattattacagatgttatgctcgcaggtccactattctggcgaccacc

SEQ ID NO: 268 - DNA, 107 bp, Artificial Sequence
  Description of Artificial Sequence Synthetic probe
gataaagaagcgtcttgacccagtgttggaggctcatcgttcctatattccacaccacttattattacagatgttatgctcgcaggtcatctggtgctccttgacgc

SEQ ID NO: 269 - DNA, 108 bp, Artificial Sequence
  Description of Artificial Sequence Synthetic probe
gcaaatttagagagtgcatgcatggttggaggctcatcgttcctatattccacaccacttattattacagatgttatgctcgcaggtcggaagaggacggcatacaac

SEQ ID NO: 270 - DNA, 108 bp, Artificial Sequence
  Description of Artificial Sequence Synthetic probe
catttcatctagaccgctcgtgtgttggaggctcatcgttcctatattccacaccacttattattacagatgttatgctcgcaggtcgcttgaagtgtatgttgggac

SEQ ID NO: 271 - DNA, 103 bp, Artificial Sequence
  Description of Artificial Sequence Synthetic probe
gtcgccctcgtgctaacgtgttggaggctcatcgttcctatattccacaccacttattattacagatgttatgctcgcaggtcggttctttgatgtaccggtt

SEQ ID NO: 272 - DNA, 109 bp, Artificial Sequence
  Description of Artificial Sequence Synthetic probe
gctgatgacggtgaagtttatcagttggaggctcatcgttcctatattccacaccacttattattacagatgttatgctcgcaggtccattatcgcacatattgaccac

SEQ ID NO: 273 - DNA, 107 bp, Artificial Sequence
  Description of Artificial Sequence Synthetic probe
gaaattagctaaagggatatcgcggttggaggctcatcgttcctatattccacaccacttattattacagatgttatgctcgcaggtcaactttccgccaatcctgc

SEQ ID NO: 274 - DNA, 107 bp, Artificial Sequence
  Description of Artificial Sequence Synthetic probe
cacctacgttctcacctgcacgttggaggctcatcgttcctatattccacaccacttattattacagatgttatgctcgcaggtcattcgatagtaccagttacgtc

SEQ ID NO: 275 - DNA, 107 bp, Artificial Sequence
  Description of Artificial Sequence Synthetic probe
gttgcttatagcgtcgctgctgttggaggctcatcgttcctatattccacaccacttattattacagatgttatgctcgcaggtcctggttatcgagaagataaagg

SEQ ID NO: 276 - DNA, 104 bp, Artificial Sequence
  Description of Artificial Sequence Synthetic probe
gtaagcgtagcgatacgttgaggttggaggctcatcgttcctatattccacaccacttattattacagatgttatgctcgcaggtcgagtgaacgcaccactgg

SEQ ID NO: 277 - DNA, 106 bp, Artificial Sequence
  Description of Artificial Sequence Synthetic probe
tcaggtagagaatactcaggcgcgttggaggctcatcgttcctatattccacaccacttattattacagatgttatgctcgcaggtccggagaaggctaggttgtc

SEQ ID NO: 278 - DNA, 105 bp, Artificial Sequence
  Description of Artificial Sequence Synthetic probe
gcaacccactcccatggtgtgttggaggctcatcgttcctatattccacaccacttattattacagatgttatgctcgcaggtccgttcttcatcagacaatctg

SEQ ID NO: 279 - DNA, 109 bp, Artificial Sequence
  Description of Artificial Sequence Synthetic probe
gccctttcaggactttgatactgggttggaggctcatcgttcctatattccacaccacttattattacagatgttatgctcgcaggtctgtacggagacggagttatcg

SEQ ID NO: 280 - DNA, 108 bp, Artificial Sequence
  Description of Artificial Sequence Synthetic probe
acactgaccgattcatcctcgtggttggaggctcatcgttcctatattccacaccacttattattacagatgttatgctcgcaggtccttgaaagtgcgttaacaacc

SEQ ID NO: 281 - DNA, 108 bp, Artificial Sequence
  Description of Artificial Sequence Synthetic probe
cggaagcccaccaagtgagtacgttggaggctcatcgttcctatattccacaccacttattattacagatgttatgctcgcaggtccgaaaccagtttgtccttagtc

SEQ ID NO: 282 - DNA, 109 bp, Artificial Sequence
  Description of Artificial Sequence Synthetic probe
accagcttgtctttagtctgagaggttggaggctcatcgttcctatattccacaccacttattattacagatgttatgctcgcaggtcctttacgacgggtcatttcac

SEQ ID NO: 283 - DNA, 112 bp, Artificial Sequence
  Description of Artificial Sequence Synthetic probe
cattggtttgttctgtttgagaggcgttggaggctcatcgttcctatattccacaccacttattattacagatgttatgctcgcaggtcgattcatcttcgtgaattgtgac

SEQ ID NO: 284 - DNA, 110 bp, Artificial Sequence
  Description of Artificial Sequence Synthetic probe
ggactttgatactggaggagtcatagttggaggctcatcgttcctatattccacaccacttattattacagatgttatgctcgcaggtctgtacggaaacggagttatcg

SEQ ID NO: 285 - DNA, 107 bp, Artificial Sequence
  Description of Artificial Sequence Synthetic probe
atgctggaggagtcgtacgtttgttggaggctcatcgttcctatattccacaccacttattattacagatgttatgctcgcaggtcgtcgcgcacactaatagattc

SEQ ID NO: 286 - DNA, 110 bp, Artificial Sequence
  Description of Artificial Sequence Synthetic probe
aactaaacctacacggaattggttcgttggaggctcatcgttcctatattccacaccacttattattacagatgttatgctcgcaggtcgcagatacacgacgtttatgt

SEQ ID NO: 287 - DNA, 109 bp, Artificial Sequence
  Description of Artificial Sequence Synthetic probe
gccgcttcacctacgttaggaagttggaggctcatcgttcctatattccacaccacttattattacagatgttatgctcgcaggtccgtaaagatgagtctttaacgtc

SEQ ID NO: 288 - DNA, 108 bp, Artificial Sequence
  Description of Artificial Sequence Synthetic probe
gacgtttgtgcgtaatctcagacgttggaggctcatcgttcctatattccacaccacttattattacagatgttatgctcgcaggtcgaggaaaccgtattcgttcgt

SEQ ID NO: 289 - DNA, 110 bp, Artificial Sequence
  Description of Artificial Sequence Synthetic probe
acaacactttaccacttgagtggggttggaggctcatcgttcctatattccacaccacttattattacagatgttatgctcgcaggtcgtaactgcccatgtcaagatac

SEQ ID NO: 290 - DNA, 108 bp, Artificial Sequence
  Description of Artificial Sequence Synthetic probe
ccacgtttagttgaaccaccgcgttggaggctcatcgttcctatattccacaccacttattattacagatgttatgctcgcaggtctcaatacgccagttgttagttc

SEQ ID NO: 291 - DNA, 111 bp, Artificial Sequence
  Description of Artificial Sequence Synthetic probe
aatcgataataagtacggtgcatccgttggaggctcatcgttcctatattccacaccacttattattacagatgttatgctcgcaggtcgaagaatacattcgcgtacatc

SEQ ID NO: 292 - DNA, 112 bp, Artificial Sequence
  Description of Artificial Sequence Synthetic probe
aagcaagatcgagtcttcatagttggttggaggctcatcgttcctatattccacaccacttattattacagatgttatgctcgcaggtcgatatacacgatacctgattcgt

SEQ ID NO: 293 - DNA, 112 bp, Artificial Sequence
  Description of Artificial Sequence Synthetic probe
ccgatattcatacgagaaggtacacgttggaggctcatcgttcctatattccacaccacttattattacagatgttatgctcgcaggtccagtaactctattgtcaaacggt

SEQ ID NO: 294 - DNA, 109 bp, Artificial Sequence
  Description of Artificial Sequence Synthetic probe
gtagtgagtcgggtgtacgtctcgttggaggctcatcgttcctatattccacaccacttattattacagatgttatgctcgcaggtctcttcgatagcagacagatagt

SEQ ID NO: 295 - DNA, 109 bp, Artificial Sequence
  Description of Artificial Sequence Synthetic probe
acctacacggaattggttctcagtgttggaggctcatcgttcctatattccacaccacttattattacagatgttatgctcgcaggtcgatacacgacgtttgtgtgta

SEQ ID NO: 296 - DNA, 108 bp, Artificial Sequence
  Description of Artificial Sequence Synthetic probe
caacatcattagcttggtcgtggggttggaggctcatcgttcctatattccacaccacttattattacagatgttatgctcgcaggtcttgcgtgttaccaactcgtc

SEQ ID NO: 297 - DNA, 105 bp, Artificial Sequence
  Description of Artificial Sequence Synthetic probe
cggcacgtccgaatcgtatcagttggaggctcatcgttcctatattccacaccacttattattacagatgttatgctcgcaggtctcgtgtcccgtatatgttgg

SEQ ID NO: 298 - DNA, 108 bp, Artificial Sequence
  Description of Artificial Sequence Synthetic probe
aatagaggcccacaagtcttgttcgttggaggctcatcgttcctatattccacaccacttattattacagatgttatgctcgcaggtccgctctccactatgggtagt

SEQ ID NO: 299 - DNA, 111 bp, Artificial Sequence
  Description of Artificial Sequence Synthetic probe
gctacattaatcactatggacagacagttggaggctcatcgttcctatattccacaccacttattattacagatgttatgctcgcaggtcgatggtcgatctatcgtctct

SEQ ID NO: 300 - DNA, 110 bp, Artificial Sequence
  Description of Artificial Sequence Synthetic probe
gaagtgttattcaaactttggtcccgttggaggctcatcgttcctatattccacaccacttattattacagatgttatgctcgcaggtccttgaacccttggttcaaggt

SEQ ID NO: 301 - DNA, 24 bp, Artificial Sequence
  Description of Artificial Sequence Synthetic probe
gaacataacgcgacgttccagctg

SEQ ID NO: 302 - DNA, 25 bp, Artificial Sequence
  Description of Artificial Sequence Synthetic probe
gcgctggcgcagtatcgtgaactgg

SEQ ID NO: 303 - DNA, 28 bp, Artificial Sequence
  Description of Artificial Sequence Synthetic probe
gctgtaatgcaagtagcgtatgcgctca

SEQ ID NO: 304 - DNA, 27 bp, Artificial Sequence
  Description of Artificial Sequence Synthetic probe
gcctgtagcaacagtaccacgaccagt

SEQ ID NO: 305 - DNA, 27 bp, Artificial Sequence
  Description of Artificial Sequence Synthetic probe
actacgctgaagctggtgacaacattg

SEQ ID NO: 306 - DNA, 20 bp, Artificial Sequence
  Description of Artificial Sequence Synthetic probe
gctggtacttacgttcagat

SEQ ID NO: 307 - DNA, 22 bp, Artificial Sequence
  Description of Artificial Sequence Synthetic probe
gcaattcttaccacagcacgaa

SEQ ID NO: 308 - DNA, 23 bp, Artificial Sequence
  Description of Artificial Sequence Synthetic probe
gcggcggcaggcggtaacgccag

SEQ ID NO: 309 - DNA, 26 bp, Artificial Sequence
  Description of Artificial Sequence Synthetic probe
gcacctacttgtccagcaccagccat

SEQ ID NO: 310 - DNA, 24 bp, Artificial Sequence
  Description of Artificial Sequence Synthetic probe
gcgcggtaacatgccatattctgc

SEQ ID NO: 311 - DNA, 23 bp, Artificial Sequence
  Description of Artificial Sequence Synthetic probe
aatcaggtcaaggaactgcaagc

SEQ ID NO: 312 - DNA, 26 bp, Artificial Sequence
  Description of Artificial Sequence Synthetic probe
gaacatatgtgtatgacgatgcgcgg

SEQ ID NO: 313 - DNA, 25 bp, Artificial Sequence
  Description of Artificial Sequence Synthetic probe
cgtgtgcgtagtgacgagttggaga

SEQ ID NO: 314 - DNA, 21 bp, Artificial Sequence
  Description of Artificial Sequence Synthetic probe
caggagttacttctgttccat

SEQ ID NO: 315 - DNA, 28 bp, Artificial Sequence
  Description of Artificial Sequence Synthetic probe
cgtaatctccattaccgatggtcagatc

SEQ ID NO: 316 - DNA, 23 bp, Artificial Sequence
  Description of Artificial Sequence Synthetic probe
cattcgacgttctggtattactt

SEQ ID NO: 317 - DNA, 25 bp, Artificial Sequence
  Description of Artificial Sequence Synthetic probe
ctgaaccacggattactggagtgtc

SEQ ID NO: 318 - DNA, 21 bp, Artificial Sequence
  Description of Artificial Sequence Synthetic probe
gcttcagaggtgttgtagtcg

SEQ ID NO: 319 - DNA, 22 bp, Artificial Sequence
  Description of Artificial Sequence Synthetic probe
accaacgtaatctctattaccg

SEQ ID NO: 320 - DNA, 20 bp, Artificial Sequence
  Description of Artificial Sequence Synthetic probe
aaggccgccaatgcctgacg

SEQ ID NO: 321 - DNA, 21 bp, Artificial Sequence
  Description of Artificial Sequence Synthetic probe
caccacgtaataatgcaccaa

SEQ ID NO: 322 - DNA, 21 bp, Artificial Sequence
  Description of Artificial Sequence Synthetic probe
gttgaggacgtattctcaatc

SEQ ID NO: 323 - DNA, 21 bp, Artificial Sequence
  Description of Artificial Sequence Synthetic probe
acggtgaacgccgttacatcc

SEQ ID NO: 324 - DNA, 24 bp, Artificial Sequence
  Description of Artificial Sequence Synthetic probe
atctagatgaagataatgaagtcg

SEQ ID NO: 325 - DNA, 21 bp, Artificial Sequence
  Description of Artificial Sequence Synthetic probe
acgcggttatctaccacggcg

SEQ ID NO: 326 - DNA, 22 bp, Artificial Sequence
  Description of Artificial Sequence Synthetic probe
aataccaccaccaatacaagca

SEQ ID NO: 327 - DNA, 20 bp, Artificial Sequence
  Description of Artificial Sequence Synthetic probe
cctgaatgacatcacagtcg

SEQ ID NO: 328 - DNA, 25 bp, Artificial Sequence
  Description of Artificial Sequence Synthetic probe
gtctcaatcatatgcaccggaatac

SEQ ID NO: 329 - DNA, 27 bp, Artificial Sequence
  Description of Artificial Sequence Synthetic probe
gtacatgtcgcttatctgccagaaggt

SEQ ID NO: 330 - DNA, 27 bp, Artificial Sequence
  Description of Artificial Sequence Synthetic probe
agaatacgatgatgtaaggtacaccta

SEQ ID NO: 331 - DNA, 21 bp, Artificial Sequence
  Description of Artificial Sequence Synthetic probe
ttgaacaattagatcacctcg

SEQ ID NO: 332 - DNA, 25 bp, Artificial Sequence
  Description of Artificial Sequence Synthetic probe
acgtattctacctccactctcgtct

SEQ ID NO: 333 - DNA, 27 bp, Artificial Sequence
  Description of Artificial Sequence Synthetic probe
cacgctccgcatcagcagcaccacgtt

SEQ ID NO: 334 - DNA, 23 bp, Artificial Sequence
  Description of Artificial Sequence Synthetic probe
gcctgttactactgtaccacgac

SEQ ID NO: 335 - DNA, 27 bp, Artificial Sequence
  Description of Artificial Sequence Synthetic probe
gcagcacttgaccgccatgagtgacca

SEQ ID NO: 336 - DNA, 26 bp, Artificial Sequence
  Description of Artificial Sequence Synthetic probe
gtgatcactgatgcaccagatgaagt

SEQ ID NO: 337 - DNA, 28 bp, Artificial Sequence
  Description of Artificial Sequence Synthetic probe
gatattattgatcatggtgccaagccaa

SEQ ID NO: 338 - DNA, 28 bp, Artificial Sequence
  Description of Artificial Sequence Synthetic probe
gctgagcgtgaaggttcatggattatta

SEQ ID NO: 339 - DNA, 28 bp, Artificial Sequence
  Description of Artificial Sequence Synthetic probe
gcatcttgtgcagcctgaatagcagcgt

SEQ ID NO: 340 - DNA, 28 bp, Artificial Sequence
  Description of Artificial Sequence Synthetic probe
aagtccataattgcttgagtgtagtcat

SEQ ID NO: 341 - DNA, 28 bp, Artificial Sequence
  Description of Artificial Sequence Synthetic probe
gcttgctggttctgcacgtagcttactg

SEQ ID NO: 342 - DNA, 28 bp, Artificial Sequence
  Description of Artificial Sequence Synthetic probe
gcagcgctgtgcaagttcaatgtattct

SEQ ID NO: 343 - DNA, 28 bp, Artificial Sequence
  Description of Artificial Sequence Synthetic probe
gtataacactcggccagcgccaaggttc

SEQ ID NO: 344 - DNA, 22 bp, Artificial Sequence
  Description of Artificial Sequence Synthetic probe
accatgcagatacaatgaacca

SEQ ID NO: 345 - DNA, 24 bp, Artificial Sequence
  Description of Artificial Sequence Synthetic probe
catcaacagcttcttgaagcattc

SEQ ID NO: 346 - DNA, 26 bp, Artificial Sequence
  Description of Artificial Sequence Synthetic probe
aacatatcacctgatattctagtatc

SEQ ID NO: 347 - DNA, 20 bp, Artificial Sequence
  Description of Artificial Sequence Synthetic probe
gctgttgcttgcggatactg

SEQ ID NO: 348 - DNA, 27 bp, Artificial Sequence
  Description of Artificial Sequence Synthetic probe
aagagctaatgcagctattgcacttat

SEQ ID NO: 349 - DNA, 24 bp, Artificial Sequence
  Description of Artificial Sequence Synthetic probe
aacaagagcagaagttacagacgt

SEQ ID NO: 350 - DNA, 25 bp, Artificial Sequence
  Description of Artificial Sequence Synthetic probe
actcgtgaagaccatgcagatacaa

SEQ ID NO: 351 - DNA, 21 bp, Artificial Sequence
  Description of Artificial Sequence Synthetic probe
accatgcagatacaatgaacc

SEQ ID NO: 352 - DNA, 27 bp, Artificial Sequence
  Description of Artificial Sequence Synthetic probe
gcatctgctgcttctattgctcctact

SEQ ID NO: 353 - DNA, 23 bp, Artificial Sequence
  Description of Artificial Sequence Synthetic probe
gcacaagctggagataacatcgg

SEQ ID NO: 354 - DNA, 25 bp, Artificial Sequence
  Description of Artificial Sequence Synthetic probe
ctctatcagcttctactgcttcttc

SEQ ID NO: 355 - DNA, 28 bp, Artificial Sequence
  Description of Artificial Sequence Synthetic probe
agatgagattcatactatcgttggagct

SEQ ID NO: 356 - DNA, 23 bp, Artificial Sequence
  Description of Artificial Sequence Synthetic probe
catcaacagcttcttgaagcatt

SEQ ID NO: 357 - DNA, 27 bp, Artificial Sequence
  Description of Artificial Sequence Synthetic probe
gtcagcaatacgccaccaagctcctat

SEQ ID NO: 358 - DNA, 27 bp, Artificial Sequence
  Description of Artificial Sequence Synthetic probe
gcgcaatagagttgtataagagtgctg

SEQ ID NO: 359 - DNA, 23 bp, Artificial Sequence
  Description of Artificial Sequence Synthetic probe
ggcataataggatggatagatga

SEQ ID NO: 360 - DNA, 26 bp, Artificial Sequence
  Description of Artificial Sequence Synthetic probe
gtacattcacatatagaccatcttaa

SEQ ID NO: 361 - DNA, 24 bp, Artificial Sequence
  Description of Artificial Sequence Synthetic probe
ccataccagtatcttggcatattg

SEQ ID NO: 362 - DNA, 24 bp, Artificial Sequence
  Description of Artificial Sequence Synthetic probe
agatgaagcacaagctggagataa

SEQ ID NO: 363 - DNA, 27 bp, Artificial Sequence
  Description of Artificial Sequence Synthetic probe
ataatcattcacctccatcattcataa

SEQ ID NO: 364 - DNA, 25 bp, Artificial Sequence
  Description of Artificial Sequence Synthetic probe
gtacattcacatatagaccatctta

SEQ ID NO: 365 - DNA, 20 bp, Artificial Sequence
  Description of Artificial Sequence Synthetic probe
actccaccaggatgttgtcc

SEQ ID NO: 366 - DNA, 28 bp, Artificial Sequence
  Description of Artificial Sequence Synthetic probe
gcaatatcaatggtatcgaaggcactat

SEQ ID NO: 367 - DNA, 23 bp, Artificial Sequence
  Description of Artificial Sequence Synthetic probe
gtgccggtctcggttactcaatg

SEQ ID NO: 368 - DNA, 24 bp, Artificial Sequence
  Description of Artificial Sequence Synthetic probe
gtacattcacatatagaccatctt

SEQ ID NO: 369 - DNA, 28 bp, Artificial Sequence
  Description of Artificial Sequence Synthetic probe
agttccttcatatgactcagttgattga

SEQ ID NO: 370 - DNA, 23 bp, Artificial Sequence
  Description of Artificial Sequence Synthetic probe
cagcagttgttgctagaggtatg

SEQ ID NO: 371 - DNA, 28 bp, Artificial Sequence
  Description of Artificial Sequence Synthetic probe
gcaattctctgttgttgtcctccactca

SEQ ID NO: 372 - DNA, 28 bp, Artificial Sequence
  Description of Artificial Sequence Synthetic probe
ctattcctgataataagtgtgtcctcat

SEQ ID NO: 373 - DNA, 28 bp, Artificial Sequence
  Description of Artificial Sequence Synthetic probe
gtaattccaattacttctagctctggtg

SEQ ID NO: 374 - DNA, 22 bp, Artificial Sequence
  Description of Artificial Sequence Synthetic probe
ccatgcagatacaatgaaccag

SEQ ID NO: 375 - DNA, 28 bp, Artificial Sequence
  Description of Artificial Sequence Synthetic probe
ccttctgccattgtagaacaagctccat

SEQ ID NO: 376 - DNA, 25 bp, Artificial Sequence
  Description of Artificial Sequence Synthetic probe
caatcatgatagaattagatggaac

SEQ ID NO: 377 - DNA, 22 bp, Artificial Sequence
  Description of Artificial Sequence Synthetic probe
agtggtgaaggtgttcaacaag

SEQ ID NO: 378 - DNA, 23 bp, Artificial Sequence
  Description of Artificial Sequence Synthetic probe
cgcctcttcagaagcggatatca

SEQ ID NO: 379 - DNA, 25 bp, Artificial Sequence
  Description of Artificial Sequence Synthetic probe
ggcataataggatggatagatgagc

SEQ ID NO: 380 - DNA, 27 bp, Artificial Sequence
  Description of Artificial Sequence Synthetic probe
agttccttcatatgactcagttgattg

SEQ ID NO: 381 - DNA, 27 bp, Artificial Sequence
  Description of Artificial Sequence Synthetic probe
gcatggtagttcgccagccgctggaac

SEQ ID NO: 382 - DNA, 28 bp, Artificial Sequence
  Description of Artificial Sequence Synthetic probe
aatatcatggtcgtgtccaggcactggc

SEQ ID NO: 383 - DNA, 27 bp, Artificial Sequence
  Description of Artificial Sequence Synthetic probe
aacttacaactacgcgcacttgaatcg

SEQ ID NO: 384 - DNA, 28 bp, Artificial Sequence
  Description of Artificial Sequence Synthetic probe
gcaagttgaggagatgctggcatgattc

SEQ ID NO: 385 - DNA, 28 bp, Artificial Sequence
  Description of Artificial Sequence Synthetic probe
gcgataattgtaatgattcgtggtgtta

SEQ ID NO: 386 - DNA, 27 bp, Artificial Sequence
  Description of Artificial Sequence Synthetic probe
actgtggcagtctatgttccaattgta

SEQ ID NO: 387 - DNA, 21 bp, Artificial Sequence
  Description of Artificial Sequence Synthetic probe
gcgtcgcttcttgcgctcgcc

SEQ ID NO: 388 - DNA, 28 bp, Artificial Sequence
  Description of Artificial Sequence Synthetic probe
gccttcacaactacgttggaaggtcttc

SEQ ID NO: 389 - DNA, 27 bp, Artificial Sequence
  Description of Artificial Sequence Synthetic probe
gccttcacaactacgttggaaggtctt

SEQ ID NO: 390 - DNA, 27 bp, Artificial Sequence
  Description of Artificial Sequence Synthetic probe
gccgctgagcggcggcaagccgatggc

SEQ ID NO: 391 - DNA, 27 bp, Artificial Sequence
  Description of Artificial Sequence Synthetic probe
gccaagcggcattctggcgccagtgga

SEQ ID NO: 392 - DNA, 28 bp, Artificial Sequence
  Description of Artificial Sequence Synthetic probe
gccgtatatcatcggcaataaccgcacg

SEQ ID NO: 393 - DNA, 27 bp, Artificial Sequence
  Description of Artificial Sequence Synthetic probe
acgagccgagataggtctgcagcgtac

SEQ ID NO: 394 - DNA, 27 bp, Artificial Sequence
  Description of Artificial Sequence Synthetic probe
gcaatatcttcaccggcagccaccgcg

SEQ ID NO: 395 - DNA, 24 bp, Artificial Sequence
  Description of Artificial Sequence Synthetic probe
aataaccttaacgtcgccaacacg

SEQ ID NO: 396 - DNA, 24 bp, Artificial Sequence
  Description of Artificial Sequence Synthetic probe
gcggaactgcttggcgtagtaagc

SEQ ID NO: 397 - DNA, 28 bp, Artificial Sequence
  Description of Artificial Sequence Synthetic probe
gcgagaccggcggcaccatcgtctccag

SEQ ID NO: 398 - DNA, 28 bp, Artificial Sequence
  Description of Artificial Sequence Synthetic probe
gcggttcacctgttcgccttcgaacacg

SEQ ID NO: 399 - DNA, 28 bp, Artificial Sequence
  Description of Artificial Sequence Synthetic probe
actccatcgccatcaaggacatggccgg

SEQ ID NO: 400 - DNA, 23 bp, Artificial Sequence
  Description of Artificial Sequence Synthetic probe
gcctgatgcactacagcgcctgg

SEQ ID NO: 401 - DNA, 23 bp, Artificial Sequence
  Description of Artificial Sequence Synthetic probe
gcgcatccaggacggcgagtacg

SEQ ID NO: 402 - DNA, 28 bp, Artificial Sequence
  Description of Artificial Sequence Synthetic probe
gctggagaacgtcaaggtggtgatcatc

SEQ ID NO: 403 - DNA, 26 bp, Artificial Sequence
  Description of Artificial Sequence Synthetic probe
acgattggagaaggcagtgtgattgg

SEQ ID NO: 404 - DNA, 20 bp, Artificial Sequence
  Description of Artificial Sequence Synthetic probe
gccgcaataccgatattcca

SEQ ID NO: 405 - DNA, 23 bp, Artificial Sequence
  Description of Artificial Sequence Synthetic probe
gtgaaggtcgtgctcctatcggt

SEQ ID NO: 406 - DNA, 23 bp, Artificial Sequence
  Description of Artificial Sequence Synthetic probe
gctggtacttgtacttatatcga

SEQ ID NO: 407 - DNA, 27 bp, Artificial Sequence
  Description of Artificial Sequence Synthetic probe
gcgcatattgcattaatggctatagat

SEQ ID NO: 408 - DNA, 28 bp, Artificial Sequence
  Description of Artificial Sequence Synthetic probe
gcaattcttaccacagcacgaagaacag

SEQ ID NO: 409 - DNA, 28 bp, Artificial Sequence
  Description of Artificial Sequence Synthetic probe
gcatcttcatacaatacttctagcttac

SEQ ID NO: 410 - DNA, 27 bp, Artificial Sequence
  Description of Artificial Sequence Synthetic probe
gcttcagcgccattaccgccaccagct

SEQ ID NO: 411 - DNA, 27 bp, Artificial Sequence
  Description of Artificial Sequence Synthetic probe
gttcacacaacgcgccgactagaatcc

SEQ ID NO: 412 - DNA, 27 bp, Artificial Sequence
  Description of Artificial Sequence Synthetic probe
gcgcacctacaatcgccattactacac

SEQ ID NO: 413 - DNA, 27 bp, Artificial Sequence
  Description of Artificial Sequence Synthetic probe
agcgcacatgtgacagcgtgtaggtta

SEQ ID NO: 414 - DNA, 28 bp, Artificial Sequence
  Description of Artificial Sequence Synthetic probe
cgaatggatatgtaccatggtcgatatc

SEQ ID NO: 415 - DNA, 28 bp, Artificial Sequence
  Description of Artificial Sequence Synthetic probe
actacaacagcaaccgcattacaatggc

SEQ ID NO: 416 - DNA, 24 bp, Artificial Sequence
  Description of Artificial Sequence Synthetic probe
agcttcagataagtacctatctga

SEQ ID NO: 417 - DNA, 22 bp, Artificial Sequence
  Description of Artificial Sequence Synthetic probe
cgtattgctcgaatacatgata

SEQ ID NO: 418 - DNA, 24 bp, Artificial Sequence
  Description of Artificial Sequence Synthetic probe
gcgaccagttgttatcgaccgtgt

SEQ ID NO: 419 - DNA, 28 bp, Artificial Sequence
  Description of Artificial Sequence Synthetic probe
caattacattgtctgttgcgtagatacc

SEQ ID NO: 420 - DNA, 27 bp, Artificial Sequence
  Description of Artificial Sequence Synthetic probe
gcaccactctatagcagtagcgtattg

SEQ ID NO: 421 - DNA, 28 bp, Artificial Sequence
  Description of Artificial Sequence Synthetic probe
acagtccgaataagatacgactattcga

SEQ ID NO: 422 - DNA, 28 bp, Artificial Sequence
  Description of Artificial Sequence Synthetic probe
agatgcaataacaggtcgaatattaatt

SEQ ID NO: 423 - DNA, 26 bp, Artificial Sequence
  Description of Artificial Sequence Synthetic probe
agatgcaataacaggtcgaatattaa

SEQ ID NO: 424 - DNA, 25 bp, Artificial Sequence
  Description of Artificial Sequence Synthetic probe
gaatcgaacggtctcattaacagat

SEQ ID NO: 425 - DNA, 20 bp, Artificial Sequence
  Description of Artificial Sequence Synthetic probe
cccgcagagtcacactcgga

SEQ ID NO: 426 - DNA, 27 bp, Artificial Sequence
  Description of Artificial Sequence Synthetic probe
gagtctctttcaacctggattagatat

SEQ ID NO: 427 - DNA, 25 bp, Artificial Sequence
  Description of Artificial Sequence Synthetic probe
atcccgcagatactaggttcttaat

SEQ ID NO: 428 - DNA, 23 bp, Artificial Sequence
  Description of Artificial Sequence Synthetic probe
cagtgggctatcctaagccaaag

SEQ ID NO: 429 - DNA, 26 bp, Artificial Sequence
  Description of Artificial Sequence Synthetic probe
acaaagcgttctaaacgattagaact

SEQ ID NO: 430 - DNA, 25 bp, Artificial Sequence
  Description of Artificial Sequence Synthetic probe
ccaatggagaagtctaaatgtccaa

SEQ ID NO: 431 - DNA, 28 bp, Artificial Sequence
  Description of Artificial Sequence Synthetic probe
cgaatcactggactacatttatatttct

SEQ ID NO: 432 - DNA, 26 bp, Artificial Sequence
  Description of Artificial Sequence Synthetic probe
ctcaagtcttgccctgatagaattat

SEQ ID NO: 433 - DNA, 26 bp, Artificial Sequence
  Description of Artificial Sequence Synthetic probe
ggtgatcgttattatgatagtacggc

SEQ ID NO: 434 - DNA, 25 bp, Artificial Sequence
  Description of Artificial Sequence Synthetic probe
actcggatggtaggtttattaaagc

SEQ ID NO: 435 - DNA, 23 bp, Artificial Sequence
  Description of Artificial Sequence Synthetic probe
acaatcgttgtcgcactgcatag

SEQ ID NO: 436 - DNA, 27 bp, Artificial Sequence
  Description of Artificial Sequence Synthetic probe
ggataatacaatcctaatacgtacgga

SEQ ID NO: 437 - DNA, 24 bp, Artificial Sequence
  Description of Artificial Sequence Synthetic probe
ctatattcaacgggtcacgggtag

SEQ ID NO: 438 - DNA, 24 bp, Artificial Sequence
  Description of Artificial Sequence Synthetic probe
aatgttattgtggttgcgtgttcg

SEQ ID NO: 439 - DNA, 25 bp, Artificial Sequence
  Description of Artificial Sequence Synthetic probe
catgtcttctagtacaggtttgccg

SEQ ID NO: 440 - DNA, 19 bp, Artificial Sequence
  Description of Artificial Sequence Synthetic probe
ctctggctcgtgggctcgg

SEQ ID NO: 441 - DNA, 21 bp, Artificial Sequence
  Description of Artificial Sequence Synthetic probe
attcgatcacgatgggctggg

SEQ ID NO: 442 - DNA, 26 bp, Artificial Sequence
  Description of Artificial Sequence Synthetic probe
caattgatttagccactacaccttac

SEQ ID NO: 443 - DNA, 24 bp, Artificial Sequence
  Description of Artificial Sequence Synthetic probe
gataaagaagcgtcttgacccagt

SEQ ID NO: 444 - DNA, 24 bp, Artificial Sequence
  Description of Artificial Sequence Synthetic probe
gcaaatttagagagtgcatgcatg

SEQ ID NO: 445 - DNA, 23 bp, Artificial Sequence
  Description of Artificial Sequence Synthetic probe
catttcatctagaccgctcgtgt

SEQ ID NO: 446 - DNA, 19 bp, Artificial Sequence
  Description of Artificial Sequence Synthetic probe
gtcgccctcgtgctaacgt

SEQ ID NO: 447 - DNA, 23 bp, Artificial Sequence
  Description of Artificial Sequence Synthetic probe
gctgatgacggtgaagtttatca

SEQ ID NO: 448 - DNA, 24 bp, Artificial Sequence
  Description of Artificial Sequence Synthetic probe
gaaattagctaaagggatatcgcg

SEQ ID NO: 449 - DNA, 21 bp, Artificial Sequence
  Description of Artificial Sequence Synthetic probe
cacctacgttctcacctgcac

SEQ ID NO: 450 - DNA, 21 bp, Artificial Sequence
  Description of Artificial Sequence Synthetic probe
gttgcttatagcgtcgctgct

SEQ ID NO: 451 - DNA, 22 bp, Artificial Sequence
  Description of Artificial Sequence Synthetic probe
gtaagcgtagcgatacgttgag

SEQ ID NO: 452 - DNA, 23 bp, Artificial Sequence
  Description of Artificial Sequence Synthetic probe
tcaggtagagaatactcaggcgc

SEQ ID NO: 453 - DNA, 20 bp, Artificial Sequence
  Description of Artificial Sequence Synthetic probe
gcaacccactcccatggtgt

SEQ ID NO: 454 - DNA, 25 bp, Artificial Sequence
  Description of Artificial Sequence Synthetic probe
aactaaacctacacggaattggttc

SEQ ID NO: 455 - DNA, 22 bp, Artificial Sequence
  Description of Artificial Sequence Synthetic probe
gccgcttcacctacgttaggaa

SEQ ID NO: 456 - DNA, 23 bp, Artificial Sequence
  Description of Artificial Sequence Synthetic probe
gacgtttgtgcgtaatctcagac

SEQ ID NO: 457 - DNA, 24 bp, Artificial Sequence
  Description of Artificial Sequence Synthetic probe
acaacactttaccacttgagtggg

SEQ ID NO: 458 - DNA, 22 bp, Artificial Sequence
  Description of Artificial Sequence Synthetic probe
ccacgtttagttgaaccaccgc

SEQ ID NO: 459 - DNA, 25 bp, Artificial Sequence
  Description of Artificial Sequence Synthetic probe
aatcgataataagtacggtgcatcc

SEQ ID NO: 460 - DNA, 25 bp, Artificial Sequence
  Description of Artificial Sequence Synthetic probe
aagcaagatcgagtcttcatagttg

SEQ ID NO: 461 - DNA, 25 bp, Artificial Sequence
  Description of Artificial Sequence Synthetic probe
ccgatattcatacgagaaggtacac

SEQ ID NO: 462 - DNA, 23 bp, Artificial Sequence
  Description of Artificial Sequence Synthetic probe
gtagtgagtcgggtgtacgtctc

SEQ ID NO: 463 - DNA, 24 bp, Artificial Sequence
  Description of Artificial Sequence Synthetic probe
acctacacggaattggttctcagt

SEQ ID NO: 464 - DNA, 24 bp, Artificial Sequence
  Description of Artificial Sequence Synthetic probe
caacatcattagcttggtcgtggg

SEQ ID NO: 465 - DNA, 21 bp, Artificial Sequence
  Description of Artificial Sequence Synthetic probe
cggcacgtccgaatcgtatca

SEQ ID NO: 466 - DNA, 24 bp, Artificial Sequence
  Description of Artificial Sequence Synthetic probe
aatagaggcccacaagtcttgttc

SEQ ID NO: 467 - DNA, 26 bp, Artificial Sequence
  Description of Artificial Sequence Synthetic probe
gctacattaatcactatggacagaca

SEQ ID NO: 468 - DNA, 25 bp, Artificial Sequence
  Description of Artificial Sequence Synthetic probe
gaagtgttattcaaactttggtccc

SEQ ID NO: 469 - DNA, 23 bp, Artificial Sequence
  Description of Artificial Sequence Synthetic probe
catcgcaccaacaacaataatcg

SEQ ID NO: 470 - DNA, 24 bp, Artificial Sequence
  Description of Artificial Sequence Synthetic probe
atcttgatattcaagtctatgacg

SEQ ID NO: 471 - DNA, 21 bp, Artificial Sequence
  Description of Artificial Sequence Synthetic probe
caatatgaagctgacgacgcg

SEQ ID NO: 472 - DNA, 20 bp, Artificial Sequence
  Description of Artificial Sequence Synthetic probe
ggtaaggcttacggtctcat

SEQ ID NO: 473 - DNA, 26 bp, Artificial Sequence
  Description of Artificial Sequence Synthetic probe
accacgttgaatatcaccttcggcat

SEQ ID NO: 474 - DNA, 26 bp, Artificial Sequence
  Description of Artificial Sequence Synthetic probe
atcttcgcactgaataataagaacat

SEQ ID NO: 475 - DNA, 21 bp, Artificial Sequence
  Description of Artificial Sequence Synthetic probe
aagatgaacaggctactgcaa

SEQ ID NO: 476 - DNA, 24 bp, Artificial Sequence
  Description of Artificial Sequence Synthetic probe
ctcgtgcgagtattccttaagtgt

SEQ ID NO: 477 - DNA, 24 bp, Artificial Sequence
  Description of Artificial Sequence Synthetic probe
gttcacacatcgccacaatatgat

SEQ ID NO: 478 - DNA, 23 bp, Artificial Sequence
  Description of Artificial Sequence Synthetic probe
ggatgataagacacatccaattc

SEQ ID NO: 479 - DNA, 24 bp, Artificial Sequence
  Description of Artificial Sequence Synthetic probe
gtccaacaactataacagaacgtc

SEQ ID NO: 480 - DNA, 27 bp, Artificial Sequence
  Description of Artificial Sequence Synthetic probe
attccattatattcaacaggattgtga

SEQ ID NO: 481 - DNA, 21 bp, Artificial Sequence
  Description of Artificial Sequence Synthetic probe
cgtatatgtagctcaagttgc

SEQ ID NO: 482 - DNA, 24 bp, Artificial Sequence
  Description of Artificial Sequence Synthetic probe
catacacttcagctataagaccat

SEQ ID NO: 483 - DNA, 22 bp, Artificial Sequence
  Description of Artificial Sequence Synthetic probe
gtataatggtggctagaggtga

SEQ ID NO: 484 - DNA, 21 bp, Artificial Sequence
  Description of Artificial Sequence Synthetic probe
aatacttacaatgcctgagga

SEQ ID NO: 485 - DNA, 22 bp, Artificial Sequence
  Description of Artificial Sequence Synthetic probe
cctgaggatgataagacacatc

SEQ ID NO: 486 - DNA, 25 bp, Artificial Sequence
  Description of Artificial Sequence Synthetic probe
acatgaactgatattagttctccaa

SEQ ID NO: 487 - DNA, 24 bp, Artificial Sequence
  Description of Artificial Sequence Synthetic probe
gtagaggacgtattcacaatcact

SEQ ID NO: 488 - DNA, 25 bp, Artificial Sequence
  Description of Artificial Sequence Synthetic probe
ccatctcatccacagttaatatatc

SEQ ID NO: 489 - DNA, 24 bp, Artificial Sequence
  Description of Artificial Sequence Synthetic probe
agcagagagaatagtaagaggaga

SEQ ID NO: 490 - DNA, 22 bp, Artificial Sequence
  Description of Artificial Sequence Synthetic probe
gtccaacaactataacagaacg

SEQ ID NO: 491 - DNA, 20 bp, Artificial Sequence
  Description of Artificial Sequence Synthetic probe
gtggtggatatcctgttacc

SEQ ID NO: 492 - DNA, 28 bp, Artificial Sequence
  Description of Artificial Sequence Synthetic probe
agcattaattatagattataatgtataa

SEQ ID NO: 493 - DNA, 23 bp, Artificial Sequence
  Description of Artificial Sequence Synthetic probe
actaatccaacttctactgctat

SEQ ID NO: 494 - DNA, 25 bp, Artificial Sequence
  Description of Artificial Sequence Synthetic probe
acataggtgcaggtagaatagtata

SEQ ID NO: 495 - DNA, 25 bp, Artificial Sequence
  Description of Artificial Sequence Synthetic probe
ataatgaataacagcaggtgtatta

SEQ ID NO: 496 - DNA, 21 bp, Artificial Sequence
  Description of Artificial Sequence Synthetic probe
aggacgtattcacaatcactg

SEQ ID NO: 497 - DNA, 20 bp, Artificial Sequence
  Description of Artificial Sequence Synthetic probe
actgaatatggttcgtctca

SEQ ID NO: 498 - DNA, 22 bp, Artificial Sequence
  Description of Artificial Sequence Synthetic probe
acataggtgcaggtagaatagt

SEQ ID NO: 499 - DNA, 20 bp, Artificial Sequence
  Description of Artificial Sequence Synthetic probe
gtaggaccgtcgtgtccaag

SEQ ID NO: 500 - DNA, 27 bp, Artificial Sequence
  Description of Artificial Sequence Synthetic probe
gtattgaaggtactattagcgatatgc

SEQ ID NO: 501 - DNA, 24 bp, Artificial Sequence
  Description of Artificial Sequence Synthetic probe
ggattattataatgcagctagaag

SEQ ID NO: 502 - DNA, 23 bp, Artificial Sequence
  Description of Artificial Sequence Synthetic probe
acataggtgcaggtagaatagta

SEQ ID NO: 503 - DNA, 27 bp, Artificial Sequence
  Description of Artificial Sequence Synthetic probe
gttatatcttcaattatacattcctgc

SEQ ID NO: 504 - DNA, 21 bp, Artificial Sequence
  Description of Artificial Sequence Synthetic probe
gcatcaccaggtgcagcaagt

SEQ ID NO: 505 - DNA, 25 bp, Artificial Sequence
  Description of Artificial Sequence Synthetic probe
agtaagagcctcttcttggtcatga

SEQ ID NO: 506 - DNA, 23 bp, Artificial Sequence
  Description of Artificial Sequence Synthetic probe
cggcatcatctaacaattcttct

SEQ ID NO: 507 - DNA, 21 bp, Artificial Sequence
  Description of Artificial Sequence Synthetic probe
taccatcttctccatgtgtat

SEQ ID NO: 508 - DNA, 23 bp, Artificial Sequence
  Description of Artificial Sequence Synthetic probe
gatgataagacacatccaattcc

SEQ ID NO: 509 - DNA, 20 bp, Artificial Sequence
  Description of Artificial Sequence Synthetic probe
cctgtaactgtccactgagc

SEQ ID NO: 510 - DNA, 24 bp, Artificial Sequence
  Description of Artificial Sequence Synthetic probe
agcaatagttccatcaggagcatc

SEQ ID NO: 511 - DNA, 22 bp, Artificial Sequence
  Description of Artificial Sequence Synthetic probe
actgaagctggatatgttggag

SEQ ID NO: 512 - DNA, 21 bp, Artificial Sequence
  Description of Artificial Sequence Synthetic probe
gccagacttccgccacaacct

SEQ ID NO: 513 - DNA, 23 bp, Artificial Sequence
  Description of Artificial Sequence Synthetic probe
gcagcagttgtacctacaactaa

SEQ ID NO: 514 - DNA, 28 bp, Artificial Sequence
  Description of Artificial Sequence Synthetic probe
gttatatcttcaattatacattcctgcg

SEQ ID NO: 515 - DNA, 24 bp, Artificial Sequence
  Description of Artificial Sequence Synthetic probe
acagcaaccgcaagttcttgacat

SEQ ID NO: 516 - DNA, 24 bp, Artificial Sequence
  Description of Artificial Sequence Synthetic probe
gttctggtagctgcttctactgta

SEQ ID NO: 517 - DNA, 23 bp, Artificial Sequence
  Description of Artificial Sequence Synthetic probe
gagtgttgtatgatagtctcggt

SEQ ID NO: 518 - DNA, 26 bp, Artificial Sequence
  Description of Artificial Sequence Synthetic probe
acatggctctggaagatgtgctgatc

SEQ ID NO: 519 - DNA, 25 bp, Artificial Sequence
  Description of Artificial Sequence Synthetic probe
ccgttgtcaatccagttagtagact

SEQ ID NO: 520 - DNA, 24 bp, Artificial Sequence
  Description of Artificial Sequence Synthetic probe
cttatcgacataatcctgataatc

SEQ ID NO: 521 - DNA, 21 bp, Artificial Sequence
  Description of Artificial Sequence Synthetic probe
aatgtattcataccgtcaagt

SEQ ID NO: 522 - DNA, 20 bp, Artificial Sequence
  Description of Artificial Sequence Synthetic probe
ctaacagtcctgccgactac

SEQ ID NO: 523 - DNA, 21 bp, Artificial Sequence
  Description of Artificial Sequence Synthetic probe
ctaacagtcctgccgactact

SEQ ID NO: 524 - DNA, 24 bp, Artificial Sequence
  Description of Artificial Sequence Synthetic probe
gaatggcaggccaagctgaaggcg

SEQ ID NO: 525 - DNA, 28 bp, Artificial Sequence
  Description of Artificial Sequence Synthetic probe
ccagaccggagtggacaacgtcgaggcg

SEQ ID NO: 526 - DNA, 21 bp, Artificial Sequence
  Description of Artificial Sequence Synthetic probe
gcatgatggtcaacaaggtgc

SEQ ID NO: 527 - DNA, 24 bp, Artificial Sequence
  Description of Artificial Sequence Synthetic probe
gtactgatattcaccatactgccg

SEQ ID NO: 528 - DNA, 21 bp, Artificial Sequence
  Description of Artificial Sequence Synthetic probe
ggtatatggcacgccaatcgc

SEQ ID NO: 529 - DNA, 23 bp, Artificial Sequence
  Description of Artificial Sequence Synthetic probe
ctcggtgaacacctcctggcacg

SEQ ID NO: 530 - DNA, 25 bp, Artificial Sequence
  Description of Artificial Sequence Synthetic probe
catgtagtgccgtagaccttcacca

SEQ ID NO: 531 - DNA, 27 bp, Artificial Sequence
  Description of Artificial Sequence Synthetic probe
ttctgcctgatggacgtctccggctcg

SEQ ID NO: 532 - DNA, 28 bp, Artificial Sequence
  Description of Artificial Sequence Synthetic probe
gcgcagcatctgacgcaggatggtctcg

SEQ ID NO: 533 - DNA, 27 bp, Artificial Sequence
  Description of Artificial Sequence Synthetic probe
atcgacgtgttccgcatcttcgacgcg

SEQ ID NO: 534 - DNA, 27 bp, Artificial Sequence
  Description of Artificial Sequence Synthetic probe
taccacatggtcgatctcgacgactgc

SEQ ID NO: 535 - DNA, 24 bp, Artificial Sequence
  Description of Artificial Sequence Synthetic probe
cttcgagtgcctgcacgagctgaa

SEQ ID NO: 536 - DNA, 23 bp, Artificial Sequence
  Description of Artificial Sequence Synthetic probe
accgataacgacgaccgcatcaa

SEQ ID NO: 537 - DNA, 19 bp, Artificial Sequence
  Description of Artificial Sequence Synthetic probe
ggacagattacaattggcg

SEQ ID NO: 538 - DNA, 22 bp, Artificial Sequence
  Description of Artificial Sequence Synthetic probe
ccattgtccaccagctgaaccg

SEQ ID NO: 539 - DNA, 23 bp, Artificial Sequence
  Description of Artificial Sequence Synthetic probe
agatctggtgaagttcgtatgat

SEQ ID NO: 540 - DNA, 26 bp, Artificial Sequence
  Description of Artificial Sequence Synthetic probe
atcagaagatgatatcgttacgtcat

SEQ ID NO: 541 - DNA, 20 bp, Artificial Sequence
  Description of Artificial Sequence Synthetic probe
gccagcaggttatacactcg

SEQ ID NO: 542 - DNA, 24 bp, Artificial Sequence
  Description of Artificial Sequence Synthetic probe
atctagatgaagataatgaagtcg

SEQ ID NO: 543 - DNA, 21 bp, Artificial Sequence
  Description of Artificial Sequence Synthetic probe
cacaataccagttgtattacg

SEQ ID NO: 544 - DNA, 24 bp, Artificial Sequence
  Description of Artificial Sequence Synthetic probe
actcttgatatattcttgtaagcg

SEQ ID NO: 545 - DNA, 27 bp, Artificial Sequence
  Description of Artificial Sequence Synthetic probe
cacgatatccaagataatgattggcta

SEQ ID NO: 546 - DNA, 28 bp, Artificial Sequence
  Description of Artificial Sequence Synthetic probe
actcattatcgactgttacatcgactga

SEQ ID NO: 547 - DNA, 24 bp, Artificial Sequence
  Description of Artificial Sequence Synthetic probe
gtgccttagattgttcagaacaat

SEQ ID NO: 548 - DNA, 20 bp, Artificial Sequence
  Description of Artificial Sequence Synthetic probe
ctctctaatatgatgtccat

SEQ ID NO: 549 - DNA, 20 bp, Artificial Sequence
  Description of Artificial Sequence Synthetic probe
ggtgctaagaggtcatcgga

SEQ ID NO: 550 - DNA, 28 bp, Artificial Sequence
  Description of Artificial Sequence Synthetic probe
ggaagaatagttattcttgataatgtat

SEQ ID NO: 551 - DNA, 20 bp, Artificial Sequence
  Description of Artificial Sequence Synthetic probe
acaatgtatcaaggccagct

SEQ ID NO: 552 - DNA, 22 bp, Artificial Sequence
  Description of Artificial Sequence Synthetic probe
cagaacgatacggtgctgtata

SEQ ID NO: 553 - DNA, 21 bp, Artificial Sequence
  Description of Artificial Sequence Synthetic probe
gttgtggctaatgtgccagtt

SEQ ID NO: 554 - DNA, 25 bp, Artificial Sequence
  Description of Artificial Sequence Synthetic probe
acagccaatgtcacctaagtcaaca

SEQ ID NO: 555 - DNA, 25 bp, Artificial Sequence
  Description of Artificial Sequence Synthetic probe
cgttgtaacgtatatgaatagttga

SEQ ID NO: 556 - DNA, 20 bp, Artificial Sequence
  Description of Artificial Sequence Synthetic probe
gccatagtgagagtagtgaa

SEQ ID NO: 557 - DNA, 22 bp, Artificial Sequence
  Description of Artificial Sequence Synthetic probe
acacatacggccatagtgagag

SEQ ID NO: 558 - DNA, 22 bp, Artificial Sequence
  Description of Artificial Sequence Synthetic probe
gctttccagggatataagacgc

SEQ ID NO: 559 - DNA, 22 bp, Artificial Sequence
  Description of Artificial Sequence Synthetic probe
actcttggtactactcactagc

SEQ ID NO: 560 - DNA, 24 bp, Artificial Sequence
  Description of Artificial Sequence Synthetic probe
aagattaatagcgtactttactcc

SEQ ID NO: 561 - DNA, 25 bp, Artificial Sequence
  Description of Artificial Sequence Synthetic probe
gaactattcatattacaccctaagg

SEQ ID NO: 562 - DNA, 24 bp, Artificial Sequence
  Description of Artificial Sequence Synthetic probe
cataagcgaactaactatcactta

SEQ ID NO: 563 - DNA, 23 bp, Artificial Sequence
  Description of Artificial Sequence Synthetic probe
cgagaaaggaaacaggatagtac

SEQ ID NO: 564 - DNA, 25 bp, Artificial Sequence
  Description of Artificial Sequence Synthetic probe
ttatcagagatacatgactcttagg

SEQ ID NO: 565 - DNA, 23 bp, Artificial Sequence
  Description of Artificial Sequence Synthetic probe
agcgaacctttatatttgaccat

SEQ ID NO: 566 - DNA, 25 bp, Artificial Sequence
  Description of Artificial Sequence Synthetic probe
tcacgacttatctactttagaaatc

SEQ ID NO: 567 - DNA, 21 bp, Artificial Sequence
  Description of Artificial Sequence Synthetic probe
ctcggttaagggaattacgac

SEQ ID NO: 568 - DNA, 24 bp, Artificial Sequence
  Description of Artificial Sequence Synthetic probe
gtgatcgttattatgatagtacgg

SEQ ID NO: 569 - DNA, 21 bp, Artificial Sequence
  Description of Artificial Sequence Synthetic probe
gaacttggtctaccgtaccac

SEQ ID NO: 570 - DNA, 20 bp, Artificial Sequence
  Description of Artificial Sequence Synthetic probe
gctgctgtaactagggtagc

SEQ ID NO: 571 - DNA, 23 bp, Artificial Sequence
  Description of Artificial Sequence Synthetic probe
tcattgattcgatctcgtaactc

SEQ ID NO: 572 - DNA, 20 bp, Artificial Sequence
  Description of Artificial Sequence Synthetic probe
tactttggaagtgccctgac

SEQ ID NO: 573 - DNA, 20 bp, Artificial Sequence
  Description of Artificial Sequence Synthetic probe
tgtaagaggccgctaacttc

SEQ ID NO: 574 - DNA, 24 bp, Artificial Sequence
  Description of Artificial Sequence Synthetic probe
ttcttgagatagtccggtataatc

SEQ ID NO: 575 - DNA, 21 bp, Artificial Sequence
  Description of Artificial Sequence Synthetic probe
aatttcctgtgtcatacacgc

SEQ ID NO: 576 - DNA, 19 bp, Artificial Sequence
  Description of Artificial Sequence Synthetic probe
cactattctggcgaccacc

SEQ ID NO: 577 - DNA, 19 bp, Artificial Sequence
  Description of Artificial Sequence Synthetic probe
atctggtgctccttgacgc

SEQ ID NO: 578 - DNA, 20 bp, Artificial Sequence
  Description of Artificial Sequence Synthetic probe
ggaagaggacggcatacaac

SEQ ID NO: 579 - DNA, 21 bp, Artificial Sequence
  Description of Artificial Sequence Synthetic probe
gcttgaagtgtatgttgggac

SEQ ID NO: 580 - DNA, 20 bp, Artificial Sequence
  Description of Artificial Sequence Synthetic probe
ggttctttgatgtaccggtt

SEQ ID NO: 581 - DNA, 22 bp, Artificial Sequence
  Description of Artificial Sequence Synthetic probe
cattatcgcacatattgaccac

SEQ ID NO: 582 - DNA, 19 bp, Artificial Sequence
  Description of Artificial Sequence Synthetic probe
aactttccgccaatcctgc

SEQ ID NO: 583 - DNA, 22 bp, Artificial Sequence
  Description of Artificial Sequence Synthetic probe
attcgatagtaccagttacgtc

SEQ ID NO: 584 - DNA, 22 bp, Artificial Sequence
  Description of Artificial Sequence Synthetic probe
ctggttatcgagaagataaagg

SEQ ID NO: 585 - DNA, 18 bp, Artificial Sequence
  Description of Artificial Sequence Synthetic probe
gagtgaacgcaccactgg

SEQ ID NO: 586 - DNA, 19 bp, Artificial Sequence
  Description of Artificial Sequence Synthetic probe
cggagaaggctaggttgtc

SEQ ID NO: 587 - DNA, 21 bp, Artificial Sequence
  Description of Artificial Sequence Synthetic probe
cgttcttcatcagacaatctg

SEQ ID NO: 588 - DNA, 21 bp, Artificial Sequence
  Description of Artificial Sequence Synthetic probe
gcagatacacgacgtttatgt

SEQ ID NO: 589 - DNA, 23 bp, Artificial Sequence
  Description of Artificial Sequence Synthetic probe
cgtaaagatgagtctttaacgtc

SEQ ID NO: 590 - DNA, 21 bp, Artificial Sequence
  Description of Artificial Sequence Synthetic probe
gaggaaaccgtattcgttcgt

SEQ ID NO: 591 - DNA, 22 bp, Artificial Sequence
  Description of Artificial Sequence Synthetic probe
gtaactgcccatgtcaagatac

SEQ ID NO: 592 - DNA, 22 bp, Artificial Sequence
  Description of Artificial Sequence Synthetic probe
tcaatacgccagttgttagttc

SEQ ID NO: 593 - DNA, 22 bp, Artificial Sequence
  Description of Artificial Sequence Synthetic probe
gaagaatacattcgcgtacatc

SEQ ID NO: 594 - DNA, 23 bp, Artificial Sequence
  Description of Artificial Sequence Synthetic probe
gatatacacgatacctgattcgt

SEQ ID NO: 595 - DNA, 23 bp, Artificial Sequence
  Description of Artificial Sequence Synthetic probe
cagtaactctattgtcaaacggt

SEQ ID NO: 596 - DNA, 22 bp, Artificial Sequence
  Description of Artificial Sequence Synthetic probe
tcttcgatagcagacagatagt

SEQ ID NO: 597 - DNA, 21 bp, Artificial Sequence
  Description of Artificial Sequence Synthetic probe
gatacacgacgtttgtgtgta

SEQ ID NO: 598 - DNA, 20 bp, Artificial Sequence
  Description of Artificial Sequence Synthetic probe
ttgcgtgttaccaactcgtc

SEQ ID NO: 599 - DNA, 20 bp, Artificial Sequence
  Description of Artificial Sequence Synthetic probe
tcgtgtcccgtatatgttgg

SEQ ID NO: 600 - DNA, 20 bp, Artificial Sequence
  Description of Artificial Sequence Synthetic probe
cgctctccactatgggtagt

SEQ ID NO: 601 - DNA, 21 bp, Artificial Sequence
  Description of Artificial Sequence Synthetic probe
gatggtcgatctatcgtctct

SEQ ID NO: 602 - DNA, 21 bp, Artificial Sequence
  Description of Artificial Sequence Synthetic probe
cttgaacccttggttcaaggt

SEQ ID NO: 603 - DNA, 19 bp, Artificial Sequence
  Description of Artificial Sequence Synthetic probe
gcagtcggtaacctcgcgc

SEQ ID NO: 604 - DNA, 27 bp, Artificial Sequence
  Description of Artificial Sequence Synthetic probe
gctgtaatgcaagtagcgtatgcgctc

SEQ ID NO: 605 - DNA, 28 bp, Artificial Sequence
  Description of Artificial Sequence Synthetic probe
cgcatatgctgaatgattatctcgttgc

SEQ ID NO: 606 - DNA, 19 bp, Artificial Sequence
  Description of Artificial Sequence Synthetic probe
gacgacagatgcaggttga

SEQ ID NO: 607 - DNA, 28 bp, Artificial Sequence
  Description of Artificial Sequence Synthetic probe
cgcctgctccagtgcatccagcacgaat

SEQ ID NO: 608 - DNA, 25 bp, Artificial Sequence
  Description of Artificial Sequence Synthetic probe
agtgcgttcaccgaatacgtgcgca

SEQ ID NO: 609 - DNA, 27 bp, Artificial Sequence
  Description of Artificial Sequence Synthetic probe
cgcatatgctgaatgattatctcgttg

SEQ ID NO: 610 - DNA, 23 bp, Artificial Sequence
  Description of Artificial Sequence Synthetic probe
gctgtggcacaggctgaacgccg

SEQ ID NO: 611 - DNA, 26 bp, Artificial Sequence
  Description of Artificial Sequence Synthetic probe
acataatctgaatctgagacaacatc

SEQ ID NO: 612 - DNA, 19 bp, Artificial Sequence
  Description of Artificial Sequence Synthetic probe
catcacgaagcccgccaca

SEQ ID NO: 613 - DNA, 18 bp, Artificial Sequence
  Description of Artificial Sequence Synthetic probe
cggaagtatccgcgcgcc

SEQ ID NO: 614 - DNA, 27 bp, Artificial Sequence
  Description of Artificial Sequence Synthetic probe
cattctctcgctttaatttattaacct

SEQ ID NO: 615 - DNA, 23 bp, Artificial Sequence
  Description of Artificial Sequence Synthetic probe
cgttgcttacgcaaccaaatatc

SEQ ID NO: 616 - DNA, 22 bp, Artificial Sequence
  Description of Artificial Sequence Synthetic probe
gcgctatctctgctctcactgc

SEQ ID NO: 617 - DNA, 27 bp, Artificial Sequence
  Description of Artificial Sequence Synthetic probe
gaacagcaaggccgccaatgcctgacg

SEQ ID NO: 618 - DNA, 23 bp, Artificial Sequence
  Description of Artificial Sequence Synthetic probe
atcttgctcaatgaggttattca

SEQ ID NO: 619 - DNA, 19 bp, Artificial Sequence
  Description of Artificial Sequence Synthetic probe
cgcatcgccgatgctcatc

SEQ ID NO: 620 - DNA, 24 bp, Artificial Sequence
  Description of Artificial Sequence Synthetic probe
atgctctccgccatcgcgttgtca

SEQ ID NO: 621 - DNA, 20 bp, Artificial Sequence
  Description of Artificial Sequence Synthetic probe
caggttatgccgctcaattc

SEQ ID NO: 622 - DNA, 28 bp, Artificial Sequence
  Description of Artificial Sequence Synthetic probe
acggtgatcttgctcaatgaggttattc

SEQ ID NO: 623 - DNA, 22 bp, Artificial Sequence
  Description of Artificial Sequence Synthetic probe
ggtgatgtcattctggttaaga

SEQ ID NO: 624 - DNA, 20 bp, Artificial Sequence
  Description of Artificial Sequence Synthetic probe
acgcactctggccacactgg

SEQ ID NO: 625 - DNA, 19 bp, Artificial Sequence
  Description of Artificial Sequence Synthetic probe
gcccttgagcggaagtatc

SEQ ID NO: 626 - DNA, 18 bp, Artificial Sequence
  Description of Artificial Sequence Synthetic probe
ttcgatcacggcacgatc

SEQ ID NO: 627 - DNA, 21 bp, Artificial Sequence
  Description of Artificial Sequence Synthetic probe
atcgaccttctggacattatc

SEQ ID NO: 628 - DNA, 21 bp, Artificial Sequence
  Description of Artificial Sequence Synthetic probe
tgatcttgctcaatgaggtta

SEQ ID NO: 629 - DNA, 21 bp, Artificial Sequence
  Description of Artificial Sequence Synthetic probe
gaggaccgaaggagctaaccg

SEQ ID NO: 630 - DNA, 25 bp, Artificial Sequence
  Description of Artificial Sequence Synthetic probe
ctcattccagaagcaacttcttctt

SEQ ID NO: 631 - DNA, 25 bp, Artificial Sequence
  Description of Artificial Sequence Synthetic probe
ggttctggaccagttgcgtgagcgc

SEQ ID NO: 632 - DNA, 22 bp, Artificial Sequence
  Description of Artificial Sequence Synthetic probe
cgctggatttcacgccataggc

SEQ ID NO: 633 - DNA, 22 bp, Artificial Sequence
  Description of Artificial Sequence Synthetic probe
cgtataggtggctaagtgcagc

SEQ ID NO: 634 - DNA, 23 bp, Artificial Sequence
  Description of Artificial Sequence Synthetic probe
gtacatactcgatcgaagcacga

SEQ ID NO: 635 - DNA, 24 bp, Artificial Sequence
  Description of Artificial Sequence Synthetic probe
aaggtcgaagcaggtacatactcg

SEQ ID NO: 636 - DNA, 23 bp, Artificial Sequence
  Description of Artificial Sequence Synthetic probe
gaagctttcatagcgtcgcctag

SEQ ID NO: 637 - DNA, 23 bp, Artificial Sequence
  Description of Artificial Sequence Synthetic probe
gaagctttcatggcatcgcctag

SEQ ID NO: 638 - DNA, 22 bp, Artificial Sequence
  Description of Artificial Sequence Synthetic probe
cgctaccggtagtattgccctt

SEQ ID NO: 639 - DNA, 22 bp, Artificial Sequence
  Description of Artificial Sequence Synthetic probe
atcgccacgttatcgctgtact

SEQ ID NO: 640 - DNA, 24 bp, Artificial Sequence
  Description of Artificial Sequence Synthetic probe
caagtactgttcctgtacgtcagc

SEQ ID NO: 641 - DNA, 19 bp, Artificial Sequence
  Description of Artificial Sequence Synthetic probe
caacgtctgcgccatcgcc

SEQ ID NO: 642 - DNA, 20 bp, Artificial Sequence
  Description of Artificial Sequence Synthetic probe
gccgcccgaaggacatcaac

SEQ ID NO: 643 - DNA, 21 bp, Artificial Sequence
  Description of Artificial Sequence Synthetic probe
cgtgctggctattgccttagg

SEQ ID NO: 644 - DNA, 24 bp, Artificial Sequence
  Description of Artificial Sequence Synthetic probe
cattaggagttgtcgtatccctca

SEQ ID NO: 645 - DNA, 21 bp, Artificial Sequence
  Description of Artificial Sequence Synthetic probe
aaattgcagttcgcgcttagc

SEQ ID NO: 646 - DNA, 20 bp, Artificial Sequence
  Description of Artificial Sequence Synthetic probe
gcgccaaacagaccaatgct

SEQ ID NO: 647 - DNA, 22 bp, Artificial Sequence
  Description of Artificial Sequence Synthetic probe
gtataggtggctaagtgcagca

SEQ ID NO: 648 - DNA, 21 bp, Artificial Sequence
  Description of Artificial Sequence Synthetic probe
gtcatcgcctcttcgtagctc

SEQ ID NO: 649 - DNA, 21 bp, Artificial Sequence
  Description of Artificial Sequence Synthetic probe
accaatacgccagtagcgaga

SEQ ID NO: 650 - DNA, 24 bp, Artificial Sequence
  Description of Artificial Sequence Synthetic probe
caatcagtgtgtttgatttgcacc

SEQ ID NO: 651 - DNA, 20 bp, Artificial Sequence
  Description of Artificial Sequence Synthetic probe
cggataacgccacgggatga

SEQ ID NO: 652 - DNA, 18 bp, Artificial Sequence
  Description of Artificial Sequence Synthetic probe
gcggcgtggtggtgtctc

SEQ ID NO: 653 - DNA, 18 bp, Artificial Sequence
  Description of Artificial Sequence Synthetic probe
gccacgtcaccagctgcg

SEQ ID NO: 654 - DNA, 19 bp, Artificial Sequence
  Description of Artificial Sequence Synthetic probe
gctcgtagcgtcgcgtctc

SEQ ID NO: 655 - DNA, 21 bp, Artificial Sequence
  Description of Artificial Sequence Synthetic probe
cagcaggtccgccaatttctc

SEQ ID NO: 656 - DNA, 23 bp, Artificial Sequence
  Description of Artificial Sequence Synthetic probe
ccatagaggactttagccacagt

SEQ ID NO: 657 - DNA, 21 bp, Artificial Sequence
  Description of Artificial Sequence Synthetic probe
catatgcagagtgagcggtcc

SEQ ID NO: 658 - DNA, 26 bp, Artificial Sequence
  Description of Artificial Sequence Synthetic probe
ccattaacttcttcaaacgatgtatg

SEQ ID NO: 659 - DNA, 22 bp, Artificial Sequence
  Description of Artificial Sequence Synthetic probe
gtgctgtcgctatggaaatgtg

SEQ ID NO: 660 - DNA, 23 bp, Artificial Sequence
  Description of Artificial Sequence Synthetic probe
gtcagtgtttacaagaaccacca

SEQ ID NO: 661 - DNA, 21 bp, Artificial Sequence
  Description of Artificial Sequence Synthetic probe
cgaaccagcttggttcccaag

SEQ ID NO: 662 - DNA, 23 bp, Artificial Sequence
  Description of Artificial Sequence Synthetic probe
gatgctgtactttgtgatgccta

SEQ ID NO: 663 - DNA, 22 bp, Artificial Sequence
  Description of Artificial Sequence Synthetic probe
gcaagaaagcccttgaatgagc

SEQ ID NO: 664 - DNA, 21 bp, Artificial Sequence
  Description of Artificial Sequence Synthetic probe
aatcaacaaactgctgccgct

SEQ ID NO: 665 - DNA, 18 bp, Artificial Sequence
  Description of Artificial Sequence Synthetic probe
ccagtctgccggcaccgc

SEQ ID NO: 666 - DNA, 19 bp, Artificial Sequence
  Description of Artificial Sequence Synthetic probe
ccgactgcccagtctgccg

SEQ ID NO: 667 - DNA, 28 bp, Artificial Sequence
  Description of Artificial Sequence Synthetic probe
gtaaatagatgatcttaatttggttcac

SEQ ID NO: 668 - DNA, 20 bp, Artificial Sequence
  Description of Artificial Sequence Synthetic probe
cacagcctgactttcgccgc

SEQ ID NO: 669 - DNA, 19 bp, Artificial Sequence
  Description of Artificial Sequence Synthetic probe
ggtggtcgataccgcctgg

SEQ ID NO: 670 - DNA, 22 bp, Artificial Sequence
  Description of Artificial Sequence Synthetic probe
catgtcgagataggaagtgtgc

SEQ ID NO: 671 - DNA, 20 bp, Artificial Sequence
  Description of Artificial Sequence Synthetic probe
caatctgccatcgcgcgatt

SEQ ID NO: 672 - DNA, 22 bp, Artificial Sequence
  Description of Artificial Sequence Synthetic probe
cgaagcaggtacatactcggtc

SEQ ID NO: 673 - DNA, 23 bp, Artificial Sequence
  Description of Artificial Sequence Synthetic probe
tagaatagcggaagctttcatgg

SEQ ID NO: 674 - DNA, 23 bp, Artificial Sequence
  Description of Artificial Sequence Synthetic probe
caagtccaatacgacgagctaaa

SEQ ID NO: 675 - DNA, 22 bp, Artificial Sequence
  Description of Artificial Sequence Synthetic probe
ggtacatactcggtcgaagcac

SEQ ID NO: 676 - DNA, 22 bp, Artificial Sequence
  Description of Artificial Sequence Synthetic probe
ggtacatactcggtcgatgcac

SEQ ID NO: 677 - DNA, 25 bp, Artificial Sequence
  Description of Artificial Sequence Synthetic probe
gtaattgaactagctaatgccgtac

SEQ ID NO: 678 - DNA, 23 bp, Artificial Sequence
  Description of Artificial Sequence Synthetic probe
caagtactgttcctgtacgtcag

SEQ ID NO: 679 - DNA, 23 bp, Artificial Sequence
  Description of Artificial Sequence Synthetic probe
tctctttcccattgtttcatggc

SEQ ID NO: 680 - DNA, 26 bp, Artificial Sequence
  Description of Artificial Sequence Synthetic probe
gtaggttatgcagttattaggttcag

SEQ ID NO: 681 - DNA, 23 bp, Artificial Sequence
  Description of Artificial Sequence Synthetic probe
gcagtaccaacatagctaaatgc

SEQ ID NO: 682 - DNA, 21 bp, Artificial Sequence
  Description of Artificial Sequence Synthetic probe
ggtcctgtggtggtttccacc

SEQ ID NO: 683 - DNA, 20 bp, Artificial Sequence
  Description of Artificial Sequence Synthetic probe
taaccgctgtggtcctgtgg

SEQ ID NO: 684 - DNA, 21 bp, Artificial Sequence
  Description of Artificial Sequence Synthetic probe
ggaagcgttgcttgccatagt

SEQ ID NO: 685 - DNA, 19 bp, Artificial Sequence
  Description of Artificial Sequence Synthetic probe
gttcggtgcaaagacgccg

SEQ ID NO: 686 - DNA, 21 bp, Artificial Sequence
  Description of Artificial Sequence Synthetic probe
cacctgatgcagaaccagcat

SEQ ID NO: 687 - DNA, 18 bp, Artificial Sequence
  Description of Artificial Sequence Synthetic probe
cagctgccgttgcgaacg

SEQ ID NO: 688 - DNA, 19 bp, Artificial Sequence
  Description of Artificial Sequence Synthetic probe
gctcagacgctggctggtc

SEQ ID NO: 689 - DNA, 20 bp, Artificial Sequence
  Description of Artificial Sequence Synthetic probe
gccagtagcagattggcggc

SEQ ID NO: 690 - DNA, 19 bp, Artificial Sequence
  Description of Artificial Sequence Synthetic probe
ccactgcagcagatgccgt

SEQ ID NO: 691 - DNA, 22 bp, Artificial Sequence
  Description of Artificial Sequence Synthetic probe
ttaatttgcttaagcggctgcg

SEQ ID NO: 692 - DNA, 19 bp, Artificial Sequence
  Description of Artificial Sequence Synthetic probe
gggaaagcgttcatcggcg

SEQ ID NO: 693 - DNA, 22 bp, Artificial Sequence
  Description of Artificial Sequence Synthetic probe
tcttatcggcgataaaccagcc

SEQ ID NO: 694 - DNA, 23 bp, Artificial Sequence
  Description of Artificial Sequence Synthetic probe
gtcggaaagttgaccagacatta

SEQ ID NO: 695 - DNA, 24 bp, Artificial Sequence
  Description of Artificial Sequence Synthetic probe
gtgaagtgaatggtcagtatgttg

SEQ ID NO: 696 - DNA, 22 bp, Artificial Sequence
  Description of Artificial Sequence Synthetic probe
cctgtcctacgagttgcatgat

SEQ ID NO: 697 - DNA, 29 bp, Artificial Sequence
  Description of Artificial Sequence Synthetic probe
caaatactaaattatacagtatcagagag

SEQ ID NO: 698 - DNA, 32 bp, Artificial Sequence
  Description of Artificial Sequence Synthetic probe
gttcttattattataagtatctattaacagtt

SEQ ID NO: 699 - DNA, 23 bp, Artificial Sequence
  Description of Artificial Sequence Synthetic probe
catcgggaaatggaagtcgttat

SEQ ID NO: 700 - DNA, 21 bp, Artificial Sequence
  Description of Artificial Sequence Synthetic probe
cgtggtttgtgctgagcaaag

SEQ ID NO: 701 - DNA, 19 bp, Artificial Sequence
  Description of Artificial Sequence Synthetic probe
gccgcccgaaggacatcaa

SEQ ID NO: 702 - DNA, 21 bp, Artificial Sequence
  Description of Artificial Sequence Synthetic probe
gcaactcatcaccatcacgga

SEQ ID NO: 703 - DNA, 20 bp, Artificial Sequence
  Description of Artificial Sequence Synthetic probe
gcgacagccatgacagacgc

SEQ ID NO: 704 - DNA, 21 bp, Artificial Sequence
  Description of Artificial Sequence Synthetic probe
aaacgactgcgttgcgatatg

SEQ ID NO: 705 - DNA, 20 bp, Artificial Sequence
  Description of Artificial Sequence Synthetic probe
atgcgaccaaacgccatcgc

SEQ ID NO: 706 - DNA, 22 bp, Artificial Sequence
  Description of Artificial Sequence Synthetic probe
gaacaggcttatgtcaactggg

SEQ ID NO: 707 - DNA, 22 bp, Artificial Sequence
  Description of Artificial Sequence Synthetic probe
acgaaccgaacaggcttatgtc

SEQ ID NO: 708 - DNA, 23 bp, Artificial Sequence
  Description of Artificial Sequence Synthetic probe
catcatagacgcggtcaaataga

SEQ ID NO: 709 - DNA, 20 bp, Artificial Sequence
  Description of Artificial Sequence Synthetic probe
caggctgtttcgggctgtga

SEQ ID NO: 710 - DNA, 23 bp, Artificial Sequence
  Description of Artificial Sequence Synthetic probe
taattcaagtgcaactctcgcaa

SEQ ID NO: 711 - DNA, 24 bp, Artificial Sequence
  Description of Artificial Sequence Synthetic probe
ggatagttacgactttctgcttca

SEQ ID NO: 712 - DNA, 27 bp, Artificial Sequence
  Description of Artificial Sequence Synthetic probe
gtcaggctaaatatagctatcttatcg

SEQ ID NO: 713 - DNA, 23 bp, Artificial Sequence
  Description of Artificial Sequence Synthetic probe
catcctaagccaagtgtagactc

SEQ ID NO: 714 - DNA, 19 bp, Artificial Sequence
  Description of Artificial Sequence Synthetic probe
cgccgcatacactattctc

SEQ ID NO: 715 - DNA, 23 bp, Artificial Sequence
  Description of Artificial Sequence Synthetic probe
ggatagccatggctacaagaata

SEQ ID NO: 716 - DNA, 22 bp, Artificial Sequence
  Description of Artificial Sequence Synthetic probe
cgtaacatcgttgctgctccat

SEQ ID NO: 717 - DNA, 20 bp, Artificial Sequence
  Description of Artificial Sequence Synthetic probe
tgtcgctaccgttgatgatt

SEQ ID NO: 718 - DNA, 22 bp, Artificial Sequence
  Description of Artificial Sequence Synthetic probe
gtaactcattcctgagggtttc

SEQ ID NO: 719 - DNA, 20 bp, Artificial Sequence
  Description of Artificial Sequence Synthetic probe
ccggaatagcggaagctttc

SEQ ID NO: 720 - DNA, 21 bp, Artificial Sequence
  Description of Artificial Sequence Synthetic probe
agacatgagctcaagtccaat

SEQ ID NO: 721 - DNA, 23 bp, Artificial Sequence
  Description of Artificial Sequence Synthetic probe
ttagctagcttgtaagcaaattg

SEQ ID NO: 722 - DNA, 21 bp, Artificial Sequence
  Description of Artificial Sequence Synthetic probe
agctagcttgtaagcaaactg

SEQ ID NO: 723 - DNA, 20 bp, Artificial Sequence
  Description of Artificial Sequence Synthetic probe
agaatatcccgacggctttc

SEQ ID NO: 724 - DNA, 20 bp, Artificial Sequence
  Description of Artificial Sequence Synthetic probe
tttacccagcgtcagattcc

SEQ ID NO: 725 - DNA, 21 bp, Artificial Sequence
  Description of Artificial Sequence Synthetic probe
tcgccagtaactggtctattc

SEQ ID NO: 726 - DNA, 20 bp, Artificial Sequence
  Description of Artificial Sequence Synthetic probe
cgcaatatcattggtggtgc

SEQ ID NO: 727 - DNA, 19 bp, Artificial Sequence
  Description of Artificial Sequence Synthetic probe
cagacgggacgtacacaac

SEQ ID NO: 728 - DNA, 22 bp, Artificial Sequence
  Description of Artificial Sequence Synthetic probe
gtaatactcctagcaccaaatc

SEQ ID NO: 729 - DNA, 20 bp, Artificial Sequence
  Description of Artificial Sequence Synthetic probe
aatactccgagcaccaaatc

SEQ ID NO: 730 - DNA, 21 bp, Artificial Sequence
  Description of Artificial Sequence Synthetic probe
gttccatagcgttaaggtttc

SEQ ID NO: 731 - DNA, 19 bp, Artificial Sequence
  Description of Artificial Sequence Synthetic probe
gatttcacgccataggctc

SEQ ID NO: 732 - DNA, 20 bp, Artificial Sequence
  Description of Artificial Sequence Synthetic probe
tcgtaactcattcctgaggg

SEQ ID NO: 733 - DNA, 19 bp, Artificial Sequence
  Description of Artificial Sequence Synthetic probe
gccatatcgataacgctgg

SEQ ID NO: 734 - DNA, 19 bp, Artificial Sequence
  Description of Artificial Sequence Synthetic probe
gcaacgtagctgccaaatc

SEQ ID NO: 735 - DNA, 19 bp, Artificial Sequence
  Description of Artificial Sequence Synthetic probe
tacccggaatagcctgctc

SEQ ID NO: 736 - DNA, 20 bp, Artificial Sequence
  Description of Artificial Sequence Synthetic probe
accgggtcaaagaattcctc

SEQ ID NO: 737 - DNA, 18 bp, Artificial Sequence
  Description of Artificial Sequence Synthetic probe
cgctgccggtcttatcac

SEQ ID NO: 738 - DNA, 19 bp, Artificial Sequence
  Description of Artificial Sequence Synthetic probe
cggctgggtgaagtaagtc

SEQ ID NO: 739 - DNA, 18 bp, Artificial Sequence
  Description of Artificial Sequence Synthetic probe
ttgaccgacagaggcaac

SEQ ID NO: 740 - DNA, 17 bp, Artificial Sequence
  Description of Artificial Sequence Synthetic probe
agtggacgtcagtgcgc

SEQ ID NO: 741 - DNA, 19 bp, Artificial Sequence
  Description of Artificial Sequence Synthetic probe
tacaccgctacagcgtaat

SEQ ID NO: 742 - DNA, 21 bp, Artificial Sequence
  Description of Artificial Sequence Synthetic probe
tcaattctttcaaagaccagc

SEQ ID NO: 743 - DNA, 17 bp, Artificial Sequence
  Description of Artificial Sequence Synthetic probe
acccgtgctgtcgctat

SEQ ID NO: 744 - DNA, 22 bp, Artificial Sequence
  Description of Artificial Sequence Synthetic probe
aaccaaaccactaggttatctt

SEQ ID NO: 745 - DNA, 21 bp, Artificial Sequence
  Description of Artificial Sequence Synthetic probe
atgcatacgtgggaatagatt

SEQ ID NO: 746 - DNA, 17 bp, Artificial Sequence
  Description of Artificial Sequence Synthetic probe
tcactgcgtgttcgctc

SEQ ID NO: 747 - DNA, 19 bp, Artificial Sequence
  Description of Artificial Sequence Synthetic probe
cgcttggcaagtactgttc

SEQ ID NO: 748 - DNA, 20 bp, Artificial Sequence
  Description of Artificial Sequence Synthetic probe
gcgttatcactgtattgcac

SEQ ID NO: 749 - DNA, 20 bp, Artificial Sequence
  Description of Artificial Sequence Synthetic probe
gctgtacttgtcatccttgt

SEQ ID NO: 750 - DNA, 17 bp, Artificial Sequence
  Description of Artificial Sequence Synthetic probe
tcgagcgcgagtctagc

SEQ ID NO: 751 - DNA, 17 bp, Artificial Sequence
  Description of Artificial Sequence Synthetic probe
cgagcgcgagtctagcc

SEQ ID NO: 752 - DNA, 17 bp, Artificial Sequence
  Description of Artificial Sequence Synthetic probe
ttgctggccaatcgtcg

SEQ ID NO: 753 - DNA, 20 bp, Artificial Sequence
  Description of Artificial Sequence Synthetic probe
caagcaggagatcaacctgc

SEQ ID NO: 754 - DNA, 17 bp, Artificial Sequence
  Description of Artificial Sequence Synthetic probe
gtgaaatccgcccgacg

SEQ ID NO: 755 - DNA, 17 bp, Artificial Sequence
  Description of Artificial Sequence Synthetic probe
tgatgcgcgtgagtcac

SEQ ID NO: 756 - DNA, 18 bp, Artificial Sequence
  Description of Artificial Sequence Synthetic probe
cggcaatctcggtgatgc

SEQ ID NO: 757 - DNA, 23 bp, Artificial Sequence
  Description of Artificial Sequence Synthetic probe
acgagctaaatcttgataaactt

SEQ ID NO: 758 - DNA, 21 bp, Artificial Sequence
  Description of Artificial Sequence Synthetic probe
agctagcttgtaagcaaactg

SEQ ID NO: 759 - DNA, 21 bp, Artificial Sequence
  Description of Artificial Sequence Synthetic probe
gaatagcatggattgcacttc

SEQ ID NO: 760 - DNA, 23 bp, Artificial Sequence
  Description of Artificial Sequence Synthetic probe
aatcttgataaactgaaatagcg

SEQ ID NO: 761 - DNA, 21 bp, Artificial Sequence
  Description of Artificial Sequence Synthetic probe
tcttgataaaccggaatagcg

SEQ ID NO: 762 - DNA, 21 bp, Artificial Sequence
  Description of Artificial Sequence Synthetic probe
ttatgacaccagtttctaggc

SEQ ID NO: 763 - DNA, 19 bp, Artificial Sequence
  Description of Artificial Sequence Synthetic probe
gcccagttgtgatgcattc

SEQ ID NO: 764 - DNA, 20 bp, Artificial Sequence
  Description of Artificial Sequence Synthetic probe
tgcggaaattctaagctgac

SEQ ID NO: 765 - DNA, 18 bp, Artificial Sequence
  Description of Artificial Sequence Synthetic probe
gactcagccgagtcaagc

SEQ ID NO: 766 - DNA, 21 bp, Artificial Sequence
  Description of Artificial Sequence Synthetic probe
aaataacaaatcacaggccac

SEQ ID NO: 767 - DNA, 20 bp, Artificial Sequence
  Description of Artificial Sequence Synthetic probe
cgcgataatggcttcattgg

SEQ ID NO: 768 - DNA, 21 bp, Artificial Sequence
  Description of Artificial Sequence Synthetic probe
tgcgcaataatagcttcattg

SEQ ID NO: 769 - DNA, 20 bp, Artificial Sequence
  Description of Artificial Sequence Synthetic probe
aaccgaagcaccatgtaatt

SEQ ID NO: 770 - DNA, 23 bp, Artificial Sequence
  Description of Artificial Sequence Synthetic probe
tcgcagacttcaatatcaatatt

SEQ ID NO: 771 - DNA, 19 bp, Artificial Sequence
  Description of Artificial Sequence Synthetic probe
aggccacgttatcactgtg

SEQ ID NO: 772 - DNA, 21 bp, Artificial Sequence
  Description of Artificial Sequence Synthetic probe
cgcagataaatcaccacaatc

SEQ ID NO: 773 - DNA, 19 bp, Artificial Sequence
  Description of Artificial Sequence Synthetic probe
ccgcagataaatcaccacg

SEQ ID NO: 774 - DNA, 17 bp, Artificial Sequence
  Description of Artificial Sequence Synthetic probe
gaacgggcgctcagacg

SEQ ID NO: 775 - DNA, 21 bp, Artificial Sequence
  Description of Artificial Sequence Synthetic probe
gtatcccgcagataaatcacc

SEQ ID NO: 776 - DNA, 17 bp, Artificial Sequence
  Description of Artificial Sequence Synthetic probe
ccagctgttcgtcaccg

SEQ ID NO: 777 - DNA, 18 bp, Artificial Sequence
  Description of Artificial Sequence Synthetic probe
tcgctcatggtaatggcg

SEQ ID NO: 778 - DNA, 17 bp, Artificial Sequence
  Description of Artificial Sequence Synthetic probe
cgttgccagtgctcgat

SEQ ID NO: 779 - DNA, 25 bp, Artificial Sequence
  Description of Artificial Sequence Synthetic probe
atactaggagaagttaataaatacg

SEQ ID NO: 780 - DNA, 18 bp, Artificial Sequence
  Description of Artificial Sequence Synthetic probe
agtgcgcaggagattagc

SEQ ID NO: 781 - DNA, 19 bp, Artificial Sequence
  Description of Artificial Sequence Synthetic probe
ataatggcctgcttctcgc

SEQ ID NO: 782 - DNA, 22 bp, Artificial Sequence
  Description of Artificial Sequence Synthetic probe
atgcaaagcgttatgaaatttc

SEQ ID NO: 783 - DNA, 19 bp, Artificial Sequence
  Description of Artificial Sequence Synthetic probe
cattagtggctgctgcaat

SEQ ID NO: 784 - DNA, 21 bp, Artificial Sequence
  Description of Artificial Sequence Synthetic probe
gttcaatcgtcaaagttgttc

SEQ ID NO: 785 - DNA, 23 bp, Artificial Sequence
  Description of Artificial Sequence Synthetic probe
caaagttaagttgtcagtttgag

SEQ ID NO: 786 - DNA, 18 bp, Artificial Sequence
  Description of Artificial Sequence Synthetic probe
agacgggacgtacacaac

SEQ ID NO: 787 - DNA, 18 bp, Artificial Sequence
  Description of Artificial Sequence Synthetic probe
tgatgcgtacgttgccac

SEQ ID NO: 788 - DNA, 20 bp, Artificial Sequence
  Description of Artificial Sequence Synthetic probe
ggacaatgagaccattggac

SEQ ID NO: 789 - DNA, 19 bp, Artificial Sequence
  Description of Artificial Sequence Synthetic probe
ttccgaaggacatcaacgc

SEQ ID NO: 790 - DNA, 19 bp, Artificial Sequence
  Description of Artificial Sequence Synthetic probe
atcgtcatggaagtgcgta

SEQ ID NO: 791 - DNA, 21 bp, Artificial Sequence
  Description of Artificial Sequence Synthetic probe
cataacatcaaacatcgaccc

SEQ ID NO: 792 - DNA, 17 bp, Artificial Sequence
  Description of Artificial Sequence Synthetic probe
taacgcgcttgctgctt

SEQ ID NO: 793 - DNA, 19 bp, Artificial Sequence
  Description of Artificial Sequence Synthetic probe
actcatcaccatcacggac

SEQ ID NO: 794 - DNA, 24 bp, Artificial Sequence
  Description of Artificial Sequence Synthetic probe
gggttattaataaagatgataggc

SEQ ID NO: 795 - DNA, 25 bp, Artificial Sequence
  Description of Artificial Sequence Synthetic probe
tttattctctaatgcgctatatatt

SEQ ID NO: 796 - DNA, 22 bp, Artificial Sequence
  Description of Artificial Sequence Synthetic probe
tgtattgctattatcgtcaacg

SEQ ID NO: 797 - DNA, 24 bp, Artificial Sequence
  Description of Artificial Sequence Synthetic probe
tcagttactgctatagaaattgat

SEQ ID NO: 798 - DNA, 27 bp, Artificial Sequence
  Description of Artificial Sequence Synthetic probe
aagatatatggtaatattccttataac

SEQ ID NO: 799 - DNA, 27 bp, Artificial Sequence
  Description of Artificial Sequence Synthetic probe
gtggtatggctgattatgatctagagt

SEQ ID NO: 800 - DNA, 25 bp, Artificial Sequence
  Description of Artificial Sequence Synthetic probe
agtatcttacctgaaattccctcac

SEQ ID NO: 801 - DNA, 24 bp, Artificial Sequence
  Description of Artificial Sequence Synthetic probe
cagtccctcgatattcagatcaga

SEQ ID NO: 802 - DNA, 21 bp, Artificial Sequence
  Description of Artificial Sequence Synthetic probe
cagctgcggtaaagctcatca

SEQ ID NO: 803 - DNA, 25 bp, Artificial Sequence
  Description of Artificial Sequence Synthetic probe
gtaacaactttcatgctctcctaaa

SEQ ID NO: 804 - DNA, 23 bp, Artificial Sequence
  Description of Artificial Sequence Synthetic probe
cgtttccagactttacgaaacac

SEQ ID NO: 805 - DNA, 26 bp, Artificial Sequence
  Description of Artificial Sequence Synthetic probe
catcatgttcatatttatcagagctc

SEQ ID NO: 806 - DNA, 21 bp, Artificial Sequence
  Description of Artificial Sequence Synthetic probe
gtttccacatggtgaacggtg

SEQ ID NO: 807 - DNA, 21 bp, Artificial Sequence
  Description of Artificial Sequence Synthetic probe
gctgtaattatgacgacgccg

SEQ ID NO: 808 - DNA, 23 bp, Artificial Sequence
  Description of Artificial Sequence Synthetic probe
gtgtatgtcagcgatttgtccat

SEQ ID NO: 809 - DNA, 21 bp, Artificial Sequence
  Description of Artificial Sequence Synthetic probe
gtccacctcgccaacaatcaa

SEQ ID NO: 810 - DNA, 21 bp, Artificial Sequence
  Description of Artificial Sequence Synthetic probe
gcgtgattatcacgttcggca

SEQ ID NO: 811 - DNA, 23 bp, Artificial Sequence
  Description of Artificial Sequence Synthetic probe
ggctcgacttcctgatgaatacg

SEQ ID NO: 812 - DNA, 24 bp, Artificial Sequence
  Description of Artificial Sequence Synthetic probe
caacgatgtatgtcaacgatttgt

SEQ ID NO: 813 - DNA, 22 bp, Artificial Sequence
  Description of Artificial Sequence Synthetic probe
ggctcggcttcctgatgaatac

SEQ ID NO: 814 - DNA, 24 bp, Artificial Sequence
  Description of Artificial Sequence Synthetic probe
cagtatttcaccttgtccgtaacc

SEQ ID NO: 815 - DNA, 27 bp, Artificial Sequence
  Description of Artificial Sequence Synthetic probe
aatgtttatatctttaacgcctaaact

SEQ ID NO: 816 - DNA, 19 bp, Artificial Sequence
  Description of Artificial Sequence Synthetic probe
ctggcccttgaggtcgcgg

SEQ ID NO: 817 - DNA, 20 bp, Artificial Sequence
  Description of Artificial Sequence Synthetic probe
gacgtagatcgggtcgagct

SEQ ID NO: 818 - DNA, 20 bp, Artificial Sequence
  Description of Artificial Sequence Synthetic probe
ggcgtactgctgcttgctca

SEQ ID NO: 819 - DNA, 20 bp, Artificial Sequence
  Description of Artificial Sequence Synthetic probe
cctgttcctgggtcgaagcc

SEQ ID NO: 820 - DNA, 25 bp, Artificial Sequence
  Description of Artificial Sequence Synthetic probe
gtttataagtgggtaaaccgtgaat

SEQ ID NO: 821 - DNA, 24 bp, Artificial Sequence
  Description of Artificial Sequence Synthetic probe
gccctttcaggactttgatactgg

SEQ ID NO: 822 - DNA, 23 bp, Artificial Sequence
  Description of Artificial Sequence Synthetic probe
acactgaccgattcatcctcgtg

SEQ ID NO: 823 - DNA, 22 bp, Artificial Sequence
  Description of Artificial Sequence Synthetic probe
cggaagcccaccaagtgagtac

SEQ ID NO: 824 - DNA, 24 bp, Artificial Sequence
  Description of Artificial Sequence Synthetic probe
accagcttgtctttagtctgagag

SEQ ID NO: 825 - DNA, 25 bp, Artificial Sequence
  Description of Artificial Sequence Synthetic probe
cattggtttgttctgtttgagaggc

SEQ ID NO: 826 - DNA, 25 bp, Artificial Sequence
  Description of Artificial Sequence Synthetic probe
ggactttgatactggaggagtcata

SEQ ID NO: 827 - DNA, 22 bp, Artificial Sequence
  Description of Artificial Sequence Synthetic probe
atgctggaggagtcgtacgttt

SEQ ID NO: 828 - DNA, 25 bp, Artificial Sequence
  Description of Artificial Sequence Synthetic probe
gagtttggacaaaccacaactagaa

SEQ ID NO: 829 - DNA, 21 bp, Artificial Sequence
  Description of Artificial Sequence Synthetic probe
cctctcgtcataagtcgaatg

SEQ ID NO: 830 - DNA, 20 bp, Artificial Sequence
  Description of Artificial Sequence Synthetic probe
ttaacaatttcgcaaccgtc

SEQ ID NO: 831 - DNA, 23 bp, Artificial Sequence
  Description of Artificial Sequence Synthetic probe
catagttaagccagtatacactc

SEQ ID NO: 832 - DNA, 20 bp, Artificial Sequence
  Description of Artificial Sequence Synthetic probe
cggtaactgatgccgtattt

SEQ ID NO: 833 - DNA, 20 bp, Artificial Sequence
  Description of Artificial Sequence Synthetic probe
acgttgtgagggtaaacaac

SEQ ID NO: 834 - DNA, 24 bp, Artificial Sequence
  Description of Artificial Sequence Synthetic probe
tagatttcataaagtctaacacac

SEQ ID NO: 835 - DNA, 21 bp, Artificial Sequence
  Description of Artificial Sequence Synthetic probe
aaacctgtcactctgaatgtt

SEQ ID NO: 836 - DNA, 20 bp, Artificial Sequence
  Description of Artificial Sequence Synthetic probe
ctcggtgagattcagaatgc

SEQ ID NO: 837 - DNA, 21 bp, Artificial Sequence
  Description of Artificial Sequence Synthetic probe
tgtcatattgtcttgccgatt

SEQ ID NO: 838 - DNA, 21 bp, Artificial Sequence
  Description of Artificial Sequence Synthetic probe
atatcaacacgggaaagacct

SEQ ID NO: 839 - DNA, 20 bp, Artificial Sequence
  Description of Artificial Sequence Synthetic probe
cttgcagatttaaccgacac

SEQ ID NO: 840 - DNA, 19 bp, Artificial Sequence
  Description of Artificial Sequence Synthetic probe
tgaaaccgggcagagtatt

SEQ ID NO: 841 - DNA, 20 bp, Artificial Sequence
  Description of Artificial Sequence Synthetic probe
attgcgtagtccaattcgtc

SEQ ID NO: 842 - DNA, 21 bp, Artificial Sequence
  Description of Artificial Sequence Synthetic probe
aggcatggtattgacttcatt

SEQ ID NO: 843 - DNA, 20 bp, Artificial Sequence
  Description of Artificial Sequence Synthetic probe
gtttacgacttgttgcatgc

SEQ ID NO: 844 - DNA, 20 bp, Artificial Sequence
  Description of Artificial Sequence Synthetic probe
atgctttggtctttctgcat

SEQ ID NO: 845 - DNA, 18 bp, Artificial Sequence
  Description of Artificial Sequence Synthetic probe
cggtcttcacctcgacac

SEQ ID NO: 846 - DNA, 19 bp, Artificial Sequence
  Description of Artificial Sequence Synthetic probe
acggaaacctcggagaatt

SEQ ID NO: 847 - DNA, 18 bp, Artificial Sequence
  Description of Artificial Sequence Synthetic probe
tgacgtcgacgtagatcg

SEQ ID NO: 848 - DNA, 16 bp, Artificial Sequence
  Description of Artificial Sequence Synthetic probe
cttcggtcaccgcgga

SEQ ID NO: 849 - DNA, 20 bp, Artificial Sequence
  Description of Artificial Sequence Synthetic probe
gaaacgagctttaggtttgc

SEQ ID NO: 850 - DNA, 21 bp, Artificial Sequence
  Description of Artificial Sequence Synthetic probe
tgtacggagacggagttatcg

SEQ ID NO: 851 - DNA, 21 bp, Artificial Sequence
  Description of Artificial Sequence Synthetic probe
cttgaaagtgcgttaacaacc

SEQ ID NO: 852 - DNA, 22 bp, Artificial Sequence
  Description of Artificial Sequence Synthetic probe
cgaaaccagtttgtccttagtc

SEQ ID NO: 853 - DNA, 21 bp, Artificial Sequence
  Description of Artificial Sequence Synthetic probe
ctttacgacgggtcatttcac

SEQ ID NO: 854 - DNA, 23 bp, Artificial Sequence
  Description of Artificial Sequence Synthetic probe
gattcatcttcgtgaattgtgac

SEQ ID NO: 855 - DNA, 21 bp, Artificial Sequence
  Description of Artificial Sequence Synthetic probe
tgtacggaaacggagttatcg

SEQ ID NO: 856 - DNA, 21 bp, Artificial Sequence
  Description of Artificial Sequence Synthetic probe
gtcgcgcacactaatagattc

SEQ ID NO: 857 - DNA, 26 bp, Artificial Sequence
  Description of Artificial Sequence Synthetic probe
gctgtcaccgtccagacgctgttggc

SEQ ID NO: 858 - DNA, 28 bp, Artificial Sequence
  Description of Artificial Sequence Synthetic probe
gactccgcagaatacggcaccgtgcgca

SEQ ID NO: 859 - DNA, 22 bp, Artificial Sequence
  Description of Artificial Sequence Synthetic probe
gctgtcctggctgcaagcctgg

SEQ ID NO: 860 - DNA, 28 bp, Artificial Sequence
  Description of Artificial Sequence Synthetic probe
gacagcagactcaccggctggttccgct

SEQ ID NO: 861 - DNA, 22 bp, Artificial Sequence
  Description of Artificial Sequence Synthetic probe
gacagaacaagttccgctccgg

SEQ ID NO: 862 - DNA, 25 bp, Artificial Sequence
  Description of Artificial Sequence Synthetic probe
gaacgtctggcgctggtcgcctgcc

SEQ ID NO: 863 - DNA, 27 bp, Artificial Sequence
  Description of Artificial Sequence Synthetic probe
aatccaggtcctgaccgttctgtccgt

SEQ ID NO: 864 - DNA, 25 bp, Artificial Sequence
  Description of Artificial Sequence Synthetic probe
gaggtggccaacaccatgtgtgacc

SEQ ID NO: 865 - DNA, 21 bp, Artificial Sequence
  Description of Artificial Sequence Synthetic probe
gaagtgccggacttctgcaga

SEQ ID NO: 866 - DNA, 20 bp, Artificial Sequence
  Description of Artificial Sequence Synthetic probe
gctaatcgcataacagctac

SEQ ID NO: 867 - DNA, 23 bp, Artificial Sequence
  Description of Artificial Sequence Synthetic probe
gctgcggtattccacggtcggcc

SEQ ID NO: 868 - DNA, 27 bp, Artificial Sequence
  Description of Artificial Sequence Synthetic probe
gaatcaattatcttcttcattattgat

SEQ ID NO: 869 - DNA, 19 bp, Artificial Sequence
  Description of Artificial Sequence Synthetic probe
gtcacacgtcacgcagtcc

SEQ ID NO: 870 - DNA, 27 bp, Artificial Sequence
  Description of Artificial Sequence Synthetic probe
gtgttactcggtagaatgctcgcaagg

SEQ ID NO: 871 - DNA, 19 bp, Artificial Sequence
  Description of Artificial Sequence Synthetic probe
cggaactgcctgctcgtat

SEQ ID NO: 872 - DNA, 24 bp, Artificial Sequence
  Description of Artificial Sequence Synthetic probe
gctctccgactcctggtacgtcag

SEQ ID NO: 873 - DNA, 28 bp, Artificial Sequence
  Description of Artificial Sequence Synthetic probe
gatgttgcgattacttcgccaactattg

SEQ ID NO: 874 - DNA, 26 bp, Artificial Sequence
  Description of Artificial Sequence Synthetic probe
gcaataccaggaaggaagtcttactg

SEQ ID NO: 875 - DNA, 24 bp, Artificial Sequence
  Description of Artificial Sequence Synthetic probe
gtatcgccacaataactgccggaa

SEQ ID NO: 876 - DNA, 20 bp, Artificial Sequence
  Description of Artificial Sequence Synthetic probe
gtgaagcgcatccggtcacc

SEQ ID NO: 877 - DNA, 18 bp, Artificial Sequence
  Description of Artificial Sequence Synthetic probe
tccgtgccttcaagcgcg

SEQ ID NO: 878 - DNA, 18 bp, Artificial Sequence
  Description of Artificial Sequence Synthetic probe
gcgtacaggccagtcagc

SEQ ID NO: 879 - DNA, 19 bp, Artificial Sequence
  Description of Artificial Sequence Synthetic probe
ccgaactgctgatggacgt

SEQ ID NO: 880 - DNA, 23 bp, Artificial Sequence
  Description of Artificial Sequence Synthetic probe
gcaagatgctgctggccacactg

SEQ ID NO: 881 - DNA, 18 bp, Artificial Sequence
  Description of Artificial Sequence Synthetic probe
cacggatacgccgcgcat

SEQ ID NO: 882 - DNA, 19 bp, Artificial Sequence
  Description of Artificial Sequence Synthetic probe
gcacaggtgctgacgtggt

SEQ ID NO: 883 - DNA, 20 bp, Artificial Sequence
  Description of Artificial Sequence Synthetic probe
acctccgttgagctgatgga

SEQ ID NO: 884 - DNA, 28 bp, Artificial Sequence
  Description of Artificial Sequence Synthetic probe
gacgccggtatatcggtatcgagctgct

SEQ ID NO: 885 - DNA, 21 bp, Artificial Sequence
  Description of Artificial Sequence Synthetic probe
gcacggcctgatggaggccgc

SEQ ID NO: 886 - DNA, 25 bp, Artificial Sequence
  Description of Artificial Sequence Synthetic probe
catcacgtaacttattgatgatatt

SEQ ID NO: 887 - DNA, 21 bp, Artificial Sequence
  Description of Artificial Sequence Synthetic probe
gcaggaacgctgcctgtggtc

SEQ ID NO: 888 - DNA, 19 bp, Artificial Sequence
  Description of Artificial Sequence Synthetic probe
ctgcggctcaactcaagca

SEQ ID NO: 889 - DNA, 20 bp, Artificial Sequence
  Description of Artificial Sequence Synthetic probe
gcattcatggcgctgatggc

SEQ ID NO: 890 - DNA, 24 bp, Artificial Sequence
  Description of Artificial Sequence Synthetic probe
actagatgacatatcatgtaagtt

SEQ ID NO: 891 - DNA, 18 bp, Artificial Sequence
  Description of Artificial Sequence Synthetic probe
aacgatatagtccgttat

SEQ ID NO: 892 - DNA, 19 bp, Artificial Sequence
  Description of Artificial Sequence Synthetic probe
gcgcgcattaatgaagcac

SEQ ID NO: 893 - DNA, 21 bp, Artificial Sequence
  Description of Artificial Sequence Synthetic probe
gctgtaattatgacgacgccg

SEQ ID NO: 894 - DNA, 26 bp, Artificial Sequence
  Description of Artificial Sequence Synthetic probe
gtcattggagaacagatgattgatgt

SEQ ID NO: 895 - DNA, 19 bp, Artificial Sequence
  Description of Artificial Sequence Synthetic probe
aacgatatagtccgttatg

SEQ ID NO: 896 - DNA, 22 bp, Artificial Sequence
  Description of Artificial Sequence Synthetic probe
atggcataggccaggtcaatat

SEQ ID NO: 897 - DNA, 173 bp, Artificial Sequence
  Description of Artificial Sequence Synthetic polynucleotide
tttgaactcgttttccgcctttaataccttattcctccagttgttgaagactgcggctgcgaatctcttgttgcagcttctcgatcacttcatttacgtccatgctgccgagtctttcccgcgacgggtgcgcacggccactttgccggcttcgacttctttgtcgccacaga

SEQ ID NO: 898 - DNA, 174 bp, Artificial Sequence
  Description of Artificial Sequence Synthetic polynucleotide
tttgaactcgttttccgcctttaataccttattcctccagttgttttagactgcggctgcgaatctcttgttgcagcttctcgataacttcatttacgtccaggctgcccaagtctttgccacgacgggtacgcacggcaactttgccggcttctacctctttgtcgccacaga

SEQ ID NO: 899 - DNA, 34 bp, Artificial Sequence
  Description of Artificial Sequence Synthetic oligonucleotide
ccagtctcttttttatacggtacattgtttttat

SEQ ID NO: 900 - DNA, 34 bp, Artificial Sequence; identical to SEQ ID NO: 899
  Description of Artificial Sequence Synthetic oligonucleotide

SEQ ID NO: 901 - DNA, 35 bp, Artificial Sequence
  Description of Artificial Sequence Synthetic oligonucleotide
ccagtctcttttttatactggtacattgtttttat

SEQ ID NO: 902 - DNA, 35 bp, Artificial Sequence; identical to SEQ ID NO: 901
  Description of Artificial Sequence Synthetic oligonucleotide

SEQ ID NO: 903 - DNA, 35 bp, Artificial Sequence; identical to SEQ ID NO: 901
  Description of Artificial Sequence Synthetic oligonucleotide

SEQ ID NO: 904 - DNA, 37 bp, Artificial Sequence
  Description of Artificial Sequence Synthetic oligonucleotide
ccttatttatcaatgttagtctcatcaattgcattac

SEQ ID NO: 905 - DNA, 37 bp, Artificial Sequence; identical to SEQ ID NO: 904
  Description of Artificial Sequence Synthetic oligonucleotide

SEQ ID NO: 906 - DNA, 37 bp, Artificial Sequence
  Description of Artificial Sequence Synthetic oligonucleotide
ccttatttatcaatattagtttcatcaattgcattac

SEQ ID NO: 907 - DNA, 37 bp, Artificial Sequence; identical to SEQ ID NO: 906
  Description of Artificial Sequence Synthetic oligonucleotide

SEQ ID NO: 908 - DNA, 37 bp, Artificial Sequence; identical to SEQ ID NO: 906
  Description of Artificial Sequence Synthetic oligonucleotide

SEQ ID NO: 909 - DNA, 37 bp, Artificial Sequence; identical to SEQ ID NO: 906
  Description of Artificial Sequence Synthetic oligonucleotide

SEQ ID NO: 910 - DNA, 37 bp, Artificial Sequence; identical to SEQ ID NO: 904
  Description of Artificial Sequence Synthetic oligonucleotide

(19) United States
(12) Patent Application Publication
(10) Pub. No.: US 2015/0344973 A1
(43) Pub. Date: Dec. 3, 2015

(54) Method and System for Detection of an Organism

(71) Applicant: Pathogenica, Inc., Cambridge, MA (US)

(72) Inventors: Philip Alexander ROLFE, Newton, MA (US); Thomas CLARKE, IV, Dracut, MA (US); Sarah MAHONEY, Cambridge, MA (US); Jack Thacher LEONARD, South Hamilton, MA (US); Sarah GRUSZKA, Cambridge, MA (US)

(21) Appl. No.: 14/395,958

(22) PCT Filed: Apr. 23, 2013
(86) PCT No.: PCT/US2013/037833

§ 371 Date: Oct. 21, 2014

Related U.S. Application Data
(60) Provisional application No. 61/637,185, filed on Apr. 23, 2012.

(51) Int. Cl. C12Q 1/68; C12Q 1/70

(52) U.S. Cl. CPC C12Q 1/6888; C12Q 1/701; C12Q 1/6895; C12Q 1/689

(57) ABSTRACT

The invention provides, inter alia, systems, compositions, kits and methods for detecting an organism, such as a microbe, microorganism, pathogen, or organism associated with Hospital Associated Infections (HAIs). The systems, compositions, kits and methods can comprise one or more probes for detecting a strain with high sensitivity, high specificity, or both. The systems, compositions, kits and methods can also be used to detect the strain within a short time frame.

### RELATED APPLICATION

This application claims the benefit of U.S. Provisional Application No. 61/637,185, filed Apr. 23, 2012. The entire teachings of the above application are incorporated herein by reference.

### BACKGROUND

Detecting, identifying, and phenotyping pathogens found in healthcare settings is critical both for diagnostic and surveillance purposes. Traditional bacterial and fungal diagnostic procedures rely on culture techniques that produce a genus or species level identification after 24-48 hours. Such tests are ordered for patients demonstrating symptoms indicative of an infection. While culture has been the standard diagnostic method for over one hundred years, its slow turnaround time means that a physician must prescribe antibiotics before knowing the identity of the organism or its drug resistances.

More recently, rapid techniques such as qPCR and mass spectrometry have allowed sub-24 hour turnaround times and enabled surveillance applications. For example, many hospitals in the United States test every patient for MRSA on admission to determine an appropriate caution level (e.g., quarantine) for patients who are at a high risk for spreading an infection to other patients. qPCR offers quick results but minimal information—a typical test only detects the presence of one or a few sequences from one organism. Testing for additional organisms or the presence of drug resistance or virulence genes adds substantially to the cost of the test.

A test that offers sub-24 hour turnaround time while identifying a large number of organisms would offer many benefits in a healthcare setting including broad-range surveillance and faster prescriptions of the most appropriate antibiotic. The present application discloses compositions, kits, and methods that can be used to detect any or several of a large set of organisms present in a sample as well as a number of families of drug resistance genes.

### SUMMARY

Provided herein are compositions, kits, and methods for identifying an organism. The organism can be a microbe, microorganism, or pathogen, such as a virus, bacterium, or fungus. In one embodiment, an organism is distinguished from another organism. In another embodiment, a strain, variant or subtype of the organism is distinguished from another strain, variant, or subtype of the same organism. For example, a strain, variant or subtype of a virus can be distinguished from another strain, variant or subtype of the same virus.

In some aspects, a probe set for identifying pathogenic organisms or strains in a sample comprising a plurality of probes that, when implemented in an assay, allows for detecting and distinguishing at least 5 different strains, variants, or subtypes of at least 3 pathogenic organisms, wherein each probe in said plurality comprises a first sequence that hybridizes to a 5′ end of a target sequence of said pathogen, a 3′ end of said pathogen, or to said target sequence is provided.

In some embodiments, pathogen strains or organisms comprise a virus, bacterium, or fungus. In some embodiments, the at least 3 pathogenic organisms include Staphylococcus aureus, Staphylococcus epidermidis, Staphylococcus saprophyticus, Acinetobacter baumanii, Clostridium difficile, Escherichia coli, Enterobacter (aerogenes, cloacae, asburiae), Enterococcus (faecium, faecalis), Klebsiella pneumoniae, Proteus mirabilis, Candida albicans, and Pseudomonas aeruginosa; or subtypes or strains thereof.

In some embodiments, the probe set can not only detect and distinguish between the at least 3 organisms but can also distinguish between common strains or subtypes of the organisms. In some embodiments, the probe set detects and distinguishes among the organisms responsible for more than 90% of the hospital acquired infections at some site.

In one aspect, a probe set for identifying the presence of drug resistance genes in the organisms in a sample comprising a plurality of probes that, when implemented in an assay, allows for detecting and distinguishing at least 3 classes of resistance genes, wherein each probe in said plurality comprises a first sequence that hybridizes to a 5′ end of a target sequence of said pathogen, a 3′ end of said pathogen, or to said target sequence is provided.

In one aspect, a kit containing any probe set described herein and the reagents and protocol to capture the target sequences of the organisms present in the input sample is provided.

In some aspects, a kit for the simultaneous detection of pathogens including three or more of the organisms listed in Table 2 is provided. In some embodiments, the kit is for research use. In some embodiments, the kit is a diagnostic kit. In some aspects, a kit for the simultaneous detection of antibiotic resistance genes including three or more of the genes listed in Table 3 is provided. In some embodiments, the kits described herein can be used to prepare DNA for massively parallel sequencing. In some embodiments, the kits described herein can provide molecular barcodes for the labeling of individual samples. In some embodiments, the kits described herein can include at least 10 of the probe sequences listed in Table 1.

In some embodiments, the kits described herein can be used to circularize single-stranded DNA probes by: (i) hybridization to a complementary target DNA sequence, (ii) extension across a gap by DNA polymerase, and (iii) ligation of the extended probe to form a single stranded, covalently closed circular DNA molecule.

In one aspect, a composition comprises a probe set for identifying pathogenic organisms or strains in a sample comprising a plurality of probes that, when implemented in an assay, allows for detecting and distinguishing three or more of the organisms listed in Table 2, wherein each probe in said plurality comprises a first sequence that hybridizes to a 5′ end of a target sequence of said pathogen, a 3′ end of said pathogen, or to said target sequence. In one embodiment, the plurality of probes, when implemented into an assay, allows for the substantially simultaneous detection and distinguishing of three or more of the antibiotic resistance genes listed in Table 3 is provided.

In one aspect, a composition comprises a probe set for identifying antibiotic resistance genes of pathogenic organisms or strains in a sample comprising a plurality of probes that, when implemented in an assay, allows for detecting and distinguishing three or more of the antibiotic resistance genes listed in Table 3, wherein each probe in said plurality comprises a first sequence that hybridizes to a 5′ end of a target sequence of said pathogen, a 3′ end of said pathogen, or to said target sequence is provided.

In one aspect, a composition comprises a probe set for identifying pathogenic organisms or strains in a sample comprising a plurality of probes that, when implemented in an assay, allows for detecting and distinguishing three or more organisms that cause Hospital Associated Infections (HAIs) at some site, wherein each probe in said plurality comprises a first sequence that hybridizes to a 5′ end of a target sequence of said pathogen, a 3′ end of said pathogen, or to said target sequence is provided. In some embodiments, the three or more organisms that cause HAIs at some site comprise organisms responsible for more than 90% of the hospital acquired infections at some site. In some embodiments, the three or more organisms that cause HAIs at some site comprise organisms responsible for more than 60% of the hospital acquired infections at some site. In some embodiments, the three or more organisms that cause HAIs at some site comprise organisms responsible for more than 30% of the hospital acquired infections at some site. In some embodiments, the site is a surgical site, catheter, ventilator, intravenous needle, respiratory tract catheter, medical device, blood, blood culture, urine, stool, fomite, wound, sputum, pure bacterial culture, mixed bacterial culture, bacterial colony, or any combination thereof.

In some embodiments, a probe set is operable to detect CARB, CMY, CTX-M, GES, IMP, KPC, NDM, ampC, OXA, PER, SHV, VEB, VIM, ermA, vanA, canB, mecA, or mexA family or classes of genes, or any combination thereof. In some embodiments, some of the genomic regions chosen as target sequences are known to be highly conserved such that each genus or species tends to contain a single version of the region, thus allowing genus or species identification. In some embodiments, some of the genomic regions chosen as target sequences are known to be highly variable such that each strain or substrain will contain a different version of the region, thus enabling strain or substrain identification and differentiation. In some embodiments, some portion of a plurality of the selected target sequences are sequenced simultaneously and then mapped to a database of reference sequences to determine the most likely identities of the organisms or genes present in the sample. In some embodiments, some portion of a plurality of the selected target sequences are sequenced simultaneously and then assembled into one or more consensus sequences. When sequencing information is gathered from the probes for antibiotic resistance genes, for plasmids, and for an organism, a distinguishing fingerprint can be derived for the pathogen, and can serve as means to identify the source and extent of an outbreak.

In one aspect, a kit comprising one or more reagents, wherein the reagents comprise a probe set according to claims 1-11, reagents for obtaining a sample, reagents for extracting nucleotides from a sample, enzymes, reagents for amplifying a region of interest, reagents for purifying nucleotides, reagents for purifying captured regions of interest, buffers, sequencing reagents, or any combination thereof, wherein the reagents allow for the capture of target sequences of three more pathogens listed in Table 2 is provided.

In one aspect, a kit comprising one or more reagents, wherein the reagents comprise a probe set according to claims 1-11, reagents for obtaining a sample, reagents for extracting nucleotides from a sample, enzymes, reagents for amplifying a region of interest, reagents for purifying nucleotides, reagents for purifying captured regions of interest, buffers, sequencing reagents, or any combination thereof, wherein the reagents allow for the capture of target sequences of three or more antibiotic resistance genes listed in Table 3 is provided.

In one aspect, a kit comprising one or more reagents, wherein the reagents comprise a probe set according to claims 1-11, reagents for obtaining a sample, reagents for extracting nucleotides from a sample, enzymes, reagents for amplifying a region of interest, reagents for purifying nucleotides, reagents for purifying captured regions of interest, buffers, sequencing reagents, protocol or any combination thereof, wherein the reagents allow for the capture of target sequences of three or more pathogens listed in Table 2 and capture of target sequences of three or more antibiotic resistance genes listed in Table 3 is provided.

In some embodiments, the reagents allow the capture reaction to be performed in a single tube. In some embodiments, the reagents allow the capture reaction to be performed in less than three hours. In some embodiments, the reagents allow the capture reaction to be performed in less than two hours. In some embodiments, the detection of the three or more pathogens occurs substantially simultaneously.

In some embodiments, the plurality of probes comprises at least 3 of the probe sequences listed in Table 1. In some embodiments, each probe comprises the first sequence that hybridizes to a 5′ end of said target sequence and a second sequence that hybridizes to a 3′ end of said target sequence. In some embodiments, the probe set can distinguish between strains or subtypes of the organisms. In some embodiments, the detection the three or more antibiotic resistance genes occurs substantially simultaneously. In some embodiments, the detection of the three or more pathogens and the three or more antibiotic resistance genes occurs substantially simultaneously.

In some embodiments, a kit allows for preparation of DNA for massively parallel sequencing. In some embodiments, a kit further comprises molecular barcodes for the labeling of individual samples.

In some embodiments, the probe set of a kit comprises at least 10 of the probe sequences listed in Table 1. In some embodiments, the probe set of a kit comprises at least 20 of the probe sequences listed in Table 1.

In some embodiments, kit reagents can be used to circularize single-stranded DNA probes by: (i) hybridization to a complementary target DNA sequence, (ii) extension across a gap by a DNA polymerase, and (iii) ligation of the extended probe to form a single stranded, covalently closed circular DNA molecule.

In one aspect, a method of identifying an organism or pathogenic strain, variant or subtype comprising: a) contacting a sample with a plurality of probes listed in Table 1, wherein said plurality of probes detects and distinguishes at least 3 different organisms or pathogenic strains listed in Table 2, or variants or subtypes thereof; b) hybridizing a 5′ end of a target sequence of said organisms or pathogenic strains, or variants or subtypes thereof, a 3′ end of said target sequence, or said target sequence with a probe of said plurality; c) sequencing said target sequence; and d) identifying from said sequencing said organisms or pathogenic strains, or variants or subtypes thereof is provided.

In one embodiment, the method is performed in less than 12 hours. In one embodiment, the identifying is performed in less than 3 hours. In one embodiment, the identifying is performed in less than 2 hours. In one embodiment, the identifying is with at least 99% specificity or sensitivity.

In one aspect, a method of stratifying a host into a therapeutic group comprising: a) contacting a sample from said host with a plurality of probes listed in Table 1, wherein each probe specifically distinguishes different non-host organisms or pathogenic strains listed in Table 2, or variants or subtypes thereof; b) hybridizing a 5′ end of a target sequence of a non-host organism or pathogen, a 3′ end of said target sequence, or said target sequence with a probe of said plurality; c) sequencing said target sequence; d) determining an identity of said non-host organism or pathogenic strain, or variant or subtype thereof, from said sequencing; and e) stratifying said host into a therapeutic group based on said identity is provided. In one embodiment, the method further comprises determining the genotype of the host from the sample.

In some embodiments, an additional non-host organism is identified. In some embodiments, an additional strain, variant or subtype of said organism or pathogen is identified. In some embodiments, the therapeutic group differs than a therapeutic group in which only one of the non-host organisms is identified. In some embodiments, the therapeutic group differs than a therapeutic group in which only one of said strains, variants, or subtypes of said pathogen is identified.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts an exemplary kit configuration, indicating the position of samples and barcoding reagents within the supplied materials within the kit.

FIG. 2 provides a matrix depiction of a subset of a probeset for discrimination of genus and species amongst many genomes of various organisms. Each column on the x-axis indicated a single probe capture region, and each row indicates a reference database genome within the genus or species labeled. Dark boxes indicate that a probe is not predicted to provide sequence for this organism, whereas white boxes indicate that this probe is predicted to bind and provide sequence enabling the detection of this organism.

FIGS. 3A-3B depict exemplary plots of data that can be used to quantify target organisms including (FIG. 3A) Acinetobacter and (FIG. 3B) S. saprophyticus. In each case, genomic DNA isolated from a culture of each organism was quantified and a dilution series of 4 orders of magnitude aliquoted. Each aliquot was sequenced in triplicate and the total sequencing reads per aliquot divided by the number of internal control reads to produce a normalized quantitation of the DNA present in the sample. The plotted results indicate a highly linear and quantitative relationship between sequenced reads detected and input DNA.

FIG. 4 depicts a graph showing that the kits described herein can resolve mixed samples containing multiple organisms. In each case, genomic DNA isolate from a culture of each organism was quantified and a aliquoted into a sample at even copy numbers, with the sample matrix indicating mixes of up to 5 distinct genomes within each sample. Each sample was sequenced in duplicate and the total sequencing reads for each individual genome per sample divided by the number of internal control reads to produce a normalized relative quantitation of the each of the genomic DNA species present in the sample. The graphed results indicate accurate detection of multiple species within a mixed DNA sample.

FIG. 5 depicts a plot demonstrating strong correlation (R²=0.98) between the log normalized counts obtained via PGM vs log normalized counts obtained via qPCR. Genomic DNA from an organism was quantified and a aliquoted as a dilution series over ˜5 orders of magnitude. Each sample was sequenced in triplicate and the total sequencing reads for the genome per sample divided by the number of internal control reads to produce a normalized relative quantitation genomic DNA species present in each aliquot. qPCR was also performed in triplicate on each sample using a genome specific primer pair, and the qPCR relative copy number then plotted against the sequencing data (PGM normalized count). The results demonstrate a linear agreement with quantitation by qPCR over >4 orders of magnitude.

FIG. 6 depicts a plot of the ratio of viral (HIV) reads to GFP against the initial template concentration in the reaction. cDNA from HIV was quantified and a aliquoted as a dilution series over ˜4 orders of magnitude. Samples were prepared in the presence of 1000 genome equivalents of human DNA isolated from cultured HEK-293 cells, or in the absence of background competing DNA. Each sample was sequenced and the total sequencing reads for the genome per sample divided by the number of GFP internal control reads to produce a normalized relative quantitation of HIV cDNA present in each aliquot. No significant difference was observed in the number of sequencing reads per sample in the presence or absence of competing background Human DNA.

FIG. 7 depicts a plot comparing the detection of cDNA from 2 HIV strains (CN009 and CN006) obtained via PGM vs. MiSeq. The plot is shown as the adjusted GFP read count against the CN009 Template count. In each case, cDNA from HIV CN009 was quantified and a aliquoted as a dilution series over ˜5 orders of magnitude. Into each CN009 aliquot, 3000 genome equivalents of CN006 genome were also added. Each sample was sequenced in duplicate and the total sequencing reads for each individual genome per sample divided by the number of internal control reads to produce a normalized relative quantitation of the each of the genomic DNA species present in the sample. The plots indicated a consistent level of CN006 detection detected per sample, and a linear detection of CN009 over >4 orders of magnitude. This also demonstrates the detection of two species at minor variant frequencies of as low as 1%.

FIG. 8 depicts a plot of sequencing counts per probe within a probeset, for replicate sample B1 against replicate sample B2. The plot demonstrates a highly linear and reproducible probeset internal performance.

FIGS. 9A-9B depict plots of the ratio of minor:major pathogen PGM reads against the percent ratio of minor:major pathogen in the reaction for (FIG. 9A) minor pathogen detected to 1% major pathogen (S. epidermidis and E. coli) and (FIG. 9B) minor pathogen detected to 10% major pathogen (S. saprophyticus and A. baumannii). In each case, genomic DNA isolated from a culture of two organisms was quantified and aliquoted into a sample at a ratio of 10:1, 1:1, 1:10 and 1:100. Each sample was sequenced in triplicate and the total sequencing reads for each individual genome per sample divided by the number of internal control reads to produce a normalized relative quantitation of the each of the genomic DNA species present in the sample. The graphed results indicate accurate detection of multiple species within a mixed DNA sample down to a minor variant level of at least 1%.

FIG. 10A describes a list of assay components within the HAI BioDetection kit.

FIG. 10B illustrates the layout of customer samples and control samples of two 8 well strips that the BioDetection assay is performed within.

FIG. 10C indicates validated performance specifications and criteria for the HAI BioDetection kit.

FIG. 11A describes the multilevel structure of the HAI BioDetection probeset.

FIG. 11B provides a matrix depiction of two probes within the BioDetection probset to illustrate discrimination of species and strain amongst many genomes of Staphlyococcus. Each column on the x-axis indicates a SNP detected by either probe 1 or probe 2 capture region, and each row indicates a reference database genome within the genus or species labeled. Black boxes indicate that a probe is not predicted to provide sequence for this organism, whereas shaded boxes indicate that this probe is predicted to bind and provide sequence enabling the detection of this organism.

FIG. 11C describes the levels of multiplexity achieved by the HAI BioDetection kit by assaying many sequence variants within a sample, compared to the single nucleotide discriminatory ability of a PCR primer.

FIG. 12 illustrates the workflow from input sample, either a purified genomic DNA from culture, e.g. DNA enriched from a swab of a patient wound site. The BioDetection kit workflow is illustrated in elapsed time (from t=12.00) and the workflow timing for each individual step is broken out to the left of the workflow. A barcoding primer set of allows 16 to 96 samples to be sequenced simultaneously in one run on a sequencing platform (in this illustration and Ion Torrent PGM, but alternatively and Illumina MiSeq or HiSeq platform). The interpretation box illustrates a computational software and graphical display of simplified data output.

FIG. 13 illustrates a graphical display of summarized sequencing results from the BioDetection kit. The graphical display is subdivided into Genus, species and strain level detection results, resistance gene information if resistance loci are detected, the readcount for samples and internal controls, and also any potential warnings due to poor sample performance. A color-coded similarity score (green=similar, yellow=moderate similarity, red=little similarity) and a similarity score absolute value, are calculated for the sequenced similarity detected by the kit and compared to the next most related organism at genus, species and strain level using a reference database of published genomic sequences, and containing previous genome sequences detected by the BioDetection kit. In the illustrated example, the sample has been demonstrated to contain both Enterococcus faecalis as the primary species, and Escherichia coli a minor species present within the sample. The samples are 65.4% and 74% homologous to the nearest neighbor strains described.

FIG. 14 illustrates a schematic comparison of the turnaround time and workflow steps to generate substrain level resolution and drug resistance typing of bacterial samples using either traditional microbiology, and combination of PCR and/or Mass spectroscopy, whole genome sequencing (WGS) or the BioDetection kit. A clear advantage is illustrated with the BioDetection kit in terms of fewer workflow steps and faster achievement of substrain resolution and drug resistance typing compared to these alternative methods.

FIG. 15A describes a collection of 38 MRSA samples were subtyped using both HAI BioDetection Kit and spa locus VNTR typing (using PCR and Sanger sequencing). Sequence regions captured using the BioDetection kit were used to construct a phylogenetic tree was constructed using sequence data, and each sample was annotated with spa-typing result for the same sample. The tree demonstrates the discrimination of samples with the same spa-type into multiple unique isolates using the BioDetection kit. Further the grouping clustering generated by the BioDetection kit largely groups according to spa-type, as would be predicted for more closely related samples.

FIG. 15B describes the number of sequence variants detected amongst 38 sequenced with the BioDetection kit, or typed by spa type, or 8 representative samples encapsulating the broad phylogenetic tree structure and then Sanger sequenced using then MLST subtyping amplicons, or the 16S ribosomal sequencing amplicon. The total number of MRSA samples uniquely discriminated by each approach is also described.

FIG. 15C describes 4 bacterial cohorts sequenced using the BioDetection kit. The table indicates the number of samples per cohort, and the number of % of the samples that were discriminated into unique isolates. The data demonstrates that the HAI kit is capable of near unique discrimination of bacterial isolates within many large cohorts.

FIG. 16A is an in silico model predicting the superiority of the present invention over VNTR approaches. Thirty-three genome sequences were extracted from references databases for which whole genome sequence assemblies were available. An in silico analysis extracted regions of these genomes that are assayed using MLVA, MLST and spa subtyping methods. The discriminatory index (defined as the % of total genomes discriminated into unique isolates) of each technique was calculated based upon the assayed regions, and compared with the regions assayed by the BioDetection kit. The BioDetection kit discriminated all samples into unique groups, and demonstrated a higher discriminatory index than other assays.

FIG. 16B is a tangelgram demonstrating better phylogeny reproduction by the BioDetection kit. The sequences extracted by the in silico analysis of Figure x2 were used to construct phylogenetic trees to describe the relationships between samples. The whole genome sequence (WGS) was used as a reference tree, and the BioDetection and MLVA constructed trees compared using a tangelgram figure. Red and pink lines represent regions of the tree that are significantly different between methods, whereas parallel grey lines illustrate relationships that are described equivalently by both methods. The figure demonstrates significant discordance between the MLVA and WGS trees, but largely comparable phylogenetic relationships described by the WGS and BioDetection trees. This data indicates that the evolutionary relationships described by the BioDetection kit are a more accurate appraisal of the whole genome relationships and evolutionary distance between samples than MLVA approaches. Similar significant discordances were observed to between WGS and spa-typing and MLST trees compared to MLVA.

FIG. 17 demonstrates detection from DNA isolated from stool, urine and sputum. Sputum, stool and urine sample derived from human individuals were spiked with genomic DNA isolated from cultured bacteria. Samples were extracted using standard DNA extraction methods, such that each sample contained some amount of gDNA plus any additional complex biomolecules that are carried through the extraction from these sample types (heme, complex polysaccharides and potential enzyme inhibitors). Each isolated DNA sample was assayed using the BioDetection kit and results are tabulated. This data demonstrates accurate species and strain detection from DNA isolated from sputum, urine and stool samples, plus identification of resistance genes present within each sample.

FIG. 18 is a summary of 707 bacterial samples sequenced and identified using the BioDetection kit. The table demonstrates the capability of the kit to detect species not listed and validated within the performance specifications, due to the broad detection and discriminatory ability of the selected sequence capture regions.

FIG. 19 shows detection of VRE from rectal swabs. A collection of n=24 positive and negative rectal swab samples screened by microbial culture were collected after primary screening at a hospital laboratory. Bound DNA released into a PBS wash solution by incubation for 1 hr at 37 degrees centrigrade, isolated using a gDNA extraction kit and then assayed using the BioDetection kit. The resulting data was tabulated to indicate the detection of organisms and drug resistance genes (plus read counts) from each sample, and comparison to the clinical surveillance by culture. This data demonstrates accurate species, strain and resistance gene detection from rectal swabs. In particular the data illustrates detection of multiple Enterococcus strains, plus vancomycin resistance, and additional co-present species on the rectal swabs, such as E. coli and K. pneumoniae. Further, sample PGCA963 demonstrates low level E. faecium and E. coli detection in a culture negative sample.

FIG. 20 describes a summary of drug resistance loci detected over n=707 clinical isolates and clinical specimens sequenced using the HAI kit. Read count for each marker exceeds a minimum of 10 reads and often incorporates detection of multiple sequences within the gene, providing high confidence detection. This table demonstrates a range of drug resistance markers confirmed for detection by the BioDetection kit.

FIG. 21 demonstrates a comparison of 2 samples sequenced using the BioDetection kit from clinical Klebsiella isolates. The sequence represents the captured sequence of a single probe within the BioDetection probeset. The pairwise sequence comparison illustrates mismatches between a single probe loci (of multiple discriminating probes) and indicates that even a single loci commonly contains multiple SNPs of high confidence discrimination between closely related species.

FIG. 22A shows high confidence SNP calling by readcount vs WGS. A cohort of 20 MRSA samples were sequenced using the BioDetection kit on an Ion Torrent PGM, and a Nextera™ whole genome sequencing approach on a MiSeq. Samples were sequenced on a Ion 316 chip (˜3.2M reads), and a single MiSeq run (˜15M reads). Reads were aligned to a reference genome and coverage compared between sequencing approaches. The plots describe the genomic coordinates (x-axis) and the log 10 sequencing read depth at each nucleotide (y-axis) for 3 individual probes. The BioDetection kit generates considerably higher readcounts (10-100 fold) at discriminatory regions between samples, enabling higher confidence SNP calling for this targeted sequencing vs the low read depth of whole genome sequencing. This also supports accurate detections for each of the SNPs by independent library constructions using different sequencing technologies.

FIG. 22B shows genomic coordinates. Two sequence alignments compare the consensus read sequence at 2 regions captured by both HAI BioDetection kit, and Nextera Nextera™ whole genome sequencing and reference genome alignment. For samples TC14, TC5 and TC4, the sequences show agreement for detection of an indel within sample TC14, and two SNPs within TC14 relative to TC4 and TC5.

### DETAILED DESCRIPTION

Approximately one out of every twenty hospitalized patients will contract a nosocomial infection, more commonly known as a hospital-acquired infection (HAI). More than 70 percent of the bacteria that cause HAIs can be resistant to at least one of the antibiotics most commonly used to treat them. Early detection can be important for controlling the spread of hospital-acquired infections. After culturing for growth and isolation of pathogens, clinical microbiology laboratories may rely on observable phenotype and simple biochemical assays to determine the bacterial type and antibiotic sensitivity. Determining the most effective antibiotic treatment for the infected patient, not the causal agent of the infection, is usually the prerogative of the physician. The resolution of conventional microbiological assays may be insufficient to determine the precise genotype underlying antibiotic resistance. Consequently, the same organism can infect multiple patients, and the spread of infection can go unnoticed for long periods.

Urinary tract infection (UTI) is the most common hospital-acquired infection. UTIs account for about 40 percent of hospital-acquired infections, and an estimated 80 percent of UTIs are associated with urinary catheters. Pneumonia is the second most common HAI. In critically ill patients, ventilator-associated pneumonia (VAP) is the most common nosocomial infection. VAP can double the risk of death, significantly increase intensive care unit (ICU) length of stay, and can add to each affected patient's hospital costs.

A key problem for microbiology labs is the turnaround time from receiving a microbial sample to determining key actionable information for patient care, such as antibiotic drug resistance within the sample, or strain identification for comparison to known high-risk strains. Existing technologies such as PCR or mass spectroscopy have allowed the turnaround time to be improved relative to classical methods for some actionable information, such as species identification, or presence of a select few drug resistance genes, but there are few practical approaches to assaying the large number of drug resistance genes or key species needed to be identified to confidently predict patient treatment.

DNA microarray offers broad detection ability for genomic loci, but is complicated by slow sample preparation and false positive and false negative sample results due to the hybridization based approach. Targeted DNA sequencing using the BioDetection kit allows the greater breadth of target detection, and higher resolution and higher accuracy discrimination due to the single base accuracy of DNA sequencing.

A second competing approach to targeted sequencing is whole genome sequencing. This approach has several disadvantages relative to the targeted sequencing approach provided by the invention. First, whole genome libraries contain many uninformative regions that are identical between the majority of isolates in a species, and thus provide no information to discriminate. These worthless reads mean that many more WGS reads are required per sample to capture informative regions, and prevent higher numbers of samples to be multiplexed into a single sequencing channel to amortize sequencing costs. Second, WGS libraries contain a representative fraction of any DNA present within a sample. As such, primary samples containing human tissue, or many uninteresting bacteria from the perspective of patient health, will comprise mainly of unwanted human or commensal bacterial reads. Efficient detection of important bacteria and drug resistance genes within a sample requires a more efficient targeted approach. Thirdly, library preparation times are slower and more laborious using WGS approaches, and the data analysis time significantly longer than that of a targeted sequencing approach in which only key informative regions are analyzed. This faster analysis reduces turnaround time and costs, and allow simplified data representations for easier understanding for clinical scientists unfamiliar with next generation sequencing data.

Provided herein are compositions, methods, systems and kits for detecting an organism, such as a pathogen, such as a pathogen that causes HAIs, as well as methods for using the system to identifying and detect the organism. The system can comprise a probe or plurality of probes. Also provided herein, are compositions, methods, systems and kits for detecting an organism, such as a pathogen, such as a pathogen that causes HAIs, and detecting and identifying antibiotic resistance genes, which, in some embodiments, can be performed simultaneously.

### Probes

In some embodiments, the invention provides panels of probes and methods of using them, where the panels include circularizing capture probes, such as molecular inversion probes. Basic design principles for circularizing probes, such as simple molecular inversion probes (MIPs) as well as related capture probes are known in the art and described in, for example: Nilsson et al., Science, 265:2085-88 (1994); Hardenbol et al., Genome Res.; 15:269-75 (2005); Akharas et al., PLOS One, 9:e915 (2007); Porecca et al., Nature Methods, 4:931-36 (2007); Deng et al., Nat. Biotechnol., 27(4):353-60 (2009); U.S. Pat. Nos. 7,700,323 and 6,858,412; and International Publications WO 2011/156795, WO/1999/049079 and WO/1995/022623, all of which are incorporated by reference in their entirety.

A system for detection of an organism, such as identifying a strain, variant or subtype of a pathogen, can comprise a mixture or probe set comprising a plurality of probes. The target organism for a particular probe may be any organism, such as a viral, bacterial, fungal, archaeal, or eukaryotic, organisms, including single cellular and multicellular eukaryotes. In particular embodiments, a target organism is a pathogen. In some embodiments, target organisms include organisms associated with or that cause HAIs, such as those organisms provided in Table 2.

In some embodiments, each single-stranded capture probe can hybridize to two complementary regions on a target DNA with a gap region in between. An enzyme, such as DNA polymerase, can be used to fill in the gap using the target as template, and stop adding nucleotides when it reaches the phosphorylated 5′-terminus of the hybridized probe. An enzyme, such as a thermostable ligase, can be used to covalently close the extended probe to form a circular molecule. Exonucleases can be used to digest away residual probe molecules. The filled-in, circularized probe can be resistant to exonuclease digestion, and can serve as template for preparation of the sequencing library by known methods, such as PCR. Sample-associated barcodes can be added and can enable multiple barcoded samples to be blended and analyzed together, such as on a DNA sequencer.

A probe can refer to a sequence that hybridizes to another sequence. The probe can be a linear, unbranched polynucleic acid. The probe can comprise two homologous probe sequences separated by a backbone sequence, where the first homologous probe sequence is at a first terminus of the nucleic acid and the second homologous probe sequence is at the second terminus to the nucleic acid, and where the probe is capable of circularizing capture of a region of interest of at least 2 nucleotides. Circularizing capture can refer to a probe becoming circularized by incorporating the sequence complementary to a region of interest.

In a preferred embodiment, the probes contain two arms, joined by a backbone, that hybridize to a target sequence. A polymerase molecule can extend the 3′ end of the probe by copying a target region into a probe molecule. A ligase molecule can circularize a probe molecule by joining the 3′ end of the copied target to the 5′ end of the original probe molecule.

In one embodiment, probe arms can hybridize to the target nucleic acid molecule, surrounding the capture region; a polymerase extension can fill in the gap between the arms and a ligase can create a circular molecule out of the extended probe. After an exonuclease digestion removes the original template molecules, primers can be used to amplify the captured probes. The primers can contain a 3′ end homologous to the backbone (forward) and its reverse complement (reverse primer). The 5′ of the primer may contain a sequencing adapter for a particular next generation sequencing platform and may also contain a barcode sequence between the 5′ and 3′ segments such that multiple samples, each amplified with primers containing a sample-specific barcode, can be multiplexed into a single sequencing run. As the two probe arms are linked by a backbone, on-target binding is energetically favorable, even when many (hundreds, thousands, or tens of thousands) of probes are present in a single reaction (compare to PCR, in which one primer of a pair may hybridize and extend at an off-target locus). As with PCR, each MIP can capture a well-defined region of the target sequence (compare to hybridization capture methods, which yield a variety of molecules centered around the target).

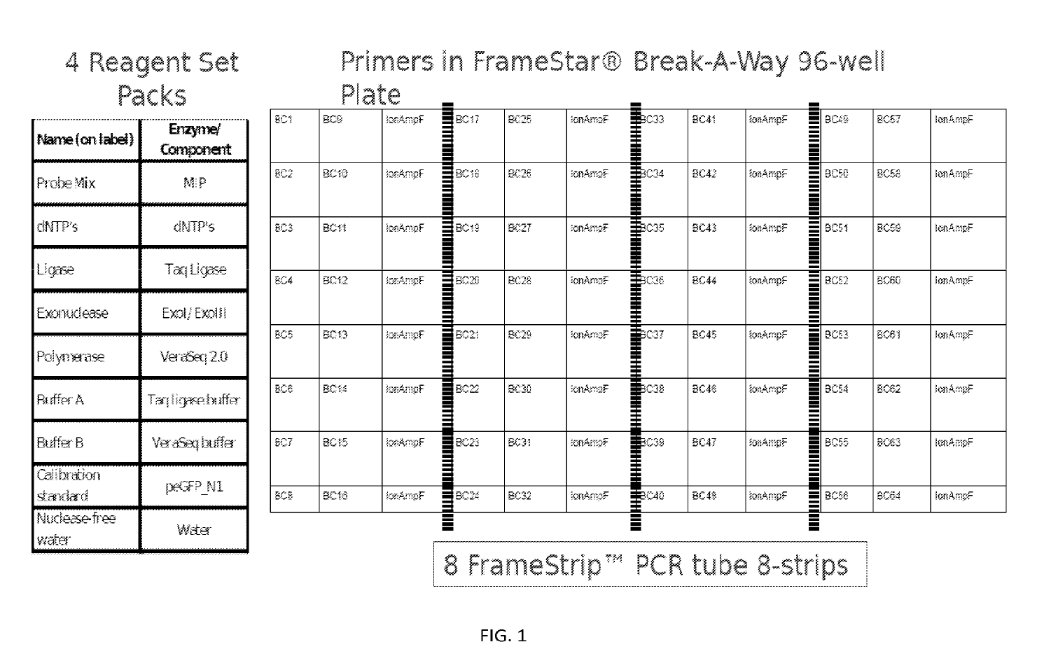

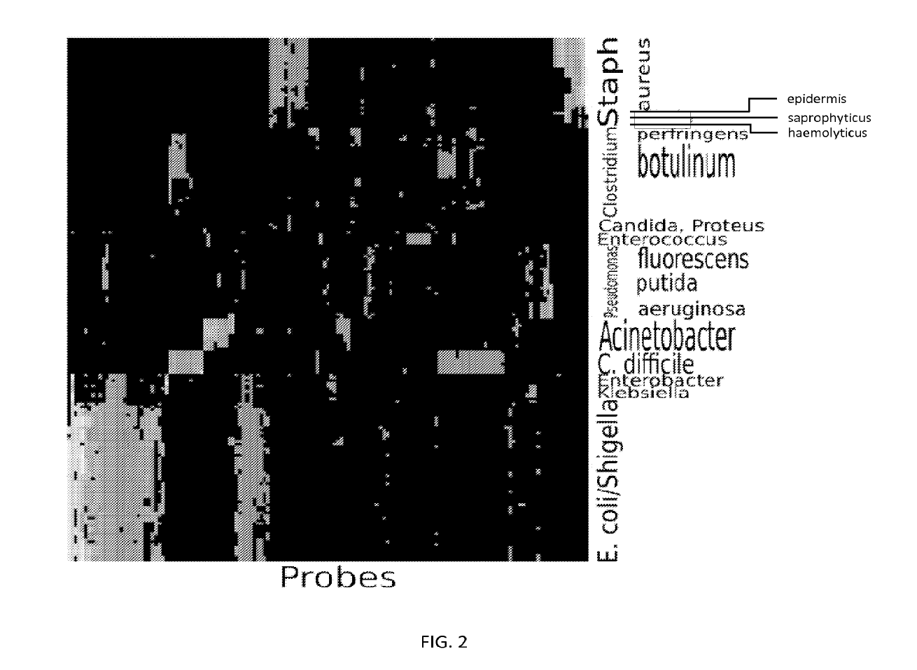

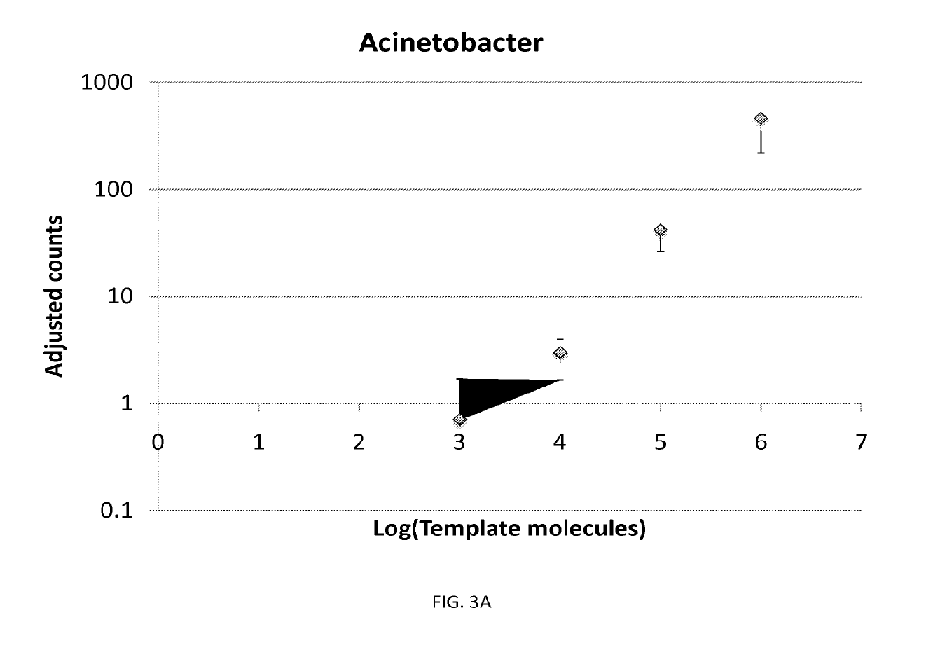

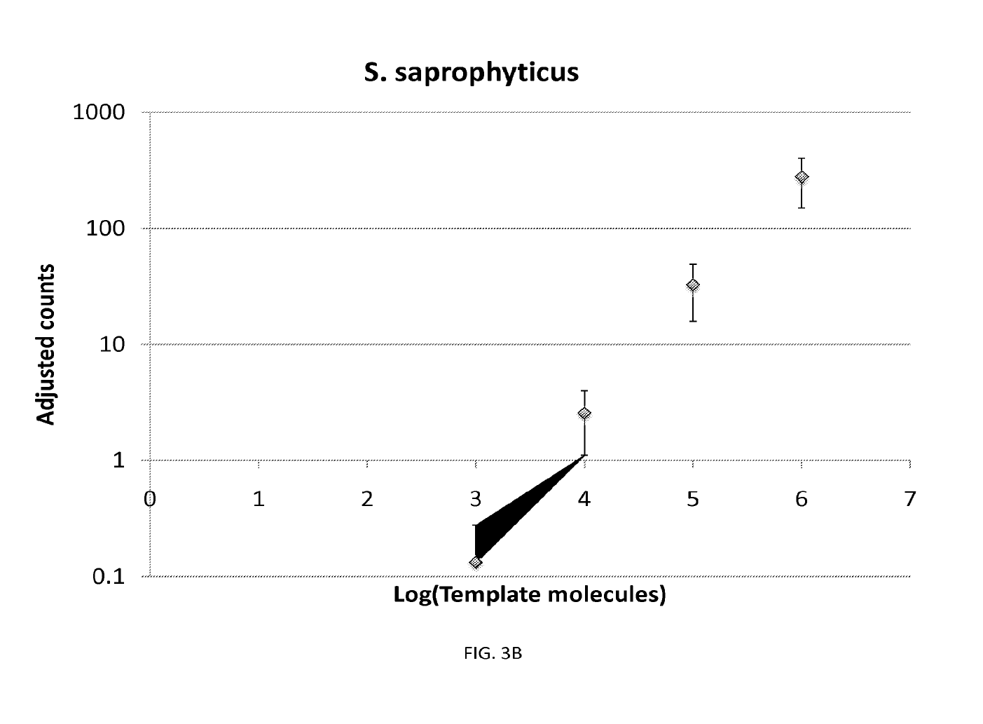

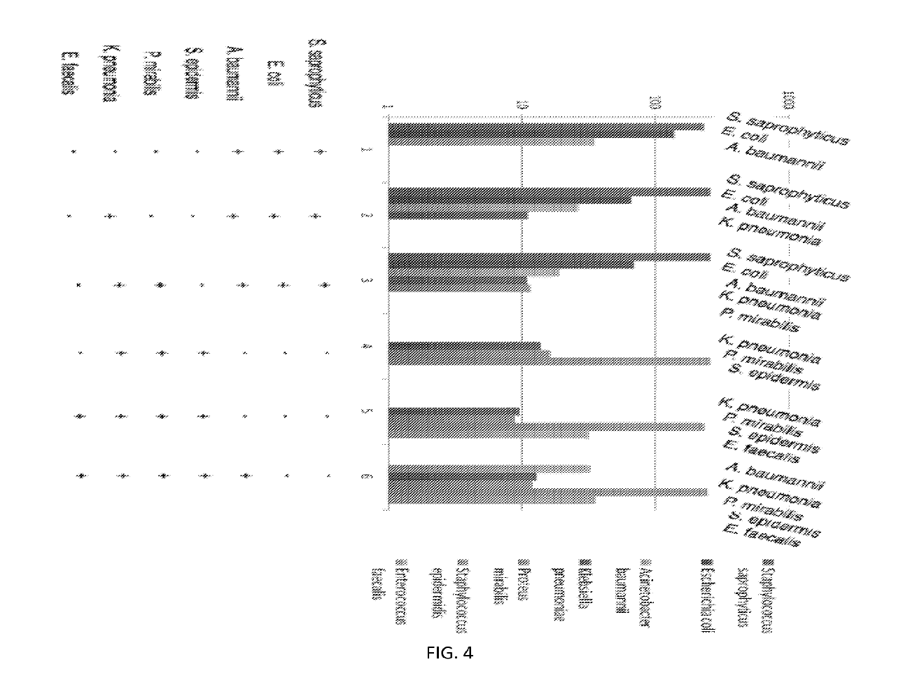

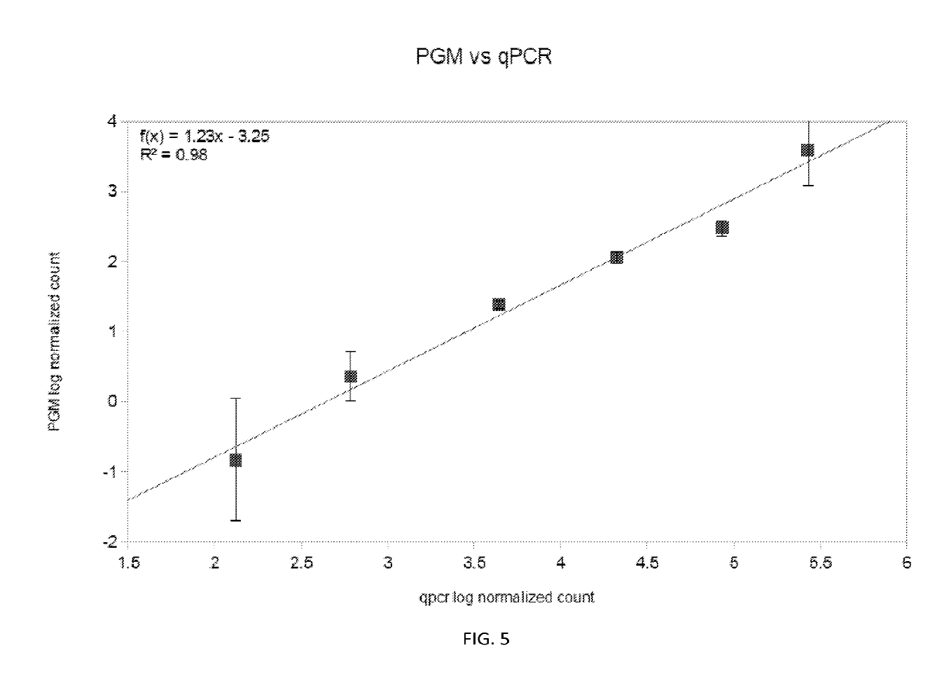

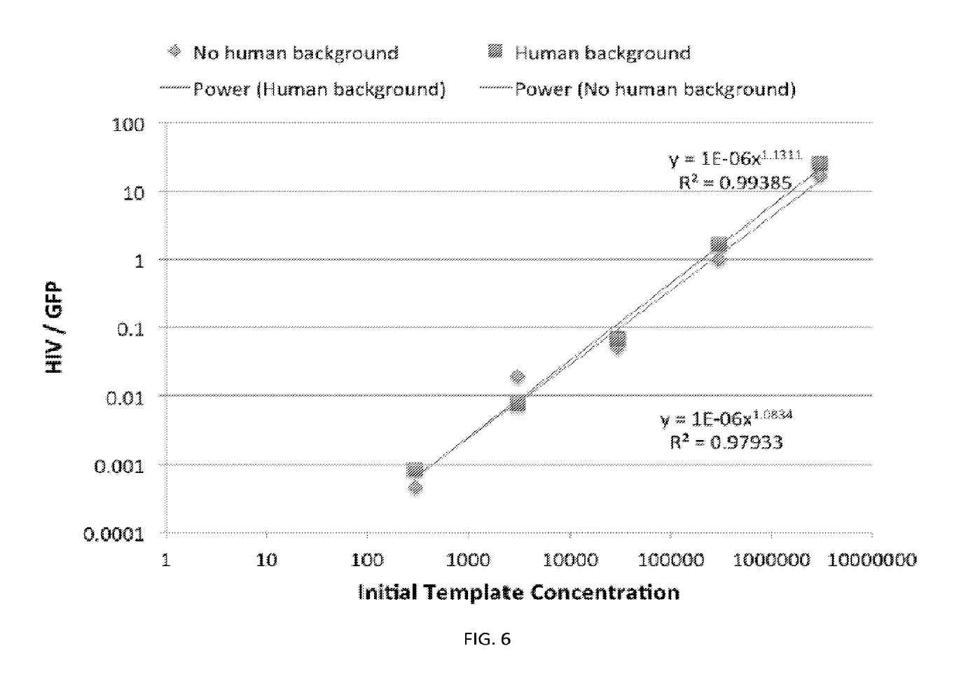

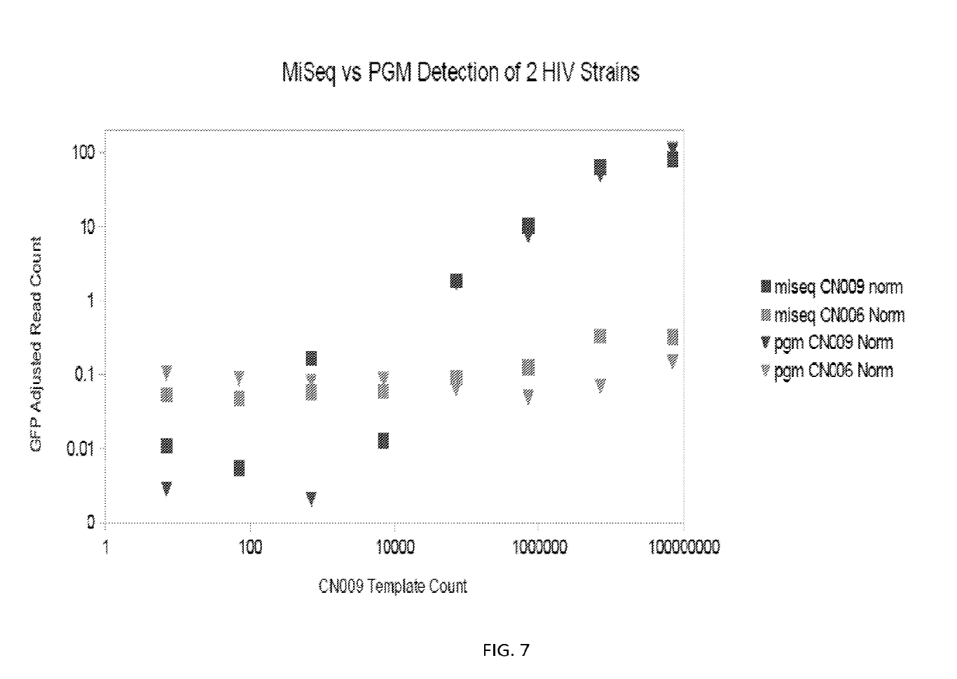

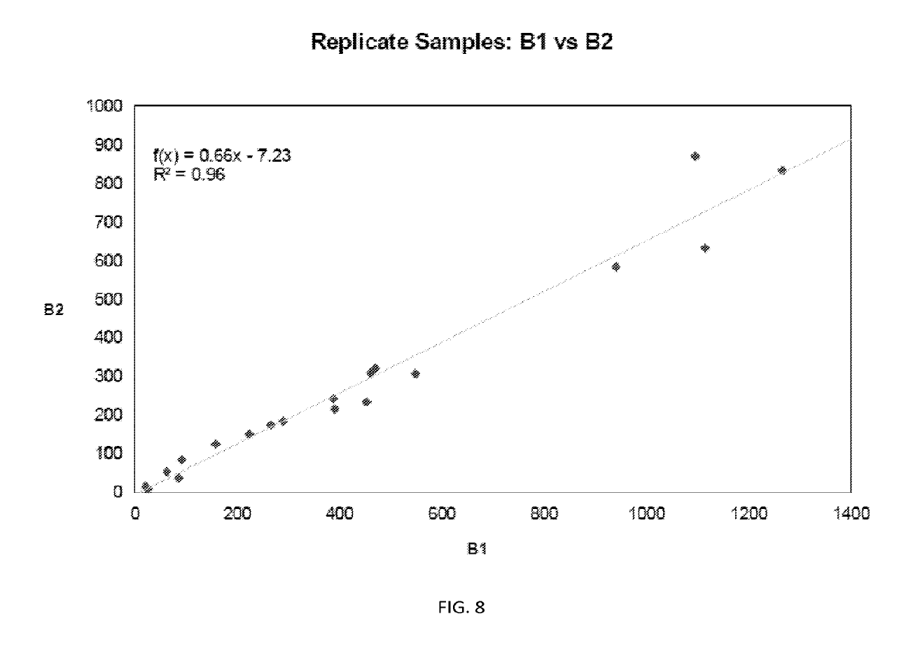

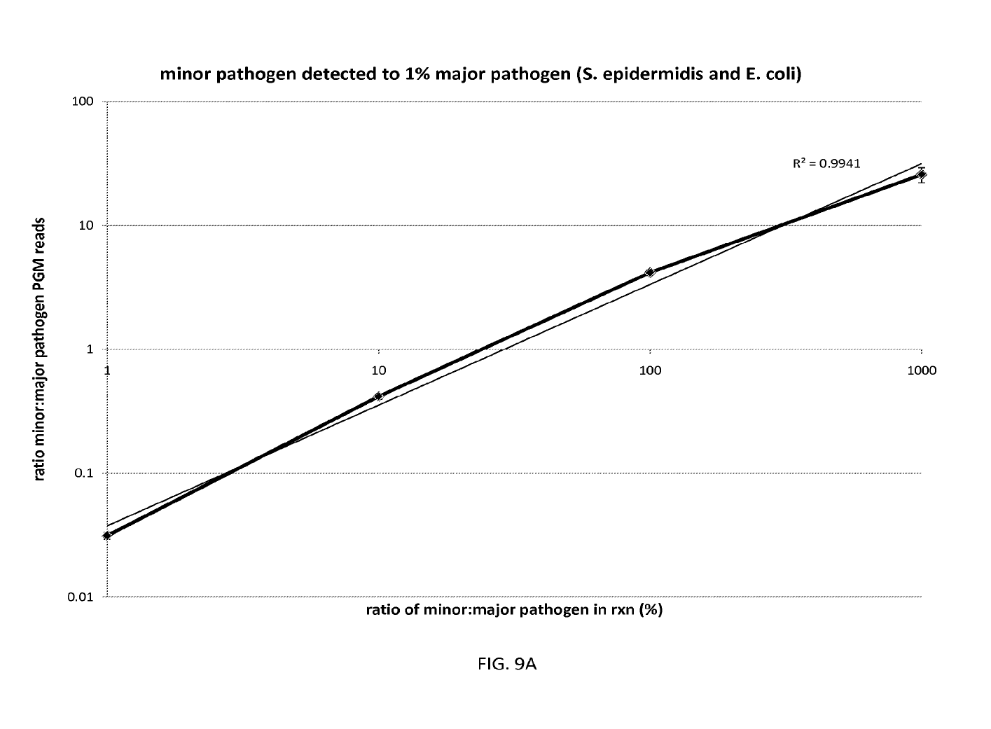

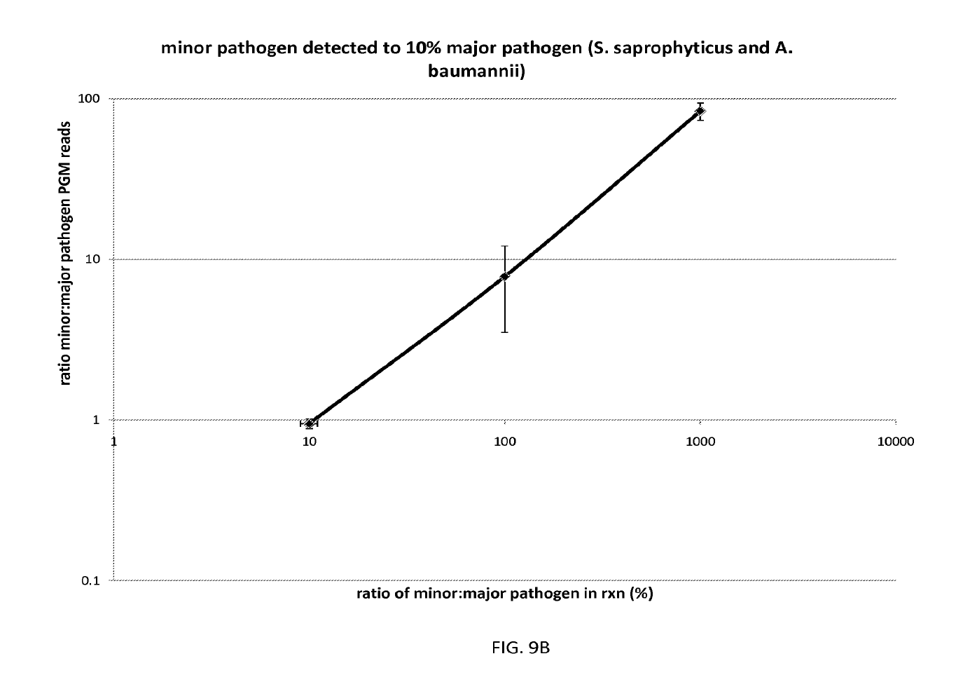

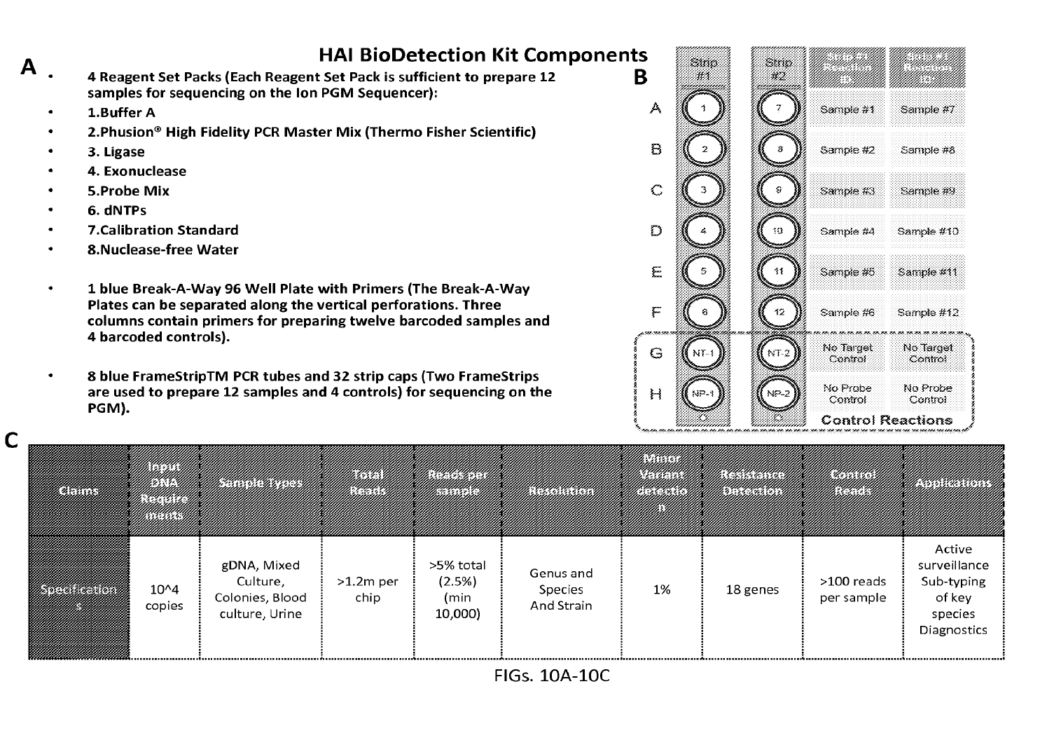

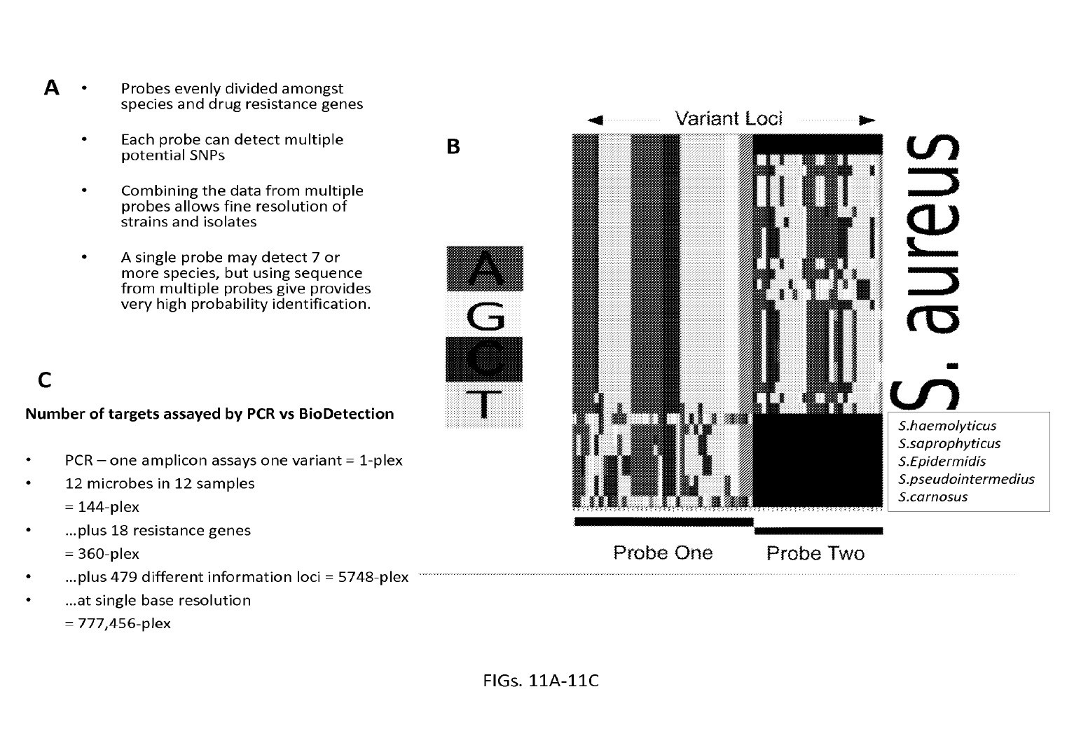

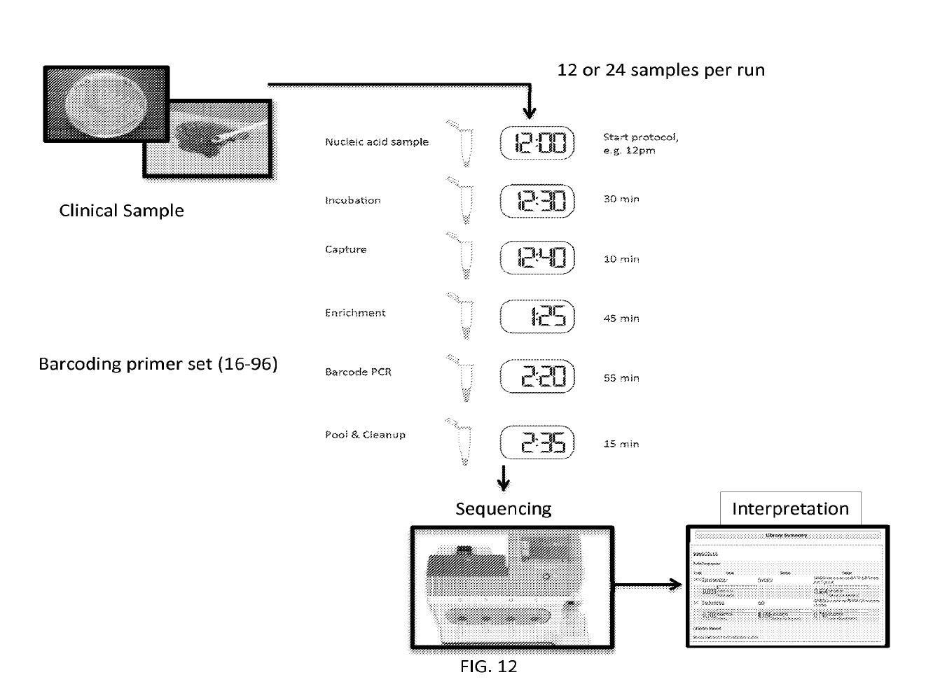

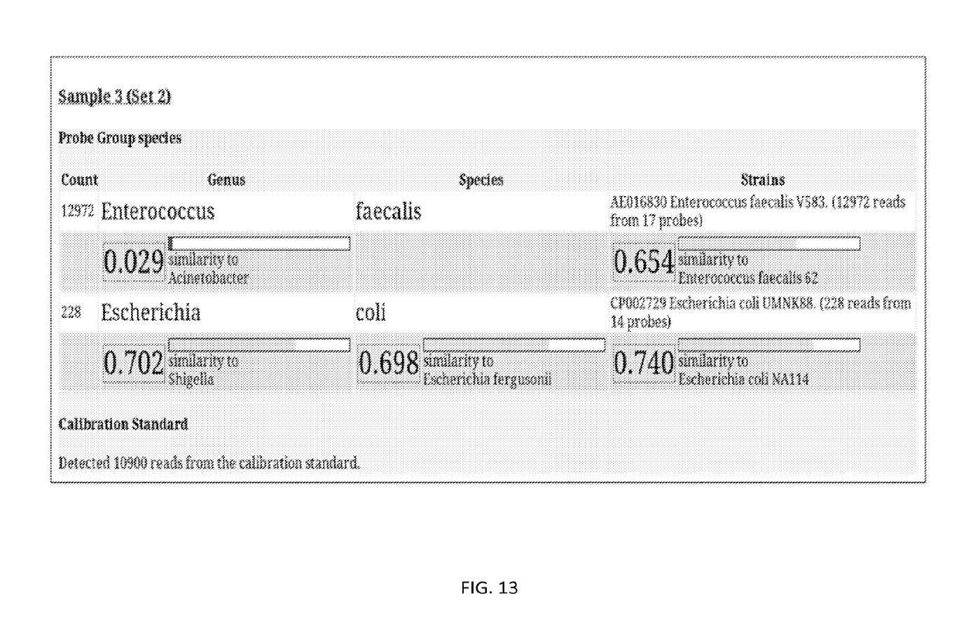

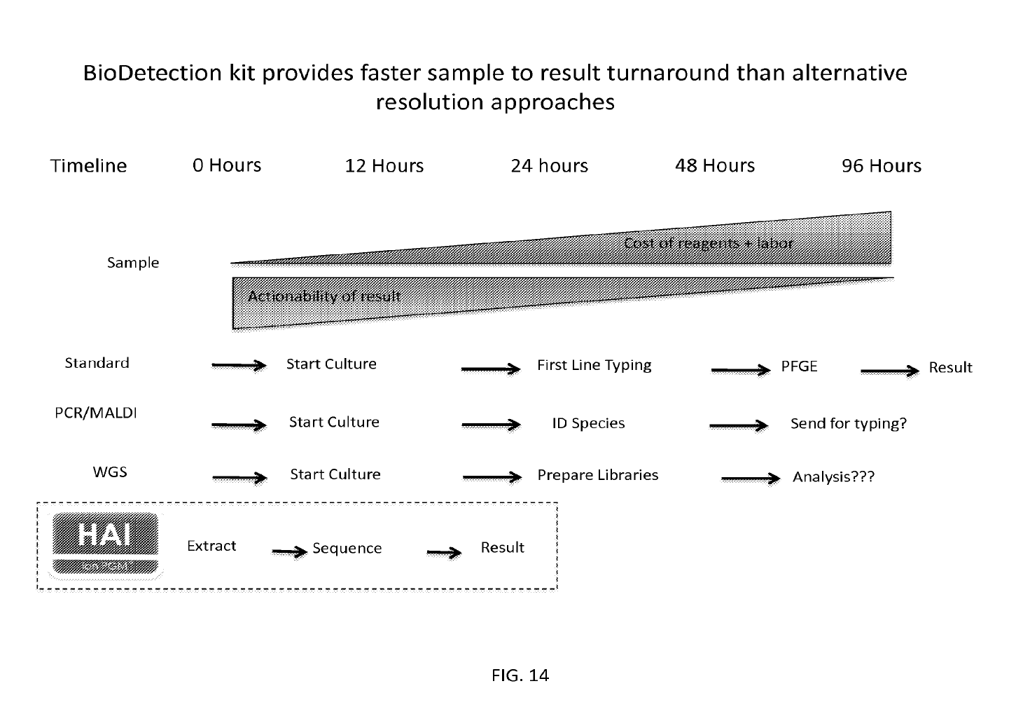

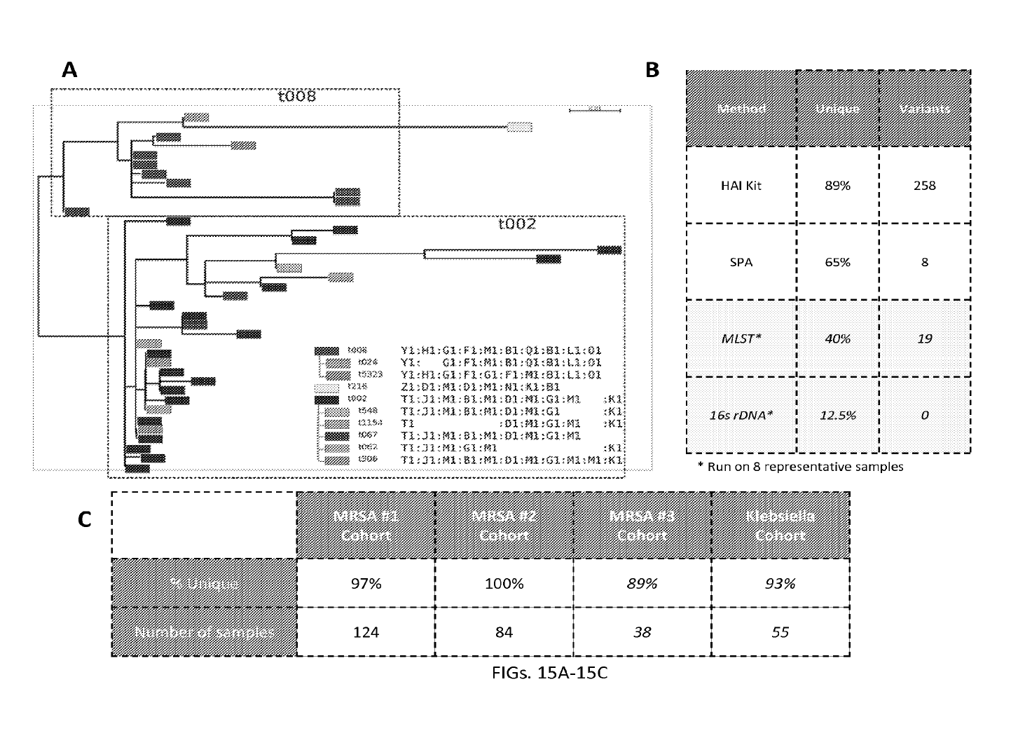

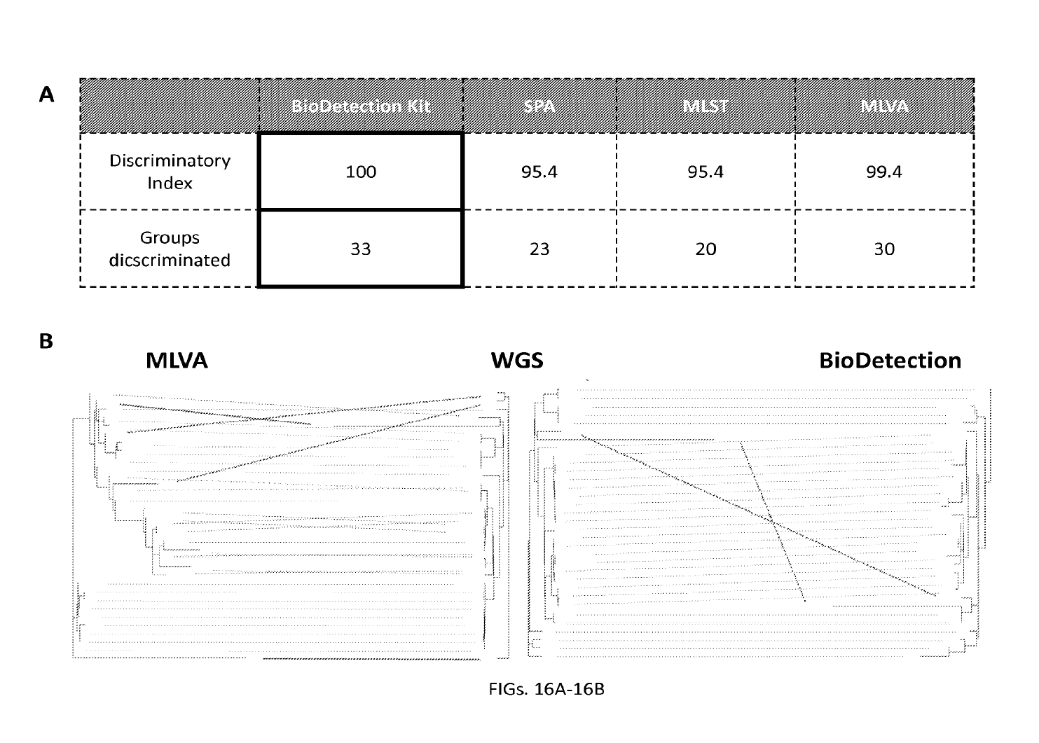

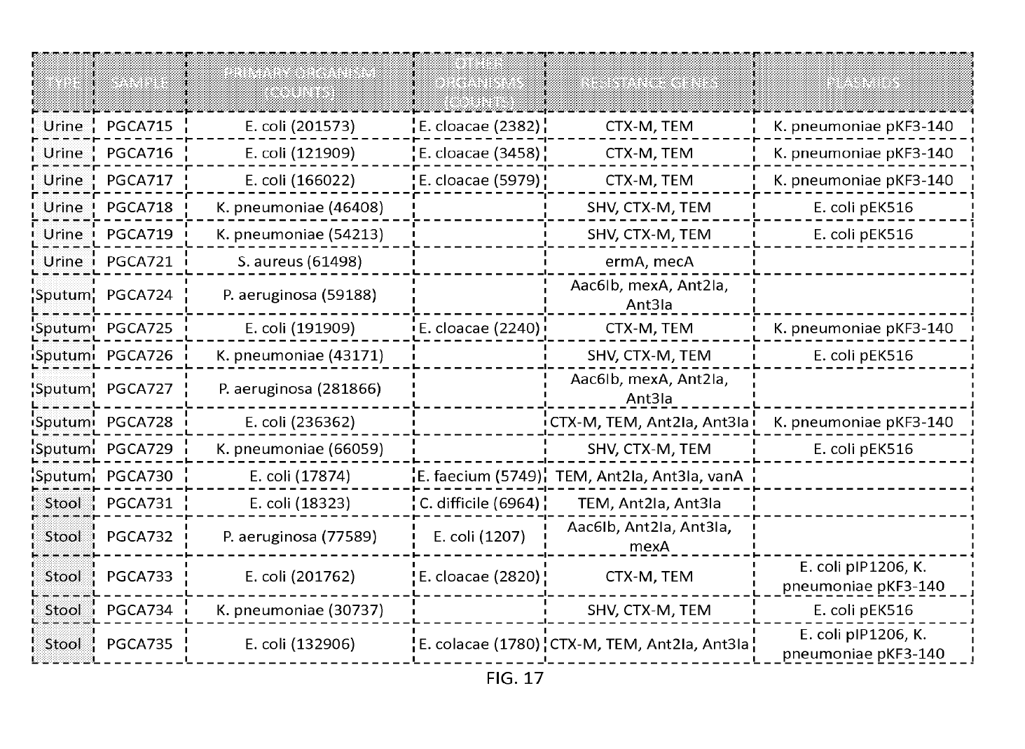

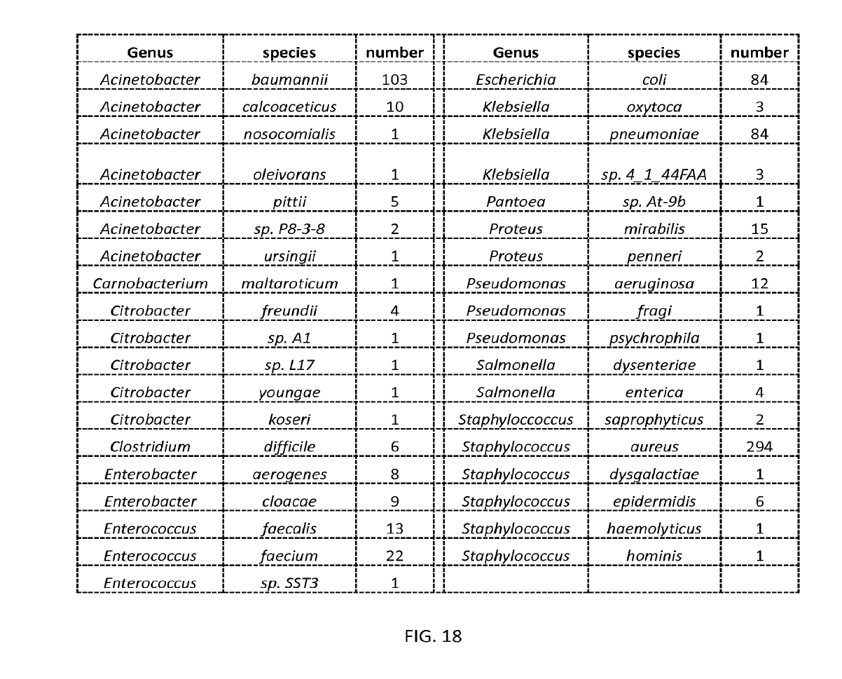

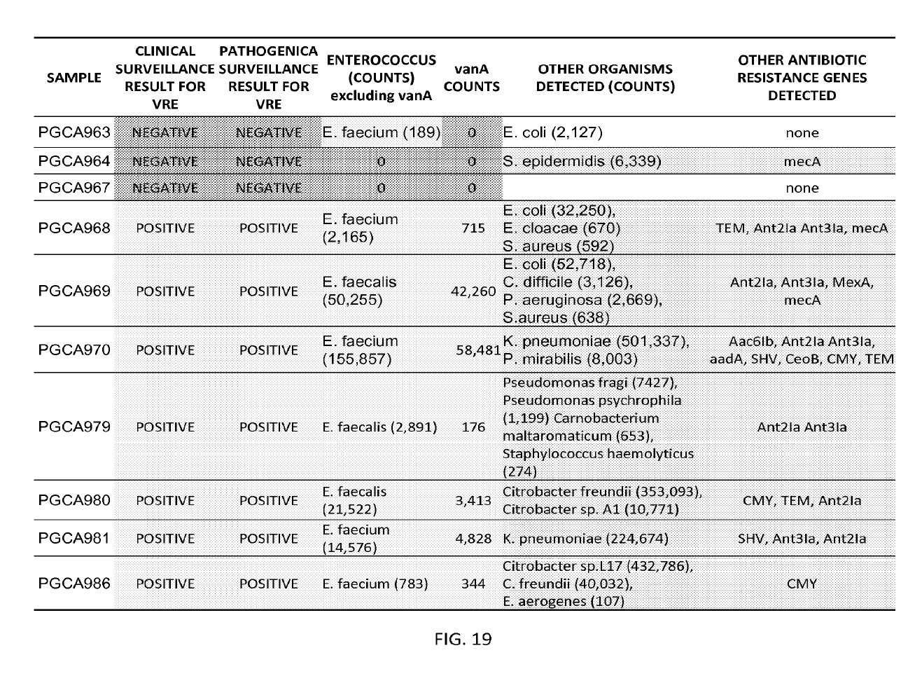

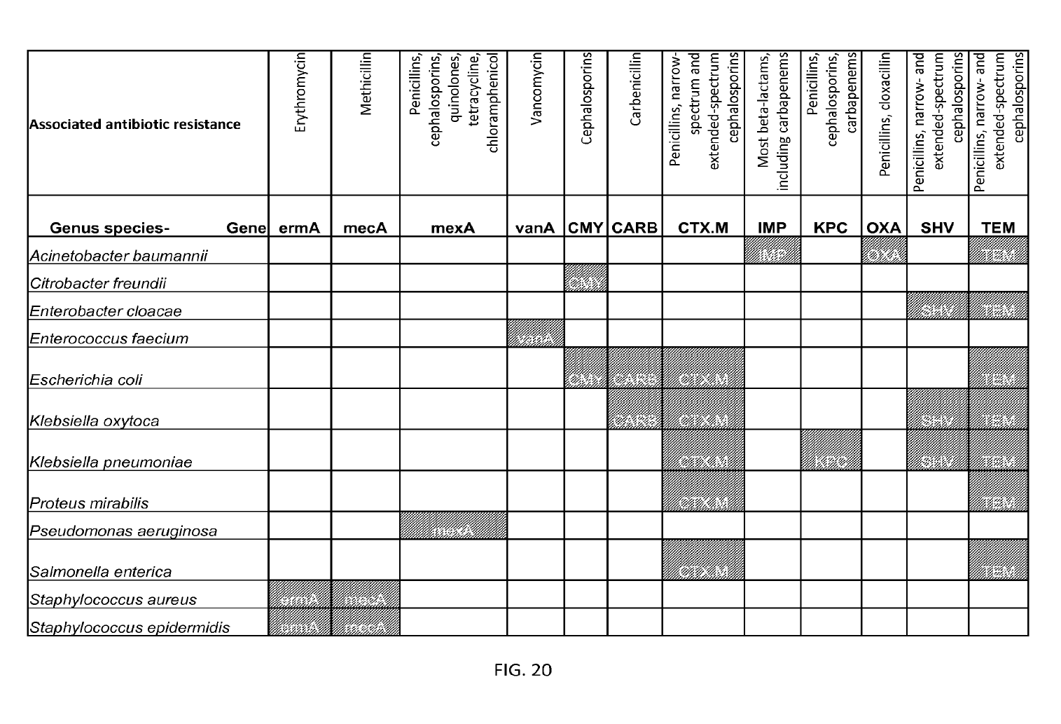

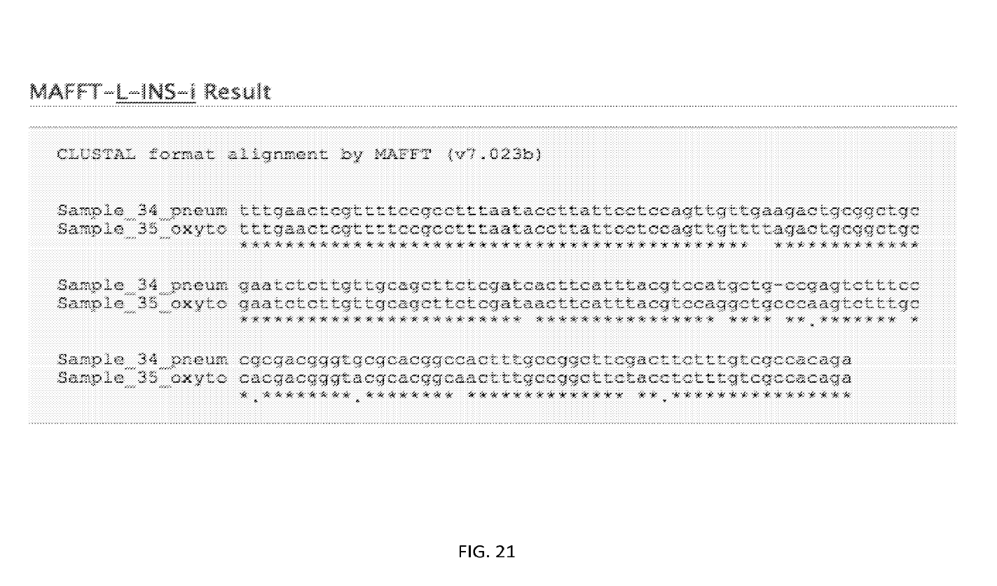

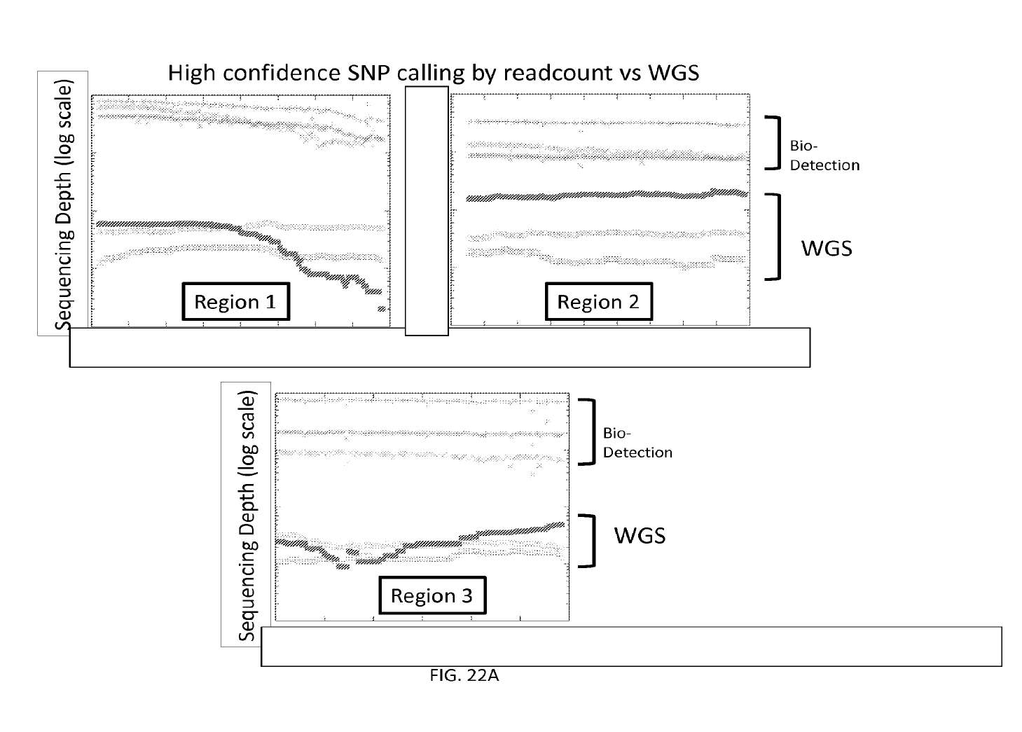

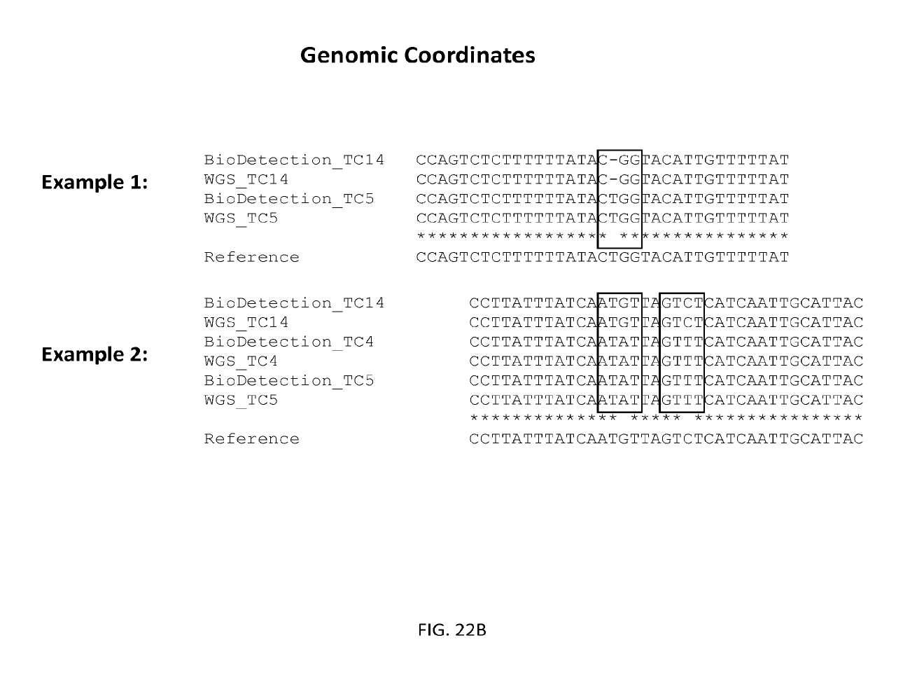

In a preferred embodiment, a backbone of a probe molecule contains the same sequence in all probes. A backbone can contain two primer binding sites that allow amplification of probe arms and a captured target sequence. In a preferred embodiment, the primers used may contain a barcode to allow multiple samples to be separated after simultaneous sequencing. In a preferred embodiment, the primers also contain 5′ ends that adapters for a next-generation sequencing platform such as the Ion Torrent PGM, Illumina MiSeq, Illumina HiSeq, Nanopore, etc (FIG. 1).

The probe set can include large number of probes, e.g., 10, 20, 30, 40, 50, 100, 200, 400, 500, 1000, 2000, 3000, 4000, 5000, 10000, 20000, 40000, 80000, or more. The probe set can include one or more probes directed to a large number of different target organisms, e.g., at least 10, 20, 40, 60, 80, 100, 150, 200, 250, 300, 400, 500, 600, 700, 800, 900, 1250, 1500, 1750, 2000, 3000, 4000, 5000, 6000, 7000, 8000, 9000, 10000, or more different target organisms. In some embodiments, a mixture including one or more probes to a plurality of target organisms contains only one probe to a target organism. In other embodiments, the mixture contains more than one probe to a target organism, e.g., about 2, 3, 4, 5, 6, 7, 8, 9, or 10 probes for a target organism. In certain embodiments, such as embodiments designed for use with patient test samples, the mixture further includes probes with homologous probe sequences that specifically hybridize to the host genome for applications such as host genotyping. In some embodiments, the mixtures of the invention further comprise sample internal calibration standards.

In one embodiment, the plurality of probes can detect at least 2, 3, 4, 5, 6, 7, 8, 9, 10, 20, 30, 40, 50, 60, 70, 80, 90, 100, 110, 120, 130, 140, 150, 160, 170, 180, 190, 200, 300, 400, 500, 600, 700, 800, 900, 1000, 1250, 1500, 1750, or 2000 different organisms or pathogens. In another embodiment, the plurality of probes can detect at least 10, 20, 30, 40, 50, 60, 70, 80, 90, 100, 110, 120, 130, 140, 150, 160, 170, 180, 190, 200, 300, 400, 500, 600, 700, 800, 900, 1250, 1500, 1750, 2000, 3000, 4000, 5000, 6000, 7000, 8000, 9000, 10000, or more different strains, variants or sub-types of a pathogen or different strains or sub-types of different pathogens. In one embodiment, the probe set identifies detects at least 2 different bacterial or fungal strains. In another embodiment, the probe set identifies at least 50 different organisms, such as 50 different pathogens, or 50 different strains or subtypes of a pathogen, such as Staphylococcus aureus.

In another embodiment, the probe set can comprise probes capable of detecting a single molecule of a pathogen, thereby detecting, distinguishing or identifying the pathogen.

Each probe in the probe set can comprise the same or different backbone size, sequence, chemistries, configuration of barcodes and sequences, specific sequences for probe enrichment, target sites for probe cleavage, hybridization arm physical and chemical properties, probe identification regions, low structure optimized design, or any combination thereof. A probe may be selected to screen key loci for pathogenicity and/or drug susceptibility, and a genetic fingerprint or genotype for each sub-strain that contains key phenotypic information is generated.

In another embodiment, the probe comprises a first sequence that hybridizes to a 5′ end of a target sequence and a second sequence that hybridizes to a 3′ end of a target sequence, wherein the target sequence can be used to identify, detect, or distinguish an organism, such as pathogen. In some embodiments, the probes in the mixture each comprise a first and second homologous probe sequence—separated by a backbone sequence—that specifically hybridize to a first and second sequence (such as sequences 3′ and/or 5′ to a target sequence, respectively) in the genome of at least one target organism. In some embodiments the first and second homologous probe sequences are not complementary to the target sequence, but ligate to the 5′ and 3′ termini of a target nucleic acid, e.g. a microRNA, and possess appropriate chemical groups for compatibility with a nucleic acid-ligating enzyme, such as phosphorylated or adenylated 5′ termini, and free 3′ hydroxyl groups. The probe can be capable of circularizing capture of a region of interest.

In some embodiments, the homologous probe sequences or the sequences of the probe that hybridize or are homologous to the 3′ and/or 5′ region of a target sequence specifically hybridizes to target sequences in the genome of their respective target organism, but do not specifically hybridize to any sequence in the genome of a predetermined set of sequenced organisms—the exclusion set. In embodiments related to probes that do not hybridize directly to the capture target, the ‘homologous probe sequences’ are designed specifically to not substantially hybridize to any sequence within a defined set of genomes, i.e., an exclusion set. In the case of biological samples from a subject, the exclusion set includes the host's genome. In particular embodiments, the exclusion set also includes a plurality of viral, eukaryotic, prokaryotic, and archaeal genomes. In more particular embodiments, the plurality of viral, eukaryotic, prokaryotic, and archaeal genomes in the exclusion set may comprise sequenced genomes from commensal, non-virulent, or nonpathogenic organisms. In still more particular embodiments, the exclusion set for all probes in a mixture share a common subset of sequenced genomes comprising, for example, a host genome and commensal, non-virulent, or non-pathogenic organisms. In general, the exclusion set varies between probes in the mixture so that each probe in the mixture does not specifically hybridize with the target sequence of any other probe in the mixture.

In some embodiments, the sequences 3′ and/or 5′ to a target sequence are separated by a region of interest (e.g., the target sequence) of at least two nucleotides. In particular embodiments, they are separated by at least 5, 6, 7, 8, 9, 10, 12, 14, 18, 20, 25, 30, 50, 75, 100, 150, 200, 300, 400, 600, 1200, 1500, 2500, or more nucleotides. In some embodiments, the first and second target sequences are separated by no more than 5, 6, 7, 8, 9, 10, 12, 14, 18, 20, 25, 30, 50, 75, 100, 150, 200, 300, 400, 600, 1200, 1500, or 2500 nucleotides.

In some embodiments, probes can be designed to capture conserved regions, and upon DNA sequencing, can reveal polymorphisms and genetic aberrations that allow for the resolution of known or novel variants or closely related strains of organisms. In some embodiments two or more probes can be used for one or more or every organism wished to be tested for, which can permit discrimination of closely related organisms, even when a sample comprises more than one organism.

In one aspect, the probes in the probe set each comprising homologous probe sequences which are substantially free of secondary structure, do not contain long strings of a single nucleotide (e.g., they have fewer than 7, 6, 5, 4, 3, or 2 consecutive identical bases), are at least about 8 bases (e.g., 8, 10, 12, 14, 16, 18, 20, 22, 24, 25, 26, 27, 28, 30, or 32 bases in length), and have a Tₘ in the range of 50-72° C. (e.g., about 53, 54, 55, 56, 57, 58, 59, 60, 61, or 62° C.). In some embodiments the first and second homologous probe sequences are about the same length and have the same Tₘ. In other embodiments, length and Tₘ of the first and second homologous probe sequences differ. The homologous probe sequences in each probe may also be selected to occur below a certain threshold number of times in the target organism's genome (e.g., fewer than 20, 10, 5, 4, 3, or 2 times).

The backbone sequence of the probes may include a detectable moiety and a primer-binding sequence. In some embodiments, the backbone sequence of the probes comprises a second primer. In particular embodiments, the detectable moiety is a barcode. In certain embodiments the backbone further comprises a cleavage site, such as a restriction endonuclease recognition sequence. In certain embodiments, the backbone contains non-WatsonCrick nucleotides, including, for example, abasic furan moieties, and the like.

In another aspect, the invention provides a kit comprising one or more sets of probes, such as one or more sets of probes from the probes provided in Table 1. In one embodiment, a kit comprises one or more reagents for obtaining a sample (e.g., swabs), reagents for extracting DNA, enzymes (such as polymerase and/or ligase to capture a region of interest), reagents for amplifying the region of interest, reagents for purifying the DNA or amplified or captured regions of interest (e.g., purification cartridge), buffers, sequencing reagents, or any combination thereof. In one embodiment, the kit may be a low throughput kit, such as a kit for a small number of samples. For example, a kit may be a low throughput kit, such as a kit for 2, 3, 4, 5, 6, 7, 8, 9, 10, 11, 12, 18, 20, 24, 28, 32, 36, 40, 42, 48, or between 8-48 samples. In another embodiment, the kit may be a high-throughput kit, such as a kit for a large number of samples. For example, a kit may be a high-throughput kit, such as a kit for 50, 60, 70, 80, 90, 100, 110, 120, 130, 140, 150, 160, 170, 180, 190, 200, 300, 400, 500, 600, 700, 800, 900, 1000, 1250, 1500, 1750, 2000, or more samples. For example, a kit may be a high-throughput kit, such as a kit for between 50-96, 50-384, 50-1536, 96-384, 96-1536, or 384-1536 samples. In some embodiments, a kit as described herein can comprise enough reagents to prepare one or more specimens for sequencing. For example, a kit as described herein can comprise enough reagents to prepare 2, 3, 4, 5, 6, 7, 8, 9, 10, 20, 30, 40, 48, 50, 60, 70, 80, 90, 96, 100, 110, 120, 130, 140, 150, 160, 170, 180, 190, 200, 300, 384, 400, 500, 600, 700, 800, 900, 1000, 1250, 1500, 1536, 1750, 2000 or more specimens for sequencing.

### Method of Using Probe

Also provided herein is a method of using one or more probes disclosed herein, such as one or more probe set, for detecting, identifying, or distinguishing one or more organisms. The method can comprise identifying a an organism with a plurality of probes can detect at least 10, 20, 30, 40, 50, 60, 70, 80, 90, 100, 110, 120, 130, 140, 150, 160, 170, 180, 190, 200, 300, 400, 500, 600, 700, 800, 900, 1000, 1250, 1500, 1750, or 2000 different pathogens. In another embodiment, the plurality of probes can detect at least 10, 20, 30, 40, 50, 60, 70, 80, 90, 100, 110, 120, 130, 140, 150, 160, 170, 180, 190, 200, 300, 400, 500, 600, 700, 800, 900, 1250, 1500, 1750, 2000, 3000, 4000, 5000, 6000, 7000, 8000, 9000, 10000, or more different strains, variants or sub-types of a pathogen or different strains or sub-types of different pathogens.

The method can comprise detecting or distinguishing different organisms, different pathogens, different strains, variants or sub-types of a pathogen or different strains, variants or sub-types of different pathogens, with at least 70% sensitivity, specificity, or both, such as with at least 71, 72, 73, 74, 75, 76, 77, 78, 79, 80, 81, 82, 83, 84, 85, 86, 87, 88, or 89% sensitivity, specificity, or both, such as with at least 90% sensitivity, specificity, or both. Each probe may detect or distinguish different organisms, different pathogens, different strains or sub-types of a pathogen or different strains or sub-types of different pathogens with at least 91, 92, 93, 94, 95, 96, 97, 98, 99 or 100% sensitivity, specificity, or both, in an assay. Alternatively, a combination of probes may be used for detecting or distinguishing different organisms, different pathogens, different strains, variants or sub-types of a pathogen or different strains, variants or sub-types of different pathogens, with at least 70% sensitivity, specificity, or both, such as with at least 71, 72, 73, 74, 75, 76, 77, 78, 79, 80, 81, 82, 83, 84, 85, 86, 87, 88, or 89% sensitivity, specificity, or both, such as with at least 90% sensitivity, specificity, or both. Furthermore, the confidence level for determining the specificity, sensitivity, or both, may be with at least 90, 91, 92, 93, 94, 95, 96, 97, 98, or 99% confidence.

In one embodiment, a method for detecting the presence of one or more target organisms is by contacting a sample suspected of containing at least one target organism with any of the probe set disclosed herein, capturing a region of interest of the at least one target organism (e.g., by polymerization and/or ligation) to form a circularized probe, and detecting the captured region of interest, thereby detecting the presence of the one or more target organisms.

In certain embodiments, the captured region of interest may be amplified to form a plurality of amplicons (e.g., by PCR). In some embodiments the sample is treated with nucleases to remove the linear nucleic acids after probe-circularizing capture of the region of interest. In some embodiments, the circularized probe is linearized, e.g., by nuclease treatment. In other embodiments the circularized probe molecule is sequenced directly by any means known in the art, without amplification. In certain embodiments, the circularized probe is contacted by an oligonucleotide that primes polymerase-mediated extension of the molecules to generate sequences complementary to that of the circularized probe, including from at least one to as many as 1 million or more concatemerized copies of the original circular probe.

In particular embodiments, the circularized probe molecule is enriched from the reaction solution by means of a secondary-capture oligonucleotide capture probe. A secondary-capture oligonucleotide capture probe may comprise a moiety designed to be captured, such as a biotin molecule, and a nucleic acid sequence designed to hybridize to at least 6 nucleotides of the circularized probe. The nucleic acid sequence designed to hybridize to at least 6 nucleotides of the circularized probe may include 1, 2, 4, 8, 16, 32 or more nucleotides of the polymerase-extended capture product.

In certain embodiments, the probe and/or captured region of interest is sequenced by any means known in the art, such as polymerase-dependent sequencing (including, dideoxy sequencing, pyrosequencing, and sequencing by synthesis) or ligase based sequencing (e.g., polony sequencing). The sequencing can be by Sanger sequencing or massive parallel sequencing, such as “next generation” (Next-gen) sequencing, second generation sequencing, or third generation sequencing. For example, sequencing can be by second generation or third generation sequencing methods, such as using commercial platforms such as Illumina, 454 (Roche), Solid, Ion Torrent PGM (Life Technologies), PacBio, Oxford, Life Technologies QDot, Nanopore, or any other available sequencing platform. Massive parallel sequencing can allow for the simultaneous sequencing of one million to several hundred millions, for example 1, 2, 3, 4, 5, 6, 7, 8, 9, 10, 20, 30, 40, 48, 50, 60, 70, 80, 90, 96, 100, 110, 120, 130, 140, 150, 160, 170, 180, 190, 200, 300, 400, 500, 600, 700, 800, or 900 million, of reads from amplified DNA clones. The reads can read any number of bases, such as 50-400 bases.

An internal nucleotide control, such as DNA at a known concentration, can be used with the methods and samples described herein. In one embodiment, an internal nucleotide control can serve as an internal calibrator, such as for determining copy number. In some embodiments, a sequencing read that aligns to the calibrator can also serve as a positive control for the performance of the assay, such as in the context of every sample.

In one aspect, the probes, methods, and kits described herein can be used to test for the presence of one or more organisms, such as those in Table 2. In one embodiment, the probes, methods, and kits described herein can be used to test for the presence of one or more antibiotic resistance genes, such as those in Table 3. In a preferred embodiment, the probes, methods, and kits described herein can be used to test for the presence of one or more organisms, such as those in Table 2, and test for the presence of one or more antibiotic resistance genes, such as those in Table 3, in parallel, such as in one sample tube, in the same sample, simultaneously, or any combination thereof. In some embodiments, in a single reaction tube, a kit can be used to test for the two or more microbes most commonly associated with hospital-acquired infections, and simultaneously tests for the presence of two or more antibiotic resistance genes. For example, a kit can be used to test for the 3, 4, 5, 6, 7, 8, 9, 10, 11, 12, 13, 14, 15, 16, 17, 18, 19, 20, or more microbes most commonly associated with hospital-acquired infections, and simultaneously tests for the presence of 3, 4, 5, 6, 7, 8, 9, 10, 11, 12, 13, 14, 15, 16, 17, 18, 19, 20, 21, 22, 23, 24, 25, 26, 27, 28, 29, 30, or more antibiotic resistance genes simultaneously. For example, in a single reaction tube, a kit can be used to test for the 12 microbes most commonly associated with hospital-acquired infections, and simultaneously tests for the presence of 18 antibiotic resistance genes.

In one embodiment, one or more organisms can be identified from a sample, such as a sample form a host and the organism being identified is a pathogen. In one embodiment, the sample is a biological sample, such as from a mammal, such as a human. In another embodiment, a genotype of the host is identified or detected from the sample or another sample from the host. The identification of one or more organisms (such as one or more pathogens, such as different pathogens or subtypes or strains of pathogens), can be used to select one or more therapeutics or treatments for the host. In another embodiment, the identification of one or more organisms (such as one or more pathogens, such as different pathogens or subtypes or strains of pathogens), can be used to stratify the host into a therapeutic group, such as for a particular drug treatment or clinical trial. In one embodiment, HPV strain identification can be used to stratify a host into a cancer therapeutic group or to select a cancer treatment.

The yet another embodiment identification of one or more organisms (such as one or more pathogens, such as different pathogens or subtypes or strains of pathogens) and the genotype of a host can be used to select one or more therapeutics or treatments for the host. In another embodiment, the identification of one or more organisms (such as one or more pathogens, such as different pathogens or subtypes or strains of pathogens) and the genotype of the host can be used to stratify the host into a therapeutic group, such as for a particular drug treatment or clinical trial.

Also provided herein is a method for identifying an organism, such as a genetic signature of an organism, a subtype or strain of a pathogen in a short timeframe or with a fast turnaround time. In another embodiment, a genotype of an individual or host can also be identified within the short time frame. For example, the identification of a pathogen in a sample or the genotype of a host can completed in less than 2, 3, 4, 5, 6, 7, 8, 9, 10, 11, or 12 hours. In one embodiment, from contacting the sample with one or more probes to identifying the organism by sequencing can be performed in less than 2, 3, 4, 5, 6, 7, 8, 9, 10, 11, or 12 hours. In yet another embodiment, from contacting the sample with the probe to identifying the organism (such as one or more pathogens) by sequencing, and transmitting the results to a health care professional (such as a clinician or physician) can be performed in less than 2, 3, 4, 5, 6, 7, 8, 9, 10, 11, or 12 hours. In yet another embodiment, from contacting the sample with the probe to identifying the organism (such as one or more pathogens) by sequencing, transmitting the results to a health care professional (such as a clinician or physician), and selection of a therapeutic can be performed in less than 2, 3, 4, 5, 6, 7, 8, 9, 10, 11, or 12 hours.

Also provided herein is a method for simultaneous quantification and identification of an organism, such as identifying one or more subtypes or substrains of a pathogen. Multiplexing is also provided herein, wherein a multiple pathogens, substrains or subtypes of pathogens, can be detected simultaneously or in a single reaction tube.

In one embodiment, conversion of sequence data to quantitative report can be performed by using selected validated parameters. Any software known in the arts can be used for any of the methods disclosed herein.

In some embodiments, an organism identified and/or quantified using the methods described herein can be the cause of an infection in a subject, such as a nosocomial infection (also known as a hospital-acquired infection (HAI)) which is an infection whose development is favored by a hospital environment. In some embodiments, an infection can be acquired by a patient during a hospital visit or one developing among hospital staff. Such infections can include, for example, fungal and bacterial infections and can be aggravated by a reduced resistance of individual patients. Organisms responsible for HAIs can survive for a long time on surfaces in the hospital and can enter or be transmitted to the body through wounds, catheters, and ventilators. In some embodiments, the route of transmission can be contact transmission (direct or indirect), droplet transmission, airborne transmission, common vehicle transmission, vector borne transmission, or any combination thereof.

People in hospitals can already be in a poor state of health, impairing their defense against bacteria. Advanced age or premature birth along with immunodeficiency, due to, for example, drugs, illness, or irradiation, present a general risk. Other diseases can present specific risks, for example, chronic obstructive pulmonary disease can increase chances of respiratory tract infection. Invasive devices, for example, intubation tubes, catheters, surgical drains, and tracheostomy tubes can bypass the body's natural lines of defense against pathogens and can provide an easy route for infection. Patients already colonized on admission can be put at greater risk when they undergo a procedure, such as an invasive procedure. A patient's treatment itself can leave the patient vulnerable to infection, for example, immunosuppression and antacid treatment can undermine the body's defenses, while antimicrobial and recurrent blood transfusions can also be risk factors.

Non-limiting examples of HAIs include Ventilator associated pneumonia (VAP), Staphylococcus aureus, Methicillin resistant Staphylococcus aureus (MRSA), Candida albicans, Pseudomonas aeruginosa, Acinetobacter baumannii, Stenotrophomonas maltophilia, Clostridium difficile, Tuberculosis, Urinary tract infection, Hospital-acquired pneumonia (HAP), Gastroenteritis, Vancomycin-resistant Enterococcus (VRE), and Legionnaires' disease. In some embodiments, HAIs can be caused by one or more of the organisms provided in Table 2.

Nucleotides, such as DNA and RNA, can be isolated from any suitable sample and detected using the probes described herein. Non-limiting examples of sample sources include catheters, medical devices, blood, blood cultures, urine, stool, fomites, wounds, sputum, pure bacterial cultures, mixed bacterial cultures, and bacterial colonies.

In some embodiments, the probe sets described herein can be used to detect and distinguish among the organisms responsible for more than 10% of the hospital acquired infections at a site. For example, the probe sets described herein can be used to detect and distinguish among the organisms responsible for more than 5%, 10%, 15%, 20%, 25%, 30%, 35%, 40%, 45%, 50%, 55%, 60%, 65%, 70%, 75%, 80%, 85%, 90%, 95%, or 100% of the hospital acquired infections at a site. In some embodiments, a site can be a surgical site, wound, tract, urinary catheter, ventilator, intravenous needle, syringe, respiratory tract, invasive device, intubation tube, catheter, surgical drain, tracheostomy tube, saline flush syringe, vial, bag, tube or any combination thereof.

### Method of Generating Probe

A further aspect of the invention provides methods of making the mixtures of probes provided by the invention. The methods comprise providing a set of reference genomes and an exclusion set of genomes. The sequence of the reference genomes can be partitioned (in silico) into n-mer strings of about 18-50 nucleotides. The partitioned n-mer strings can be screened to eliminate redundant sequences, sequences with secondary structure, repetitive sequences (e.g., strings with more than 4 consecutive identical nucleotides), and sequences with a Tₘ outside of a predetermined range (e.g., outside of 50-72° C.). The screened n-mers can be further screened to identify homologous probe sequences by eliminating n-mers that specifically hybridize to a sequence in the genome in the exclusion set of genomes (e.g., if a pairwise alignment contains 19 of 20 matches in an n-mer, such as a 25-mer) or occurs in the genome of the target organism more than a specified number of times. The screening may also remove n-mers that are present in more than or less than a specified number of the reference genomes. The screening may also remove n-mers that will not interact favorably with enzymes to be used with the probe sequences. For example, a particular polymerase may work with higher efficiency if the last 3′ base of the probe is a G or C. Similarly, a particular ligase may work more efficiently on certain bases at the ligation junction. For example, Ampligase (Epicentre) will ligate a gap between AG and GT at least 10 times more efficiently than a gap between TC and CC.

In particular embodiments, a homologous probe sequence may occur only once in the genome of the target organism. For target organisms with a single-stranded genome, the homologous probe sequence may occur only once in the complement of the genome of the target organism. In one embodiment, where a sequenced variant of the target organism is available (e.g., the same species, genus, or serovar), the homologous probe sequences can be filtered so as to specifically hybridize to the genome of the additional sequenced variant(s) resulting in a probe that groups related organisms. In an alternate embodiment, the homologous probe sequences can be filtered so as to not specifically hybridize to the genome of the sequenced variant (e.g., the sequenced variant is part of the exclusion set), resulting in a probe that discriminates between related organisms. These filter processes can be iterated for each target organism to be detected by the particular mixture. In some embodiments, the candidate homologous probe sequences can be screened to eliminate those that will specifically hybridize with other probes in the mixture.

Probe selection can be based on a database of different pathogens, strains of a pathogen, or both, such as a database comprising more than 10 different pathogens, strains of a pathogen, or both. For example, probe selection can be based a database comprising more than 10, 20, 30, 40, 50, 60, 70, 80, 90, 100, 110, 120, 130, 140, 150, 160, 170, 180, 190, 200, 300, 400, 500, 600, 700, 800, 900, 1000, 1250, 1500, 1750, 2000, or more different pathogens, strains of a pathogen, or both. In some embodiments, probe selection can be based on a database of different pathogens, strains of a pathogen, or both, that are known to cause HAIs, such as a database comprising more than 10 different pathogens, strains of a pathogen, or both, that are known to cause HAIs. For example, probe selection can be based a database comprising more than 10, 20, 30, 40, 50, 60, 70, 80, 90, 100, 110, 120, 130, 140, 150, 160, 170, 180, 190, 200, 300, 400, 500, 600, 700, 800, 900, 1000, 1250, 1500, 1750, 2000, or more different pathogens, strains of a pathogen, or both, that are known to cause HAIs, and optionally with additional strains or sub-types of other pathogens. In one embodiment, probes for organisms associated with HAIs are selected by partitioning all available genomes of organisms associated with HAIs into one or more subsets based on sequence similarity. For each subset candidate probe sets are generated that capture all strains. A filter can then be applied for specificity against human/microbial/viral/fungal genomes.

Some of clinical tests based on the methods disclosed herein rely on the ability to determine or approximate the number of input template molecules (genomes) in a sample. A two step method can be used to calculate the number of template molecules in a sample from the sequencing read counts. 1) Each sample sequenced can have a known quantity of a control sequence added to it. One embodiment employs GFP as the control sequence. It is contemplated to use several control sequences added in different quantities. The first step in analyzing sequencing reads can be to normalize the counts based on the number of reads that came from the control sequence. This normalization accounts for the fact that more material from sample A than from sample B may have been put into the sequencing reaction. 2) Since different MIPs (or primer pairs or hybridization capture probes) might work with different efficiencies, the second step of the quantification process can be to normalize between probes. In one embodiment, this normalization relies on experiments in which fixed amounts of different templates were sequenced and might reveal, e.g., that a probe against one strain or organism produces 2 circularized MIPs per template but a probe against anther strain or organism produces 3. Thus, the count for the first probe might be multiplied by 33.3 and the count for the second probe divided by 50 to produce comparable load counts for the two strains.

Some embodiments use a mixed quantity of GFP as the control sequence and a variable quantity of one or more organisms or strains. Some samples may contain only GFP and template DNA while others also included a human background. After the sequencing reads are separated by sample, the method can calculate the ratio of reads, such as viral (HPV-18, HIV-CN006, and HIV-CN009) reads, to GFP and plots that ratio against the number of template molecules in the reaction. Those plots indicate generally excellent agreement between the viral/GFP ratio and the input template quantity.

Compared to other assays, high throughput sequencing offers a relatively unique ability to detect and genotype the pathogen DNA and the human DNA in a sample from a single reaction. In current clinical practice, genotyping the pathogen and human may require multiple tests, potentially doubling (or more) the expense compared to simply detecting a pathogen. The methods disclosed herein enable simultaneous genotyping with minimal added cost and often no added labor. Other selection/enrichment technologies would also enable these tests.

The methods disclosed herein provide for simultaneously detecting or genotyping multiple pathogens.

For example, the methods provide for: coinfection of HIV and HCV, simultaneously genotyping/quantifying HIV while testing for diseases common in immunocompromised patients. Doctors typically only test for diseases like Candida, CMV, etc upon presentation of some other symptom. However, if the tests can be added at minimal cost, this might be a unique market and feature for Pathogenica's product, for example, HPV and other STIs. There is an interest in testing for HPV and other STIs, primarily chlamydia and gonorrhea to simplify screening, especially in patient populations with limited access to doctors. There is also an interest in testing for these diseases as additional risk factors for cervical cancer.

### Probe Panel

Table 1 lists the probe arm sequences in one embodiment of the present invention designed to detect a variety pathogenic organisms, such as those provided in Table 2, from a sample. Non limiting examples include Staphylococcus aureus, Staphylococcus epidermidis, Staphylococcus saprophyticus, Acinetobacter baumanii, Clostridium difficile, Escherichia coli, Enterobacter (aerogenes, cloacae, asburiae), Enterococcus (faecium, faecalis), Klebsiella pneumoniae, Proteus mirabilis, Candida albicans, and Pseudomonas aeruginosa. The probe set can also be used to detect many common drug resistance genes, including, but not limited to CARB, CMY, CTX-M, GES, IMP, KPC, NDM, Other ampC, OXA, PER, SHV, VEB, VIM, ermA, vanA, vanB, mecA, and mexA,

Tables 1 and 3-14 provide regions of interest (leftmost columns, using the format of descriptor (e.g., organism or gene, if applicable)_reference accession number (if applicable)_first nucleotide of capture region_last nucleotide of capture region. For example, the probe “acinetobacter_NC_{—}010611_{—}627997_{—}628164” is directed to acineobacter, and is predicted to be capable of capturing nucleotides corresponding to nucleotides 627997 to 628164 of the reference sequence NC_{—}010611. Reference accession sequences can be obtained from, for example, the NCBI Entrez portal. Tables 3, 5, 7, 9, 11, and 13 provide the regions of interest and corresponding annotated genes within that region. Tables 4, 6, 8, 10, 12, and 14, in turn, provide particular exemplary oligonucleic acid sequences—provided as pairs that can be used in a MIP or adapted for use as conventional PCR primers—predicted to capture the region of interest listed in the first column of the. “Binding region 1” in Tables 4, 6, 8, 10, 12, and 14 correspond to the 5′, or ligation arm, of a MIP probe and “Binding region 2” corresponds to the 3′, or extension arm of a MIP probe. In some embodiments, substantially similar sequences to the regions of interest provided in Tables 1 and 3-14 can be used. In some embodiments, the substantially similar sequences wherein the substantially similar sequences are 60, 65, 70, 75, 80, 85, 90, 95, 96, 97, 98, 99, 99.5, or 100% identical to the sequence of the regions of interest. In other embodiments, the substantially similar sequences have endpoints within 100, 90, 80, 70, 60, 50, 40, 35, 30, 25, 20, 19, 18, 17, 16, 15, 14, 13, 12, 11, 10, 9, 8, 7, 6, 5, 4, 3, 2, 1, or 0 nucleotides upstream or downstream of either of the endpoints of the regions of interest. In still other embodiments, the substantially similar sequences are 60, 65, 70, 75, 80, 85, 90, 95, 96, 97, 98, 99, 99.5, or 100% identical to the sequence of the regions of interest and have endpoints within 100, 90, 80, 70, 60, 50, 40, 35, 30, 25, 20, 19, 18, 17, 16, 15, 14, 13, 12, 11, 10, 9, 8, 7, 6, 5, 4, 3, 2, 1, or 0 nucleotides upstream or downstream of either of the endpoints of the regions of interest. In still more particular embodiments, the particular exemplified endpoints and binding regions are use, e.g., as pairs of binding regions in either a single MIP capture probe, or as pairs of conventional PCR primers, e.g., using the reverse complement of the ligation arm.

Subsets of the regions of interest or particular exemplary binding regions in tables Tables 1 and 3-14 can be used concordant with the present invention, e.g., 10, 20, 30, 40, 50, 60, 70, 80, 90, 95, 96, 97, 98, 99, or 100% of the regions of interest or binding regions in the tables, e.g.:

oligonucleic acid molecules capable of i) amplifying, geometrically by polymerase chain reaction or ii) circularizing capture of 1, 2, 3, 4, 5, 10, 15, 16, or all 17, of the regions of interest provided in column 1 of Table 3, or substantially similar sequences;

oligonucleic acid molecules capable of i) amplifying, geometrically by polymerase chain reaction or ii) circularizing capture of 1, 2, 3, 4, 5, 10, 15, 20, 30, 50, 100, or all 134, of the regions of interest provided in column 1 of Table 5, or substantially similar sequences, such as:

oligonucleic acid molecules capable of i) amplifying, geometrically by polymerase chain reaction or ii) circularizing capture of, 1, 2, 3, 4, 5, 10, or all 13, of the regions of interest provided in column 1 of Table 7, or substantially similar sequences;

oligonucleic acid molecules capable amplifying, geometrically by polymerase chain reaction, or circularizing capture of, 1, 2, 3, 4, 5, 10, 20, 40, 60, 80, or all 85, of the regions of interest provided in column 1 of Table 9, or substantially similar sequences;

oligonucleic acid molecules capable of i) amplifying, geometrically by polymerase chain reaction or ii) circularizing capture of, 1, 2, 3, 4, 5, 10, 20, 25, or all 29 of the regions of interest provided in column 1 of Table 11, or substantially similar sequences;

oligonucleic acid molecules capable of i) amplifying, geometrically by polymerase chain reaction or ii) circularizing capture of, 1, 2, 3, 4, 5, 10, 15, or all 20, of the regions of interest provided in column 1 of Table 13, or substantially similar sequences;

oligonucleic acid molecules comprising 1, 2, 4, 6, 8, 10, 15, 20, 25, 30, or all 34 of the sequences, or reverse complements thereof, provided in the second or third column of table 4;

oligonucleic acid molecules comprising 1, 2, 4, 6, 8, 10, 20, 50, 100, 150, 200, 250, or all 268 of the sequences, or reverse complements thereof, provided in the second or third column of table 6;

oligonucleic acid molecules comprising 1, 2, 4, 6, 8, 10, 15, 20, 25, or all 26 of the sequences, or reverse complements thereof, provided in the second or third column of table 8;

oligonucleic acid molecules comprising 1, 2, 4, 6, 8, 10, 20, 50, 100, 150, or all 170 of the sequences, or reverse complements thereof, provided in the second or third column of table 10;

oligonucleic acid molecules comprising 1, 2, 4, 6, 8, 10, 20, 30, 40, 50, or all 56 of the sequences, or reverse complements thereof, provided in the second or third column of table 12;

oligonucleic acid molecules comprising 1, 2, 4, 6, 8, 10, 20, 30, or all 40 of the sequences, or reverse complements thereof, provided in the second or third column of table 14, as well as any combinations of the foregoing.

Table 1 provides particular probes assembled as molecular inversion probes (MIPs) capable of circularizing capture of the indicated region of interest in the leftmost column. These exemplary probes share a common backbone sequence of GTTGGAGGCTCATCGTTCCTATATTCCACACCACTTATTATTACAGATGTTATGCT CGCAGGTC, except for the peGFP_N1_{—}730_{—}925 probe, which uses the backbone GTTGGAGGCTCATCGTTCCTATATTCCTGACTCCTCATTGATGATTACAGATGTTA TGCTCGCAGGTC. Alternative backbone sequences can readily be used. Conventional PCR primer pairs can be adapted from these MIP probes by omitting the intervening backbone sequence and providing the reverse complement of the ligation arm (5′) probe. Tables 4, 6, 8, 10, 12, and 14 provide subsets of the probes in Table 1 where the individual arms are provided in the second and third columns, respectively. Tables 4, 6, 8, 10, 12, and 14 collectively provide the same probe arms that are present in Table 1.

TABLE 1
A particular embodiment of the probe sets provided by the invention

| Name | Sequence |
|---|---|
| peGFP_N1_730_925 | /5Phos/GTGGTATGGCTGATTATGATCTAGAGTGTTGGAGGCTCATCGTTCCTATA |
| | TTCCTGACTCCTCATTGATGATTACAGATGTTATGCTCGCAGGTCGAGTTTGGACAA |
| | ACCACAACTAGAA |
| plasmids_NC_010660_187035_187205 | /5Phos/GCTGTCACCGTCCAGACGCTGTTGGCGTTGGAGGCTCATCGTTCCTATAT |
| | TCCACACCACTTATTATTACAGATGTTATGCTCGCAGGTCTCCGTGCCTTCAAGCGCG |
| plasmids_NC_014232_5501_5677 | /5Phos/GACTCCGCAGAATACGGCACCGTGCGCAGTTGGAGGCTCATCGTTCCTAT |
| | ATTCCACACCACTTATTATTACAGATGTTATGCTCGCAGGTCGCGTACAGGCCAGTC |
| | AGC |
| plasmids_NC_011980_58308_58487 | /5Phos/GCAGTCGGTAACCTCGCGCGTTGGAGGCTCATCGTTCCTATATTCCACAC |
| | CACTTATTATTACAGATGTTATGCTCGCAGGTCGCGCTATCTCTGCTCTCACTGC |
| plasmids_NC_011838_178818_178996 | /5Phos/GCTGTCCTGGCTGCAAGCCTGGGTTGGAGGCTCATCGTTCCTATATTCCA |
| | CACCACTTATTATTACAGATGTTATGCTCGCAGGTCCCGAACTGCTGATGGACGT |
| plasmids_FN554767_13017_13190 | /5Phos/GACAGCAGACTCACCGGCTGGTTCCGCTGTTGGAGGCTCATCGTTCCTAT |
| | ATTCCACACCACTTATTATTACAGATGTTATGCTCGCAGGTCGCAAGATGCTGCTGG |
| | CCACACTG |
| plasmids_NC_013655_115365_115542 | /5Phos/GACAGAACAAGTTCCGCTCCGGGTTGGAGGCTCATCGTTCCTATATTCCA |
| | CACCACTTATTATTACAGATGTTATGCTCGCAGGTCCACGGATACGCCGCGCAT |
| plasmids_NC_013950_90185_90338 | /5Phos/GAGGACCGAAGGAGCTAACCGGTTGGAGGCTCATCGTTCCTATATTCCAC |
| | ACCACTTATTATTACAGATGTTATGCTCGCAGGTCCGCCGCATACACTATTCTC |
| plasmids_NC_015599_37281_37455 | /5Phos/GCTGTAATGCAAGTAGCGTATGCGCTCGTTGGAGGCTCATCGTTCCTATA |
| | TTCCACACCACTTATTATTACAGATGTTATGCTCGCAGGTCGAACAGCAAGGCCGCC |
| | AATGCCTGACG |
| plasmids_NC_013951_69899_70067 | /5Phos/GAACGTCTGGCGCTGGTCGCCTGCCGTTGGAGGCTCATCGTTCCTATATT |
| | CCACACCACTTATTATTACAGATGTTATGCTCGCAGGTCGCACAGGTGCTGACGTGGT |
| plasmids_NC_007351_37979_38146 | /5Phos/CGCATATGCTGAATGATTATCTCGTTGCGTTGGAGGCTCATCGTTCCTAT |
| | ATTCCACACCACTTATTATTACAGATGTTATGCTCGCAGGTCATCTTGCTCAATGAG |
| | GTTATTCA |
| plasmids_FN822749_1846_2009 | /5Phos/GACGACAGATGCAGGTTGAGTTGGAGGCTCATCGTTCCTATATTCCACAC |
| | CACTTATTATTACAGATGTTATGCTCGCAGGTCCGCATCGCCGATGCTCATC |
| plasmids_NC_004851_143949_144109 | /5Phos/CGCCTGCTCCAGTGCATCCAGCACGAATGTTGGAGGCTCATCGTTCCTAT |
| | ATTCCACACCACTTATTATTACAGATGTTATGCTCGCAGGTCATGCTCTCCGCCATC |
| | GCGTTGTCA |
| plasmids_NC_010558_156799_156957 | /5Phos/AGTGCGTTCACCGAATACGTGCGCAGTTGGAGGCTCATCGTTCCTATATT |
| | CCACACCACTTATTATTACAGATGTTATGCTCGCAGGTCCAGGTTATGCCGCTCAAT |
| | TC |
| plasmids_NC_007635_38395_38566 | /5Phos/AATCCAGGTCCTGACCGTTCTGTCCGTGTTGGAGGCTCATCGTTCCTATA |
| | TTCCACACCACTTATTATTACAGATGTTATGCTCGCAGGTCACCTCCGTTGAGCTGA |
| | TGGA |
| plasmids_NC_009787_17946_18116 | /5Phos/GAGGTGGCCAACACCATGTGTGACCGTTGGAGGCTCATCGTTCCTATATT |
| | CCACACCACTTATTATTACAGATGTTATGCTCGCAGGTCGACGCCGGTATATCGGTA |
| | TCGAGCTGCT |
| plasmids_NC_012547_53585_53752 | /5Phos/CGCATATGCTGAATGATTATCTCGTTGGTTGGAGGCTCATCGTTCCTATA |
| | TTCCACACCACTTATTATTACAGATGTTATGCTCGCAGGTCACGGTGATCTTGCTCA |
| | ATGAGGTTATTC |
| plasmids_NC_006671_56259_56438 | /5Phos/GAAGTGCCGGACTTCTGCAGAGTTGGAGGCTCATCGTTCCTATATTCCAC |
| | ACCACTTATTATTACAGATGTTATGCTCGCAGGTCGCACGGCCTGATGGAGGCCGC |
| plasmids_NC_014385_53151_53310 | /5Phos/GCTAATCGCATAACAGCTACGTTGGAGGCTCATCGTTCCTATATTCCACA |
| | CCACTTATTATTACAGATGTTATGCTCGCAGGTCCATCACGTAACTTATTGATGATA |
| | TT |
| plasmids_FN649418_57169_57339 | /5Phos/GCTGCGGTATTCCACGGTCGGCCGTTGGAGGCTCATCGTTCCTATATTCC |
| | ACACCACTTATTATTACAGATGTTATGCTCGCAGGTCGCAGGAACGCTGCCTGTGGTC |
| plasmids_NC_005011_8620_8785 | /5Phos/GAATCAATTATCTTCTTCATTATTGATGTTGGAGGCTCATCGTTCCTATA |
| | TTCCACACCACTTATTATTACAGATGTTATGCTCGCAGGTCCTGCGGCTCAACTCAA |
| | GCA |
| plasmids_NC_014843_98413_98578 | /5Phos/GTCACACGTCACGCAGTCCGTTGGAGGCTCATCGTTCCTATATTCCACAC |
| | CACTTATTATTACAGATGTTATGCTCGCAGGTCGCATTCATGGCGCTGATGGC |
| plasmids_NC_008490_5165_5334 | /5Phos/GTGTTACTCGGTAGAATGCTCGCAAGGGTTGGAGGCTCATCGTTCCTATA |
| | TTCCACACCACTTATTATTACAGATGTTATGCTCGCAGGTCACTAGATGACATATCA |
| | TGTAAGTT |
| plasmids_NC_015963_147516_147686 | /5Phos/CGGAACTGCCTGCTCGTATGTTGGAGGCTCATCGTTCCTATATTCCACAC |
| | CACTTATTATTACAGATGTTATGCTCGCAGGTCAACGATATAGTCCGTTAT |
| plasmids_NC_007365_100545_100708 | /5Phos/GCTCTCCGACTCCTGGTACGTCAGGTTGGAGGCTCATCGTTCCTATATTC |
| | CACACCACTTATTATTACAGATGTTATGCTCGCAGGTCGCGCGCATTAATGAAGCAC |
| plasmids_NC_009838_104163_104332 | /5Phos/GATGTTGCGATTACTTCGCCAACTATTGGTTGGAGGCTCATCGTTCCTAT |
| | ATTCCACACCACTTATTATTACAGATGTTATGCTCGCAGGTCGCTGTAATTATGACG |
| | ACGCCG |
| plasmids_NC_013452_4052_4209 | /5Phos/CTCATTCCAGAAGCAACTTCTTCTTGTTGGAGGCTCATCGTTCCTATATT |
| | CCACACCACTTATTATTACAGATGTTATGCTCGCAGGTCGGATAGCCATGGCTACAA |
| | GAATA |
| plasmids_NC_010409_39768_39935 | /5Phos/GCAATACCAGGAAGGAAGTCTTACTGGTTGGAGGCTCATCGTTCCTATAT |
| | TCCACACCACTTATTATTACAGATGTTATGCTCGCAGGTCGTCATTGGAGAACAGAT |
| | GATTGATGT |
| plasmids_NC_014233_50337_50492 | /5Phos/GTATCGCCACAATAACTGCCGGAAGTTGGAGGCTCATCGTTCCTATATTC |
| | CACACCACTTATTATTACAGATGTTATGCTCGCAGGTCAACGATATAGTCCGTTATG |
| plasmids_NC_013950_91008_91174 | /5Phos/GCTGTGGCACAGGCTGAACGCCGGTTGGAGGCTCATCGTTCCTATATTCC |
| | ACACCACTTATTATTACAGATGTTATGCTCGCAGGTCGGTGATGTCATTCTGGTTAA |
| | GA |
| plasmids_NC_002698_168967_169123 | /5Phos/ACATAATCTGAATCTGAGACAACATCGTTGGAGGCTCATCGTTCCTATAT |
| | TCCACACCACTTATTATTACAGATGTTATGCTCGCAGGTCACGCACTCTGGCCACAC |
| | TGG |
| plasmids_NC_013362_56651_56805 | /5Phos/GTGAAGCGCATCCGGTCACCGTTGGAGGCTCATCGTTCCTATATTCCACA |
| | CCACTTATTATTACAGATGTTATGCTCGCAGGTCATGGCATAGGCCAGGTCAATAT |
| plasmids_NC_014208_52313_52469 | /5Phos/GGTTCTGGACCAGTTGCGTGAGCGCGTTGGAGGCTCATCGTTCCTATATT |
| | CCACACCACTTATTATTACAGATGTTATGCTCGCAGGTCCGTAACATCGTTGCTGCT |
| | CCAT |
| betalactamase_AB372224_738_905 | /5Phos/CGCTGGATTTCACGCCATAGGCGTTGGAGGCTCATCGTTCCTATATTCCA |
| | CACCACTTATTATTACAGATGTTATGCTCGCAGGTCTGTCGCTACCGTTGATGATT |
| betalactamase_EF685371_398_548 | /5Phos/CGTATAGGTGGCTAAGTGCAGCGTTGGAGGCTCATCGTTCCTATATTCCA |
| | CACCACTTATTATTACAGATGTTATGCTCGCAGGTCGTAACTCATTCCTGAGGGTTTC |
| betalactamase_DQ149247_231_371 | /5Phos/GTACATACTCGATCGAAGCACGAGTTGGAGGCTCATCGTTCCTATATTCC |
| | ACACCACTTATTATTACAGATGTTATGCTCGCAGGTCCCGGAATAGCGGAAGCTTTC |
| betalactamase_AY750911_244_414 | /5Phos/AAGGTCGAAGCAGGTACATACTCGGTTGGAGGCTCATCGTTCCTATATTC |
| | CACACCACTTATTATTACAGATGTTATGCTCGCAGGTCAGACATGAGCTCAAGTCCA |
| | AT |
| betalactamase_DQ519087_417_575 | /5Phos/GAAGCTTTCATAGCGTCGCCTAGGTTGGAGGCTCATCGTTCCTATATTCC |
| | ACACCACTTATTATTACAGATGTTATGCTCGCAGGTCTTAGCTAGCTTGTAAGCAAA |
| | TTG |
| betalactamase_AM231719_379_537 | /5Phos/GAAGCTTTCATGGCATCGCCTAGGTTGGAGGCTCATCGTTCCTATATTCC |
| | ACACCACTTATTATTACAGATGTTATGCTCGCAGGTCAGCTAGCTTGTAAGCAAACTG |
| betalactamase_Y14156_663_819 | /5Phos/CGCTACCGGTAGTATTGCCCTTGTTGGAGGCTCATCGTTCCTATATTCCA |
| | CACCACTTATTATTACAGATGTTATGCTCGCAGGTCAGAATATCCCGACGGCTTTC |
| betalactamase_JN227085_763_931 | /5Phos/ATCGCCACGTTATCGCTGTACTGTTGGAGGCTCATCGTTCCTATATTCCA |
| | CACCACTTATTATTACAGATGTTATGCTCGCAGGTCTTTACCCAGCGTCAGATTCC |
| betalactamase_EU259884_1030_1170 | /5Phos/CAAGTACTGTTCCTGTACGTCAGCGTTGGAGGCTCATCGTTCCTATATTC |
| | CACACCACTTATTATTACAGATGTTATGCTCGCAGGTCTCGCCAGTAACTGGTCTAT |
| | TC |
| betalactamase_HQ913565_578_730 | /5Phos/CAACGTCTGCGCCATCGCCGTTGGAGGCTCATCGTTCCTATATTCCACAC |
| | CACTTATTATTACAGATGTTATGCTCGCAGGTCCGCAATATCATTGGTGGTGC |
| betalactamase_AY524988_385_552 | /5Phos/GCCGCCCGAAGGACATCAACGTTGGAGGCTCATCGTTCCTATATTCCACA |
| | CCACTTATTATTACAGATGTTATGCTCGCAGGTCCAGACGGGACGTACACAAC |
| CARB_AF030945_646_795 | /5Phos/CGTGCTGGCTATTGCCTTAGGGTTGGAGGCTCATCGTTCCTATATTCCAC |
| | ACCACTTATTATTACAGATGTTATGCTCGCAGGTCGTAATACTCCTAGCACCAAATC |
| CARB_U14749_1227_1390 | /5Phos/CATTAGGAGTTGTCGTATCCCTCAGTTGGAGGCTCATCGTTCCTATATTC |
| | CACACCACTTATTATTACAGATGTTATGCTCGCAGGTCAATACTCCGAGCACCAAATC |
| CARB_AF313471_2731_2906 | /5Phos/AAATTGCAGTTCGCGCTTAGCGTTGGAGGCTCATCGTTCCTATATTCCAC |
| | ACCACTTATTATTACAGATGTTATGCTCGCAGGTCGTTCCATAGCGTTAAGGTTTC |
| CMY_DQ463751_613_790 | /5Phos/GCGCCAAACAGACCAATGCTGTTGGAGGCTCATCGTTCCTATATTCCACA |
| | CCACTTATTATTACAGATGTTATGCTCGCAGGTCGATTTCACGCCATAGGCTC |
| CMY_EF685371_397_552 | /5Phos/GTATAGGTGGCTAAGTGCAGCAGTTGGAGGCTCATCGTTCCTATATTCCA |
| | CACCACTTATTATTACAGATGTTATGCTCGCAGGTCTCGTAACTCATTCCTGAGGG |
| CMY_EU515251_583_733 | /5Phos/GTCATCGCCTCTTCGTAGCTCGTTGGAGGCTCATCGTTCCTATATTCCAC |
| | ACCACTTATTATTACAGATGTTATGCTCGCAGGTCGCCATATCGATAACGCTGG |
| CMY_X92508_126_301 | /5Phos/AGTATCTTACCTGAAATTCCCTCACGTTGGAGGCTCATCGTTCCTATATT |
| | CCACACCACTTATTATTACAGATGTTATGCTCGCAGGTCCCTCTCGTCATAAGTCGA |
| | ATG |
| CMY_AB061794_343_489 | /5Phos/CATCACGAAGCCCGCCACAGTTGGAGGCTCATCGTTCCTATATTCCACAC |
| | CACTTATTATTACAGATGTTATGCTCGCAGGTCGCCCTTGAGCGGAAGTATC |
| CMY_JN714478_1882_2055 | /5Phos/ACCAATACGCCAGTAGCGAGAGTTGGAGGCTCATCGTTCCTATATTCCAC |
| | ACCACTTATTATTACAGATGTTATGCTCGCAGGTCGCAACGTAGCTGCCAAATC |
| CMY_X91840_1872_2046 | /5Phos/CAATCAGTGTGTTTGATTTGCACCGTTGGAGGCTCATCGTTCCTATATTC |
| | CACACCACTTATTATTACAGATGTTATGCTCGCAGGTCTACCCGGAATAGCCTGCTC |
| CTXM_EF219134_13713_13858 | /5Phos/CGGATAACGCCACGGGATGAGTTGGAGGCTCATCGTTCCTATATTCCACA |
| | CCACTTATTATTACAGATGTTATGCTCGCAGGTCACCGGGTCAAAGAATTCCTC |
| CTXM_HQ398215_802_947 | /5Phos/GCGGCGTGGTGGTGTCTCGTTGGAGGCTCATCGTTCCTATATTCCACACC |
| | ACTTATTATTACAGATGTTATGCTCGCAGGTCCGCTGCCGGTCTTATCAC |
| CTXM_AM982522_639_788 | /5Phos/GCCACGTCACCAGCTGCGGTTGGAGGCTCATCGTTCCTATATTCCACACC |
| | ACTTATTATTACAGATGTTATGCTCGCAGGTCCGGCTGGGTGAAGTAAGTC |
| GES_HM173356_1163_1321 | /5Phos/GCTCGTAGCGTCGCGTCTCGTTGGAGGCTCATCGTTCCTATATTCCACAC |
| | CACTTATTATTACAGATGTTATGCTCGCAGGTCTTGACCGACAGAGGCAAC |
| GES_AF156486_1754_1905 | /5Phos/CAGCAGGTCCGCCAATTTCTCGTTGGAGGCTCATCGTTCCTATATTCCAC |
| | ACCACTTATTATTACAGATGTTATGCTCGCAGGTCAGTGGACGTCAGTGCGC |
| GES_HQ874631_571_748 | /5Phos/CCATAGAGGACTTTAGCCACAGTGTTGGAGGCTCATCGTTCCTATATTCC |
| | ACACCACTTATTATTACAGATGTTATGCTCGCAGGTCTACACCGCTACAGCGTAAT |
| GES_FJ820124_1174_1338 | /5Phos/CATATGCAGAGTGAGCGGTCCGTTGGAGGCTCATCGTTCCTATATTCCAC |
| | ACCACTTATTATTACAGATGTTATGCTCGCAGGTCTCAATTCTTTCAAAGACCAGC |
| IMG_DQ361087_489_645 | /5Phos/CCATTAACTTCTTCAAACGATGTATGGTTGGAGGCTCATCGTTCCTATAT |
| | TCCACACCACTTATTATTACAGATGTTATGCTCGCAGGTCACCCGTGCTGTCGCTAT |
| IMG_JN848782_301_475 | /5Phos/GTGCTGTCGCTATGGAAATGTGGTTGGAGGCTCATCGTTCCTATATTCCA |
| | CACCACTTATTATTACAGATGTTATGCTCGCAGGTCAACCAAACCACTAGGTTATCTT |
| IMG_EF192154_182_328 | /5Phos/GTCAGTGTTTACAAGAACCACCAGTTGGAGGCTCATCGTTCCTATATTCC |
| | ACACCACTTATTATTACAGATGTTATGCTCGCAGGTCATGCATACGTGGGAATAGATT |
| IMG_AY033653_1343_1500 | /5Phos/CGGAAGTATCCGCGCGCCGTTGGAGGCTCATCGTTCCTATATTCCACACC |
| | ACTTATTATTACAGATGTTATGCTCGCAGGTCTTCGATCACGGCACGATC |
| IMG_AF318077_871_1047 | /5Phos/CGAACCAGCTTGGTTCCCAAGGTTGGAGGCTCATCGTTCCTATATTCCAC |
| | ACCACTTATTATTACAGATGTTATGCTCGCAGGTCTCACTGCGTGTTCGCTC |
| IMG_AF318077_515_657 | /5Phos/GATGCTGTACTTTGTGATGCCTAGTTGGAGGCTCATCGTTCCTATATTCC |
| | ACACCACTTATTATTACAGATGTTATGCTCGCAGGTCCGCTTGGCAAGTACTGTTC |
| KPC_HM066995_226_375 | /5Phos/GCAAGAAAGCCCTTGAATGAGCGTTGGAGGCTCATCGTTCCTATATTCCA |
| | CACCACTTATTATTACAGATGTTATGCTCGCAGGTCGCGTTATCACTGTATTGCAC |
| KPC_GQ140348_624_799 | /5Phos/AATCAACAAACTGCTGCCGCTGTTGGAGGCTCATCGTTCCTATATTCCAC |
| | ACCACTTATTATTACAGATGTTATGCTCGCAGGTCGCTGTACTTGTCATCCTTGT |
| KPC_EU729727_683_840 | /5Phos/CCAGTCTGCCGGCACCGCGTTGGAGGCTCATCGTTCCTATATTCCACACC |
| | ACTTATTATTACAGATGTTATGCTCGCAGGTCTCGAGCGCGAGTCTAGC |
| KPC_FJ234412_691_839 | /5Phos/CCGACTGCCCAGTCTGCCGGTTGGAGGCTCATCGTTCCTATATTCCACAC |
| | CACTTATTATTACAGATGTTATGCTCGCAGGTCCGAGCGCGAGTCTAGCC |
| NDM_JN104597_64_211 | /5Phos/GTAAATAGATGATCTTAATTTGGTTCACGTTGGAGGCTCATCGTTCCTAT |
| | ATTCCACACCACTTATTATTACAGATGTTATGCTCGCAGGTCTTGCTGGCCAATCGT |
| | CG |
| NDM_FN396876_2744_2885 | /5Phos/CACAGCCTGACTTTCGCCGCGTTGGAGGCTCATCGTTCCTATATTCCACA |
| | CCACTTATTATTACAGATGTTATGCTCGCAGGTCCAAGCAGGAGATCAACCTGC |
| NDM_FN396876_2958_3117 | /5Phos/GGTGGTCGATACCGCCTGGGTTGGAGGCTCATCGTTCCTATATTCCACAC |
| | CACTTATTATTACAGATGTTATGCTCGCAGGTCGTGAAATCCGCCCGACG |
| NDM_JN104597_314_465 | /5Phos/CATGTCGAGATAGGAAGTGTGCGTTGGAGGCTCATCGTTCCTATATTCCA |
| | CACCACTTATTATTACAGATGTTATGCTCGCAGGTCTGATGCGCGTGAGTCAC |
| NDM_FN396876_2382_2548 | /5Phos/CAATCTGCCATCGCGCGATTGTTGGAGGCTCATCGTTCCTATATTCCACA |
| | CCACTTATTATTACAGATGTTATGCTCGCAGGTCCGGCAATCTCGGTGATGC |
| OXA_EF650035_239_388 | /5Phos/CGAAGCAGGTACATACTCGGTCGTTGGAGGCTCATCGTTCCTATATTCCA |
| | CACCACTTATTATTACAGATGTTATGCTCGCAGGTCACGAGCTAAATCTTGATAAAC |
| | TT |
| OXA_EU019535_389_537 | /5Phos/TAGAATAGCGGAAGCTTTCATGGGTTGGAGGCTCATCGTTCCTATATTCC |
| | ACACCACTTATTATTACAGATGTTATGCTCGCAGGTCAGCTAGCTTGTAAGCAAACTG |
| OXA_EF650035_423_594 | /5Phos/CAAGTCCAATACGACGAGCTAAAGTTGGAGGCTCATCGTTCCTATATTCC |
| | ACACCACTTATTATTACAGATGTTATGCTCGCAGGTCGAATAGCATGGATTGCACTTC |
| OXA_DQ309276_232_380 | /5Phos/GGTACATACTCGGTCGAAGCACGTTGGAGGCTCATCGTTCCTATATTCCA |
| | CACCACTTATTATTACAGATGTTATGCTCGCAGGTCAATCTTGATAAACTGAAATAG |
| | CG |
| OXA_DQ445683_232_380 | /5Phos/GGTACATACTCGGTCGATGCACGTTGGAGGCTCATCGTTCCTATATTCCA |
| | CACCACTTATTATTACAGATGTTATGCTCGCAGGTCTCTTGATAAACCGGAATAGCG |
| OXA_X75562_201_366 | /5Phos/GTAATTGAACTAGCTAATGCCGTACGTTGGAGGCTCATCGTTCCTATATT |
| | CCACACCACTTATTATTACAGATGTTATGCTCGCAGGTCTTATGACACCAGTTTCTA |
| | GGC |
| OXA_M55547_995_1154 | /5Phos/CAAGTACTGTTCCTGTACGTCAGGTTGGAGGCTCATCGTTCCTATATTCC |
| | ACACCACTTATTATTACAGATGTTATGCTCGCAGGTCGCCCAGTTGTGATGCATTC |
| OXA_AY445080_313_469 | /5Phos/TCTCTTTCCCATTGTTTCATGGCGTTGGAGGCTCATCGTTCCTATATTCC |
| | ACACCACTTATTATTACAGATGTTATGCTCGCAGGTCTGCGGAAATTCTAAGCTGAC |
| PER_Z21957_217_371 | /5Phos/GTAGGTTATGCAGTTATTAGGTTCAGGTTGGAGGCTCATCGTTCCTATAT |
| | TCCACACCACTTATTATTACAGATGTTATGCTCGCAGGTCGACTCAGCCGAGTCAAGC |
| PER_HQ713678_6002_6167 | /5Phos/GCAGTACCAACATAGCTAAATGCGTTGGAGGCTCATCGTTCCTATATTCC |
| | ACACCACTTATTATTACAGATGTTATGCTCGCAGGTCAAATAACAAATCACAGGCCAC |
| PER_GQ396303_667_844 | /5Phos/GGTCCTGTGGTGGTTTCCACCGTTGGAGGCTCATCGTTCCTATATTCCAC |
| | ACCACTTATTATTACAGATGTTATGCTCGCAGGTCCGCGATAATGGCTTCATTGG |
| PER_X93314_954_1122 | /5Phos/TAACCGCTGTGGTCCTGTGGGTTGGAGGCTCATCGTTCCTATATTCCACA |
| | CCACTTATTATTACAGATGTTATGCTCGCAGGTCTGCGCAATAATAGCTTCATTG |
| PER_HQ713678_4517_4674 | /5Phos/GGAAGCGTTGCTTGCCATAGTGTTGGAGGCTCATCGTTCCTATATTCCAC |
| | ACCACTTATTATTACAGATGTTATGCTCGCAGGTCAACCGAAGCACCATGTAATT |
| PER_HQ713678_5074_5219 | /5Phos/GTTCGGTGCAAAGACGCCGGTTGGAGGCTCATCGTTCCTATATTCCACAC |
| | CACTTATTATTACAGATGTTATGCTCGCAGGTCTCGCAGACTTCAATATCAATATT |
| PER_GQ396303_254_399 | /5Phos/CACCTGATGCAGAACCAGCATGTTGGAGGCTCATCGTTCCTATATTCCAC |
| | ACCACTTATTATTACAGATGTTATGCTCGCAGGTCAGGCCACGTTATCACTGTG |
| SHV_AY661885_656_806 | /5Phos/CAGCTGCCGTTGCGAACGGTTGGAGGCTCATCGTTCCTATATTCCACACC |
| | ACTTATTATTACAGATGTTATGCTCGCAGGTCCGCAGATAAATCACCACAATC |
| SHV_AF535128_587_761 | /5Phos/GCTCAGACGCTGGCTGGTCGTTGGAGGCTCATCGTTCCTATATTCCACAC |
| | CACTTATTATTACAGATGTTATGCTCGCAGGTCCCGCAGATAAATCACCACG |
| SHV_U92041_406_579 | /5Phos/GCCAGTAGCAGATTGGCGGCGTTGGAGGCTCATCGTTCCTATATTCCACA |
| | CCACTTATTATTACAGATGTTATGCTCGCAGGTCGAACGGGCGCTCAGACG |
| SHV_AY288915_617_764 | /5Phos/CCACTGCAGCAGATGCCGTGTTGGAGGCTCATCGTTCCTATATTCCACAC |
| | CACTTATTATTACAGATGTTATGCTCGCAGGTCGTATCCCGCAGATAAATCACC |
| SHV_HQ637576_88_245 | /5Phos/TTAATTTGCTTAAGCGGCTGCGGTTGGAGGCTCATCGTTCCTATATTCCA |
| | CACCACTTATTATTACAGATGTTATGCTCGCAGGTCCCAGCTGTTCGTCACCG |
| SHV_AF535128_188_362 | /5Phos/GGGAAAGCGTTCATCGGCGGTTGGAGGCTCATCGTTCCTATATTCCACAC |
| | CACTTATTATTACAGATGTTATGCTCGCAGGTCTCGCTCATGGTAATGGCG |
| SHV_X98102_763_913 | /5Phos/TCTTATCGGCGATAAACCAGCCGTTGGAGGCTCATCGTTCCTATATTCCA |
| | CACCACTTATTATTACAGATGTTATGCTCGCAGGTCCGTTGCCAGTGCTCGAT |
| TEM_X64523_2037_2191 | /5Phos/CAGTCCCTCGATATTCAGATCAGAGTTGGAGGCTCATCGTTCCTATATTC |
| | CACACCACTTATTATTACAGATGTTATGCTCGCAGGTCTTAACAATTTCGCAACCGTC |
| TEM_J01749_2068_2239 | /5Phos/CAGCTGCGGTAAAGCTCATCAGTTGGAGGCTCATCGTTCCTATATTCCAC |
| | ACCACTTATTATTACAGATGTTATGCTCGCAGGTCCATAGTTAAGCCAGTATACACTC |
| TEM_GQ149347_3605_3747 | /5Phos/GTCGGAAAGTTGACCAGACATTAGTTGGAGGCTCATCGTTCCTATATTCC |
| | ACACCACTTATTATTACAGATGTTATGCTCGCAGGTCATACTAGGAGAAGTTAATAA |
| | ATACG |
| TEM_U36911_4374_4551 | /5Phos/CATTCTCTCGCTTTAATTTATTAACCTGTTGGAGGCTCATCGTTCCTATA |
| | TTCCACACCACTTATTATTACAGATGTTATGCTCGCAGGTCATCGACCTTCTGGACA |
| | TTATC |
| TEM_AF091113_1529_1699 | /5Phos/GTAACAACTTTCATGCTCTCCTAAAGTTGGAGGCTCATCGTTCCTATATT |
| | CCACACCACTTATTATTACAGATGTTATGCTCGCAGGTCCGGTAACTGATGCCGTAT |
| | TT |
| TEM_GU371926_11801_11944 | /5Phos/GTGAAGTGAATGGTCAGTATGTTGGTTGGAGGCTCATCGTTCCTATATTC |
| | CACACCACTTATTATTACAGATGTTATGCTCGCAGGTCAGTGCGCAGGAGATTAGC |
| TEM_J01749_766_908 | /5Phos/CCTGTCCTACGAGTTGCATGATGTTGGAGGCTCATCGTTCCTATATTCCA |
| | CACCACTTATTATTACAGATGTTATGCTCGCAGGTCATAATGGCCTGCTTCTCGC |
| TEM_J01749_1634_1783 | /5Phos/CGTTTCCAGACTTTACGAAACACGTTGGAGGCTCATCGTTCCTATATTCC |
| | ACACCACTTATTATTACAGATGTTATGCTCGCAGGTCACGTTGTGAGGGTAAACAAC |
| TEM_U36911_7596_7762 | /5Phos/CGTTGCTTACGCAACCAAATATCGTTGGAGGCTCATCGTTCCTATATTCC |
| | ACACCACTTATTATTACAGATGTTATGCTCGCAGGTCTGATCTTGCTCAATGAGGTTA |
| TEM_U36911_6901_7069 | /5Phos/CATCATGTTCATATTTATCAGAGCTCGTTGGAGGCTCATCGTTCCTATAT |
| | TCCACACCACTTATTATTACAGATGTTATGCTCGCAGGTCTAGATTTCATAAAGTCT |
| | AACACAC |
| TEM_GU371926_33909_34082 | /5Phos/GTTTCCACATGGTGAACGGTGGTTGGAGGCTCATCGTTCCTATATTCCAC |
| | ACCACTTATTATTACAGATGTTATGCTCGCAGGTCAAACCTGTCACTCTGAATGTT |
| VEB_EU259884_6947_7094 | /5Phos/CAAATACTAAATTATACAGTATCAGAGAGGTTGGAGGCTCATCGTTCCTA |
| | TATTCCACACCACTTATTATTACAGATGTTATGCTCGCAGGTCATGCAAAGCGTTAT |
| | GAAATTTC |
| VEB_EF136375_596_738 | /5Phos/GTTCTTATTATTATAAGTATCTATTAACAGTTGTTGGAGGCTCATCGTTC |
| | CTATATTCCACACCACTTATTATTACAGATGTTATGCTCGCAGGTCCATTAGTGGCT |
| | GCTGCAAT |
| VEB_EF420108_234_380 | /5Phos/CATCGGGAAATGGAAGTCGTTATGTTGGAGGCTCATCGTTCCTATATTCC |
| | ACACCACTTATTATTACAGATGTTATGCTCGCAGGTCGTTCAATCGTCAAAGTTGTTC |
| VEB_AF010416_89_230 | /5Phos/CGTGGTTTGTGCTGAGCAAAGGTTGGAGGCTCATCGTTCCTATATTCCAC |
| | ACCACTTATTATTACAGATGTTATGCTCGCAGGTCCAAAGTTAAGTTGTCAGTTTGAG |
| VIM_AY524988_385_552 | /5Phos/GCCGCCCGAAGGACATCAAGTTGGAGGCTCATCGTTCCTATATTCCACAC |
| | CACTTATTATTACAGATGTTATGCTCGCAGGTCAGACGGGACGTACACAAC |
| VIM_Y18050_3464_3614 | /5Phos/GCAACTCATCACCATCACGGAGTTGGAGGCTCATCGTTCCTATATTCCAC |
| | ACCACTTATTATTACAGATGTTATGCTCGCAGGTCTGATGCGTACGTTGCCAC |
| VIM_AY635904_58_203 | /5Phos/GCGACAGCCATGACAGACGCGTTGGAGGCTCATCGTTCCTATATTCCACA |
| | CCACTTATTATTACAGATGTTATGCTCGCAGGTCGGACAATGAGACCATTGGAC |
| VIM_HM750249_275_454 | /5Phos/AAACGACTGCGTTGCGATATGGTTGGAGGCTCATCGTTCCTATATTCCAC |
| | ACCACTTATTATTACAGATGTTATGCTCGCAGGTCTTCCGAAGGACATCAACGC |
| VIM_AJ536835_313_481 | /5Phos/ATGCGACCAAACGCCATCGCGTTGGAGGCTCATCGTTCCTATATTCCACA |
| | CCACTTATTATTACAGATGTTATGCTCGCAGGTCATCGTCATGGAAGTGCGTA |
| VIM_EU118148_131_300 | /5Phos/GAACAGGCTTATGTCAACTGGGGTTGGAGGCTCATCGTTCCTATATTCCA |
| | CACCACTTATTATTACAGATGTTATGCTCGCAGGTCCATAACATCAAACATCGACCC |
| VIM_DQ143913_921_1063 | /5Phos/ACGAACCGAACAGGCTTATGTCGTTGGAGGCTCATCGTTCCTATATTCCA |
| | CACCACTTATTATTACAGATGTTATGCTCGCAGGTCTAACGCGCTTGCTGCTT |
| VIM_EU118148_2821_2961 | /5Phos/GCTGTAATTATGACGACGCCGGTTGGAGGCTCATCGTTCCTATATTCCAC |
| | ACCACTTATTATTACAGATGTTATGCTCGCAGGTCCTCGGTGAGATTCAGAATGC |
| VIM_EU118148_1060_1229 | /5Phos/CATCATAGACGCGGTCAAATAGAGTTGGAGGCTCATCGTTCCTATATTCC |
| | ACACCACTTATTATTACAGATGTTATGCTCGCAGGTCACTCATCACCATCACGGAC |
| van_DQ018710.1_6481_6652 | /5Phos/GTGTATGTCAGCGATTTGTCCATGTTGGAGGCTCATCGTTCCTATATTCC |
| | ACACCACTTATTATTACAGATGTTATGCTCGCAGGTCTGTCATATTGTCTTGCCGATT |
| van_DQ018710.1_6764_6926 | /5Phos/GTCCACCTCGCCAACAATCAAGTTGGAGGCTCATCGTTCCTATATTCCAC |
| | ACCACTTATTATTACAGATGTTATGCTCGCAGGTCATATCAACACGGGAAAGACCT |
| van_AY926880.1_3640_3785 | /5Phos/GCGTGATTATCACGTTCGGCAGTTGGAGGCTCATCGTTCCTATATTCCAC |
| | ACCACTTATTATTACAGATGTTATGCTCGCAGGTCCTTGCAGATTTAACCGACAC |
| van_FJ545640.1_517_690 | /5Phos/GGCTCGACTTCCTGATGAATACGGTTGGAGGCTCATCGTTCCTATATTCC |
| | ACACCACTTATTATTACAGATGTTATGCTCGCAGGTCTGAAACCGGGCAGAGTATT |
| van_AE017171.1_34715_34859 | /5Phos/CAACGATGTATGTCAACGATTTGTGTTGGAGGCTCATCGTTCCTATATTC |
| | CACACCACTTATTATTACAGATGTTATGCTCGCAGGTCATTGCGTAGTCCAATTCGTC |
| van_NC_008821.1_11898_12045 | /5Phos/CAGGCTGTTTCGGGCTGTGAGTTGGAGGCTCATCGTTCCTATATTCCACA |
| | CCACTTATTATTACAGATGTTATGCTCGCAGGTCGGGTTATTAATAAAGATGATAGGC |
| van_FJ349556.1_5601_5765 | /5Phos/GGCTCGGCTTCCTGATGAATACGTTGGAGGCTCATCGTTCCTATATTCCA |
| | CACCACTTATTATTACAGATGTTATGCTCGCAGGTCAGGCATGGTATTGACTTCATT |
| mecA_AY820253.1_1431_1608 | /5Phos/TAATTCAAGTGCAACTCTCGCAAGTTGGAGGCTCATCGTTCCTATATTCC |
| | ACACCACTTATTATTACAGATGTTATGCTCGCAGGTCTTTATTCTCTAATGCGCTAT |
| | ATATT |
| mecA_AY952298.1_130_302 | /5Phos/GGATAGTTACGACTTTCTGCTTCAGTTGGAGGCTCATCGTTCCTATATTC |
| | CACACCACTTATTATTACAGATGTTATGCTCGCAGGTCTGTATTGCTATTATCGTCA |
| | ACG |
| mecA_AM048806.2_1574_1720 | /5Phos/CAGTATTTCACCTTGTCCGTAACCGTTGGAGGCTCATCGTTCCTATATTC |
| | CACACCACTTATTATTACAGATGTTATGCTCGCAGGTCGTTTACGACTTGTTGCATGC |
| mecA_EF692630.1_239_405 | /5Phos/AATGTTTATATCTTTAACGCCTAAACTGTTGGAGGCTCATCGTTCCTATA |
| | TTCCACACCACTTATTATTACAGATGTTATGCTCGCAGGTCATGCTTTGGTCTTTCT |
| | GCAT |
| mex_AF092566.1_371_520 | /5Phos/CTGGCCCTTGAGGTCGCGGGTTGGAGGCTCATCGTTCCTATATTCCACAC |
| | CACTTATTATTACAGATGTTATGCTCGCAGGTCCGGTCTTCACCTCGACAC |
| mex_AF092566.1_50_193 | /5Phos/GACGTAGATCGGGTCGAGCTGTTGGAGGCTCATCGTTCCTATATTCCACA |
| | CCACTTATTATTACAGATGTTATGCTCGCAGGTCACGGAAACCTCGGAGAATT |
| mex_CP000438.1_487178_487357 | /5Phos/GGCGTACTGCTGCTTGCTCAGTTGGAGGCTCATCGTTCCTATATTCCACA |
| | CCACTTATTATTACAGATGTTATGCTCGCAGGTCTGACGTCGACGTAGATCG |
| mex_NZ_AAQW01000001.1_461304_461466 | /5Phos/CCTGTTCCTGGGTCGAAGCCGTTGGAGGCTCATCGTTCCTATATTCCACA |
| | CCACTTATTATTACAGATGTTATGCTCGCAGGTCCTTCGGTCACCGCGGA |
| erm_NC_002745.2_871803_871973 | /5Phos/GTCAGGCTAAATATAGCTATCTTATCGGTTGGAGGCTCATCGTTCCTATA |
| | TTCCACACCACTTATTATTACAGATGTTATGCTCGCAGGTCTCAGTTACTGCTATAG |
| | AAATTGAT |
| erm_NC_002745.2_871666_871841 | /5Phos/CATCCTAAGCCAAGTGTAGACTCGTTGGAGGCTCATCGTTCCTATATTCC |
| | ACACCACTTATTATTACAGATGTTATGCTCGCAGGTCAAGATATATGGTAATATTCC |
| | TTATAAC |
| erm_EU047809.1_79_229 | /5Phos/GTTTATAAGTGGGTAAACCGTGAATGTTGGAGGCTCATCGTTCCTATATT |
| | CCACACCACTTATTATTACAGATGTTATGCTCGCAGGTCGAAACGAGCTTTAGGTTT |
| | GC |
| acinetobacter_NC_010611_627997_628164 | /5Phos/GCAGCACTTGACCGCCATGAGTGACCAGTTGGAGGCTCATCGTTCCTATA |
| | TTCCACACCACTTATTATTACAGATGTTATGCTCGCAGGTCCATCGCACCAACAACA |
| | ATAATCG |
| acinetobacter_NC_010611_2417580_2417755 | /5Phos/GTGATCACTGATGCACCAGATGAAGTGTTGGAGGCTCATCGTTCCTATAT |
| | TCCACACCACTTATTATTACAGATGTTATGCTCGCAGGTCATCTTGATATTCAAGTC |
| | TATGACG |
| acinetobacter_CP002522_11753_11931 | /5Phos/GATATTATTGATCATGGTGCCAAGCCAAGTTGGAGGCTCATCGTTCCTAT |
| | ATTCCACACCACTTATTATTACAGATGTTATGCTCGCAGGTCCAATATGAAGCTGAC |
| | GACGCG |
| acinetobacter_NC_011586_3908329_3908508 | /5Phos/GCTGAGCGTGAAGGTTCATGGATTATTAGTTGGAGGCTCATCGTTCCTAT |
| | ATTCCACACCACTTATTATTACAGATGTTATGCTCGCAGGTCGGTAAGGCTTACGGT |
| | CTCAT |
| acinetobacter_NC_010611_145181_145340 | /5Phos/GCATCTTGTGCAGCCTGAATAGCAGCGTGTTGGAGGCTCATCGTTCCTAT |
| | ATTCCACACCACTTATTATTACAGATGTTATGCTCGCAGGTCACCACGTTGAATATC |
| | ACCTTCGGCAT |
| acinetobacter_NC_010611_3854494_3854662 | /5Phos/AAGTCCATAATTGCTTGAGTGTAGTCATGTTGGAGGCTCATCGTTCCTAT |
| | ATTCCACACCACTTATTATTACAGATGTTATGCTCGCAGGTCATCTTCGCACTGAAT |
| | AATAAGAACAT |
| acinetobacter_NC_010400_56216_56383 | /5Phos/GCTTGCTGGTTCTGCACGTAGCTTACTGGTTGGAGGCTCATCGTTCCTAT |
| | ATTCCACACCACTTATTATTACAGATGTTATGCTCGCAGGTCAAGATGAACAGGCTA |
| | CTGCAA |
| acinetobacter_NC_010611_1454960_1455136 | /5Phos/GCAGCGCTGTGCAAGTTCAATGTATTCTGTTGGAGGCTCATCGTTCCTAT |
| | ATTCCACACCACTTATTATTACAGATGTTATGCTCGCAGGTCCTCGTGCGAGTATTC |
| | CTTAAGTGT |
| acinetobacter_NC_009085_255964_256143 | /5Phos/GTATAACACTCGGCCAGCGCCAAGGTTCGTTGGAGGCTCATCGTTCCTAT |
| | ATTCCACACCACTTATTATTACAGATGTTATGCTCGCAGGTCGTTCACACATCGCCA |
| | CAATATGAT |
| clostridium_NC_013974_3097606_3097772 | /5Phos/ACCATGCAGATACAATGAACCAGTTGGAGGCTCATCGTTCCTATATTCCA |
| | CACCACTTATTATTACAGATGTTATGCTCGCAGGTCGGATGATAAGACACATCCAAT |
| | TC |
| clostridium_FN665653_103469_103631 | /5Phos/CATCAACAGCTTCTTGAAGCATTCGTTGGAGGCTCATCGTTCCTATATTC |
| | CACACCACTTATTATTACAGATGTTATGCTCGCAGGTCGTCCAACAACTATAACAGA |
| | ACGTC |
| clostridium_NC_013974_117188_117346 | /5Phos/AACATATCACCTGATATTCTAGTATCGTTGGAGGCTCATCGTTCCTATAT |
| | TCCACACCACTTATTATTACAGATGTTATGCTCGCAGGTCATTCCATTATATTCAAC |
| | AGGATTGTGA |
| clostridium_NC_013316_3012882_3013047 | /5Phos/GCTGTTGCTTGCGGATACTGGTTGGAGGCTCATCGTTCCTATATTCCACA |
| | CCACTTATTATTACAGATGTTATGCTCGCAGGTCCGTATATGTAGCTCAAGTTGC |
| clostridium_FN668375_1212250_1212413 | /5Phos/AAGAGCTAATGCAGCTATTGCACTTATGTTGGAGGCTCATCGTTCCTATA |
| | TTCCACACCACTTATTATTACAGATGTTATGCTCGCAGGTCCATACACTTCAGCTAT |
| | AAGACCAT |
| clostridium_NC_013315_3754484_3754640 | /5Phos/AACAAGAGCAGAAGTTACAGACGTGTTGGAGGCTCATCGTTCCTATATTC |
| | CACACCACTTATTATTACAGATGTTATGCTCGCAGGTCGTATAATGGTGGCTAGAGG |
| | TGA |
| clostridium_FN665654_3239860_3240039 | /5Phos/ACTCGTGAAGACCATGCAGATACAAGTTGGAGGCTCATCGTTCCTATATT |
| | CCACACCACTTATTATTACAGATGTTATGCTCGCAGGTCAATACTTACAATGCCTGA |
| | GGA |
| clostridium_FN668941_3228320_3228491 | /5Phos/ACCATGCAGATACAATGAACCGTTGGAGGCTCATCGTTCCTATATTCCAC |
| | ACCACTTATTATTACAGATGTTATGCTCGCAGGTCCCTGAGGATGATAAGACACATC |
| clostridium_NC_013974_1962664_1962825 | /5Phos/GCATCTGCTGCTTCTATTGCTCCTACTGTTGGAGGCTCATCGTTCCTATA |
| | TTCCACACCACTTATTATTACAGATGTTATGCTCGCAGGTCACATGAACTGATATTA |
| | GTTCTCCAA |
| clostridium_NC_003366_2769687_2769851 | /5Phos/GCACAAGCTGGAGATAACATCGGGTTGGAGGCTCATCGTTCCTATATTCC |
| | ACACCACTTATTATTACAGATGTTATGCTCGCAGGTCGTAGAGGACGTATTCACAAT |
| | CACT |
| clostridium_FN665653_127741_127918 | /5Phos/CTCTATCAGCTTCTACTGCTTCTTCGTTGGAGGCTCATCGTTCCTATATT |
| | CCACACCACTTATTATTACAGATGTTATGCTCGCAGGTCCCATCTCATCCACAGTTA |
| | ATATATC |
| clostridium_NC_013316_2259929_2260107 | /5Phos/AGATGAGATTCATACTATCGTTGGAGCTGTTGGAGGCTCATCGTTCCTAT |
| | ATTCCACACCACTTATTATTACAGATGTTATGCTCGCAGGTCAGCAGAGAGAATAGT |
| | AAGAGGAGA |
| clostridium_NC_009089_94774_94937 | /5Phos/CATCAACAGCTTCTTGAAGCATTGTTGGAGGCTCATCGTTCCTATATTCC |
| | ACACCACTTATTATTACAGATGTTATGCTCGCAGGTCGTCCAACAACTATAACAGAA |
| | CG |
| clostridium_NC_013315_2044225_2044389 | /5Phos/GTCAGCAATACGCCACCAAGCTCCTATGTTGGAGGCTCATCGTTCCTATA |
| | TTCCACACCACTTATTATTACAGATGTTATGCTCGCAGGTCGTGGTGGATATCCTGT |
| | TACC |
| clostridium_NC_013315_2299408_2299586 | /5Phos/GCGCAATAGAGTTGTATAAGAGTGCTGGTTGGAGGCTCATCGTTCCTATA |
| | TTCCACACCACTTATTATTACAGATGTTATGCTCGCAGGTCAGCATTAATTATAGAT |
| | TATAATGTATAA |
| clostridium_FN668941_3244255_3244408 | /5Phos/GGCATAATAGGATGGATAGATGAGTTGGAGGCTCATCGTTCCTATATTCC |
| | ACACCACTTATTATTACAGATGTTATGCTCGCAGGTCACTAATCCAACTTCTACTGC |
| | TAT |
| clostridium_NC_013316_3610909_3611065 | /5Phos/GTACATTCACATATAGACCATCTTAAGTTGGAGGCTCATCGTTCCTATAT |
| | TCCACACCACTTATTATTACAGATGTTATGCTCGCAGGTCACATAGGTGCAGGTAGA |
| | ATAGTATA |
| clostridium_FN665653_1104859_1105031 | /5Phos/CCATACCAGTATCTTGGCATATTGGTTGGAGGCTCATCGTTCCTATATTC |
| | CACACCACTTATTATTACAGATGTTATGCTCGCAGGTCATAATGAATAACAGCAGGT |
| | GTATTA |
| clostridium_NC_03366_2753681_2753838 | /5Phos/AGATGAAGCACAAGCTGGAGATAAGTTGGAGGCTCATCGTTCCTATATTC |
| | CACACCACTTATTATTACAGATGTTATGCTCGCAGGTCAGGACGTATTCACAATCAC |
| | TG |
| clostridium_FN665653_710906_711080 | /5Phos/ATAATCATTCACCTCCATCATTCATAAGTTGGAGGCTCATCGTTCCTATA |
| | TTCCACACCACTTATTATTACAGATGTTATGCTCGCAGGTCACTGAATATGGTTCGT |
| | CTCA |
| clostridium_NC_009089_3706562_3706720 | /5Phos/GTACATTCACATATAGACCATCTTAGTTGGAGGCTCATCGTTCCTATATT |
| | CCACACCACTTATTATTACAGATGTTATGCTCGCAGGTCACATAGGTGCAGGTAGAA |
| | TAGT |
| clostridium_NC_013316_137282_1372968 | /5Phos/ACTCCACCAGGATGTTGTCCGTTGGAGGCTCATCGTTCCTATATTCCACA |
| | CCACTTATTATTACAGATGTTATGCTCGCAGGTCGTAGGACCGTCGTGTCCAAG |
| clostridium_FN665652_676696_676895 | /5Phos/GCAATATCAATGGTATCGAAGGCACTATGTTGGAGGCTCATCGTTCCTAT |
| | ATTCCACACCACTTATTATTACAGATGTTATGCTCGCAGGTCGTATTGAAGGTACTA |
| | TTAGCGATATGC |
| clostridium_NC_013316_2641651_2641808 | /5Phos/GTGCCGGTCTCGGTTACTCAATGGTTGGAGGCTCATCGTTCCTATATTCC |
| | ACACCACTTATTATTACAGATGTTATGCTCGCAGGTCGGATTATTATAATGCAGCTA |
| | GAAG |
| clostridium_FN668375_3595870_3596026 | /5Phos/GTACATTCACATATAGACCATCTTGTTGGAGGCTCATCGTTCCTATATTC |
| | CACACCACTTATTATTACAGATGTTATGCTCGCAGGTCACATAGGTGCAGGTAGAAT |
| | AGTA |
| clostridium_FN668941_1105700_1105868 | /5Phos/AGTTCCTTCATATGACTCAGTTGATTGAGTTGGAGGCTCATCGTTCCTAT |
| | ATTCCACACCACTTATTATTACAGATGTTATGCTCGCAGGTCGTTATATCTTCAATT |
| | ATACATTCCTGC |
| clostridium_NC_013974_2505182_2505359 | /5Phos/CAGCAGTTGTTGCTAGAGGTATGGTTGGAGGCTCATCGTTCCTATATTCC |
| | ACACCACTTATTATTACAGATGTTATGCTCGCAGGTCGCATCACCAGGTGCAGCAAGT |
| clostridium_NC_013315_1077126_1077298 | /5Phos/GCAATTCTCTGTTGTTGTCCTCCACTCAGTTGGAGGCTCATCGTTCCTAT |
| | ATTCCACACCACTTATTATTACAGATGTTATGCTCGCAGGTCAGTAAGAGCCTCTTC |
| | TTGGTCATGA |
| clostridium_NC_009089_2182303_2182482 | /5Phos/CTATTCCTGATAATAAGTGTGTCCTCATGTTGGAGGCTCATCGTTCCTAT |
| | ATTCCACACCACTTATTATTACAGATGTTATGCTCGCAGGTCCGGCATCATCTAACA |
| | ATTCTTCT |
| clostridium_FN665652_1909777_1909942 | /5Phos/GTAATTCCAATTACTTCTAGCTCTGGTGGTTGGAGGCTCATCGTTCCTAT |
| | ATTCCACACCACTTATTATTACAGATGTTATGCTCGCAGGTCTACCATCTTCTCCAT |
| | GTGTAT |
| clostridium_NC_013316_3300896_3301062 | /5Phos/CCATGCAGATACAATGAACCAGGTTGGAGGCTCATCGTTCCTATATTCCA |
| | CACCACTTATTATTACAGATGTTATGCTCGCAGGTCGATGATAAGACACATCCAATT |
| | CC |
| clostridium_NC_013316_871338_871499 | /5Phos/CCTTCTGCCATTGTAGAACAAGCTCCATGTTGGAGGCTCATCGTTCCTAT |
| | ATTCCACACCACTTATTATTACAGATGTTATGCTCGCAGGTCCCTGTAACTGTCCAC |
| | TGAGC |
| clostridium_NC_013316_3608873_3609047 | /5Phos/CAATCATGATAGAATTAGATGGAACGTTGGAGGCTCATCGTTCCTATATT |
| | CCACACCACTTATTATTACAGATGTTATGCTCGCAGGTCAGCAATAGTTCCATCAGG |
| | AGCATC |
| clostridium_FN665654_3717059_3717221 | /5Phos/AGTGGTGAAGGTGTTCAACAAGGTTGGAGGCTCATCGTTCCTATATTCCA |
| | CACCACTTATTATTACAGATGTTATGCTCGCAGGTCACTGAAGCTGGATATGTTGGAG |
| clostridium_NC_013315_2010489_2010657 | /5Phos/CGCCTCTTCAGAAGCGGATATCAGTTGGAGGCTCATCGTTCCTATATTCC |
| | ACACCACTTATTATTACAGATGTTATGCTCGCAGGTCGCCAGACTTCCGCCACAACCT |
| clostridium_NC_013315_3236301_3236474 | /5Phos/GGCATAATAGGATGGATAGATGAGCGTTGGAGGCTCATCGTTCCTATATT |
| | CCACACCACTTATTATTACAGATGTTATGCTCGCAGGTCGCAGCAGTTGTACCTACA |
| | ACTAA |
| clostridium_NC_013315_1095924_1096090 | /5Phos/AGTTCCTTCATATGACTCAGTTGATTGGTTGGAGGCTCATCGTTCCTATA |
| | TTCCACACCACTTATTATTACAGATGTTATGCTCGCAGGTCGTTATATCTTCAATTA |
| | TACATTCCTGCG |
| enterobacter_NC_014121_4735453_4735632 | /5Phos/GCATGGTAGTTCGCCAGCCGCTGGAACGTTGGAGGCTCATCGTTCCTATA |
| | TTCCACACCACTTATTATTACAGATGTTATGCTCGCAGGTCACAGCAACCGCAAGTT |
| | CTTGACAT |
| enterobacter_NC_015663_1014187_1014345 | /5Phos/AATATCATGGTCGTGTCCAGGCACTGGCGTTGGAGGCTCATCGTTCCTAT |
| | ATTCCACACCACTTATTATTACAGATGTTATGCTCGCAGGTCGTTCTGGTAGCTGCT |
| | TCTACTGTA |
| enterobacter_FP929040_3448334_3448513 | /5Phos/AACTTACAACTACGCGCACTTGAATCGGTTGGAGGCTCATCGTTCCTATA |
| | TTCCACACCACTTATTATTACAGATGTTATGCTCGCAGGTCGAGTGTTGTATGATAG |
| | TCTCGGT |
| enterobacter_NC_009436_4051820_4051985 | /5Phos/GCAAGTTGAGGAGATGCTGGCATGATTCGTTGGAGGCTCATCGTTCCTAT |
| | ATTCCACACCACTTATTATTACAGATGTTATGCTCGCAGGTCACATGGCTCTGGAAG |
| | ATGTGCTGATC |
| enterococcus_FP929058_1738439_1738606 | /5Phos/GCGATAATTGTAATGATTCGTGGTGTTAGTTGGAGGCTCATCGTTCCTAT |
| | ATTCCACACCACTTATTATTACAGATGTTATGCTCGCAGGTCCCGTTGTCAATCCAG |
| | TTAGTAGACT |
| enterococcus_CP002621_1819224_1819388 | /5Phos/ACTGTGGCAGTCTATGTTCCAATTGTAGTTGGAGGCTCATCGTTCCTATA |
| | TTCCACACCACTTATTATTACAGATGTTATGCTCGCAGGTCCTTATCGACATAATCC |
| | TGATAATC |
| enterococcus_FP929058_904007_904173 | /5Phos/GCGTCGCTTCTTGCGCTCGCCGTTGGAGGCTCATCGTTCCTATATTCCAC |
| | ACCACTTATTATTACAGATGTTATGCTCGCAGGTCAATGTATTCATACCGTCAAGT |
| enterococcus_FP929058_551757_551920 | /5Phos/GCCTTCACAACTACGTTGGAAGGTCTTCGTTGGAGGCTCATCGTTCCTAT |
| | ATTCCACACCACTTATTATTACAGATGTTATGCTCGCAGGTCCTAACAGTCCTGCCG |
| | ACTAC |
| enterococcus_NC_004668_1122345_1122507 | /5Phos/GCCTTCACAACTACGTTGGAAGGTCTTGTTGGAGGCTCATCGTTCCTATA |
| | TTCCACACCACTTATTATTACAGATGTTATGCTCGCAGGTCCTAACAGTCCTGCCGA |
| | CTACT |
| klebsiella_NC_009648_2885456_2885620 | /5Phos/GCCGCTGAGCGGCGGCAAGCCGATGGCGTTGGAGGCTCATCGTTCCTATA |
| | TTCCACACCACTTATTATTACAGATGTTATGCTCGCAGGTCGAATGGCAGGCCAAGC |
| | TGAAGGCG |
| klebsiella_NC_009648_3899012_3899182 | /5Phos/GCCAAGCGGCATTCTGGCGCCAGTGGAGTTGGAGGCTCATCGTTCCTATA |
| | TTCCACACCACTTATTATTACAGATGTTATGCTCGCAGGTCCCAGACCGGAGTGGAC |
| | AACGTCGAGGCG |
| klebsiella_NC_009648_4980596_4980757 | /5Phos/GCCGTATATCATCGGCAATAACCGCACGGTTGGAGGCTCATCGTTCCTAT |
| | ATTCCACACCACTTATTATTACAGATGTTATGCTCGCAGGTCGCATGATGGTCAACA |
| | AGGTGC |
| klebsiella_NC_009648_3266359_3266519 | /5Phos/ACGAGCCGAGATAGGTCTGCAGCGTACGTTGGAGGCTCATCGTTCCTATA |
| | TTCCACACCACTTATTATTACAGATGTTATGCTCGCAGGTCGTACTGATATTCACCA |
| | TACTGCCG |
| klebsiella_NC_012731_2557467_2557634 | /5Phos/GCAATATCTTCACCGGCAGCCACCGCGGTTGGAGGCTCATCGTTCCTATA |
| | TTCCACACCACTTATTATTACAGATGTTATGCTCGCAGGTCGGTATATGGCACGCCA |
| | ATCGC |
| klebsiella_NC_012731_4857136_4857315 | /5Phos/AATAACCTTAACGTCGCCAACACGGTTGGAGGCTCATCGTTCCTATATTC |
| | CACACCACTTATTATTACAGATGTTATGCTCGCAGGTCCTCGGTGAACACCTCCTGG |
| | CACG |
| proteus_NC_010554_547938_548117 | /5Phos/GCGGAACTGCTTGGCGTAGTAAGCGTTGGAGGCTCATCGTTCCTATATTC |
| | CACACCACTTATTATTACAGATGTTATGCTCGCAGGTCCATGTAGTGCCGTAGACCT |
| | TCACCA |
| pseudomonas_NC_008463_658500_658676 | /5Phos/GCGAGACCGGCGGCACCATCGTCTCCAGGTTGGAGGCTCATCGTTCCTAT |
| | ATTCCACACCACTTATTATTACAGATGTTATGCTCGCAGGTCTTCTGCCTGATGGAC |
| | GTCTCCGGCTCG |
| pseudomonas_NC_008463_753931_754099 | /5Phos/GCGGTTCACCTGTTCGCCTTCGAACACGGTTGGAGGCTCATCGTTCCTAT |
| | ATTCCACACCACTTATTATTACAGATGTTATGCTCGCAGGTCGCGCAGCATCTGACG |
| | CAGGATGGTCTCG |
| pseudomonas_NC_009656_6431649_6431828 | /5Phos/ACTCCATCGCCATCAAGGACATGGCCGGGTTGGAGGCTCATCGTTCCTAT |
| | ATTCCACACCACTTATTATTACAGATGTTATGCTCGCAGGTCATCGACGTGTTCCGC |
| | ATCTTCGACGCG |
| pseudomonas_NC_008463_560357_560534 | /5Phos/GCCTGATGCACTACAGCGCCTGGGTTGGAGGCTCATCGTTCCTATATTCC |
| | ACACCACTTATTATTACAGATGTTATGCTCGCAGGTCTACCACATGGTCGATCTCGA |
| | CGACTGC |
| pseudomonas_NC_010322_5224859_5225023 | /5Phos/GCGCATCCAGGACGGCGAGTACGGTTGGAGGCTCATCGTTCCTATATTCC |
| | ACACCACTTATTATTACAGATGTTATGCTCGCAGGTCCTTCGAGTGCCTGCACGAGC |
| | TGAA |
| pseudomonas_NC_008463_4839746_4839924 | /5Phos/GCTGGAGAACGTCAAGGTGGTGATCATCGTTGGAGGCTCATCGTTCCTAT |
| | ATTCCACACCACTTATTATTACAGATGTTATGCTCGCAGGTCACCGATAACGACGAC |
| | CGCATCAA |
| staph_FN433596_2844085_2844263 | /5Phos/ACGATTGGAGAAGGCAGTGTGATTGGGTTGGAGGCTCATCGTTCCTATAT |
| | TCCACACCACTTATTATTACAGATGTTATGCTCGCAGGTCGGACAGATTACAATTGG |
| | CG |
| staph_NC_009632_1198350_1198529 | /5Phos/GCCGCAATACCGATATTCCAGTTGGAGGCTCATCGTTCCTATATTCCACA |
| | CCACTTATTATTACAGATGTTATGCTCGCAGGTCCCATTGTCCACCAGCTGAACCG |
| staph_FN433596_2521244_2521419 | /5Phos/GTGAAGGTCGTGCTCCTATCGGTGTTGGAGGCTCATCGTTCCTATATTCC |
| | ACACCACTTATTATTACAGATGTTATGCTCGCAGGTCAGATCTGGTGAAGTTCGTAT |
| | GAT |
| staph_NC_009487_430842_431017 | /5Phos/GCTGGTACTTGTACTTATATCGAGTTGGAGGCTCATCGTTCCTATATTCC |
| | ACACCACTTATTATTACAGATGTTATGCTCGCAGGTCATCAGAAGATGATATCGTTA |
| | CGTCAT |
| staph_NC_009782_2086681_2086849 | /5Phos/GCGCATATTGCATTAATGGCTATAGATGTTGGAGGCTCATCGTTCCTATA |
| | TTCCACACCACTTATTATTACAGATGTTATGCTCGCAGGTCGCCAGCAGGTTATACA |
| | CTCG |
| staph_NC_009782_58256_58423 | /5Phos/GCAATTCTTACCACAGCACGAAGAACAGGTTGGAGGCTCATCGTTCCTAT |
| | ATTCCACACCACTTATTATTACAGATGTTATGCTCGCAGGTCATCTAGATGAAGATA |
| | ATGAAGTCG |
| staph_NC_013450_991049_991222 | /5Phos/GCATCTTCATACAATACTTCTAGCTTACGTTGGAGGCTCATCGTTCCTAT |
| | ATTCCACACCACTTATTATTACAGATGTTATGCTCGCAGGTCCACAATACCAGTTGT |
| | ATTACG |
| staph_NC_013450_1360842_1361008 | /5Phos/GCTTCAGCGCCATTACCGCCACCAGCTGTTGGAGGCTCATCGTTCCTATA |
| | TTCCACACCACTTATTATTACAGATGTTATGCTCGCAGGTCACTCTTGATATATTCT |
| | TGTAAGCG |
| staph_AM990992_2526026_2526192 | /5Phos/GTTCACACAACGCGCCGACTAGAATCCGTTGGAGGCTCATCGTTCCTATA |
| | TTCCACACCACTTATTATTACAGATGTTATGCTCGCAGGTCCACGATATCCAAGATA |
| | ATGATTGGCTA |
| staph_NC_010079_361284_361447 | /5Phos/GCGCACCTACAATCGCCATTACTACACGTTGGAGGCTCATCGTTCCTATA |
| | TTCCACACCACTTATTATTACAGATGTTATGCTCGCAGGTCACTCATTATCGACTGT |
| | TACATCGACTGA |
| staph_NC_007795_2085723_2085901 | /5Phos/AGCGCACATGTGACAGCGTGTAGGTTAGTTGGAGGCTCATCGTTCCTATA |
| | TTCCACACCACTTATTATTACAGATGTTATGCTCGCAGGTCGTGCCTTAGATTGTTC |
| | AGAACAAT |
| staph_NC_009641_23125_23297 | /5Phos/CGAATGGATATGTACCATGGTCGATATCGTTGGAGGCTCATCGTTCCTAT |
| | ATTCCACACCACTTATTATTACAGATGTTATGCTCGCAGGTCCTCTCTAATATGATG |
| | TCCAT |
| staph_FN433596_2144570_2144734 | /5Phos/ACTACAACAGCAACCGCATTACAATGGCGTTGGAGGCTCATCGTTCCTAT |
| | ATTCCACACCACTTATTATTACAGATGTTATGCTCGCAGGTCGGTGCTAAGAGGTCA |
| | TCGGA |
| staph_NC_009782_54857_55020 | /5Phos/AGCTTCAGATAAGTACCTATCTGAGTTGGAGGCTCATCGTTCCTATATTC |
| | CACACCACTTATTATTACAGATGTTATGCTCGCAGGTCGGAAGAATAGTTATTCTTG |
| | ATAATGTAT |
| staph_AM990992_1656616_1656789 | /5Phos/CGTATTGCTCGAATACATGATAGTTGGAGGCTCATCGTTCCTATATTCCA |
| | CACCACTTATTATTACAGATGTTATGCTCGCAGGTCACAATGTATCAAGGCCAGCT |
| staph_NC_007793_44227_44395 | /5Phos/GCGACCAGTTGTTATCGACCGTGTGTTGGAGGCTCATCGTTCCTATATTC |
| | CACACCACTTATTATTACAGATGTTATGCTCGCAGGTCCAGAACGATACGGTGCTGT |
| | ATA |
| staph_NC_009641_1102949_1103116 | /5Phos/CAATTACATTGTCTGTTGCGTAGATACCGTTGGAGGCTCATCGTTCCTAT |
| | ATTCCACACCACTTATTATTACAGATGTTATGCTCGCAGGTCGTTGTGGCTAATGTG |
| | CCAGTT |
| staph_NC_009641_1137731_1137898 | /5Phos/GCACCACTCTATAGCAGTAGCGTATTGGTTGGAGGCTCATCGTTCCTATA |
| | TTCCACACCACTTATTATTACAGATGTTATGCTCGCAGGTCACAGCCAATGTCACCT |
| | AAGTCAACA |
| staph_FN433596_2715713_2715871 | /5Phos/ACAGTCCGAATAAGATACGACTATTCGAGTTGGAGGCTCATCGTTCCTAT |
| | ATTCCACACCACTTATTATTACAGATGTTATGCTCGCAGGTCCGTTGTAACGTATAT |
| | GAATAGTTGA |
| staph_NC_009782_606652_606825 | /5Phos/AGATGCAATAACAGGTCGAATATTAATTGTTGGAGGCTCATCGTTCCTAT |
| | ATTCCACACCACTTATTATTACAGATGTTATGCTCGCAGGTCGCCATAGTGAGAGTA |
| | GTGAA |
| staph_FN433596_657625_657803 | /5Phos/AGATGCAATAACAGGTCGAATATTAAGTTGGAGGCTCATCGTTCCTATAT |
| | TCCACACCACTTATTATTACAGATGTTATGCTCGCAGGTCACACATACGGCCATAGT |
| | GAGAG |
| species_NC_004741_4338803_4338982 | /5Phos/GAACATAACGCGACGTTCCAGCTGGTTGGAGGCTCATCGTTCCTATATTC |
| | CACACCACTTATTATTACAGATGTTATGCTCGCAGGTCGCTTCAGAGGTGTTGTAGT |
| | CG |
| species_NC_009648_4535521_4535683 | /5Phos/GCGCTGGCGCAGTATCGTGAACTGGGTTGGAGGCTCATCGTTCCTATATT |
| | CCACACCACTTATTATTACAGATGTTATGCTCGCAGGTCACCAACGTAATCTCTATT |
| | ACCG |
| species_NC_010410_3677607_3677782 | /5Phos/GCTGTAATGCAAGTAGCGTATGCGCTCAGTTGGAGGCTCATCGTTCCTAT |
| | ATTCCACACCACTTATTATTACAGATGTTATGCTCGCAGGTCAAGGCCGCCAATGCC |
| | TGACG |
| species_CP001844_589057_589217 | /5Phos/GCCTGTAGCAACAGTACCACGACCAGTGTTGGAGGCTCATCGTTCCTATA |
| | TTCCACACCACTTATTATTACAGATGTTATGCTCGCAGGTCCACCACGTAATAATGC |
| | ACCAA |
| species_CP002110_2761329_2761492 | /5Phos/ACTACGCTGAAGCTGGTGACAACATTGGTTGGAGGCTCATCGTTCCTATA |
| | TTCCACACCACTTATTATTACAGATGTTATGCTCGCAGGTCGTTGAGGACGTATTCT |
| | CAATC |
| species_NC_010473_3546640_3546818 | /5Phos/GCTGGTACTTACGTTCAGATGTTGGAGGCTCATCGTTCCTATATTCCACA |
| | CCACTTATTATTACAGATGTTATGCTCGCAGGTCACGGTGAACGCCGTTACATCC |
| species_CP001844_57304_57465 | /5Phos/GCAATTCTTACCACAGCACGAAGTTGGAGGCTCATCGTTCCTATATTCCA |
| | CACCACTTATTATTACAGATGTTATGCTCGCAGGTCATCTAGATGAAGATAATGAAG |
| | TCG |
| species_NC_012731_1975396_1975559 | /5Phos/GCGGCGGCAGGCGGTAACGCCAGGTTGGAGGCTCATCGTTCCTATATTCC |
| | ACACCACTTATTATTACAGATGTTATGCTCGCAGGTCACGCGGTTATCTACCACGGCG |
| species_NC_003923_198857_199024 | /5Phos/GCACCTACTTGTCCAGCACCAGCCATGTTGGAGGCTCATCGTTCCTATAT |
| | TCCACACCACTTATTATTACAGATGTTATGCTCGCAGGTCAATACCACCACCAATAC |
| | AAGCA |
| species_NC_010400_52102_52263 | /5Phos/GCGCGGTAACATGCCATATTCTGCGTTGGAGGCTCATCGTTCCTATATTC |
| | CACACCACTTATTATTACAGATGTTATGCTCGCAGGTCCCTGAATGACATCACAGTCG |
| species_NC_010473_3310005_3310164 | /5Phos/AATCAGGTCAAGGAACTGCAAGCGTTGGAGGCTCATCGTTCCTATATTCC |
| | ACACCACTTATTATTACAGATGTTATGCTCGCAGGTCGTCTCAATCATATGCACCGG |
| | AATAC |
| species_FP929058_3022053_3022226 | /5Phos/GAACATATGTGTATGACGATGCGCGGGTTGGAGGCTCATCGTTCCTATAT |
| | TCCACACCACTTATTATTACAGATGTTATGCTCGCAGGTCGTACATGTCGCTTATCT |
| | GCCAGAAGGT |
| species_NC_009085_1010393_1010556 | /5Phos/CGTGTGCGTAGTGACGAGTTGGAGAGTTGGAGGCTCATCGTTCCTATATT |
| | CCACACCACTTATTATTACAGATGTTATGCTCGCAGGTCAGAATACGATGATGTAAG |
| | GTACACCTA |
| species_CP002621_172633_172802 | /5Phos/CAGGAGTTACTTCTGTTCCATGTTGGAGGCTCATCGTTCCTATATTCCAC |
| | ACCACTTATTATTACAGATGTTATGCTCGCAGGTCTTGAACAATTAGATCACCTCG |
| species_FP929040_442484_442653 | /5Phos/CGTAATCTCCATTACCGATGGTCAGATCGTTGGAGGCTCATCGTTCCTAT |
| | ATTCCACACCACTTATTATTACAGATGTTATGCTCGCAGGTCACGTATTCTACCTCC |
| | ACTCTCGTCT |
| species_NC_003923_1334345_1334501 | /5Phos/CATTCGACGTTCTGGTATTACTTGTTGGAGGCTCATCGTTCCTATATTCC |
| | ACACCACTTATTATTACAGATGTTATGCTCGCAGGTCCACGCTCCGCATCAGCAGCA |
| | CCACGTT |
| species_NC_009085_1010678_1010853 | /5Phos/CTGAACCACGGATTACTGGAGTGTCGTTGGAGGCTCATCGTTCCTATATT |
| | CCACACCACTTATTATTACAGATGTTATGCTCGCAGGTCGCCTGTTACTACTGTACC |
| | ACGAC |
| pseudomonas_NC_008463_4756080_4756240 | /5Phos/GAATCGAACGGTCTCATTAACAGATGTTGGAGGCTCATCGTTCCTATATT |
| | CCACACCACTTATTATTACAGATGTTATGCTCGCAGGTCGCTTTCCAGGGATATAAG |
| | ACGC |
| pseudomonas_NC_002516_1063894_1064077 | /5Phos/CCCGCAGAGTCACACTCGGAGTTGGAGGCTCATCGTTCCTATATTCCACA |
| | CCACTTATTATTACAGATGTTATGCTCGCAGGTCACTCTTGGTACTACTCACTAGC |
| pseudomonas_NC_008463_3182693_3182865 | /5Phos/GAGTCTCTTTCAACCTGGATTAGATATGTTGGAGGCTCATCGTTCCTATA |
| | TTCCACACCACTTATTATTACAGATGTTATGCTCGCAGGTCAAGATTAATAGCGTAC |
| | TTTACTCC |
| pseudomonas_NC_009656_2819490_2819655 | /5Phos/ATCCCGCAGATACTAGGTTCTTAATGTTGGAGGCTCATCGTTCCTATATT |
| | CCACACCACTTATTATTACAGATGTTATGCTCGCAGGTCGAACTATTCATATTACAC |
| | CCTAAGG |
| pseudomonas_NC_008463_3184022_3184185 | /5Phos/CAGTGGGCTATCCTAAGCCAAAGGTTGGAGGCTCATCGTTCCTATATTCC |
| | ACACCACTTATTATTACAGATGTTATGCTCGCAGGTCCATAAGCGAACTAACTATCA |
| | CTTA |
| pseudomonas_NC_002516_1065937_1066093 | /5Phos/ACAAAGCGTTCTAAACGATTAGAACTGTTGGAGGCTCATCGTTCCTATAT |
| | TCCACACCACTTATTATTACAGATGTTATGCTCGCAGGTCCGAGAAAGGAAACAGGA |
| | TAGTAC |
| pseudomonas_NC_002516_1067833_1068007 | /5Phos/CCAATGGAGAAGTCTAAATGTCCAAGTTGGAGGCTCATCGTTCCTATATT |
| | CCACACCACTTATTATTACAGATGTTATGCTCGCAGGTCTTATCAGAGATACATGAC |
| | TCTTAGG |
| pseudomonas_NC_008463_3182351_3182508 | /5Phos/CGAATCACTGGACTACATTTATATTTCTGTTGGAGGCTCATCGTTCCTAT |
| | ATTCCACACCACTTATTATTACAGATGTTATGCTCGCAGGTCAGCGAACCTTTATAT |
| | TTGACCAT |
| pseudomonas_NC_008463_3184314_3184473 | /5Phos/CTCAAGTCTTGCCCTGATAGAATTATGTTGGAGGCTCATCGTTCCTATAT |
| | TCCACACCACTTATTATTACAGATGTTATGCTCGCAGGTCTCACGACTTATCTACTT |
| | TAGAAATC |
| pseudomonas_AP012280_3765216_3765383 | /5Phos/GGTGATCGTTATTATGATAGTACGGCGTTGGAGGCTCATCGTTCCTATAT |
| | TCCACACCACTTATTATTACAGATGTTATGCTCGCAGGTCCTCGGTTAAGGGAATTA |
| | CGAC |
| pseudomonas_AP012280_3765033_3765192 | /5Phos/ACTCGGATGGTAGGTTTATTAAAGCGTTGGAGGCTCATCGTTCCTATATT |
| | CCACACCACTTATTATTACAGATGTTATGCTCGCAGGTCGTGATCGTTATTATGATA |
| | GTACGG |
| enterococcus_NZ_GG703715_13422_13573 | /5Phos/ACAATCGTTGTCGCACTGCATAGGTTGGAGGCTCATCGTTCCTATATTCC |
| | ACACCACTTATTATTACAGATGTTATGCTCGCAGGTCGAACTTGGTCTACCGTACCAC |
| enterococcus_NZ_GG703582_76982_77140 | /5Phos/GGATAATACAATCCTAATACGTACGGAGTTGGAGGCTCATCGTTCCTATA |
| | TTCCACACCACTTATTATTACAGATGTTATGCTCGCAGGTCGCTGCTGTAACTAGGG |
| | TAGC |
| enterococcus_NZ_GL455004_28219_28381 | /5Phos/CTATATTCAACGGGTCACGGGTAGGTTGGAGGCTCATCGTTCCTATATTC |
| | CACACCACTTATTATTACAGATGTTATGCTCGCAGGTCTCATTGATTCGATCTCGTA |
| | ACTC |
| enterococcus_NZ_GG703720_94699_94852 | /5Phos/AATGTTATTGTGGTTGCGTGTTCGGTTGGAGGCTCATCGTTCCTATATTC |
| | CACACCACTTATTATTACAGATGTTATGCTCGCAGGTCTACTTTGGAAGTGCCCTGAC |
| enterococcus_NZ_GG703715_15795_15951 | /5Phos/CATGTCTTCTAGTACAGGTTTGCCGGTTGGAGGCTCATCGTTCCTATATT |
| | CCACACCACTTATTATTACAGATGTTATGCTCGCAGGTCTGTAAGAGGCCGCTAACT |
| | TC |
| enterococcus_NZ_GL455899_32848_32984 | /5Phos/CTCTGGCTCGTGGGCTCGGGTTGGAGGCTCATCGTTCCTATATTCCACAC |
| | CACTTATTATTACAGATGTTATGCTCGCAGGTCTTCTTGAGATAGTCCGGTATAATC |
| enterococcus_NZ_GG692918_325104_325257 | /5Phos/ATTCGATCACGATGGGCTGGGGTTGGAGGCTCATCGTTCCTATATTCCAC |
| | ACCACTTATTATTACAGATGTTATGCTCGCAGGTCAATTTCCTGTGTCATACACGC |
| enterococcus_NC_004668_920608_920750 | /5Phos/CAATTGATTTAGCCACTACACCTTACGTTGGAGGCTCATCGTTCCTATAT |
| | TCCACACCACTTATTATTACAGATGTTATGCTCGCAGGTCCACTATTCTGGCGACCA |
| | CC |
| enterococcus_NZ_GG703575_78829_78963 | /5Phos/GATAAAGAAGCGTCTTGACCCAGTGTTGGAGGCTCATCGTTCCTATATTC |
| | CACACCACTTATTATTACAGATGTTATGCTCGCAGGTCATCTGGTGCTCCTTGACGC |
| enterococcus_NZ_GL455931_26355_26493 | /5Phos/GCAAATTTAGAGAGTGCATGCATGGTTGGAGGCTCATCGTTCCTATATTC |
| | CACACCACTTATTATTACAGATGTTATGCTCGCAGGTCGGAAGAGGACGGCATACAAC |
| enterococcus_NZ_GG669058_207026_207172 | /5Phos/CATTTCATCTAGACCGCTCGTGTGTTGGAGGCTCATCGTTCCTATATTCC |
| | ACACCACTTATTATTACAGATGTTATGCTCGCAGGTCGCTTGAAGTGTATGTTGGGAC |
| proteus_NZ_GG661998_111187_111342 | /5Phos/GTCGCCCTCGTGCTAACGTGTTGGAGGCTCATCGTTCCTATATTCCACAC |
| | CACTTATTATTACAGATGTTATGCTCGCAGGTCGGTTCTTTGATGTACCGGTT |
| proteus_NC_010554_2037943_2038091 | /5Phos/GCTGATGACGGTGAAGTTTATCAGTTGGAGGCTCATCGTTCCTATATTCC |
| | ACACCACTTATTATTACAGATGTTATGCTCGCAGGTCCATTATCGCACATATTGACC |
| | AC |
| proteus_NZ_GG668576_810893_811054 | /5Phos/GAAATTAGCTAAAGGGATATCGCGGTTGGAGGCTCATCGTTCCTATATTC |
| | CACACCACTTATTATTACAGATGTTATGCTCGCAGGTCAACTTTCCGCCAATCCTGC |
| proteus_NZ_GG668594_760_939 | /5Phos/CACCTACGTTCTCACCTGCACGTTGGAGGCTCATCGTTCCTATATTCCAC |
| | ACCACTTATTATTACAGATGTTATGCTCGCAGGTCATTCGATAGTACCAGTTACGTC |
| proteus_NZ_GG668579_22072_22234 | /5Phos/GTTGCTTATAGCGTCGCTGCTGTTGGAGGCTCATCGTTCCTATATTCCAC |
| | ACCACTTATTATTACAGATGTTATGCTCGCAGGTCCTGGTTATCGAGAAGATAAAGG |
| proteus_NC_010554_2448957_2449119 | /5Phos/GTAAGCGTAGCGATACGTTGAGGTTGGAGGCTCATCGTTCCTATATTCCA |
| | CACCACTTATTATTACAGATGTTATGCTCGCAGGTCGAGTGAACGCACCACTGG |
| proteus_NC_010554_3033758_3033936 | /5Phos/TCAGGTAGAGAATACTCAGGCGCGTTGGAGGCTCATCGTTCCTATATTCC |
| | ACACCACTTATTATTACAGATGTTATGCTCGCAGGTCCGGAGAAGGCTAGGTTGTC |
| proteus_NC_010554_454391_454540 | /5Phos/GCAACCCACTCCCATGGTGTGTTGGAGGCTCATCGTTCCTATATTCCACA |
| | CCACTTATTATTACAGATGTTATGCTCGCAGGTCCGTTCTTCATCAGACAATCTG |
| gyrB_NC_015663_1455472_1455621 | /5Phos/GCCCTTTCAGGACTTTGATACTGGGTTGGAGGCTCATCGTTCCTATATTC |
| | CACACCACTTATTATTACAGATGTTATGCTCGCAGGTCTGTACGGAGACGGAGTTAT |
| | CG |
| gyrB_NC_010410_4215_4366 | /5Phos/ACACTGACCGATTCATCCTCGTGGTTGGAGGCTCATCGTTCCTATATTCC |
| | ACACCACTTATTATTACAGATGTTATGCTCGCAGGTCCTTGAAAGTGCGTTAACAACC |
| gyrB_NC_005773_4904_5052 | /5Phos/CGGAAGCCCACCAAGTGAGTACGTTGGAGGCTCATCGTTCCTATATTCCA |
| | CACCACTTATTATTACAGATGTTATGCTCGCAGGTCCGAAACCAGTTTGTCCTTAGTC |
| gyrB_NC_016514_5343_5487 | /5Phos/ACCAGCTTGTCTTTAGTCTGAGAGGTTGGAGGCTCATCGTTCCTATATTC |
| | CACACCACTTATTATTACAGATGTTATGCTCGCAGGTCCTTTACGACGGGTCATTTC |
| | AC |
| gyrB_NC_016603_2631439_2631616 | /5Phos/CATTGGTTTGTTCTGTTTGAGAGGCGTTGGAGGCTCATCGTTCCTATATT |
| | CCACACCACTTATTATTACAGATGTTATGCTCGCAGGTCGATTCATCTTCGTGAATT |
| | GTGAC |
| gyrB_NC_009436_4366_4524 | /5Phos/GGACTTTGATACTGGAGGAGTCATAGTTGGAGGCTCATCGTTCCTATATT |
| | CCACACCACTTATTATTACAGATGTTATGCTCGCAGGTCTGTACGGAAACGGAGTTA |
| | TCG |
| gyrB_NC_009512_4203_4373 | /5Phos/ATGCTGGAGGAGTCGTACGTTTGTTGGAGGCTCATCGTTCCTATATTCCA |
| | CACCACTTATTATTACAGATGTTATGCTCGCAGGTCGTCGCGCACACTAATAGATTC |
| pseudomonas_NC_009085_307050_307218 | /5Phos/AACTAAACCTACACGGAATTGGTTCGTTGGAGGCTCATCGTTCCTATATT |
| | CCACACCACTTATTATTACAGATGTTATGCTCGCAGGTCGCAGATACACGACGTTTA |
| | TGT |
| pseudomonas_NC_009085_308225_308377 | /5Phos/GCCGCTTCACCTACGTTAGGAAGTTGGAGGCTCATCGTTCCTATATTCCA |
| | CACCACTTATTATTACAGATGTTATGCTCGCAGGTCCGTAAAGATGAGTCTTTAACG |
| | TC |
| pseudomonas_NC_016612_1674334_1674490 | /5Phos/GACGTTTGTGCGTAATCTCAGACGTTGGAGGCTCATCGTTCCTATATTCC |
| | ACACCACTTATTATTACAGATGTTATGCTCGCAGGTCGAGGAAACCGTATTCGTTCGT |
| pseudomonas_NC_016603_3425179_3425337 | /5Phos/ACAACACTTTACCACTTGAGTGGGGTTGGAGGCTCATCGTTCCTATATTC |
| | CACACCACTTATTATTACAGATGTTATGCTCGCAGGTCGTAACTGCCCATGTCAAGA |
| | TAC |
| pseudomonas_NC_016603_3427629_3427808 | /5Phos/CCACGTTTAGTTGAACCACCGCGTTGGAGGCTCATCGTTCCTATATTCCA |
| | CACCACTTATTATTACAGATGTTATGCTCGCAGGTCTCAATACGCCAGTTGTTAGTTC |
| pseudomonas_NC_010410_3543925_3544088 | /5Phos/AATCGATAATAAGTACGGTGCATCCGTTGGAGGCTCATCGTTCCTATATT |
| | CCACACCACTTATTATTACAGATGTTATGCTCGCAGGTCGAAGAATACATTCGCGTA |
| | CATC |
| pseudomonas_NC_005966_304936_305079 | /5Phos/AAGCAAGATCGAGTCTTCATAGTTGGTTGGAGGCTCATCGTTCCTATATT |
| | CCACACCACTTATTATTACAGATGTTATGCTCGCAGGTCGATATACACGATACCTGA |
| | TTCGT |
| pseudomonas_NC_008593_226005_226171 | /5Phos/CCGATATTCATACGAGAAGGTACACGTTGGAGGCTCATCGTTCCTATATT |
| | CCACACCACTTATTATTACAGATGTTATGCTCGCAGGTCCAGTAACTCTATTGTCAA |
| | ACGGT |
| pseudomonas_NC_016514_213592_213738 | /5Phos/GTAGTGAGTCGGGTGTACGTCTCGTTGGAGGCTCATCGTTCCTATATTCC |
| | ACACCACTTATTATTACAGATGTTATGCTCGCAGGTCTCTTCGATAGCAGACAGATA |
| | GT |
| pseudomonas_NC_005966_303883_304054 | /5Phos/ACCTACACGGAATTGGTTCTCAGTGTTGGAGGCTCATCGTTCCTATATTC |
| | CACACCACTTATTATTACAGATGTTATGCTCGCAGGTCGATACACGACGTTTGTGTG |
| | TA |
| enterobacter_NC_014618_3997909_3998085 | /5Phos/CAACATCATTAGCTTGGTCGTGGGGTTGGAGGCTCATCGTTCCTATATTC |
| | CACACCACTTATTATTACAGATGTTATGCTCGCAGGTCTTGCGTGTTACCAACTCGTC |
| enterobacter_NZ_GL892086_61549_615324 | /5Phos/CGGCACGTCCGAATCGTATCAGTTGGAGGCTCATCGTTCCTATATTCCAC |
| | ACCACTTATTATTACAGATGTTATGCTCGCAGGTCTCGTGTCCCGTATATGTTGG |
| enterobacter_NZ_GL892086_1664663_1664834 | /5Phos/AATAGAGGCCCACAAGTCTTGTTCGTTGGAGGCTCATCGTTCCTATATTC |
| | CACACCACTTATTATTACAGATGTTATGCTCGCAGGTCCGCTCTCCACTATGGGTAGT |
| enterobacter_NZ_GG704865_427821_427978 | /5Phos/GCTACATTAATCACTATGGACAGACAGTTGGAGGCTCATCGTTCCTATAT |
| | TCCACACCACTTATTATTACAGATGTTATGCTCGCAGGTCGATGGTCGATCTATCGT |
| | CTCT |
| enterobacter_NZ_GL892087_1610708_1610874 | /5Phos/GAAGTGTTATTCAAACTTTGGTCCCGTTGGAGGCTCATCGTTCCTATATT |
| | CCACACCACTTATTATTACAGATGTTATGCTCGCAGGTCCTTGAACCCTTGGTTCAA |
| | GGT |

TABLE 2
A list of organisms for which the methods and kits described herein have been
validated to detect using the compositions described herein

Acinetobacter baumannii 1656-2
Acinetobacter baumannii AB0057
Acinetobacter baumannii AB307-0294
Acinetobacter baumannii ACICU
Acinetobacter baumannii ATCC 17978
Acinetobacter baumannii AYE
Acinetobacter baumannii MDR-ZJ06
Acinetobacter baumannii SDF
Acinetobacter baumannii TCDC-AB0715
Acinetobacter calcoaceticus PHEA-2
Acinetobacter sp. ADP1
Acinetobacter sp. DR1
Clostridium acetobutylicum ATCC 824
Clostridium acetobutylicum DSM 1731
Clostridium acetobutylicum EA 2018
Clostridium beijerinckii NCIMB 8052
Clostridium botulinum A2 str. Kyoto
Clostridium botulinum A3 str. Loch Maree
Clostridium botulinum A str. ATCC 19397
Clostridium botulinum A str. ATCC 3502
Clostridium botulinum A str. Hall
Clostridium botulinum B1 str. Okra
Clostridium botulinum Ba4 str. 657
Clostridium botulinum BKT015925
Clostridium botulinum B str. Eklund 17B
Clostridium botulinum E3 str. Alaska E43
Clostridium botulinum F str. 230613
Clostridium botulinum F str. Langeland
Clostridium botulinum H04402 065
Clostridium cellulolyticum H10
Clostridium cellulovorans 743B
Clostridium clariflavum DSM 19732
Clostridium difficile 630
Clostridium difficile BI1
Clostridium difficile BI9
Clostridium difficile CD196
Clostridium difficile strain 2007855
Clostridium difficile strain CF5
Clostridium difficile strain M120
Clostridium difficile M68
Clostridium difficile R20291
Clostridium kluyveri DSM 555
Clostridium kluyveri NBRC 12016
Clostridium lentocellum DSM 5427
Clostridium ljungdahlii DSM 13528
Clostridium novyi NT
Clostridium perfringens ATCC 13124
Clostridium perfringens SM101
Clostridium perfringens str. 13
Clostridium phytofermentans ISDg
Clostridium saccharolyticum-like K10
Clostridium saccharolyticum WM1
Clostridium sp. SY8519
Clostridium sticklandii DSM 519
Clostridium tetani E88
Clostridium thermocellum ATCC 27405
Clostridium thermocellum DSM 1313
Enterobacter aerogenes KCTC 2190
Enterobacter asburiae LF7a
Enterobacter cloacae SCF1
Enterobacter cloacae subsp.cloacae ATCC 13047
Enterobacter cloacae subsp. cloacae NCTC 9394
Enterobacter sp. 638
Enterococcus faecalis 62
Enterococcus faecalis OG1RF
Enterococcus faecalis V583
Enterococcus sp. 7L76
Escherichia coli 042
Escherichia coli 536
Escherichia coli 55989
Escherichia coli ABU 83972
Escherichia coli APEC O1
Escherichia coli ATCC 8739
Escherichia coli BL21(DE3)
Escherichia coli ‘BL21-Gold(DE3)pLysS AG'
Escherichia coli B str. REL606
Escherichia coli BW2952
Escherichia coli CFT073
Escherichia coli DH1 (ME8569)
Escherichia coli E24377A
Escherichia coli ED1a
Escherichia coli ETEC H10407
Escherichia coli HS
Escherichia coli IAI1
Escherichia coli IAI39
Escherichia coli IHE3034
Escherichia coli KO11
Escherichia coli LF82
Escherichia coli NA114
Escherichia coli O103: H2 str. 12009
Escherichia coli O111:H-str. 11128
Escherichia coli O127:H6 str. E2348/69
Escherichia coli O157:H7 str. EC4115
Escherichia coli O157:H7 str. EDL933
Escherichia coli O157:H7 str. Sakai
Escherichia coli O157:H7 str. TW14359
Escherichia coli O26:H11 str. 11368
Escherichia coli O55:H7 str. CB9615
Escherichia coli O7:K1 str. CE10
Escherichia coli O83:H1 str. NRG 857C
Escherichia coli S88
Escherichia coli SE11
Escherichia coli SE15
Escherichia coli SMS-3-5
Escherichia coli str. ‘clone D i14’
Escherichia coli str. ‘clone D i2’
Escherichia coli str. K-12 substr. DH10B
Escherichia coli str. K-12 substr. MDS42
Escherichia coli str. K-12 substr. MG1655
Escherichia coli str. K12 substr. W3110
Escherichia coli UM146
Escherichia coli UMN026
Escherichia coli UMNK88
Escherichia coli UTI89
Escherichia coli W
Escherichia fergusonii ATCC 35469
Klebsiella pneumoniae 342
Klebsiella pneumoniae KCTC 2242
Klebsiella pneumoniae NTUH-K2044
Klebsiella pneumoniae subsp. pneumoniae MGH
78578
Klebsiella variicola At-22
Proteus mirabilis HI4320
Pseudomonas aeruginosa LESB58
Pseudomonas aeruginosa M18
Pseudomonas aeruginosa NCGM2.S1
Pseudomonas aeruginosa PA7
Pseudomonas aeruginosa PAO1
Pseudomonas aeruginosa UCBPP-PA14
Pseudomonas brassicacearum subsp. brassicacearum
NFM421
Pseudomonas entomophila L48
Pseudomonas fluorescens F113
Pseudomonas fluorescens Pf0-1
Pseudomonas fluorescens Pf-5
Pseudomonas fluorescens SBW25
Pseudomonas fulva 12-X
Pseudomonas mendocina NK-01
Pseudomonas mendocina ymp
Pseudomonas putida BIRD-1
Pseudomonas putida F1
Pseudomonas putida F1
Pseudomonas putida GB-1
Pseudomonas putida KT2440
Pseudomonas putida S16
Pseudomonas putida W619
Pseudomonas stutzeri A1501
Pseudomonas stutzeri ATCC 17588 = LMG 11199
Pseudomonas stutzeri DSM 4166
Pseudomonas syringae pv. phaseolicola 1448A
Pseudomonas syringae pv. syringae B728a
Pseudomonas syringae pv. tomato str. DC3000
Shigella boydii CDC 3083-94
Shigella boydii Sb227
Shigella dysenteriae Sd197
Shigella flexneri 2002017
Shigella flexneri 2a str. 2457T
Shigella flexneri 2a str. 301
Shigella flexneri 5 str. 8401
Shigella sonnei Ss046
Staphylococcus aureus
Staphylococcus carnosus subsp. carnosus
Staphylococcus epidermidis
Staphylococcus haemolyticus JCSC1435
Staphylococcus lugdunensis
Staphylococcus pseudintermedius
Staphylococcus saprophyticus subsp.
Staphylococcus aureus
Staphylococcus saprophyticus
Staphylococcus epidermis
Acinetobacter baumannii
Enterococcus faecalis
Enterobacter cloacae
Enterobacter aerogenes
Enterococcus faecium
Candida albicans
Klebsiella pneumoniae
Escherichia coli
Clostridium difficile
Proteus mirabilis
Pseudomonas aeruginosa

TABLE 3
Genus level regions can be used for coarse discrimination of organisms.

| Probe Coordinates | Gene |
|---|---|
| species_NC_004741_43388 | rpsC, S4416, 30S ribosomal protein S3 |
| 03_4338982 | |
| species_NC_009648_45355 | atpA, KPN_04139, F0F1 ATP synthase subunit alpha |
| 21_4535683 | |
| species_NC_010410_36776 | int, ABAYE3575, integrase/recombinase (E2 protein) |
| 07_3677782 | |
| species_CP001844_589057_ | tufA, SA2981_0525, Translation elongation factor Tu |
| 589217 | |
| species_CP002110_276132 | tuf, HMPREF0772_12641, elongation factor EF1A |
| 9_2761492 | |
| species_NC_010473_35466 | rp1B, ECDH10B_3492, 50S ribosomal protein L2 |
| 40_3546818 | |
| species_CP001844_57304_ | tnpB, SA2981_0055, Transposase B from transposon |
| 57465 | Tn554 tnpB, SA2981_1617, Transposase B from |
| | transposon Tn554 |
| species_NC_012731_19753 | putA, KP1_2030, trifunctional transcriptional |
| 96_1975559 | regulator/proline dehydrogenase/pyrroline-5- |
| | carboxylate dehydrogenase |
| species_NC_003923_19885 | MW0166, hypothetical protein |
| 7_199024 | |
| species_NC_010400_52102_ | rph, ABSDF0051, ribonuclease PH |
| 52263 | |
| species_NC_010473_33100 | rpoD, ECDH10B_3242, RNA polymerase sigma factor RpoD |
| 05_3310164 | |
| species_FP929058_302205 | ENT_30090, GTP cyclohydrolase I |
| 3_3022226 | |
| species_NC_009085_10103 | A1S_0279, elongation factor Tu |
| 93_1010556 | |
| species_CP002621_172633_ | rplO, OG1RF_10170, 50S ribosomal protein L15 |
| 172802 | |
| species_FP929040_442484_ | ENC_04200, proton translocating ATP synthase, F1 |
| 442653 | alpha subunit |
| species N0_003923_13343 | katA, MW1221, catalase |
| 45_133401 | |
| species_NC 009085_10106 | A1S_0279, elongation factor Tu |
| 78_101053 | |

TABLE 4
Genus level probes

| Probe Coordinates | Binding region 1 | Binding region 2 |
|---|---|---|
| species_NC_004741_4338803_4338982 | GAACATAACGCGACGTTCCAGCTG | GCTTCAGAGGTGTTGTAGTCG |
| species_NC_009648_4535521_4535683 | GCGCTGGCGCAGTATCGTGAACTGG | ACCAACGTAATCTCTATTACCG |
| species_NC_010410_3677607_3677782 | GCTGTAATGCAAGTAGCGTATGCGCTCA | AAGGCCGCCAATGCCTGACG |
| species_CP001844_589057_589217 | GCCTGTAGCAACAGTACCACGACCAGT | CACCACGTAATAATGCACCAA |
| species_CP002110_2761329_2761492 | ACTACGCTGAAGCTGGTGACAACATTG | GTTGAGGACGTATTCTCAATC |
| species_NC_010473_3546640_3546818 | GCTGGTACTTACGTTCAGAT | ACGGTGAACGCCGTTACATCC |
| species_CP001844_57304_57465 | GCAATTCTTACCACAGCACGAA | ATCTAGATGAAGATAATGAAGTCG |
| species_NC_012731_1975396_1975559 | GCGGCGGCAGGCGGTAACGCCAG | ACGCGGTTATCTACCACGGCG |
| species_NC_003923_198857_199024 | GCACCTACTTGTCCAGCACCAGCCAT | AATACCACCACCAATACAAGCA |
| species_NC_010400_52102_52263 | GCGCGGTAACATGCCATATTCTGC | CCTGAATGACATCACAGTCG |
| species_NC_010473_3310005_3310164 | AATCAGGTCAAGGAACTGCAAGC | GTCTCAATCATATGCACCGGAATAC |
| species_FP929058_3022053_3022226 | GAACATATGTGTATGACGATGCGCGG | GTACATGTCGCTTATCTGCCAGAAG |
| | | GT |
| species_NC_009085_1010393_1010556 | CGTGTGCGTAGTGACGAGTTGGAGA | AGAATACGATGATGTAAGGTACACC |
| | | TA |
| species_CP002621_172633_172802 | CAGGAGTTACTTCTGTTCCAT | TTGAACAATTAGATCACCTCG |
| species_FP929040_442484_442653 | CGTAATCTCCATTACCGATGGTCAGATCC | ACGTATTCTACCTCCACTCTCGTCT |
| species_NC_003923_1334345_1334501 | CATTCGACGTTCTGGTATTACTT | CACGCTCCGCATCAGCAGCACCACG |
| | | TT |
| species_NC_009085_1010678_1010853 | CTGAACCACGGATTACTGGAGTGTC | GCCTGTTACTACTGTACCACGAC |

TABLE 5
Species/strain level regions can be used for discrimination at the level of
species and strains.

| Probe Coordinates | Gene |
|---|---|
| acinetobacter_NC_010 | ACICU_00572, pyridine nucleotide transhydrogenase |
| 611_627997_628164 | (proton pump) subunit alpha (part2) |
| acinetobacter_NC_010 | pepN, ACICU_02288, aminopeptidase N |
| 611_2417580_2417755 | trpC, ACICU_02557, indole-3-glycerol-phosphate |
| | synthase |
| acinetobacter_CP0025 | recF, ABTW07_0010, recombination protein F |
| 22_11753_11931 | |
| acinetobacter_NC_011 | gshB, AB57_3788, glutathione synthetase |
| 586_3908329_3908508 | |
| acinetobacter_NC_010 | ACICU_00129, NAD-dependent aldehyde dehydrogenase |
| 611_145181_145340 | |
| acinetobacter_NC_010 | ACICU_03630, A/G-specific DNA glycosylase |
| 611_3854494_3854662 | |
| acinetobacter_NC_010 | nadC, ABSDF0056, nicotinate-nucleotide |
| 400_56216_56383 | pyrophosphorylase (quinolinate |
| | phosphoribosyltransferase) |
| acinetobacter_NC_010 | near ACICU_01347, carbonic anhydrase |
| 611_1454960_1455136 | |
| acinetobacter_NC_009 | A1S_0230, phosphoglyceromutase |
| 085_255964_256143 | |
| clostridium_NC_01397 | |
| 4_3097606_3097772 | |
| clostridium_FN665653_ | |
| 103469_103631 | |
| clostridium_NC_01397 | |
| 4_117188_117346 | |
| clostridium_NC_01331 | nifJ, CDR20291_2570, pyruvate-flavodoxin |
| 6_3012882_3013047 | oxidoreductase |
| clostridium_FN668375_ | |
| 1212250_1212413 | |
| clostridium_NC_01331 | pykF, CD196_3170, pyruvate kinase |
| 5_3754484_3754640 | |
| clostridium_FN665654_ | |
| 3239860_3240039 | |
| clostridium_FN668941_ | |
| 3228320_3228491 | |
| clostridium_NC_01397 | |
| 4_1962664_1962825 | |
| clostridium_NC_00336 | tuf, CPE2407, elongation factor Tu |
| 6_2769687_2769851 | |
| clostridium_FN665653_ | |
| 127741_127918 | |
| clostridium_NC_01331 | clpB, CDR20291_1933, chaperone |
| 6_2259929_2260107 | |
| clostridium_NC_00908 | rpoC, CD0067, DNA-directed RNA polymerase subunit |
| 9_94774_94937 | beta' |
| clostridium_NC_01331 | CD196_1764, cell surface protein |
| 5_2044225_2044389 | |
| clostridium_NC_01331 | near CD196_1987, multiprotein-complex assembly |
| 5_2299408_2299586 | protein |
| clostridium_FN668941_ | |
| 3244255_3244408 | |
| clostridium_NC_01331 | gpmI, CDR20291_3027, phosphoglyceromutase |
| 6_3610909_3611065 | |
| clostridium_FN665653_ | |
| 1104859_1105031 | |
| clostridium_NC_00336 | tuf, CPE2407, elongation factor Tu |
| 6_2753681_2753838 | |
| clostridium_FN665653_ | |
| 710906_711080 | |
| clostridium_NC_00908 | gpmI, CD3171, phosphoglyceromutase |
| 9_3706562_3706720 | |
| clostridium_NC_01331 | dnaF, CDR20291_1146, DNA polymerase III PolC-type |
| 6_1372812_1372968 | |
| clostridium_FN665652_ | |
| 676696_676895 | |
| clostridium_NC_01331 | CDR20291_2249 |
| 6_2641651_2641808 | |
| clostridium_FN668375_ | |
| 3595870_3596026 | |
| clostridium_FN668941_ | |
| 1105700_1105868 | |
| clostridium_NC_01397 | |
| 4_2505182_2505359 | |
| clostridium_NC_01331 | potA, CD196_0900, spermidine/putrescine ABC |
| 5_1077126_1077298 | transporter ATP-binding protein |
| clostridium_NC_00908 | CD1878A |
| 9_2182303_2182482 | |
| clostridium_FN665652_ | |
| 1909777_1909942 | |
| clostridium_NC_01331 | ntpB, CDR20291_2788, V-type ATP synthase subunit B |
| 6_3300896_3301062 | |
| clostridium_NC_01331 | spoVAD, CDR20291_0703, stage V sporulation protein AD |
| 6_871338_871499 | |
| clostridium_NC_01331 | bclA2, CDR20291_3090, exosporium glycoprotein |
| 6_3608873_3609047 | eno, CDR20291_3026, enolase |
| clostridium_FN665654_ | |
| 3717059_3717221 | |
| clostridium_NC_01331 | CD196_1739, hypothetical protein |
| 5_2010489_2010657 | |
| clostridium_NC_01331 | adhE, CD196_2753, bifunctional acetaldehyde- |
| 5_3236301_3236474 | CoA/alcohol dehydrogenase CD196_2095, sodium:solute |
| | symporter spoVD, CD196_2497, stage V sporulation |
| | protein D (sporulation specific penicillin-binding |
| | protein) |
| clostridium_NC_01331 | CD196_0911, N-acetylmuramoyl-L-alanine amidase |
| 5_1095924_1096090 | |
| enterobacter_NC_0141 | ECL_04612, 50S ribosomal subunit protein L13 |
| 21_4735453_4735632 | |
| enterobacter_NC_0156 | EAE_24795, hemagluttinin domain-containing |
| 63_1014187_1014345 | protein, rp1R, EAE_04875, 50S ribosomal protein L18 |
| enterobacter_FP92904 | |
| 0_3448334_3448513 | |
| enterobacter_NC_0094 | rplD, Ent638_3750, 50S ribosomal protein L4 |
| 36_4051820_4051985 | |
| enterococcus_FP92905 | ENT_17660, hypothetical protein |
| 8_1738439_1738606 | |
| enterococcus_CP00262 | OG1RF_11736, group 2 glycosyl transferase |
| 1_1819224_1819388 | |
| enterococcus_FP92905 | near ENT_09350, Uncharacterized protein conserved in |
| 8_904007_904173 | bacteria |
| enterococcus_FP92905 | |
| 8_551757_551920 | |
| enterococcus_NC_0046 | |
| 68_1122345_1122507 | |
| klebsiella_NC_009648_ | mqo, KPN_02629, malate:quinone oxidoreductase |
| 2885456_2885620 | |
| klebsiella_NC_009648_ | garL, KPN_03538, alpha-dehydro-beta-deoxy-D-glucarate |
| 3899012_3899182 | aldolase |
| klebsiella_NC_009648_ | frdB, KPN_04552, fumarate reductase iron-sulfur |
| 4980596_4980757 | subunit |
| klebsiella_NC_009648 | KPN_02970, integral transmembrane protein; acridine |
| 3266359_3266519 | resistance |
| klebsiella_NC_012731_ | KP1_2672, putative malate dehydrogenase |
| 2557467_2557634 | |
| klebsiella_NC_012731_ | glpR, KP1_5123, DNA-binding transcriptional repressor |
| 4857136_4857315 | GlpR |
| proteus_NC_010554_54 | PMI0497, phage terminase large subunit |
| 7938_548117 | |
| pseudomonas_NC_00846 | PA14_07660, hypothetical protein |
| 3_658500_658676 | |
| pseudomonas_NC_00846 | rpoC, PA14_08780, DNA-directed RNA polymerase subunit |
| 3_753931_754099 | beta' |
| pseudomonas_NC_00965 | oadA, PSPA7_6223, pyruvate carboxylase subunit B |
| 6_6431649_6431828 | |
| pseudomonas_NC_00846 | PA14_06330, serine/threonine protein kinase |
| 3_560357_560534 | |
| pseudomonas_NC_01032 | PputGB1_0612, arginine decarboxylase |
| 2_5224859_5225023 | PputGB1_4676, ketol-acid reductoisomerase |
| pseudomonas_NC_00846 | dadA, PA14_70040, D-amino acid dehydrogenase small |
| 3_4839746_483924 | subunit ung, PA14_54590, uracil-DNA glycosylase |
| staph_FN433596_28440 | SATW20_26770, putative acetyltransferase |
| 85_2844263 | |
| staph_NC_009632_1198 | SaurJH1_1177, branched-chain alpha-keto acid |
| 350_1198529 | dehydrogenase subunit E2 |
| staph_FN433596_25212 | rplB, SATW20_23810, 50S ribosomal protein L2 |
| 44_2521419 | |
| staph_NC_009487_4308 | SaurJH9_0396, hypothetical protein |
| 42_431017 | |
| staph_NC_009782_2086 | SAHV_1928, truncated amidase |
| 681_2086849 | |
| staph_NC_009782_5825 | tnpB, SAHV_1645, transposase B |
| 6_58423 | |
| staph_NC_013450_9910 | SAAV_0970, ribosomal large subunit pseudouridine |
| 49_991222 | synthase D |
| staph_NC_013450_1360 | opuD1, SAAV_1329, BCCT family osmoprotectant |
| 842_1361008 | transporter |
| staph_AM990992_25260 | SAPIG2450, nitrate reductase, alpha subunit |
| 26_2526192 | |
| staph_NC 010079_3612 | near USA300HOU_0330, PfoR family transcriptional |
| 84_361447 | regulator |
| staph_NC_007795_2085 | SAOUHSC_02251, hypothetical protein |
| 723_2085901 | |
| staph_NC_009641_2312 | purA, NWMN_0016, adenylosuccinate synthetase |
| 5_23297 | |
| staph_FN433596_21445 | hlb, SATW20_19320, phospholipase C precursor |
| 70_2144734 | (pseudogene), SATW20_19830, phage protein |
| staph_NC_009782_5485 | SAHV_0049, hypothetical protein |
| 7_55020 | |
| staph_AM990992_16566 | proC, SAPIG1569, pyrroline-5-carboxylate reductase |
| 16_1656789 | |
| staph_NC_007793_4422 | SAUSA300_0036, hypothetical protein |
| 7_44395 | |
| staph_NC_009641_1102 | NWMN_0995, phage anti-repressor protein |
| 949_1103116 | |
| staph_NC_009641_1137 | NWMN_0310, phage tail fiber |
| 731_1137898 | |
| staph_FN433596_27157 | SATW20_25670, putative amino acid permease |
| 13_2715871 | |
| staph_NC_009782_6066 | rpoB, SAHV_0540, DNA-directed RNA polymerase subunit |
| 52_606825 | beta |
| staph_FN433596_65762 | rpoB, SATW20_06120, DNA-directed RNA polymerase beta |
| 5_657803 | chain protein |
| pseudomonas_NC_00846 | |
| 3_4756080_4756240 | |
| pseudomonas_NC_00251 | |
| 6_1063894_1064077 | |
| pseudomonas_NC_00846 | PA14_35780, hypothetical protein |
| 3_3182693_3182865 | |
| pseudomonas_NC_00965 | PSPA7_0044, filamentous hemagglutinin |
| 6_2819490_2819655 | |
| pseudomonas_NC_00846 | PA14_35790, homospermidine synthase |
| 3_3184022_3184185 | |
| pseudomonas_NC_00251 | PA0984, colicin immunity protein |
| 6_1065937_1066093 | |
| pseudomonas_NC_00251 | near pyoS5, PA0985, pyocin S5 |
| 6_1067833_1068007 | |
| pseudomonas_NC_00846 | PA14_35780, hypothetical protein |
| 3_3182351_3182508 | |
| pseudomonas_NC_00846 | PA14_35790, homospermidine synthase |
| 3_3184314_3184473 | |
| pseudomonas_AP012280_ | |
| 3765216_3765383 | |
| pseudomonas_AP012280_ | |
| 3765033_3765192 | |
| enterococcus_NZ_GG70 | |
| 3715_13422_13573 | |
| enterococcus_NZ_GG70 | |
| 3582_76982_77140 | |
| enterococcus_NZ_GL45 | |
| 5004_28219_28381 | |
| enterococcus_NZ_GG70 | |
| 3720_94699_94852 | |
| enterococcus_NZ_GG70 | |
| 3715_15795_15951 | |
| enterococcus_NZ_GL45 | |
| 5899_32848_32984 | |
| enterococcus_NZ_GG69 | |
| 2918_325104_325257 | |
| enterococcus_NC_0046 | EF0957, maltose phosphorylase |
| 68_920608_920750 | |
| enterococcus_NZ_GG70 | |
| 3575_78829_78963 | |
| enterococcus_NZ_GL45 | |
| 5931_26355_26493 | |
| enterococcus_NZ_GG66 | |
| 9058_207026_207172 | |
| proteus_NZ_GG661998_ | |
| 111187_111342 | |
| proteus_NC_010554_20 | lepA, PMI1890, GTP-binding protein LepA |
| 37943_2038091 | |
| proteus_NZ_GG668576_ | |
| 810893_811054 | |
| proteus_NZ_GG668594_ | |
| 760_939 | |
| proteus_NZ_GG668579_ | |
| 22072_22234 | |
| proteus_NC_010554_24 | PMIr002 |
| 48957_2449119 | |
| proteus_NC_010554_30 | PMIr002 |
| 33758_3033936 | |
| proteus_NC_010554_45 | PMIr006 |
| 4391_454540 | |
| pseudomonas_NC_00908 | rpoB, A1S_0287, DNA-directed RNA polymerase subunit |
| 5_307050_307218 | beta |
| pseudomonas_NC_00908 | rpoB, A1S_0287, DNA-directed RNA polymerase subunit |
| 5_308225_308377 | beta |
| pseudomonas_NC_01661 | rpoB, KOX_07910, DNA-directed RNA polymerase subunit |
| 2_1674334_1674490 | beta |
| pseudomonas_NC_01660 | rpoB, BDGL_003192, RNA polymerase subunit B |
| 3_3425179_3425337 | |
| pseudomonas_NC_01660 | rpoB, BDGL_003193, DNA-directed RNA polymerase subunit |
| 3_3427629_3427808 | beta |
| pseudomonas_NC_01041 | rpoB, ABAYE3489, DNA-directed RNA polymerase subunit |
| 0_3543925_3544088 | beta |
| pseudomonas_NC_00596 | rpoB, ACIAD0307, DNA-directed RNA polymerase subunit |
| 6_304936_305079 | beta |
| pseudomonas_NC_00859 | rpoB, NT01CX_1107, DNA-directed RNA polymerase subunit |
| 3_226005_226171 | beta |
| pseudomonas_NC_01651 | rpoB, EcWSU1_00211, DNA-directed RNA polymerase |
| 4_213592_213738 | subunit beta |
| pseudomonas_NC_00596 | rpoB, ACIAD0307, DNA-directed RNA polymerase subunit |
| 6_303883_304054 | beta |
| enterobacter_NC_0146 | Entcl_3718, two component transcriptional regulator, |
| 18_3997909_3998085 | winged helix family |
| enterobacter_NZ_GL89 | |
| 2086_615149_615324 | |
| enterobacter_NZ_GL89 | |
| 2086_1664663_1664834 | |
| enterobacter_NZ_GG70 | |
| 4865_427821_427978 | |
| enterobacter_NZ_GL89 | |
| 2087_1610708_1610874 | |

TABLE 6
Species/strain level probes

| Probe Coordinates | Binding region 1 | Binding region 2 |
|---|---|---|
| acinetobacter_NC_010611_627997_628164 | GCAGCACTTGACCGCCATGAGTGACCA | CATCGCACCAACAACAATAATCG |
| acinetobacter_NC_010611_2417580_2417755 | GTGATCACTGATGCACCAGATGAAGT | ATCTTGATATTCAAGTCTATGACG |
| acinetobacter_CP002522_11753_11931 | GATATTATTGATCATGGTGCCAAGCCAA | CAATATGAAGCTGACGACGCG |
| acinetobacter_NC_011586_3908329_3908508 | GCTGAGCGTGAAGGTTCATGGATTATTA | GGTAAGGCTTACGGTCTCAT |
| acinetobacter_NC_010611_145181_145340 | GCATCTTGTGCAGCCTGAATAGCAGCGT | ACCACGTTGAATATCACCTTCGG |
| | | CAT |
| acinetobacter_NC_010611_3854494_3854662 | AAGTCCATAATTGCTTGAGTGTAGTCAT | ATCTTCGCACTGAATAATAAGAA |
| | | CAT |
| acinetobacter_NC_010400_56216_56383 | GCTTGCTGGTTCTGCACGTAGCTTACTG | AAGATGAACAGGCTACTGCAA |
| acinetobacter_NC_010611_1454960_1455136 | GCAGCGCTGTGCAAGTTCAATGTATTCT | CTCGTGCGAGTATTCCTTAAGTGT |
| acinetobacter_NC_009085_255964_256143 | GTATAACACTCGGCCAGCGCCAAGGTTC | GTTCACACATCGCCACAATATGAT |
| clostridium_NC_013974_3097606_3097772 | ACCATGCAGATACAATGAACCA | GGATGATAAGACACATCCAATTC |
| clostridium_FN665653_103469_103631 | CATCAACAGCTTCTTGAAGCATTCC | GTCCAACAACTATAACAGAACGTC |
| clostridium_NC_013974_117188_117346 | AACATATCACCTGATATTCTAGTATC | ATTCCATTATATTCAACAGGATT |
| | | GTGA |
| clostridium_NC_013316_3012882_3013047 | GCTGTTGCTTGCGGATACTG | CGTATATGTAGCTCAAGTTGC |
| clostridium_FN668375_1212250_1212413 | AAGAGCTAATGCAGCTATTGCACTTAT | CATACACTTCAGCTATAAGACCAT |
| clostridium_NC_013315_3754484_3754640 | AACAAGAGCAGAAGTTACAGACGT | GTATAATGGTGGCTAGAGGTGA |
| clostridium_FN665654_3239860_3240039 | ACTCGTGAAGACCATGCAGATACAA | AATACTTACAATGCCTGAGGA |
| clostridium_FN668941_3228320_3228491 | ACCATGCAGATACAATGAACC | CCTGAGGATGATAAGACACATC |
| clostridium_NC_013974_1962664_1962825 | GCATCTGCTGCTTCTATTGCTCCTACT | ACATGAACTGATATTAGTTCTCC |
| | | AA |
| clostridium_NC_003366_2769687_2769851 | GCACAAGCTGGAGATAACATCGG | GTAGAGGACGTATTCACAATCACT |
| clostridium_FN665653_127741_127918 | CTCTATCAGCTTCTACTGCTTCTTC | CCATCTCATCCACAGTTAATATA |
| | | TC |
| clostridium_NC_013316_2259929_2260107 | AGATGAGATTCATACTATCGTTGGAGCT | AGCAGAGAGAATAGTAAGAGGAGA |
| clostridium_NC_009089_94774_94937 | CATCAACAGCTTCTTGAAGCATT | GTCCAACAACTATAACAGAACG |
| clostridium_NC_013315_2044225_2044389 | GTCAGCAATACGCCACCAAGCTCCTAT | GTGGTGGATATCCTGTTACC |
| clostridium_NC_013315_2299408_2299586 | GCGCAATAGAGTTGTATAAGAGTGCTG | AGCATTAATTATAGATTATAATG |
| | | TATAA |
| clostridium_FN668941_3244255_3244408 | GGCATAATAGGATGGATAGATGA | ACTAATCCAACTTCTACTGCTAT |
| clostridium_NC_013316_3610909_3611065 | GTACATTCACATATAGACCATCTTAA | ACATAGGTGCAGGTAGAATAGTA |
| | | TA |
| clostridium_FN665653_1104859_1105031 | CCATACCAGTATCTTGGCATATTG | ATAATGAATAACAGCAGGTGTAT |
| | | TA |
| clostridium_NC_003366_2753681_2753838 | AGATGAAGCACAAGCTGGAGATAA | AGGACGTATTCACAATCACTG |
| clostridium_FN665653_710906_711080 | ATAATCATTCACCTCCATCATTCATAA | ACTGAATATGGTTCGTCTCA |
| clostridium_NC_009089_3706562_3706720 | GTACATTCACATATAGACCATCTTA | ACATAGGTGCAGGTAGAATAGT |
| clostridium_NC_013316_1372812_1372968 | ACTCCACCAGGATGTTGTCC | GTAGGACCGTCGTGTCCAAG |
| clostridium_FN665652_676696_676895 | GCAATATCAATGGTATCGAAGGCACTAT | GTATTGAAGGTACTATTAGCGAT |
| | | ATGC |
| clostridium_NC_013316_2641651_2641808 | GTGCCGGTCTCGGTTACTCAATG | GGATTATTATAATGCAGCTAGAAG |
| clostridium_FN668375_3595870_3596026 | GTACATTCACATATAGACCATCTT | ACATAGGTGCAGGTAGAATAGTA |
| clostridium_FN668941_1105700_1105868 | AGTTCCTTCATATGACTCAGTTGATTGA | GTTATATCTTCAATTATACATTC |
| | | CTGC |
| clostridium_NC_013974_2505182_2505359 | CAGCAGTTGTTGCTAGAGGTATG | GCATCACCAGGTGCAGCAAGT |
| clostridium_NC_013315_1077126_1077298 | GCAATTCTCTGTTGTTGTCCTCCACTCA | AGTAAGAGCCTCTTCTTGGTCAT |
| | | GA |
| clostridium_NC_009089_2182303_2182482 | CTATTCCTGATAATAAGTGTGTCCTCAT | CGGCATCATCTAACAATTCTTCT |
| clostridium_FN665652_1909777_1909942 | GTAATTCCAATTACTTCTAGCTCTGGTG | TACCATCTTCTCCATGTGTAT |
| clostridium_NC_013316_3300896_3301062 | CCATGCAGATACAATGAACCAG | GATGATAAGACACATCCAATTCC |
| clostridium_NC_013316_871338_871499 | CCTTCTGCCATTGTAGAACAAGCTCCAT | CCTGTAACTGTCCACTGAGC |
| clostridium_NC_013316_3608873_3609047 | CAATCATGATAGAATTAGATGGAAC | AGCAATAGTTCCATCAGGAGCATC |
| clostridium_FN665654_3717059_3717221 | AGTGGTGAAGGTGTTCAACAAG | ACTGAAGCTGGATATGTTGGAG |
| clostridium_NC_013315_2010489_2010657 | CGCCTCTTCAGAAGCGGATATCA | GCCAGACTTCCGCCACAACCT |
| clostridium_NC_013315_3236301_3236474 | GGCATAATAGGATGGATAGATGAGC | GCAGCAGTTGTACCTACAACTAA |
| clostridium_NC_013315_1095924_1096090 | AGTTCCTTCATATGACTCAGTTGATTG | GTTATATCTTCAATTATACATTC |
| | | CTGCG |
| enterobacter_NC_014121_4735453_4735632 | GCATGGTAGTTCGCCAGCCGCTGGAAC | ACAGCAACCGCAAGTTCTTGACAT |
| enterobacter_NC_015663_1014187_1014345 | AATATCATGGTCGTGTCCAGGCACTGGC | GTTCTGGTAGCTGCTTCTACTGTA |
| enterobacter_FP929040_3448334_3448513 | AACTTACAACTACGCGCACTTGAATCG | GAGTGTTGTATGATAGTCTCGGT |
| enterobacter_NC_009436_4051820_4051985 | GCAAGTTGAGGAGATGCTGGCATGATTC | ACATGGCTCTGGAAGATGTGCTG |
| | | ATC |
| enterococcus_FP929058_1738439_1738606 | GCGATAATTGTAATGATTCGTGGTGTTA | CCGTTGTCAATCCAGTTAGTAGA |
| | | CT |
| enterococcus_CP002621_1819224_1819388 | ACTGTGGCAGTCTATGTTCCAATTGTA | CTTATCGACATAATCCTGATAATC |
| enterococcus_FP929058_904007_904173 | GCGTCGCTTCTTGCGCTCGCC | AATGTATTCATACCGTCAAGT |
| enterococcus_FP929058_551757_551920 | GCCTTCACAACTACGTTGGAAGGTCTTC | CTAACAGTCCTGCCGACTAC |
| enterococcus_NC_004668_1122345_1122507 | GCCTTCACAACTACGTTGGAAGGTCTT | CTAACAGTCCTGCCGACTACT |
| klebsiella_NC_009648_2885456_2885620 | GCCGCTGAGCGGCGGCAAGCCGATGGC | GAATGGCAGGCCAAGCTGAAGGCG |
| klebsiella_NC_009648_3899012_3899182 | GCCAAGCGGCATTCTGGCGCCAGTGGA | CCAGACCGGAGTGGACAACGTCG |
| | | AGGCG |
| klebsiella_NC_009648_4980596_4980757 | GCCGTATATCATCGGCAATAACCGCACG | GCATGATGGTCAACAAGGTGC |
| klebsiella_NC_009648_3266359_3266519 | ACGAGCCGAGATAGGTCTGCAGCGTAC | GTACTGATATTCACCATACTGCCG |
| klebsiella_NC_012731_2557467_2557634 | GCAATATCTTCACCGGCAGCCACCGCG | GGTATATGGCACGCCAATCGC |
| klebsiella_NC_012731_4857136_4857315 | AATAACCTTAACGTCGCCAACACG | CTCGGTGAACACCTCCTGGCACG |
| proteus_NC_010554_547938_548117 | GCGGAACTGCTTGGCGTAGTAAGC | CATGTAGTGCCGTAGACCTTCAC |
| | | CA |
| pseudomonas_NC_008463_658500_658676 | GCGAGACCGGCGGCACCATCGTCTCCAG | TTCTGCCTGATGGACGTCTCCGG |
| | | CTCG |
| pseudomonas_NC_008463_753931_754099 | GCGGTTCACCTGTTCGCCTTCGAACACG | GCGCAGCATCTGACGCAGGATGG |
| | | TCTCG |
| pseudomonas_NC_009656_6431649_6431828 | ACTCCATCGCCATCAAGGACATGGCCGG | ATCGACGTGTTCCGCATCTTCGA |
| | | CGCG |
| pseudomonas_NC_008463_560357_560534 | GCCTGATGCACTACAGCGCCTGG | TACCACATGGTCGATCTCGACGA |
| | | CTGC |
| pseudomonas_NC_010322_5224859_5225023 | GCGCATCCAGGACGGCGAGTACG | CTTCGAGTGCCTGCACGAGCTGAA |
| pseudomonas_NC_008463_4839746_4839924 | GCTGGAGAACGTCAAGGTGGTGATCATC | ACCGATAACGACGACCGCATCAA |
| staph_FN433596_2844085_2844263 | ACGATTGGAGAAGGCAGTGTGATTGG | GGACAGATTACAATTGGCG |
| staph_NC_009632_1198350_1198529 | GCCGCAATACCGATATTCCA | CCATTGTCCACCAGCTGAACCG |
| staph_FN433596_2521244_2521419 | GTGAAGGTCGTGCTCCTATCGGT | AGATCTGGTGAAGTTCGTATGAT |
| staph_NC_009487_430842_431017 | GCTGGTACTTGTACTTATATCGA | ATCAGAAGATGATATCGTTACGT |
| | | CAT |
| staph_NC_009782_2086681_2086849 | GCGCATATTGCATTAATGGCTATAGAT | GCCAGCAGGTTATACACTCG |
| staph_NC_009782_58256_58423 | GCAATTCTTACCACAGCACGAAGAACAG | ATCTAGATGAAGATAATGAAGTCG |
| staph_NC_013450_991049_991222 | GCATCTTCATACAATACTTCTAGCTTAC | CACAATACCAGTTGTATTACG |
| staph_NC_013450_1360842_1361008 | GCTTCAGCGCCATTACCGCCACCAGCT | ACTCTTGATATATTCTTGTAAGCG |
| staph_AM990992_2526026_2526192 | GTTCACACAACGCGCCGACTAGAATCC | CACGATATCCAAGATAATGATTG |
| | | GCTA |
| staph_NC_010079_361284_361447 | GCGCACCTACAATCGCCATTACTACAC | ACTCATTATCGACTGTTACATCG |
| | | ACTGA |
| staph_NC 007795_2085723_2085901 | AGCGCACATGTGACAGCGTGTAGGTTA | GTGCCTTAGATTGTTCAGAACAAT |
| staph_NC_009641_23125_23297 | CGAATGGATATGTACCATGGTCGATATC | CTCTCTAATATGATGTCCAT |
| staph_FN433596_2144570_2144734 | ACTACAACAGCAACCGCATTACAATGGC | GGTGCTAAGAGGTCATCGGA |
| staph_NC_009782_54857_55020 | AGCTTCAGATAAGTACCTATCTGA | GGAAGAATAGTTATTCTTGATAA |
| | | TGTAT |
| staph_AM990992_1656616_1656789 | CGTATTGCTCGAATACATGATA | ACAATGTATCAAGGCCAGCT |
| staph_NC_007793_44227_44395 | GCGACCAGTTGTTATCGACCGTGT | CAGAACGATACGGTGCTGTATA |
| staph_NC_009641_1102949_1103116 | CAATTACATTGTCTGTTGCGTAGATACC | GTTGTGGCTAATGTGCCAGTT |
| staph_NC_009641_1137731_1137898 | GCACCACTCTATAGCAGTAGCGTATTG | ACAGCCAATGTCACCTAAGTCAA |
| | | CA |
| staph_FN433596_2715713_2715871 | ACAGTCCGAATAAGATACGACTATTCGA | CGTTGTAACGTATATGAATAGTT |
| | | GA |
| staph_NC_009782_606652_606825 | AGATGCAATAACAGGTCGAATATTAATT | GCCATAGTGAGAGTAGTGAA |
| staph_FN433596_657625_657803 | AGATGCAATAACAGGTCGAATATTAA | ACACATACGGCCATAGTGAGAG |
| pseudomonas_NC_008463_4756080_4756240 | GAATCGAACGGTCTCATTAACAGAT | GCTTTCCAGGGATATAAGACGC |
| pseudomonas_NC_002516_1063894_1064077 | CCCGCAGAGTCACACTCGGA | ACTCTTGGTACTACTCACTAGC |
| pseudomonas_NC_008463_3182693_3182865 | GAGTCTCTTTCAACCTGGATTAGATAT | AAGATTAATAGCGTACTTTACTCC |
| pseudomonas_NC_009656_2819490_2819655 | ATCCCGCAGATACTAGGTTCTTAAT | GAACTATTCATATTACACCCTAA |
| | | GG |
| pseudomonas_NC_008463_3184022_3184185 | CAGTGGGCTATCCTAAGCCAAAG | CATAAGCGAACTAACTATCACTTA |
| pseudomonas_NC_002516_1065937_1066093 | ACAAAGCGTTCTAAACGATTAGAACT | CGAGAAAGGAAACAGGATAGTAC |
| pseudomonas_NC_002516_1067833_1068007 | CCAATGGAGAAGTCTAAATGTCCAA | TTATCAGAGATACATGACTCTTA |
| | | GG |
| pseudomonas_NC_008463_3182351_3182508 | CGAATCACTGGACTACATTTATATTTCT | AGCGAACCTTTATATTTGACCAT |
| pseudomonas_NC_008463_3184314_3184473 | CTCAAGTCTTGCCCTGATAGAATTAT | TCACGACTTATCTACTTTAGAAA |
| | | TC |
| pseudomonas_AP012280_3765216_3765383 | GGTGATCGTTATTATGATAGTACGGC | CTCGGTTAAGGGAATTACGAC |
| pseudomonas_AP012280_3765033_3765192 | ACTCGGATGGTAGGTTTATTAAAGC | GTGATCGTTATTATGATAGTACGG |
| enterococcus_NZ_GG703715_13422_13573 | ACAATCGTTGTCGCACTGCATAG | GAACTTGGTCTACCGTACCAC |
| enterococcus_NZ_GG703582_76982_77140 | GGATAATACAATCCTAATACGTACGGA | GCTGCTGTAACTAGGGTAGC |
| enterococcus_NZ_GL455004_28219_28381 | CTATATTCAACGGGTCACGGGTAG | TCATTGATTCGATCTCGTAACTC |
| enterococcus_NZ_GG703720_94699_94852 | AATGTTATTGTGGTTGCGTGTTCG | TACTTTGGAAGTGCCCTGAC |
| enterococcus_NZ_GG703715_15795_15951 | CATGTCTTCTAGTACAGGTTTGCCG | TGTAAGAGGCCGCTAACTTC |
| enterococcus_NZ_GL455899_32848_32984 | CTCTGGCTCGTGGGCTCGG | TTCTTGAGATAGTCCGGTATAATC |
| enterococcus_NZ_GG692918_325104_325257 | ATTCGATCACGATGGGCTGGG | AATTTCCTGTGTCATACACGC |
| enterococcus_NC_004668_920608_920750 | CAATTGATTTAGCCACTACACCTTAC | CACTATTCTGGCGACCACC |
| enterococcus_NZ_GG703575_78829_78963 | GATAAAGAAGCGTCTTGACCCAGT | ATCTGGTGCTCCTTGACGC |
| enterococcus NZ_GL455931_26355_26493 | GCAAATTTAGAGAGTGCATGCATG | GGAAGAGGACGGCATACAAC |
| enterococcus_NZ_GG669058_207026_207172 | CATTTCATCTAGACCGCTCGTGT | GCTTGAAGTGTATGTTGGGAC |
| proteus_NZ_GG661998_111187_111342 | GTCGCCCTCGTGCTAACGT | GGTTCTTTGATGTACCGGTT |
| proteus_NC_010554_2037943_2038091 | GCTGATGACGGTGAAGTTTATCA | CATTATCGCACATATTGACCAC |
| proteus_NZ_GG668576_810893_811054 | GAAATTAGCTAAAGGGATATCGCG | AACTTTCCGCCAATCCTGC |
| proteus NZ_GG668594_760_939 | CACCTACGTTCTCACCTGCAC | ATTCGATAGTACCAGTTACGTC |
| proteus_NZ_GG668579_22072_22234 | GTTGCTTATAGCGTCGCTGCT | CTGGTTATCGAGAAGATAAAGG |
| proteus NC_010554_2448957_2449119 | GTAAGCGTAGCGATACGTTGAG | GAGTGAACGCACCACTGG |
| proteus_NC_010554_3033758_3033936 | TCAGGTAGAGAATACTCAGGCGC | CGGAGAAGGCTAGGTTGTC |
| proteus_NC_010554_454391_454540 | GCAACCCACTCCCATGGTGT | CGTTCTTCATCAGACAATCTG |
| pseudomonas_NC_009085_307050_307218 | AACTAAACCTACACGGAATTGGTTC | GCAGATACACGACGTTTATGT |
| pseudomonas_NC_009085_308225_308377 | GCCGCTTCACCTACGTTAGGAA | CGTAAAGATGAGTCTTTAACGTC |
| pseudomonas_NC_016612_1674334_1674490 | GACGTTTGTGCGTAATCTCAGAC | GAGGAAACCGTATTCGTTCGT |
| pseudomonas_NC_016603_3425179_3425337 | ACAACACTTTACCACTTGAGTGGG | GTAACTGCCCATGTCAAGATAC |
| pseudomonas_NC_016603_3427629_3427808 | CCACGTTTAGTTGAACCACCGC | TCAATACGCCAGTTGTTAGTTC |
| pseudomonas_NC_010410_3543925_3544088 | AATCGATAATAAGTACGGTGCATCC | GAAGAATACATTCGCGTACATC |
| pseudomonas_NC_005966_304936_305079 | AAGCAAGATCGAGTCTTCATAGTTG | GATATACACGATACCTGATTCGT |
| pseudomonas_NC_008593_226005_226171 | CCGATATTCATACGAGAAGGTACAC | CAGTAACTCTATTGTCAAACGGT |
| pseudomonas_NC_016514_213592_213738 | GTAGTGAGTCGGGTGTACGTCTC | TCTTCGATAGCAGACAGATAGT |
| pseudomonas_NC_005966_303883_304054 | ACCTACACGGAATTGGTTCTCAGT | GATACACGACGTTTGTGTGTA |
| enterobacter_NC_014618_3997909_3998085 | CAACATCATTAGCTTGGTCGTGGG | TTGCGTGTTACCAACTCGTC |
| enterobacter_NZ_GL892086_615149_615324 | CGGCACGTCCGAATCGTATCA | TCGTGTCCCGTATATGTTGG |
| enterobacter_NZ_GL892086_1664663_1664834 | AATAGAGGCCCACAAGTCTTGTTC | CGCTCTCCACTATGGGTAGT |
| enterobacter_NZ_GG704865_427821_427978 | GCTACATTAATCACTATGGACAGACA | GATGGTCGATCTATCGTCTCT |
| enterobacter_NZ_GL892087_1610708_1610874 | GAAGTGTTATTCAAACTTTGGTCCC | CTTGAACCCTTGGTTCAAGGT |

TABLE 7
Marker regions are highly polymorphic regions (like, e.g., VNTRs) that provide
fine resolution.

| Probe Coordinates | Gene |
|---|---|
| plasmids_NC_011980_58308_58487 | insA, MM1_0111, IS1 protein InsA, MM1_0112, IS1 |
| | protein InsB |
| plasmids_NC_015599_37281_37455 | intIl, pN3_046, integrase |
| plasmids_NC_007351_37979_38146 | SSPP128, IS431 transposase |
| plasmids_FN822749_1846_2009 | ETEC1392/75_p75_00003, putative IS100 |
| | transposase |
| plasmids_NC_004851_143949_144109 | CP0039, IS629 ORF2 |
| plasmids_NC_010558_156799_156957 | IS1-insB, IPF_205, IS1-insB |
| plasmids_NC_012547_53585_53752 | tnpA, PGO1_p15, putative transposase TnpA |
| plasmids_NC_013950_91008_91174 | tnpR, pKF94_116, TnpR |
| plasmids_NC_002698_168967_169123 | insB, pWR501_0054, IS1 transposase |
| CMY_AB061794_343_489 | intI1, DNA integrase |
| IMG_AY033653_1343_1500 | intI1, DNA integrase |
| TEM_U36911_4374_4551 | |
| TEM_U36911_7596_7762 | |

TABLE 8
Marker probes

| Probe Coordinates | Binding region 1 | Binding region 2 |
|---|---|---|
| plasmids_NC_011980_58308_58487 | GCAGTCGGTAACCTCGCGC | GCGCTATCTCTGCTCTCACTGC |
| plasmids_NC_015599_37281_37455 | GCTGTAATGCAAGTAGCGTATGCGCTC | GAACAGCAAGGCCGCCAATGCCTGACG |
| plasmids_NC_007351_37979_38146 | CGCATATGCTGAATGATTATCTCGTTGC | ATCTTGCTCAATGAGGTTATTCA |
| plasmids_FN822749_1846_2009 | GACGACAGATGCAGGTTGA | CGCATCGCCGATGCTCATC |
| plasmids_NC_004851_143949_144109 | CGCCTGCTCCAGTGCATCCAGCACGAAT | ATGCTCTCCGCCATCGCGTTGTCA |
| plasmids_NC_010558_156799_156957 | AGTGCGTTCACCGAATACGTGCGCA | CAGGTTATGCCGCTCAATTC |
| plasmids_NC_012547_53585_53752 | CGCATATGCTGAATGATTATCTCGTTG | ACGGTGATCTTGCTCAATGAGGTTATTC |
| plasmids_NC_013950_91008_91174 | GCTGTGGCACAGGCTGAACGCCG | GGTGATGTCATTCTGGTTAAGA |
| plasmids_NC_002698_168967_169123 | ACATAATCTGAATCTGAGACAACATC | ACGCACTCTGGCCACACTGG |
| CMY_AB061794_343_489 | CATCACGAAGCCCGCCACA | GCCCTTGAGCGGAAGTATC |
| IMG_AY033653_1343_1500 | CGGAAGTATCCGCGCGCC | TTCGATCACGGCACGATC |
| TEM_U36911_4374_4551 | CATTCTCTCGCTTTAATTTATTAACCT | ATCGACCTTCTGGACATTATC |
| TEM_U36911_7596_7762 | CGTTGCTTACGCAACCAAATATC | TGATCTTGCTCAATGAGGTTA |

TABLE 9
Resistance regions can be used to detect one or more genes associated with
resistance to antimicrobial compounds, such as antibiotic resistance genes.

| Probe Coordinates | Gene |
|---|---|
| plasmids_NC_013950_90185_90338 | pKF94_115, beta-lactamase |
| plasmids_NC_013452_4052_4209 | SAAV_b4 tetracycline resistance protein |
| plasmids_NC_014208_52313_52469 | pKOX105p23, VIM-1, pKOX105p24, IntIA |
| | pKOX105p67, truncated AadA |
| betalactamase_AB372224_738_905 | blaCMY-39, class C beta-lactamase CMY-39 |
| betalactamase_EF685371_398_548 | beta-lactamase CMY-29 |
| betalactamase_DQ149247_231_371 | bla-OXA-86, OXA-86 |
| betalactamase_AY750911_244_414 | bla-oxa-69, beta-lactamase OXA-69 |
| betalactamase_DQ519087_417_575 | blaOXA-93, beta-lactamase OXA-93 |
| betalactamase_AM231719_379_537 | blaOXA-90, class D beta lactamase |
| betalactamase_Y14156_663_819 | CTX-M-4, beta lactamase |
| betalactamase_JN227085_763_931 | blaCTX-M-117, CTX-M-117 beta-lactamase |
| betalactamase_EU259884_1030_11 | aacA4, AacA4 aminoglycoside (6') |
| 70 | acetyltransferase |
| betalactamase_HQ913565_578_730 | blaCTX-M-106, beta-lactamase CTX-M-106 |
| betalactamase_AY524988_385_552 | blaVIM-9, VIM-9 |
| CARB_AF030945_646_795 | CARB-6, class A beta-lactamase |
| CARB_U14749_1227_1390 | blaCARB-4, CARB-4 precursor |
| CARB_AF313471_2731_2906 | aadA1a, AAD(3″) aminoglycoside (3″) |
| | adenylyltransferase |
| CMY_DQ463751_613_790 | blaCMY-23, hypothetical CMY-23 protein |
| | precursor |
| CMY_EF685371_397_552 | beta-lactamase CMY-29 |
| CMY_EU515251_583_733 | blaCMY-40, AmpC beta-lactamase |
| CMY_JN714478_1882_2055 | blaCMY-66, AmpC beta-lactamase CMY-66 |
| CMY_X91840_1872_2046 | bla CMY-2, extended spectrum beta-lactamase |
| CTXM_EF219134_13713_13858 | AadA2 aminoglycoside adenylytransferase; |
| | confers resistance to streptomycin and |
| | spectinomycin |
| CTXM_HQ398215_802_947 | blaCTX-M-98, beta-lactamase CTX-M-102 |
| CTXM_AM982522_639_788 | blaCTX-M-78, CTX-M-78 beta-lactamase |
| GES_HM173356_1163_1321 | blaGES-16, carbapenem-hydrolyzing extended- |
| | spectrum beta lactamase GES-16 |
| GES_AF156486_1754 1905 | ges-1, beta-lactamase GES-1 |
| GES_HQ874631_571_748 | extended-spectrum beta-lactamase GES-17 |
| GES_FJ820124_1174_1338 | beta-lactamase GES10 |
| IMG_DQ361087_489_645 | blaIMP-22, metallo-beta-lactamase IMP-22 |
| IMG_JN848782_301_475 | blaIMP-33, metallo-beta-lactamase IMP-33 |
| IMG_EF192154_182_328 | blaIMP-24, metallo-beta-lactamase IMP-24 |
| IMG_AF318077_871_1047 | aacC4, aminoglycoside-N- |
| | acetyltransferase |
| IMG_AF318077_515_657 | aacC4, aminoglycoside 6'-N- |
| | acetyltransferase |
| KPC_HM066995_226_375 | b1aKPC, beta-lactamase KPC-11 |
| KPC_GQ140348_624_799 | KPC-10, beta-lactamase KPC-10 |
| KPC_EU729727_683_840 | carbapenem-hydrolyzing beta-lactamase KPC- |
| | 7 |
| KPC_FJ234412_691_839 | blaKPC-8, beta-lactamase KPC-8 |
| NDM_JN104597_64_211 | blaNDM-5, NDM-5 metallo-beta-lactamase |
| NDM_FN396876_2744_2885 | blaNDM-1, metallo-beta-lactamase |
| NDM_FN396876_2958_3117 | blaNDM-1, metallo-beta-lactamase |
| NDM_JN104597_314_465 | blaNDM-5, NDM-5 metallo-beta-lactamase |
| NDM_FN396876_2382_2548 | blaNDM-1, metallo-beta-lactamase |
| OXA_EF650035_239_388 | bla-OXA-109, beta-lactamase OXA-109 |
| OXA_EU019535_389_537 | bla-OXA-80, beta-lactamase OXA-80 |
| OXA_EF650035_423_594 | bla-OXA-109, beta-lactamase OXA-109 |
| OXA_DQ309276_232_380 | bla-OXA-84, beta-lactamase OXA-84 |
| OXA_DQ445683_232_380 | bla-OXA-89, oxacillinase OXA-89 |
| OXA_X75562_201_366 | OXA-7, beta lactamase OXA-7 |
| OXA_M55547_995_1154 | tnpR, aac, Aac |
| OXA_AY445080_313_469 | blaOXA-56, restricted-spectrum beta- |
| | lactamase OXA-56 |
| PER_Z21957_217_371 | PER-1, extended-spectrum beta-lactamase |
| | PER-1 |
| PER_HQ713678_6002_6167 | blaPER-7, blaPER-7 |
| PER_GQ396303_667_844 | blaPER-6, extended-spectrum beta-lactamase |
| | PER-6 |
| PER_X93314_954_1122 | bla(per-2), extended-spectrum beta- |
| | lactamase |
| PER_HQ713678_4517_4674 | transposase |
| PER_HQ713678_5074_5219 | transposase |
| PER_GQ396303_254_399 | blaPER-6, extended-spectrum beta-lactamase |
| | PER-6 |
| SHV_AY661885_656_806 | blaSHV-30, beta-lactamase SHV-30 |
| SHV_AF535128_587_761 | blaSHV-40, beta-lactamase SHV-40 |
| SHV_U92041_406_579 | SHV-8, beta-lactamase |
| SHV_AY288915_617_764 | blaSHV-50, beta-lactamase SHV-50 |
| SHV_HQ637576_88_245 | blaSHV-135, beta-lactamase SHV-135 |
| SHV_AF535128_188_362 | blaSHV-40, beta-lactamase SHV-40 |
| SHV_X98102_763_913 | blaSHV-2a, beta-lactamase SHV-2a |
| TEM_GQ149347_3605_3747 | near kanamycin resistance protein |
| TEM_GU371926_11801_11944 | traN, TraN |
| TEM_J01749_766_908 | tet, tetracycline resistance protein |
| VEB_EU259884_6947_7094 | blaVEB-6, VEB-6 extended-spectrum beta- |
| | lactamase |
| VEB_EF136375_596_738 | blaVEB-4, extended-spectrum beta-lactamase |
| | VEB-4 |
| VEB_EF420108_234_380 | blaVEB-5, extended spectrum beta-lactamase |
| | VEB-5 |
| VEB_AF010416_89_230 | veb-1, extended spectrum beta-lactamase |
| VIM_AY524988_385_552 | blaVIM-9, VIM-9 |
| VIM_Y18050_3464_3614 | blaVIM, beta-lactamase VIM-1 |
| VIM_AY635904_58_203 | blaVIM-11, metallo-beta-lactamase |
| VIM_HM750249_275_454 | bla, metallo-beta-lactamase VIM-25 |
| VIM_AJ536835_313_481 | blaVIM-7, metallo-b-lactamase |
| VIM_EU118148_131_300 | near intI1, DNA integrase INTI1 |
| VIM_DQ143913_921_1063 | near intI1, IntI1 |
| VIM_EU118148_1060_1229 | blaVIM-17, metallo-beta-lactamase VIM-17 |
| van_NC_008821.1_11898_12045 | vanB, pVEF236, D-alanine--D-lactate ligase |
| mecA_AY820253.1_1431_1608 | mecA, PBP2a-like protein |
| mecA_AY952298.1_130_302 | Pbp2′ |
| erm_NC_002745.2_871803_871973 | |
| erm_NC_002745.2_871666_871841 | ermA, SA1951, rRNA methylase Erm(A) |

TABLE 10
Resistance probes

| Probe Coordinates | Binding region 1 | Binding region 2 |
|---|---|---|
| plasmids_NC_013950_90185_90338 | GAGGACCGAAGGAGCTAACCG | CGCCGCATACACTATTCTC |
| plasmids_NC_013452_4052_4209 | CTCATTCCAGAAGCAACTTCTTCTT | GGATAGCCATGGCTACAAGAATA |
| plasmids_NC_014208_52313_52469 | GGTTCTGGACCAGTTGCGTGAGCGC | CGTAACATCGTTGCTGCTCCAT |
| betalactamase_AB372224_738_905 | CGCTGGATTTCACGCCATAGGC | TGTCGCTACCGTTGATGATT |
| betalactamase_EF685371_398_548 | CGTATAGGTGGCTAAGTGCAGC | GTAACTCATTCCTGAGGGTTTC |
| betalactamase_DQ149247_231_371 | GTACATACTCGATCGAAGCACGA | CCGGAATAGCGGAAGCTTTC |
| betalactamase_AY750911_244_414 | AAGGTCGAAGCAGGTACATACTCG | AGACATGAGCTCAAGTCCAAT |
| betalactamase_DQ519087_417_575 | GAAGCTTTCATAGCGTCGCCTAG | TTAGCTAGCTTGTAAGCAAATTG |
| betalactamase_AM231719_379_537 | GAAGCTTTCATGGCATCGCCTAG | AGCTAGCTTGTAAGCAAACTG |
| betalactamase_Y14156_663_819 | CGCTACCGGTAGTATTGCCCTT | AGAATATCCCGACGGCTTTC |
| betalactamase_JN227085_763_931 | ATCGCCACGTTATCGCTGTACT | TTTACCCAGCGTCAGATTCC |
| betalactamase_EU259884_1030_1170 | CAAGTACTGTTCCTGTACGTCAGC | TCGCCAGTAACTGGTCTATTC |
| betalactamase_HQ913565_578_730 | CAACGTCTGCGCCATCGCC | CGCAATATCATTGGTGGTGC |
| betalactamase_AY524988_385_552 | GCCGCCCGAAGGACATCAAC | CAGACGGGACGTACACAAC |
| CARB_AF030945_646_795 | CGTGCTGGCTATTGCCTTAGG | GTAATACTCCTAGCACCAAATC |
| CARB_U14749_1227_1390 | CATTAGGAGTTGTCGTATCCCTCA | AATACTCCGAGCACCAAATC |
| CARB_AF313471_2731_2906 | AAATTGCAGTTCGCGCTTAGC | GTTCCATAGCGTTAAGGTTTC |
| CMY_DQ463751_613_790 | GCGCCAAACAGACCAATGCT | GATTTCACGCCATAGGCTC |
| CMY_EF685371_397_552 | GTATAGGTGGCTAAGTGCAGCA | TCGTAACTCATTCCTGAGGG |
| CMY_EU515251_583_733 | GTCATCGCCTCTTCGTAGCTC | GCCATATCGATAACGCTGG |
| CMY_JN714478_1882_2055 | ACCAATACGCCAGTAGCGAGA | GCAACGTAGCTGCCAAATC |
| CMY_X91840_1872_2046 | CAATCAGTGTGTTTGATTTGCACC | TACCCGGAATAGCCTGCTC |
| CTXM_EF219134_13713_13858 | CGGATAACGCCACGGGATGA | ACCGGGTCAAAGAATTCCTC |
| CTXM_HQ398215_802_947 | GCGGCGTGGTGGTGTCTC | CGCTGCCGGTCTTATCAC |
| CTXM_AM982522_639_788 | GCCACGTCACCAGCTGCG | CGGCTGGGTGAAGTAAGTC |
| GES_HM173356_1163_1321 | GCTCGTAGCGTCGCGTCTC | TTGACCGACAGAGGCAAC |
| GES_AF156486_1754_1905 | CAGCAGGTCCGCCAATTTCTC | AGTGGACGTCAGTGCGC |
| GES_HQ874631_571_748 | CCATAGAGGACTTTAGCCACAGT | TACACCGCTACAGCGTAAT |
| GES_FJ820124_1174_1338 | CATATGCAGAGTGAGCGGTCC | TCAATTCTTTCAAAGACCAGC |
| IMG_DQ361087_489_645 | CCATTAACTTCTTCAAACGATGTATG | ACCCGTGCTGTCGCTAT |
| IMG_JN848782_301_475 | GTGCTGTCGCTATGGAAATGTG | AACCAAACCACTAGGTTATCTT |
| IMG_EF192154_182_328 | GTCAGTGTTTACAAGAACCACCA | ATGCATACGTGGGAATAGATT |
| IMG_AF318077_871_1047 | CGAACCAGCTTGGTTCCCAAG | TCACTGCGTGTTCGCTC |
| IMG_AF318077_515_657 | GATGCTGTACTTTGTGATGCCTA | CGCTTGGCAAGTACTGTTC |
| KPC_HM066995_226_375 | GCAAGAAAGCCCTTGAATGAGC | GCGTTATCACTGTATTGCAC |
| KPC_GQ140348_624_799 | AATCAACAAACTGCTGCCGCT | GCTGTACTTGTCATCCTTGT |
| KPC_EU729727_683_840 | CCAGTCTGCCGGCACCGC | TCGAGCGCGAGTCTAGC |
| KPC_FJ234412_691_839 | CCGACTGCCCAGTCTGCCG | CGAGCGCGAGTCTAGCC |
| NDM_JN104597_64_211 | GTAAATAGATGATCTTAATTTGGTTCAC | TTGCTGGCCAATCGTCG |
| NDM_FN396876_2744_2885 | CACAGCCTGACTTTCGCCGC | CAAGCAGGAGATCAACCTGC |
| NDM_FN396876_2958_3117 | GGTGGTCGATACCGCCTGG | GTGAAATCCGCCCGACG |
| NDM_JN104597_314_465 | CATGTCGAGATAGGAAGTGTGC | TGATGCGCGTGAGTCAC |
| NDM_FN396876_2382_2548 | CAATCTGCCATCGCGCGATT | CGGCAATCTCGGTGATGC |
| OXA_EF650035_239_388 | CGAAGCAGGTACATACTCGGTC | ACGAGCTAAATCTTGATAAACTT |
| OXA_EU019535_389_537 | TAGAATAGCGGAAGCTTTCATGG | AGCTAGCTTGTAAGCAAACTG |
| OXA_EF650035_423_594 | CAAGTCCAATACGACGAGCTAAA | GAATAGCATGGATTGCACTTC |
| OXA_DQ309276_232_380 | GGTACATACTCGGTCGAAGCAC | AATCTTGATAAACTGAAATAGCG |
| OXA_DQ445683_232_380 | GGTACATACTCGGTCGATGCAC | TCTTGATAAACCGGAATAGCG |
| OXA_X75562_201_366 | GTAATTGAACTAGCTAATGCCGTAC | TTATGACACCAGTTTCTAGGC |
| OXA_M55547_995_1154 | CAAGTACTGTTCCTGTACGTCAG | GCCCAGTTGTGATGCATTC |
| OXA_AY445080_313_469 | TCTCTTTCCCATTGTTTCATGGC | TGCGGAAATTCTAAGCTGAC |
| PER_Z21957_217_371 | GTAGGTTATGCAGTTATTAGGTTCAG | GACTCAGCCGAGTCAAGC |
| PER_HQ713678_6002_6167 | GCAGTACCAACATAGCTAAATGC | AAATAACAAATCACAGGCCAC |
| PER_GQ396303_667_844 | GGTCCTGTGGTGGTTTCCACC | CGCGATAATGGCTTCATTGG |
| PER_X93314_954_1122 | TAACCGCTGTGGTCCTGTGG | TGCGCAATAATAGCTTCATTG |
| PER_HQ713678_4517_4674 | GGAAGCGTTGCTTGCCATAGT | AACCGAAGCACCATGTAATT |
| PER_HQ713678_5074_5219 | GTTCGGTGCAAAGACGCCG | TCGCAGACTTCAATATCAATATT |
| PER_GQ396303_254_399 | CACCTGATGCAGAACCAGCAT | AGGCCACGTTATCACTGTG |
| SHV_AY661885_656_806 | CAGCTGCCGTTGCGAACG | CGCAGATAAATCACCACAATC |
| SHV_AF535128_587_761 | GCTCAGACGCTGGCTGGTC | CCGCAGATAAATCACCACG |
| SHV_U92041_406_579 | GCCAGTAGCAGATTGGCGGC | GAACGGGCGCTCAGACG |
| SHV_AY288915_617_764 | CCACTGCAGCAGATGCCGT | GTATCCCGCAGATAAATCACC |
| SHV_HQ637576_88_245 | TTAATTTGCTTAAGCGGCTGCG | CCAGCTGTTCGTCACCG |
| SHV_AF535128_188_362 | GGGAAAGCGTTCATCGGCG | TCGCTCATGGTAATGGCG |
| SHV_X98102_763_913 | TCTTATCGGCGATAAACCAGCC | CGTTGCCAGTGCTCGAT |
| TEM_GQ149347_3605_3747 | GTCGGAAAGTTGACCAGACATTA | ATACTAGGAGAAGTTAATAAATACG |
| TEM_GU371926_11801_11944 | GTGAAGTGAATGGTCAGTATGTTG | AGTGCGCAGGAGATTAGC |
| TEM_J01749_766_908 | CCTGTCCTACGAGTTGCATGAT | ATAATGGCCTGCTTCTCGC |
| VEB_EU259884_6947_7094 | CAAATACTAAATTATACAGTATCAGAG | ATGCAAAGCGTTATGAAATTTC |
| | AG | |
| VEB_EF136375_596_738 | GTTCTTATTATTATAAGTATCTATTAA | CATTAGTGGCTGCTGCAAT |
| | CAGTT | |
| VEB_EF420108_234_380 | CATCGGGAAATGGAAGTCGTTAT | GTTCAATCGTCAAAGTTGTTC |
| VEB_AF010416_89_230 | CGTGGTTTGTGCTGAGCAAAG | CAAAGTTAAGTTGTCAGTTTGAG |
| VIM_AY524988_385_552 | GCCGCCCGAAGGACATCAA | AGACGGGACGTACACAAC |
| VIM_Y18050_3464_3614 | GCAACTCATCACCATCACGGA | TGATGCGTACGTTGCCAC |
| VIM_AY635904_58_203 | GCGACAGCCATGACAGACGC | GGACAATGAGACCATTGGAC |
| VIM_HM750249_275_454 | AAACGACTGCGTTGCGATATG | TTCCGAAGGACATCAACGC |
| VIM_AJ536835_313_481 | ATGCGACCAAACGCCATCGC | ATCGTCATGGAAGTGCGTA |
| VIM_EU118148_131_300 | GAACAGGCTTATGTCAACTGGG | CATAACATCAAACATCGACCC |
| VIM_DQ143913_921_1063 | ACGAACCGAACAGGCTTATGTC | TAACGCGCTTGCTGCTT |
| VIM_EU118148_1060_1229 | CATCATAGACGCGGTCAAATAGA | ACTCATCACCATCACGGAC |
| van_NC_008821.1_11898_12045 | CAGGCTGTTTCGGGCTGTGA | GGGTTATTAATAAAGATGATAGGC |
| mecA_AY820253.1_1431_1608 | TAATTCAAGTGCAACTCTCGCAA | TTTATTCTCTAATGCGCTATATATT |
| mecA_AY952298.1_130_302 | GGATAGTTACGACTTTCTGCTTCA | TGTATTGCTATTATCGTCAACG |
| erm_NC_002745.2_871803_871973 | GTCAGGCTAAATATAGCTATCTTATCG | TCAGTTACTGCTATAGAAATTGAT |
| erm_NC_002745.2_871666_871841 | CATCCTAAGCCAAGTGTAGACTC | AAGATATATGGTAATATTCCTTATA |
| | | AC |

TABLE 11
Additional regions may be used for additional discrimination and
characterization of organisms.

| Probe Coordinates | Gene |
|---|---|
| peGFP_N1_730_925 | |
| CMY_X92508_126_301 | |
| TEM_X64523_2037_21 | near tnpR, resolvase |
| 91 | |
| TEM_J01749_2068_22 | near ROP protein |
| 39 | |
| TEM_AF091113_1529_ | |
| 1699 | |
| TEM_J01749_1634_17 | |
| 83 | |
| TEM_U36911_6901_70 | |
| 69 | |
| TEM_GU371926_33909_ | klcA, KlcA |
| 34082 | |
| VIM_EU118148_2821_ | qacEdeltal, quarternary ammomium compound-resistance |
| 2961 | protein QacEdeltal sull, dihydropteroate synthase SUL1 |
| van_DQ018710.1_648 | |
| 1_6652 | |
| van_DQ018710.1_676 | |
| 4_6926 | |
| van_AY926880.1_364 | |
| 0_3785 | |
| van_FJ545640.1_517_ | |
| 690 | |
| van_AE017171.1_347 | |
| 15_34859 | |
| van_FJ349556.1_560 | |
| 1_5765 | |
| mecA_AM048806.2_15 | |
| 74_1720 | |
| mecA_EF692630.1_23 | |
| 9_405 | |
| mex_AF092566.1_371_ | |
| 520 | |
| mex_AF092566.1_50_ | |
| 193 | |
| mex_CP000438.1_487 | |
| 178_487357 | |
| mex_NZ_AAQW0100000 | |
| 1.1_461304_461466 | |
| erm_EU047809.1_79_ | |
| 229 | |
| gyrB_NC_015663_145 | EAE_24795, hemagluttinin domain-containing |
| 5472_1455621 | protein, gyrB, EAE_07020, DNA gyrase subunit B |
| gyrB_NC_010410_421 | gyrB, ABAYE0004, DNA gyrase, subunit B |
| 5_4366 | |
| gyrB_NC_005773_490 | gyrB, PSPPH_0004, DNA gyrase subunit B |
| 4_5052 | |
| gyrB_NC_016514_534 | gyrB, EcWSU1_00004, DNA gyrase subunit B |
| 3_5487 | |
| gyrB_NC_016603_263 | gyrB, BDGL_002434, DNA gyrase, subunit B |
| 1439_2631616 | |
| gyrB_NC_009436_436 | gyrB, Ent638_0004, DNA gyrase subunit B |
| 6_4524 | |
| gyrB_NC_009512_420 | gyrB, Pput_0004, DNA gyrase subunit B |
| 3_4373 | |

TABLE 12
Additional arms

| Probe Coordinates | Binding region 1 | Binding region 2 |
|---|---|---|
| peGFP_N1_730_925 | GTGGTATGGCTGATTATGATCTAGAGT | GAGTTTGGACAAACCACAACTAGAA |
| CMY_X92508_126_301 | AGTATCTTACCTGAAATTCCCTCAC | CCTCTCGTCATAAGTCGAATG |
| TEM_X64523_2037_2191 | CAGTCCCTCGATATTCAGATCAGA | TTAACAATTTCGCAACCGTC |
| TEM_J01749_2068_2239 | CAGCTGCGGTAAAGCTCATCA | CATAGTTAAGCCAGTATACACTC |
| TEM_AF091113_1529_1699 | GTAACAACTTTCATGCTCTCCTAAA | CGGTAACTGATGCCGTATTT |
| TEM_J01749_1634_1783 | CGTTTCCAGACTTTACGAAACAC | ACGTTGTGAGGGTAAACAAC |
| TEM_U36911_6901_7069 | CATCATGTTCATATTTATCAGAGCTC | TAGATTTCATAAAGTCTAACACAC |
| TEM_GU371926_33909_34082 | GTTTCCACATGGTGAACGGTG | AAACCTGTCACTCTGAATGTT |
| VIM_EU118148_2821_2961 | GCTGTAATTATGACGACGCCG | CTCGGTGAGATTCAGAATGC |
| van_DQ018710.1_6481_6652 | GTGTATGTCAGCGATTTGTCCAT | TGTCATATTGTCTTGCCGATT |
| van_DQ018710.1_6764_6926 | GTCCACCTCGCCAACAATCAA | ATATCAACACGGGAAAGACCT |
| van_AY926880.1_3640_3785 | GCGTGATTATCACGTTCGGCA | CTTGCAGATTTAACCGACAC |
| van_FJ545640.1_517_690 | GGCTCGACTTCCTGATGAATACG | TGAAACCGGGCAGAGTATT |
| van_AE017171.1_34715_34859 | CAACGATGTATGTCAACGATTTGT | ATTGCGTAGTCCAATTCGTC |
| van_FJ349556.1_5601_5765 | GGCTCGGCTTCCTGATGAATAC | AGGCATGGTATTGACTTCATT |
| mecA_AM048806.2_1574_1720 | CAGTATTTCACCTTGTCCGTAACC | GTTTACGACTTGTTGCATGC |
| mecA_EF692630.1_239_405 | AATGTTTATATCTTTAACGCCTAAACT | ATGCTTTGGTCTTTCTGCAT |
| mex_AF092566.1_371_520 | CTGGCCCTTGAGGTCGCGG | CGGTCTTCACCTCGACAC |
| mex_AF092566.1_50_193 | GACGTAGATCGGGTCGAGCT | ACGGAAACCTCGGAGAATT |
| mex_CP000438.1_487178_487357 | GGCGTACTGCTGCTTGCTCA | TGACGTCGACGTAGATCG |
| mex_NZ_AAQW01000001.1_461304_461466 | CCTGTTCCTGGGTCGAAGCC | CTTCGGTCACCGCGGA |
| erm_EU047809.1_79_229 | GTTTATAAGTGGGTAAACCGTGAAT | GAAACGAGCTTTAGGTTTGC |
| gyrB_NC_015663_1455472_1455621 | GCCCTTTCAGGACTTTGATACTGG | TGTACGGAGACGGAGTTATCG |
| gyrB_NC_010410_4215_4366 | ACACTGACCGATTCATCCTCGTG | CTTGAAAGTGCGTTAACAACC |
| gyrB_NC_005773_4904_5052 | CGGAAGCCCACCAAGTGAGTAC | CGAAACCAGTTTGTCCTTAGTC |
| gyrB_NC_016514_5343_5487 | ACCAGCTTGTCTTTAGTCTGAGAG | CTTTACGACGGGTCATTTCAC |
| gyrB_NC_016603_2631439_2631616 | CATTGGTTTGTTCTGTTTGAGAGGC | GATTCATCTTCGTGAATTGTGAC |
| gyrB_NC_009436_4366_4524 | GGACTTTGATACTGGAGGAGTCATA | TGTACGGAAACGGAGTTATCG |
| gyrB_NC_009512_4203_4373 | ATGCTGGAGGAGTCGTACGTTT | GTCGCGCACACTAATAGATTC |

TABLE 13
Plasmid regions can be used for identification purposes and can evidence
horizontal gene transfer.

| Probe Coordinates | Gene |
|---|---|
| plasmids_NC_010660_187 | |
| 035_187205 | |
| plasmids_NC_014232_550 | parB1, ETEC1392/75_p1018_014, putative ParB plasmid |
| 1_5677 | stabilisation protein |
| plasmids_NC_011838_178 | pCAR12_p001, putative ABC transporter |
| 818_178996 | subunit, tnpAb, pCAR12_p172, transposase |
| plasmids_FN554767_1301 | EC042_pAA016, site-specific recombinase |
| 7_13190 | |
| plasmids_NC_013655_115 | ECSF_P1-0138, hypothetical protein |
| 365_115542 | |
| plasmids_NC_013951_698 | |
| 99_70067 | |
| plasmids_NC_007635_383 | pCoo017, resD, pCoo052, putative resolvase |
| 95_38566 | |
| plasmids_NC_009787_179 | EcE24377A_C0013, putative methylase |
| 46_18116 | |
| plasmids_NC_006671_562 | near yfcB, O2R_81, YfcB |
| 59_56438 | |
| plasmids_NC_014385_531 | |
| 51_53310 | |
| plasmids_FN649418_5716 | ETEC_p948_0010, IS66-family transposase |
| 9_57339 | |
| plasmids_NC_005011_862 | blaR1, MWP012, bla regulator protein blaRl |
| 0_8785 | |
| plasmids_NC_014843_984 | yfhA, p3521_p111, YfhA |
| 13_98578 | |
| plasmids_NC_008490_516 | |
| 5_5334 | |
| plasmids_NC_015963_147 | Entas_4593, integrase catalytic subunit |
| 516_147686 | |
| plasmids_NC_007365_100 | resD, LH0122, site-specific recombinase |
| 545_100708 | |
| plasmids_NC_009838_104 | qacEdelta1, APECO1_O1R94, quaternary ammonium |
| 163_104332 | compound resistance protein |
| plasmids_NC_010409_397 | pVM01_p034, insertion sequence 2 OrfA protein |
| 68_39935 | |
| plasmids_NC_014233_503 | ETEC1392/75_p557_00068 |
| 37_50492 | |
| plasmids_NC_013362_566 | ECO26_p2-76, conjugal transfer nickase/helicase TraI |
| 51_56805 | |

TABLE 14
Plasmid arms

| Probe Coordinates | Binding region 1 | Binding region 2 |
|---|---|---|
| plasmids_NC_010660_187035_187205 | GCTGTCACCGTCCAGACGCTGTTGGC | TCCGTGCCTTCAAGCGCG |
| plasmids_NC_014232_5501_5677 | GACTCCGCAGAATACGGCACCGTGCGCA | GCGTACAGGCCAGTCAGC |
| plasmids_NC_011838_178818_178996 | GCTGTCCTGGCTGCAAGCCTGG | CCGAACTGCTGATGGACGT |
| plasmids_FN554767_13017_13190 | GACAGCAGACTCACCGGCTGGTTCCGCT | GCAAGATGCTGCTGGCCACACTG |
| plasmids_NC_013655_115365_115542 | GACAGAACAAGTTCCGCTCCGG | CACGGATACGCCGCGCAT |
| plasmids_NC_013951_69899_70067 | GAACGTCTGGCGCTGGTCGCCTGCC | GCACAGGTGCTGACGTGGT |
| plasmids_NC_007635_38395_38566 | AATCCAGGTCCTGACCGTTCTGTCCGT | ACCTCCGTTGAGCTGATGGA |
| plasmids_NC_009787_17946_18116 | GAGGTGGCCAACACCATGTGTGACC | GACGCCGGTATATCGGTATCGAGCT |
| | | GCT |
| plasmids_NC_006671_56259_56438 | GAAGTGCCGGACTTCTGCAGA | GCACGGCCTGATGGAGGCCGC |
| plasmids_NC_014385_53151_53310 | GCTAATCGCATAACAGCTAC | CATCACGTAACTTATTGATGATATT |
| plasmids_FN649418_57169_57339 | GCTGCGGTATTCCACGGTCGGCC | GCAGGAACGCTGCCTGTGGTC |
| plasmids_NC_005011_8620_8785 | GAATCAATTATCTTCTTCATTATTGAT | CTGCGGCTCAACTCAAGCA |
| plasmids_NC_014843_98413_98578 | GTCACACGTCACGCAGTCC | GCATTCATGGCGCTGATGGC |
| plasmids_NC_008490_5165_5334 | GTGTTACTCGGTAGAATGCTCGCAAGG | ACTAGATGACATATCATGTAAGTT |
| plasmids_NC_015963_147516_147686 | CGGAACTGCCTGCTCGTAT | AACGATATAGTCCGTTAT |
| plasmids_NC_007365_100545_100708 | GCTCTCCGACTCCTGGTACGTCAG | GCGCGCATTAATGAAGCAC |
| plasmids_NC_009838_104163_104332 | GATGTTGCGATTACTTCGCCAACTATTG | GCTGTAATTATGACGACGCCG |
| plasmids_NC_010409_39768_39935 | GCAATACCAGGAAGGAAGTCTTACTG | GTCATTGGAGAACAGATGATTGATGT |
| plasmids_NC_014233_50337_50492 | GTATCGCCACAATAACTGCCGGAA | AACGATATAGTCCGTTATG |
| plasmids_NC_013362_56651_56805 | GTGAAGCGCATCCGGTCACC | ATGGCATAGGCCAGGTCAATAT |

TABLE 15
A list of antibiotic resistance genes for which probes can be used to identify,
distinguish and/or sequence
Source Sample ID
CARB
CMY
CTX-M
GES
IMP
KPC
NDM
Other ampC
OXA
PER
SHV
VEB
VIM
ermA
vanA
vanB
mecA
mexA

In some embodiments, the oligonucleic acid probes provided by the invention are molecular inversion probes (MIP). Advantages that the MIP probes described herein offer over PCR include:

1) Multiplexing: there are published studies using 10k+ inversion probes to genotype humans including: http://www.ncbi.nlm.nih.gov/pubmed/17934468 (Porreca et. al.), 55k probes http://www.ncbi.nlm nih gov/pmc/articles/PMC2715272/?tool=pubmed 30k probes http://www.ncbi.nlm.nih.gov/pubmed/19329998 10k probes.

This offers a huge capability to expand panels. First uses might be to capture more rare strains/variants that work poorly with current PCR primers. Later uses might involve genotyping HIV and human loci as well as testing for diseases common in HIV patients—such a test can still be performed in a single tube with minimal per-test increase in reagents cost.

2) Specificity: the probes described herein are less likely to produce off-target products because the two probe arms must bind together. This provides a thermodynamic advantage for on-target binding compared to mis-priming. Furthermore, the exonuclease step will eliminate extension products that occur when only a single probe arm binds.

PCR primers can create long extension products that serve as templates for mis-priming in later rounds. This is particularly a problem when there's lots of background (e.g. human) DNA compared to the target sequence; such as when the exonuclease step didn't remove all of the template and the amplification/barcoding primers misprimed against human DNA. This ends up wasting reads and would have been worse had enrichment for the circularized probes was not being performed. Preventing such reads in a PCR-only system is difficult.

3) Design optimization: the large published datasets provide good training data for a probe picking algorithm. These large datasets can be useful for picking probe sets that will work reliably and with uniform efficiency. Furthermore, we can generate a set of 10k+ probes on a microarray to generate datasets using preferred enzymes. Currently being tested is the entire set of 10k+ probes in a single reaction and then analyzing the read counts to see what made a good probe and what didn't.

Understanding the probe behavior is important for pathogens as it helps to understand the sensitivity and specificity, particularly when considering rare strains or the possibility of previously unknown strains. Pathogenica has thermodynamic models of probe behavior that provide quantitative predictions of how well a probe will work against a target.

4) Simplicity: the probe protocol can be one-tube all the way through, adding reagents until all of the samples are pooled. PCR protocols often require multiple tubes to purify intermediate or final product from the template (e.g., Ampliseq requires 7, PCR+ Nextera likely requires 3+). Also being used are standard reagents (enzymes+oligos) and equipment (thermal cycler).

The following references are incorporated by reference in their entirety: Roberts R R, et al., “Costs attributable to healthcare-acquired infection in hospitalized adults and a comparison of economic methods,” Medical Care, 48(11):1026-1035, November 2010; Scott, R. D., II., “The Direct Medical Costs of Healthcare-Associated Infections in U.S. Hospitals and the Benefits of Prevention,” U.S. Centers for Disease Control and Prevention, March 2009; and Edwards, J. R., et al., National Healthcare Safety Network (NHSN) report: data summary for 2006 through 2008, issued December 2009, American Journal of Infection Control. 37:783-805, December 2009.

It should be understood that for all numerical bounds describing some parameter in this application, such as “about,” “at least,” “less than,” and “more than,” the description also necessarily encompasses any range bounded by the recited values. Accordingly, for example, the description at least 1, 2, 3, 4, or 5 also describes, inter alia, the ranges 1-2, 1-3, 1-4, 1-5, 2-3, 2-4, 2-5, 3-4, 3-5, and 4-5, et cetera.

For all patents, applications, or other reference cited herein, such as non-patent literature and reference sequence information, it should be understood that it is incorporated by reference in its entirety for all purposes as well as for the proposition that is recited. Where any conflict exits between a document incorporated by reference and the present application, this application will control. All information associated with reference gene sequences disclosed in this application, such as GeneIDs, Unigene IDs, or HomoloGene ID, or accession numbers (typically referencing NCBI accession numbers), including, for example, genomic loci, genomic sequences, functional annotations, allelic variants, and reference mRNA (including, e.g., exon boundaries or response elements) and protein sequences (such as conserved domain structures) are hereby incorporated by reference in their entirety.

Headings used in this application are for convenience only and do not affect the interpretation of this application.

Preferred features of each of the aspects provided by the invention are applicable to all of the other aspects of the invention mutatis mutandis and, without limitation, are exemplified by the dependent claims and also encompass combinations and permutations of individual features (e.g., elements, including numerical ranges and exemplary embodiments) of particular embodiments and aspects of the invention including the working examples. For example, particular experimental parameters exemplified in the working examples can be adapted for use in the claimed invention piecemeal without departing from the invention. For example, for material is that are disclosed, while specific reference of each various individual and collective combinations and permutation of these compounds may not be explicitly disclosed, each is specifically contemplated and described herein. Thus, if a class of elements A, B, and C are disclosed as well as a class of elements D, E, and F and an example of a combination of elements, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, elements of a composition of matter and steps of method of making or using the compositions.

The forgoing aspects of the invention, as recognized by the person having ordinary skill in the art following the teachings of the specification, can be claimed in any combination or permutation to the extent that they are novel and non-obvious over the prior art—thus to the extent an element is described in one or more references known to the person having ordinary skill in the art, they may be excluded from the claimed invention by, inter alia, a negative proviso or disclaimer of the feature or combination of features.

While this invention has been particularly shown and described with references to example embodiments thereof, it will be understood by those skilled in the art that various changes in form and details may be made therein without departing from the scope of the invention encompassed by the appended claims.

### Examples

Procedure:
1) Remove the DxSeq Kit from the −20° C. freezer.
2) Remove one Reagent Set Pack from the DxSeq Kit, and place the tubes on ice.
3) Remove two blue FrameStrips and matching strip caps from the kit. [The Break-A-Way Plate with primers is not needed at this point in the protocol.]
4) Label the FrameStrips and the strip caps #1 and #2 with a permanent marker. [Both the FrameStrips and strip caps should be labeled to avoid cross-contamination during subsequent handling steps.]
5) Return the kit to the −20° C. freezer for later use.
6) After the components have thawed, pulse-spin any droplets from the cap or sidewalls to the bottom of the tubes using a microcentrifuge.
7) Using barrier pipette tips, prepare 75 μL Hybridization Master Mix for 12 samples and 2 controls, as follows:
   a. 22.5 μL 10× Buffer A
   b. 15 μL MIP Probe mixture
   c. 37.5 μl of nuclease-free water
8) Using barrier pipette tips, pipette 5 μL of Hybridization Master Mix into wells A-G of two blue FrameStrip PCR 8-strips (n=14 wells). [Do not pipette Hybridization Master Mix into wells H: these are reserved for negative controls.]
9) Being very careful not to cross-contaminate the wells, add 10 μL of each DNA sample to the A-F wells of the two FrameStrips (n=12 wells). [Do not pipette your DNA samples into the G & H wells: these four wells are reserved for control reactions.]
10) Add 10 μL of nuclease-free water to the G wells (n=2 wells). These will serve as the “no target DNA” negative controls.
11) Add 13.5 of nuclease-free water and 1.5 of 10× Buffer A to the H wells (n=2 wells). These will serve as the “no probe” negative controls.
12) Seal the two FrameStrips with the flat strip caps.
13) Vortex the sealed FrameStrips briefly to mix the contents; and then pulse-spin down the contents in a microcentrifuge with a rotor that accommodates 8-well strip PCR tubes.
14) Enter the following program into a thermocycler, using the heated lid option.

| | | |
|---|---|---|
| a. | 94° C., 10 min | Hybridization |
| b. | Ramp to 60° C., 0.1° C./sec | |
| c. | 60° C., 10 min | |
| d. | 60° C. hold | |
| e. | 60° C., 10 min | Extension |
| f. | 15° C. hold | |
| g. | 94° C., 2 min | Exonuclease cleanup |
| h. | 37° C. hold | |
| i. | 37° C., 30 min | |
| j. | 94° C., 15 min | |
| k. | 4° C. hold | |

15) Place the sealed FrameStrips in the thermocycler; and begin the hybridization portion of the MIP Program.
16) While the hybridization is underway, prepare the Polymerase/Ligase Master Mix on ice:
   a. 5 μL Polymerase
   b. 5 μL 10× Buffer A
   c. 1 μL Ligase
   d. 1.25 μL dNTPs
   e. 37.75 μL nuclease-free water
17) When the hybridization reaction reaches the 60° C. hold step (approximately 26 minutes into the program), add 2 μL of the Polymerase/Ligase Master Mix to every well (n=16 wells).
18) Reseal the FrameStrips with the same strip caps as before and mix. [Special care needs to be taken not to cross-contaminate the samples.]
19) Advance the thermocycler to the next step in the MIP Program (60° C. for 10 min).
20) When the thermocycler reaches the 15° C. hold step, advance the thermocycler to the next step (94° C. for 2 min) in the MIP Program.
21) When the thermocycler reaches the 37° C. hold step, immediately add 1 μL of Exonuclease to each sample.
22) Reseal the FrameStrips with the same strip caps as before and mix. [Special care needs to be taken not to cross-contaminate the samples.]
23) Advance the thermocycler to the next step (37° C. for 30 min) in the MIP Program.
24) While the reactions are incubating at 37° C., prepare the amplification mix:
   a. (components of the PCR reaction)
25) Remove the Purple Break-A-Way 96 Well Plate containing PCR primers from the −20° C. freezer. Break off three columns from the left side of the plate.
26) Return the unused portion of the Break-A-Way 96 Well Plate to the freezer (before the primers thaw).
27) When the thermocycler reaches the 4° C. hold, add 2.5 μL of tube-specific barcoding primer and 29.5 μL of amplification mix.
28) Begin the PCR Amplification Program on the thermocycler:
   a. 94° C., 3 min
   b. 30 cycles of:
      i) 94° C., 15 sec
      ii) 60° C., 15 sec
      iii) 72° C., 30 sec
   c. 72° C., 4 min
   d. 4° C. hold
29) Purify the PCR amplicons using AMPure beads (Beckman Coulter).
30) Proceed to the IonTorrent Template preparation workflow.

Pathogenica Software installed on the Ion Torrent PGM reports the results.

1. A probe set for detecting pathogenic organisms or strains in a sample, comprising at least 2, 3, 4, 5, 6, 7, 8, 9, 10, 20, 40, 50, 100, 150, 200, 250, 300, 350, 400, 450, 500, 550, 600, or more oligonucleic acid molecules that, when implemented in an assay, detect and distinguish at least 1, 2, 3, 4, 5, 6, 7, 8, 9, 10, or more different strains, variants, or subtypes of at least 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, or more pathogenic organisms selected from virus, bacterium, fungi, and combinations thereof, wherein each oligonucleic acid molecule in the set comprises a first sequence that specifically hybridizes to a target sequence adjacent to a region of interest in at least one of the pathogenic organisms.

2. The probe set of claim 1, wherein the set comprises oligonucleic acid molecules further comprising a second sequence that specifically hybridizes to a second target sequence adjacent to the region of interest, wherein the oligonucleic acid molecules are capable of circularizing capture of the region of interest, and further wherein the first and second target sequences are separated by at least one nucleotide.

3. The probe set of claim 1, wherein the set of oligonucleic acid molecules comprises pairs of oligonucleic acid molecules suitable for geometric amplification of the region of interest by polymerase chain reaction.

4. The probe set of claim 1, wherein the pathogenic organisms include any three or more of Staphylococcus aureus, Staphylococcus epidermis, Staphylococcus saprophyticus, Acinetobacter baumanii, Clostridium difficile, Escherichia coli, Enterobacter (aerogenes, cloacae, asburiae, and combinations thereof), Enterococcus (faecium and/or faecalis), Klebsiella pneumoniae, Proteus mirabilis, Candida albicans, and Pseudomonas aeruginosa; or subtypes or strains thereof.

5.-7. (canceled)

8. The probe set of claim 1, wherein the probe set comprises:
a) oligonucleic acid molecules capable of i) amplifying, geometrically by polymerase chain reaction or ii) circularizing capture of, 1, 2, 3, 4, 5, 10, 15, 16, or all 17, of the regions of interest provided in column 1 of Table 3, or substantially similar sequences;
b) oligonucleic acid molecules capable of i) amplifying, geometrically by polymerase chain reaction or ii) circularizing capture of, 1, 2, 3, 4, 5, 10, 15, 20, 30, 50, 100, or all 134, of the regions of interest provided in column 1 of Table 5, or substantially similar sequences;
c) oligonucleic acid molecules capable of i) amplifying, geometrically by polymerase chain reaction or ii) circularizing capture of, 1, 2, 3, 4, 5, 10, or all 13, of the regions of interest provided in column 1 of Table 7, or substantially similar sequences;
d) oligonucleic acid molecules capable amplifying, geometrically by polymerase chain reaction, or circularizing capture of, 1, 2, 3, 4, 5, 10, 20, 40, 60, 80, or all 85, of the regions of interest provided in column 1 of Table 9, or substantially similar sequences;
e) oligonucleic acid molecules capable of i) amplifying, geometrically by polymerase chain reaction or ii) circularizing capture of, 1, 2, 3, 4, 5, 10, 20, 25, or all 29 of the regions of interest provided in column 1 of Table 11, or substantially similar sequences;
f) oligonucleic acid molecules capable of i) amplifying, geometrically by polymerase chain reaction or ii) circularizing capture of, 1, 2, 3, 4, 5, 10, 15, or all 20, of the regions of interest provided in column 1 of Table 13, or substantially similar sequences; or
g) a combination of 1, 2, 3, 4, 5, or all 6 of a), b), c), d), e) and f).

9. The probe set of claim 8, wherein the substantially similar sequences are 60, 65, 70, 75, 80, 85, 90, 95, 96, 97, 98, 99, 99.5, or 100% identical the sequence of the regions of interest indicated by the probe name in column 1 of Table 3, 5, 7, 9, 11, or 13; or alternatively, or additionally, wherein the substantially similar sequences have endpoints within 100, 90, 80, 70, 60, 50, 40, 35, 30, 25, 20, 19, 18, 17, 16, 15, 14, 13, 12, 11, 10, 9, 8, 7, 6, 5, 4, 3, 2, 1, or 0 nucleotides upstream or downstream of either of the endpoints of the regions of interest in column 1 of Table 3, 5, 7, 9, 11, or 13.

10. The probe set of claim 1, wherein the probe set comprises:
a) oligonucleic acid molecules comprising 1, 2, 4, 6, 8, 10, 15, 20, 25, 30, or all 34 of the sequences, or reverse complements thereof, provided in the second or third column of Table 4;
b) oligonucleic acid molecules comprising 1, 2, 4, 6, 8, 10, 20, 50, 100, 150, 200, 250, or all 268 of the sequences, or reverse complements thereof, provided in the second or third column of Table 6;
c) oligonucleic acid molecules comprising 1, 2, 4, 6, 8, 10, 15, 20, 25, or all 26 of the sequences, or reverse complements thereof, provided in the second or third column of Table 8;
d) oligonucleic acid molecules comprising 1, 2, 4, 6, 8, 10, 20, 50, 100, 150, or all 170 of the sequences, or reverse complements thereof, provided in the second or third column of Table 10;
e) oligonucleic acid molecules comprising 1, 2, 4, 6, 8, 10, 20, 30, 40, 50, or all 56 of the sequences, or reverse complements thereof, provided in the second or third column of Table 12;
f) oligonucleic acid molecules comprising 1, 2, 4, 6, 8, 10, 20, 30, or all 40 of the sequences, or reverse complements thereof, provided in the second or third column of Table 14; or
g) a combination of 1, 2, 3, 4, 5, or all 6 of a), b), c), d), e), and f).

11. The probe set of claim 1, wherein the probe set detects resistance genes of any one of the CARB, CMY, CTX-M, GES, IMP, KPC, NDM, ampC, OXA, PER, SHV, VEB, VIM, ermA, vanA, canB, mecA, mexA family of genes, or any combination of 1, 2, 3, 4, 5, 6, 7, 8, 9, 10, 11, 12, 13, 14, 15, 16, 17, or all 18 of these families of genes.

12. A probe set comprising:
a) oligonucleic acid molecules comprising 1, 2, 4, 6, 8, 10, 15, 20, 25, 30, or all 34 of the sequences, or reverse complements thereof, provided in the second or third column of Table 4;
b) oligonucleic acid molecules comprising 1, 2, 4, 6, 8, 10, 20, 50, 100, 150, 200, 250, or all 268 of the sequences, or reverse complements thereof, provided in the second or third column of Table 6;
c) oligonucleic acid molecules comprising 1, 2, 4, 6, 8, 10, 15, 20, 25, or all 26 of the sequences, or reverse complements thereof, provided in the second or third column of Table 8;
d) oligonucleic acid molecules comprising 1, 2, 4, 6, 8, 10, 20, 50, 100, 150, or all 170 of the sequences, or reverse complements thereof, provided in the second or third column of Table 10;
e) oligonucleic acid molecules comprising 1, 2, 4, 6, 8, 10, 20, 30, 40, 50, or all 56 of the sequences, or reverse complements thereof, provided in the second or third column of Table 12; and
f) oligonucleic acid molecules comprising 1, 2, 4, 6, 8, 10, 20, 30, or all 40 of the sequences, or reverse complements thereof, provided in the second or third column of Table 14.

13. A probe set comprising oligonucleic acid molecules comprising 10, 20, 30, 40, 50, 60, 70, 80, 90, 95, 99, or 100% of the sequences provided in the second column of Table 1.

14.-19. (canceled)

20. A method of detecting one or more organisms, comprising contacting a sample with the probe set of claim 1 to capture one or more regions of interest of the one or more organisms, wherein capturing a region of interest for the one or more organisms indicates the presence of the one or more organisms in the sample.

21. The method of claim 20, wherein the one or more organisms comprise a pathogen.

22. The method of claim 20, wherein the sample is a nucleic acid sample isolated from a biological sample obtained from a human subject, wherein the biological sample is obtained from a surgical site, catheter, ventilator, intravenous needle, respiratory tractcatheter, medical device, blood, blood culture, urine, stool, fomite, wound, sputum, pure bacterial culture, mixed bacterial culture, bacterial colony, or any combination thereof.

23. (canceled)

24. The method of claim 22, further comprising obtaining a genotype for the human subject.

25.-29. (canceled)

30. The method of claim 20, wherein the capture reaction is performed in less than three hours.

31. The method of claim 20, wherein massive parallel sequencing is performed to sequence 50,000 to 900 hundred million reads from amplified DNA clones.

32. The method of claim 20, wherein the reads are between about 50-2000 nucleotides in length.

33. (canceled)

34. The method of claim 20, comprising simultaneously detecting both viruses and fungi.

35. The method of claim 20, wherein the one or more regions of interest are predicted, in a single bacterial reference, to differ by >1 SNP from >2 other reference genomes, thereby enabling discrimination of this one genome from >2 others for the same species.

36. The method of claim 20, wherein one or more pathogens are detected.

37.-38. (canceled)

39. A system comprising a non-transient computer readable medium containing instructions that, when executed by a processor, cause the processor to perform steps comprising:
comparing one or more captured regions of interest captured by the method of claim 20 to a reference database to identify the one or more organisms present in the sample; and, optionally
displaying an identity of the one or more organisms present in the sample and/or a therapeutic recommendation based on the results of the comparison.

40.-45. (canceled)
